(19) United States
(12) Patent Application Publication
(10) Pub. No.: US 2025/0313567 A1
(43) Pub. Date: Oct. 9, 2025

(54) IMIDAZOPYRIMIDINES AND TRIAZOLOPYRIMIDINES AS A2A / A2B INHIBITORS

(71) Applicant: Incyte Corporation, Wilmington, DE (US)

(72) Inventors: Xiaozhao Wang, Moorestown, NJ (US); Pei Gan, Claymont, DE (US); Heeoon Han, Elkins Park, PA (US); Taisheng Huang, Wilmington, DE (US); Matthew S. McCammant, Newark, DE (US); Chao Qi, Newark, DE (US); Ding-Quan Qian, Newark, DE (US); Liangxing Wu, Wilmington, DE (US); Wenqing Yao, Chadds Ford, PA (US); Zhiyong Yu, Wilmington, DE (US); Fenglei Zhang, Berwyn, PA (US); Le Zhao, Wilmington, DE (US); Chunhong He, Boothwyn, PA (US)

(21) Appl. No.: 19/088,394

(22) Filed: Mar. 24, 2025

Related U.S. Application Data
(63) Continuation of application No. 18/300,349, filed on Apr. 13, 2023.
Application No. 18/300,349 is a division of application No. 17/077,713, filed on Oct. 22, 2020, now Pat. No. 11,673,894.
Application No. 17/077,713 is a continuation of application No. 16/285,787, filed on Feb. 26, 2019, now abandoned.
(60) Provisional application No. 62/635,926, filed on Feb. 27, 2018.
(60) Provisional application No. 62/718,216, filed on Aug. 13, 2018.
(60) Provisional application No. 62/721,312, filed on Aug. 22, 2018.
(60) Provisional application No. 62/793,015, filed on Jan. 16, 2019.

(51) Int. Cl. C07D 487/04; A61P 9/00; A61P 25/00; A61P 29/00; A61P 35/00; C07D 491/056; C07D 519/00

(52) U.S. Cl. CPC C07D 487/04; A61P 9/00; A61P 25/00; A61P 29/00; A61P 35/00; C07D 491/056; C07D 519/00

(57) ABSTRACT

This application relates to compounds of Formula (I):

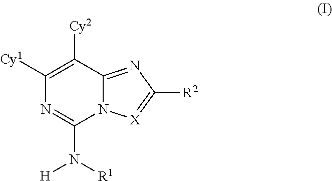

or pharmaceutically acceptable salts or stereoisomers thereof, which modulate the activity of adenosine receptors, such as subtypes A2A and A2B receptors, and are useful in the treatment of diseases related to the activity of adenosine receptors including, for example, cancer, inflammatory diseases, cardiovascular diseases, and neurodegenerative diseases.

### TECHNICAL FIELD

The present invention provides imidazopyrimidine and triazolopyrimidine compounds that modulate the activity of adenosine receptors, such as subtypes A2A and A2B, and are useful in the treatment of diseases related to the activity of adenosine receptors including, for example, cancer, inflammatory diseases, cardiovascular diseases, and neurodegenerative diseases.

### BACKGROUND

Adenosine is an extracellular signaling molecule that can modulate immune responses through many immune cell types. Adenosine was first recognized as a physiologic regulator of coronary vascular tone by Drury and Szent-Györgyu (Sachdeva, S. and Gupta, M. Saudi Pharmaceutical Journal, 2013, 21, 245-253), however it was not until 1970 that Sattin and Rall showed that adenosine regulates cell function via occupancy of specific receptors on the cell surface (Sattin, A., and Rall, T. W., 1970. Mol. Pharmacol. 6, 13-23; Hasko', G., at al., 2007, Pharmacol. Ther. 113, 264-275).

Adenosine plays a vital role in various other physiological functions. It is involved in the synthesis of nucleic acids, when linked to three phosphate groups; it forms ATP, the integral component of the cellular energy system. Adenosine can be generated by the enzymatic breakdown of extracellular ATP, or can be also released from injured neurons and glial cells by passing the damaged plasma membrane (Tautenhahn, M. et al. Neuropharmacology, 2012, 62, 1756-1766). Adenosine produces various pharmacological effects, both in periphery and in the central nervous system, through an action on specific receptors localized on cell membranes (Matsumoto, T. et al. Pharmacol. Res., 2012, 65, 81-90). Alternative pathways for extracellular adenosine generation have been described. These pathways include the production of adenosine from nicotinamide dinucleotide (NAD) instead of ATP by the concerted action of CD38, CD203a and CD73. CD73-independent production of adenosine can also occur by other phosphates such as alkaline phosphatase or prostate-specific phosphatase.

There are four known subtypes of adenosine receptor in humans including A1, A2A, A2B, and A3 receptors. A1 and A2A are high affinity receptors, whereas A2B and A3 are low affinity receptors. Adenosine and its agonists can act via one or more of these receptors and can modulate the activity of adenylate cyclase, the enzyme responsible for increasing cyclic AMP (cAMP). The different receptors have differential stimulatory and inhibitory effects on this enzyme. Increased intracellular concentrations of cAMP can suppress the activity of immune and inflammatory cells (Livingston, M. et al., Inflamm. Res., 2004, 53, 171-178).

The A2A adenosine receptor can signal in the periphery and the CNS, with agonists explored as anti-inflammatory drugs and antagonists explored for neurodegenerative diseases (Carlsson, J. et al., J. Med. Chem., 2010, 53, 3748-3755). In most cell types the A2A subtype inhibits intracellular calcium levels whereas the A2B potentiates them. The A2A receptor generally appears to inhibit inflammatory response from immune cells (Borrmann, T. et al., J. Med. Chem., 2009, 52(13), 3994-4006).

A2B receptors are highly expressed in the gastrointestinal tract, bladder, lung and on mast cells (Antonioli, L. et al., Nature Reviews Cancer, 2013, 13, 842-857). The A2B receptor, although structurally closely related to the A2A receptor and able to activate adenylate cyclase is functionally different. It has been postulated that this subtype may utilize signal transduction systems other than adenylate cyclase (Livingston, M. et al., Inflamm. Res., 2004, 53, 171-178). Among all the adenosine receptors, the A2B adenosine receptor is a low affinity receptor that is thought to remain silent under physiological conditions and to be activated in consequence of increased extracellular adenosine levels (Ryzhov, S. et al. Neoplasia, 2008, 10, 987-995). Activation of A2B adenosine receptor can stimulate adenylate cyclase and phospholipase C through activation of Gs and Gq proteins, respectively. Coupling to mitogen activated protein kinases has also been described (Borrmann, T. et al., J. Med. Chem., 2009, 52(13), 3994-4006).

In the immune system, engagement of adenosine signaling can be a critical regulatory mechanism that protects tissues against excessive immune reactions. Adenosine can negatively modulate immune responses through many immune cell types, including T-cells, natural-killer cells, macrophages, dendritic cells, mast cells and myeloid-derived suppressor cells (Allard, B. et al. Current Opinion in Pharmacology, 2016, 29, 7-16).

In tumors, this pathway is hijacked by tumor micro-environments and sabotages the antitumor capacity of immune system, promoting cancer progression. In the tumor micro-environment, adenosine was mainly generated from extracellular ATP by CD39 and CD73. Multiple cell types can generate adenosine by expressing CD39 and CD73. This is the case for tumor cells, T-effector cells, T-regulatory cells, tumor associated macrophages, myeloid derived suppressive cells (MDSCs), endothelial cells, cancer-associated fibroblast (CAFs) and mesenchymal stromal/stem cells (MSCs). Hypoxia, inflammation and other immune-suppressive signaling in tumor micro-environment can induce expression of CD39, CD73 and subsequent adenosine production. As a result, adenosine level in solid tumors is unusually high compared to normal physiological conditions.

A2A are mostly expressed on lymphoid-derived cells, including T-effector cells, T regulatory cells and nature killing cells. Blocking A2A receptor can prevent downstream immunosuppressive signals that temporarily inactivate T cells. A2B receptors are mainly expressed on monocyte-derived cells including dendritic cells, tumor-associated macrophages, myeloid derived suppressive cells (MDSCs), and mesenchymal stromal/stem cells (MSCs). Blocking A2B receptor in preclinical models can suppress tumor growth, block metastasis, and increase the presentation of tumor antigens.

In tens of safety profile of ADORA2A/ADORA2B (A2A/A2B) blockage, the A2A and A2B receptor knockout mice are all viable, showing no growth abnormalities and are fertile (Allard, B. et al. Current Opinion in Pharmacology, 2016, 29, 7-16). A2A KO mice displayed increased levels of pro-inflammatory cytokines only upon challenge with LPS and no evidence of inflammation at baseline (Antonioli, L. et al., Nature Reviews Cancer, 2013, 13, 842-857). A2B KO mice exhibited normal platelet, red blood, and white cell counts but increased inflammation at baseline (TNF-alpha, IL-6) in naive A2B KO mice (Antonioli, L. et al., Nature Reviews Cancer, 2013, 13, 842-857). Exaggerated production of TNF-alpha and IL-6 was detected following LPS treatment. A2B KO mice also exhibited increased vascular adhesion molecules that mediate inflammation as well leukocyte adhesion/rolling; enhanced mast-cell activation; increased sensitivity to IgE-mediated anaphylaxis and increased vascular leakage and neutrophil influx under hypoxia (Antonioli, L. et al., Nature Reviews Cancer, 2013, 13, 842-857).

In summary, there is a need to develop new adenosine receptor selective ligands, such as for subtypes A2A and A2B, for the treatment of diseases such as cancer, inflammatory diseases, cardiovascular diseases and neurodegenerative diseases. This application is directed to this need and others.

### SUMMARY

The present invention relates to, inter alia, compounds of Formula (I):

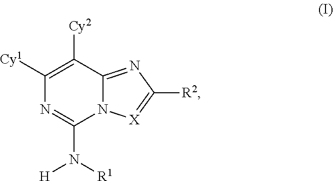

or pharmaceutically acceptable salts thereof, wherein constituent members are defined herein.

The present invention further provides pharmaceutical compositions comprising a compound of Formula (I), or a pharmaceutically acceptable salt thereof, and a pharmaceutically acceptable carrier.

The present invention further provides methods of inhibiting an activity of an adenosine receptor, comprising contacting the receptor with a compound of Formula (I), or a pharmaceutically acceptable salt thereof.

The present invention further provides methods of treating a disease or a disorder associated with abnormal expression of adenosine receptors, comprising administering to said patient a therapeutically effective amount of a compound of Formula (I), or a pharmaceutically acceptable salt thereof.

The present invention further provides a compound of Formula (I), or a pharmaceutically acceptable salt thereof, for use in any of the methods described herein.

The present invention further provides use of a compound of Formula (I), or a pharmaceutically acceptable salt thereof, for the preparation of a medicament for use in any of the methods described herein.

### DETAILED DESCRIPTION

### Compounds

The present invention relates to, inter alia, compounds of Formula (I):

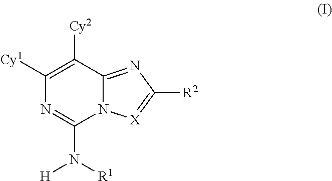

or pharmaceutically acceptable salts thereof; wherein:
X is N or CR³;
R¹ is selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a1}, C(O)R^{b1}, C(O)NR^{c1}R^{d1}, C(O)OR^{a1}, C(═NR^{e1})R^{b1}, C(═NR^{e1})NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R¹ are each optionally substituted with 1, 2, 3, 4, 5, 6, 7, or 8 independently selected R^{B} substituents;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{e2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{e2}C(O)OR^{a2}, NR^{c2}C(O)NR^{c2}R^{d2}, C(═NR^{e2})R^{b2}, C(═NR^{e2})NR^{e2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
R³ is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3} NR^{c3}C(O)OR^{a3}, NR^{c3}C(O)NR^{c3}R^{d3}C(═NR^{e3})R^{b3}, C(═NR^{b3})NR^{c3}R^{d3}, NR^{e3}C(═NR^{e3})NR^{c3}R^{d3}, NR^{c3}C(═NR^{b3})R^{b3}, NR^{c3}S(O)NR^{c3}R^{d3}, NR^{c3}S(O)R^{b3}NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3} S(O)₂R^{b3}, S(O)₂NR^{c3}R^{d3} OS(O)(═NR^{b3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R³ are each optionally substituted with 1, 2, 3, 4, 5, 6, 7, or 8 independently selected R^{D} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} substituents;
provided that when X is N and Cy¹ is 4-14 membered heterocycloalkyl, then the 4-14 membered heterocycloalkyl of Cy¹ is other than unsubstituted morpholinyl;
provided that Cy¹ is not pyridin-4-yl optionally substituted with 1, 2, 3, or 4 independently selected R^{E} substituents;
provided that Cy¹ is not pyrimidin-4-yl optionally substituted with 1, 2, or 3, independently selected R^{E} substituents;
provided that Cy¹ is not quinolin-4-yl optionally substituted with 1, 2, 3, 4, 5, or 6 independently selected R^{E} substituents;
Cy² is C₆₋₁₄ aryl, C₃₋₁₄cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a1}, R^{c1}, R^{d1}, R^{a2}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a1}, R^{c1}, R^{d1}, R^{a2}, R^{c2}, R^{d2}, R^{a3}, R^{c3}, and R^{d3} are each optionally substituted with 1, 2, 3, 4, 5, 6, 7, or 8 independently selected R^{G} substituents;
or, any R^{c1} and R^{d1} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c3} and R^{d3} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b1}, R^{b2}, and R^{b3} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b1}, R^{b2}, and R^{b3} are each optionally substituted with 1, 2, 3, 4, 5, 6, 7, or 8 independently selected R^{G} substituents;
each R^{e1}, R^{e2}, and R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2}, R^{g2}, R^{f3}, and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2}, Rⁱ², R^{h3}, and Rⁱ³ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2}, R^{k2}, R^{j3}, and R^{k3} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{B}, R^{C}, R^{D}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4} SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{B}, R^{C}, R^{D}, R^{E}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR⁵, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{H} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy; or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{e6}R^{d6}, C(═NR^{e6})R^{b6}, C(═NR^{e6})NR^{c6}R^{d6}, NR^{e6}C(═NR^{e6})NR^{c6}R^{d6}, NR^{e6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments,
X is N or CR³;
R¹ is selected from H, C₁₋₆ alkyl, C₂₋₆ alkenyl, C₂₋₆ alkynyl, and C₁₋₆ haloalkyl;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{c2})R^{b2}, C(═NR^{c2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
R³ is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3} NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{b3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3} S(O)₂R^{b3}, S(O)₂NR^{c3}R^{d3} OS(O)(═NR^{e3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R³ are each optionally substituted with 1, 2, 3, 4, 5, 6, 7, or 8 independently selected R^{D} substituents;
Cy¹ is C₆₋₁₄ aryl, C₃₋₁₄cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} substituents;
provided that when X is N and Cy¹ is 4-14 membered heterocycloalkyl, then the 4-14 membered heterocycloalkyl of Cy¹ is other than unsubstituted morpholinyl;
Cy² is C₆₋₁₄ aryl, C₃₋₁₄cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a2}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a2}, R^{c2}, R^{d2}, R^{a3}, R^{c3}, and R^{d3} are each optionally substituted with 1, 2, 3, 4, 5, 6, 7, or 8 independently selected R^{G} substituents; or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c3} and R^{d3} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b2}, and R^{b3} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b2} and R^{b3} are each optionally substituted with 1, 2, 3, 4, 5, 6, 7, or 8 independently selected R^{G} substituents;
each R^{e2} and R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2}, R^{g2}, R^{f3}, and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2}, Rⁱ², R^{h3}, and Rⁱ³ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2}, Rⁱ², R^{j3}, and R^{k3} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{C}, R^{D}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR⁴, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{C}, R^{D}, R^{E}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR⁵, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{e5}R^{d6}, C(═NR^{e6})R^{b6}, C(═NR^{e6})NR^{c6}R^{d6}, NR^{e6}C(═NR^{e6})NR^{c6}R^{d6}, NR^{e6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments,
X is CR³;
R¹ is selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a1}, C(O)R^{b1}, C(O)NR^{c1}R^{d1}, C(O)OR^{a1}, C(═NR^{e1})R^{b1}, C(═NR^{e1})NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R¹ are each optionally substituted with 1, 2, 3, 4, 5, 6, 7, or 8 independently selected R^{B} substituents;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{e2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
R³ is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{b3})NR^{c3}R^{d3}, NR^{c3}C(═NR^{e3})NR^{c3}R^{d3}, NR^{c3}C(═NR^{b3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3} S(O)₂R^{b3}, S(O)₂NR^{c3}R^{d3} OS(O)(═NR^{e3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R³ are each optionally substituted with 1, 2, 3, 4, 5, 6, 7, or 8 independently selected R^{D} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} substituents;
provided that Cy¹ is not pyridin-4-yl optionally substituted with 1, 2, 3, or 4 independently selected R^{E} substituents;
provided that Cy¹ is not pyrimidin-4-yl optionally substituted with 1, 2, or 3, independently selected R^{E} substituents;
provided that Cy¹ is not quinoline-4-yl optionally substituted with 1, 2, 3, 4, 5, or 6 independently selected R^{E} substituents;
Cy² is C₆₋₁₄ aryl, C₃₋₁₄cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a1}, R^{c1}, R^{d1}, R^{a2}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a1}, R^{c1}, R^{d1}, R^{a2}, R^{c2}, R^{d2}, R^{a3}, R^{c3}, and R^{d3} are each optionally substituted with 1, 2, 3, 4, 5, 6, 7, or 8 independently selected R^{G} substituents;
or, any R^{c1} and R^{d1} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c3} and R^{d3} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b1}, R^{b2}, and R^{b3} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b1}, R^{b2}, and R^{b3} are each optionally substituted with 1, 2, 3, 4, 5, 6, 7, or 8 independently selected R^{G} substituents;
each R^{e1}, R^{e2}, and R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2}, R^{g2}, R^{f3}, and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2}, Rⁱ², R^{h3}, and Rⁱ³ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2}, Rⁱ², R^{j3}, and R^{k3} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{B}, R^{C}, R^{D}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4} SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃-14 cycloalkyl-C₁₋₆ alkyl-, (5-14 membered heteroaryl)-C₁₋₆ alkyl-, and (4-14 membered heterocycloalkyl)-C₁₋₆ alkyl- of R^{B}, R^{C}, R^{D}, R^{E}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy; or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy; or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{e7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments,
X is CR³;
R¹ is selected from H, C₁₋₆ alkyl, C₂₋₆ alkenyl, C₂₋₆ alkynyl, and C₁₋₆ haloalkyl;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{c2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
R³ is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3} NR^{c3}C(O)OR^{a3}, NR^{c3}C(O)NR^{c3}R^{d3}, C(═NR^{e3})R^{b3}, C(═NR^{b3})NR^{c3}R^{d3}, NR^{c3}C(═NR^{e3})NR^{c3}R^{d3}, NR^{c3}C(═NR^{b3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3} S(O)₂R^{b3}, S(O)₂NR^{c3}R^{d3} OS(O)(═NR^{b3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R³ are each optionally substituted with 1, 2, 3, 4, 5, 6, 7, or 8 independently selected R^{D} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} or R^{M} substituents;
Cy² is C₆₋₁₄ aryl, C₃₋₁₄cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a2}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a2}, R^{c2}, R^{d2}, R^{a3}, R^{c3} and R^{d3} are each optionally substituted with 1, 2, 3, 4, 5, 6, 7, or 8 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c3} and R^{d3} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b2} and R^{b3} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b2} and R^{b3} are each optionally substituted with 1, 2, 3, 4, 5, 6, 7, or 8 independently selected R^{G} substituents;
each R^{e2} and R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2}, R^{g2}, R^{f3}, and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2}, Rⁱ², R^{h3}, and Rⁱ³ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2}, Rⁱ², R^{h3}, and Rⁱ³ is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{C}, R^{D}, R^{E}, R^{M}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4} SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{C}, R^{D}, R^{E}, R^{M}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4} are each optionally substituted with 1, 2, 3, 4, 5, 6, 7, or 8 independently selected R^{E} substituents;
or, any R^{c4} and R^{d4} attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R¹ is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{e7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments:
X is N;
R¹ is selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a1}, C(O)R^{b1}, C(O)NR^{c1}R^{d1}, C(O)OR^{a1}, C(═NR^{e1})R^{b1}, C(═NR^{e1})NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R¹ are each optionally substituted with 1, 2, 3, 4, 5, 6, 7, or 8 independently selected R^{B} substituents;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{b2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} substituents;
provided that when Cy¹ is 4-14 membered heterocycloalkyl, then the 4-14 membered heterocycloalkyl of Cy¹ is other than unsubstituted morpholinyl;
Cy² is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a1}, R^{c1}, R^{d1}, R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a1}, R^{c1}, R^{d1}, R^{a2}, R^{c2}, and R^{d2} are each optionally substituted with 1, 2, 3, 4, 5, 6, 7, or 8 independently selected R^{G} substituents;
or, any R^{c1} and R^{d1} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b1} and R^{b2} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b1}, R^{b2}, and R^{b3} are each optionally substituted with 1, 2, 3, 4, 5, 6, 7, or 8 independently selected R^{G} substituents;
each R^{e1} and R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2} and R^{g2} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{B}, R^{C}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR⁴, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{c4})R^{b4}, C(═NR^{c4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃-14 cycloalkyl-C₁₋₆ alkyl-, (5-14 membered heteroaryl)-C₁₋₆ alkyl-, and (4-14 membered heterocycloalkyl)-C₁₋₆ alkyl- of R^{B}, R^{C}, R^{E}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{H} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and wherein any heteroaryl group of any of the above-recited substituents optionally comprises an N-oxide on any ring-forming nitrogen.

In some embodiments:
X is N;
R¹ is selected from H, C₁₋₆ alkyl, C₂₋₆ alkenyl, C₂₋₆ alkynyl, and C₁₋₆ haloalkyl;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{b2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} or R^{M} substituents;
Cy² is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a2}, R^{c2}, and R^{d2} are each optionally substituted with 1, 2, 3, 4, 5, 6, 7, or 8 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b2} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b2} are each optionally substituted with 1, 2, 3, 4, 5, 6, 7, or 8 independently selected R^{G} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2} and R^{g2} is independently selected from H, C₁₋₆ alkyl, C₁₋₆ haloalkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{C}, R^{E}, R^{M}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR⁴, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{C}, R^{E}, R^{M}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{b5})NR^{c5}R^{d5}, NR^{c5}C(═NR^{c5})NR^{c5}R^{d5}, NR_{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments,
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7};
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
or any R^{c7} and R^{d7} attached to the same N atom, together with the N atom to which they are attached, form a 5- or 6-membered heteroaryl or a 4-14-membered heterocycloalkyl group; and
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-.

In some embodiments,
each R¹ is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6};
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
or any R^{c6} and R^{d6} attached to the same N atom, together with the N atom to which they are attached, form a 5- or 6-membered heteroaryl or a 4-14-membered heterocycloalkyl group; and
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-.

In some embodiments, each R^{H} is independently selected from D, halo, oxo, C₁₋₆ alkyl, C₁₋₆ haloalkyl, C₂₋₆ alkenyl, C₂₋₆ alkynyl, CN, C₁₋₆ alkoxy, C₁₋₆ haloalkoxy, amino, C₁₋₆ alkylamino, di-C₁₋₆ alkylamino, C₁₋₆ alkylsulfonyl, aminosulfonyl, C₁₋₆ alkylaminosulfonyl, di-C₁₋₆ alkylaminosulfonyl, and C₁₋₆ alkylsulfonylamino; wherein said C₁₋₆ alkyl is optionally substituted by 1, 2, 3, 4, 5, 6, 7, or 8 independently selected halogens.

In some embodiments, each R^{a5}, R^{c5}, and R^{d5} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a5}, R^{c5}, and R^{d5} are each optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, and C₁₋₆ haloalkoxy;
each R^{f5} and R^{g5} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
each R^{h5} and Rⁱ⁵ is independently selected from H, C₁₋₆ haloalkyl, C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
each R^{j5} and R^{k5} is independently selected from OH, C₁₋₆ alkoxy, and C₁₋₆ haloalkoxy; and
each R^{I} is D, halo, oxo, C₁₋₆ alkyl, C₁₋₆ haloalkyl, C₂₋₆ alkenyl, C₂₋₆ alkynyl, CN, C₁₋₆ alkoxy, C₁₋₆ haloalkoxy, amino, C₁₋₆ alkylamino, di-C₁₋₆ alkylamino, C₁₋₆ alkylsulfonyl, aminosulfonyl, C₁₋₆ alkylaminosulfonyl, di-C₁₋₆ alkylaminosulfonyl, and C₁₋₆ alkylsulfonylamino.

In some embodiments, each R^{I} and R^{J} is independently selected from D, halo, oxo, C₁₋₆ alkyl, C₁₋₆ haloalkyl, C₂₋₆ alkenyl, C₂₋₆ alkynyl, CN, C₁₋₆ alkoxy, C₁₋₆ haloalkoxy, amino, C₁₋₆ alkylamino, di-C₁₋₆ alkylamino, C₁₋₆ alkylsulfonyl, aminosulfonyl, C₁₋₆ alkylaminosulfonyl, di-C₁₋₆ alkylaminosulfonyl, and C₁₋₆ alkylsulfonylamino; and
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, and C₂₋₆ alkynyl.

In some embodiments:
X is N;
R¹ is selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a1}, C(O)R^{b1}, C(O)NR^{c1}R^{d1}, C(O)OR^{a1}, C(═NR^{e1})R^{b1}, C(═NR^{e1})NR^{c1}R^{d1}, S(O)R^{b1}, S(O)NR^{c1}R^{d1}, S(O)₂R^{b1}, and S(O)₂NR^{c1}R^{d1}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R¹ are each optionally substituted with 1, 2, 3, 4, 5, 6, 7, or 8 independently selected R^{B} substituents;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{a2} NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} substituents;
provided that when Cy¹ is 4-14 membered heterocycloalkyl, then the 4-14 membered heterocycloalkyl of Cy¹ is other than unsubstituted morpholinyl;
provided that Cy¹ is not pyridin-4-yl optionally substituted with 1, 2, 3, or 4 independently selected R^{E} substituents;
provided that Cy¹ is not pyrimidin-4-yl optionally substituted with 1, 2, or 3, independently selected R^{E} substituents;
provided that Cy¹ is not quinolin-4-yl optionally substituted with 1, 2, 3, 4, 5, or 6 independently selected R^{E} substituents;
Cy² is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a}, R^{c1}, R^{d1}, R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a1}, R^{c1}, R^{d1}, R^{a2}, R^{c2}, and R^{d2} are each optionally substituted with 1, 2, 3, 4, 5, 6, 7, or 8 independently selected R^{G} substituents;
or, any R^{c1} and R^{d1} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b1} and R^{b2} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b1}, R^{b2}, and R^{b3} are each optionally substituted with 1, 2, 3, 4, 5, 6, 7, or 8 independently selected R^{G} substituents;
each R^{e1} and R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2} and R^{g2} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{B}, R^{C}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR⁴, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{B}, R^{C}, R^{E}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{e6}C(O)OR^{a6}, NR^{c6}C(O)NR^{e5}R^{d6}, C(═NR^{e6})R^{b6}, C(═NR^{e6})NR^{e6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments:
X is N;
R¹ is selected from H, C₁₋₆ alkyl, C₂₋₆ alkenyl, C₂₋₆ alkynyl, and C₁₋₆ haloalkyl;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{a2} NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
Cy¹ is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{E} or R^{M} substituents;
provided that when Cy¹ is 4-14 membered heterocycloalkyl, then the 4-14 membered heterocycloalkyl of Cy¹ is other than unsubstituted morpholinyl;
Cy² is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a2}, R^{c2}, and R^{d2} are each optionally substituted with 1, 2, 3, 4, 5, 6, 7, or 8 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b2} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b2} are each optionally substituted with 1, 2, 3, 4, 5, 6, 7, or 8 independently selected R^{G} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2} and R^{g2} is independently selected from H, C₁₋₆ alkyl, C₁₋₆ haloalkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{C}, R^{E}, R^{M}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR⁴, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{C}, R^{E}, R^{M}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{b5})NR^{c5}R^{d5}, NR^{c5}C(═NR^{b5})NR^{c5}R^{d5}, NR_{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments:
X is N;
R¹ is selected from H, C₁₋₆ alkyl, C₂₋₆ alkenyl, C₂₋₆ alkynyl, and C₁₋₆ haloalkyl;
R² is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{b2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
Cy¹ is C₆₋₁₄ aryl, C₃₋₁₄ cycloalkyl, or 5-14 membered heteroaryl, wherein the C₆₋₁₄ aryl, C₃₋₁₄ cycloalkyl, or 5-14 membered heteroaryl is optionally substituted with 1, 2, 3, 4, 5, 6, 7, or 8 independently selected R^{E} or R^{M} substituents;
Cy² is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a2}, R^{c2}, and R^{d2} are each optionally substituted with 1, 2, 3, 4, 5, 6, 7, or 8 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b2} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b2} are each optionally substituted with 1, 2, 3, 4, 5, 6, 7, or 8 independently selected R^{G} substituents;
each R^{e2} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2} and R^{g2} is independently selected from H, C₁₋₆ alkyl, C₁₋₆ haloalkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2} and Rⁱ² is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2} and R^{k2} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{C}, R^{E}, R^{M}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR⁴, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{C}, R^{E}, R^{M}, R^{F}, and R^{G} are each optionally substituted with 1, 2, 3, 4, 5, 6, 7, or 8 independently selected R^{E} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{E} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{E} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{E} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a5}, SR^{a5}, NHOR^{a5}, C(O)R^{b5}, C(O)NR^{c5}R^{a5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}NR^{c5}C(O)OR^{d5}, NR^{c5}C(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{a5} S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{H} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6}NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{e6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{e6})R^{b6}, NR^{c6}S(O)₂NR^{c6}R^{d6} S(O)R^{b6}, S(O)NR^{c6}R^{d6} S(O)₂R^{b6}, S(O)₂NR^{c6}R^{d6}, OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{I} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy; E, or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a7}, SR^{a7}, NHOR^{a7}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{a7} S(O)₂R^{b7}, S(O)₂NR^{c7}R^{d7}, OS(O)(═NR^{e7})R^{b7}, OS(O)₂R^{b7}, SF₅, P(O)R^{j7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

In some embodiments, the compound of Formula (I) is a compound of Formula (II):

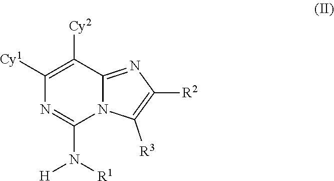

or a pharmaceutically acceptable salt thereof.

In some embodiments, the compound of Formula (I) is a compound of Formula (III):

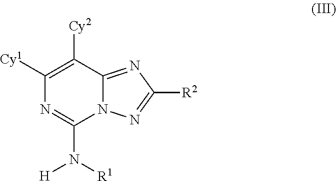

or a pharmaceutically acceptable salt thereof.

In some embodiments, Cy¹ is C₆₋₁₄ aryl, wherein the C₆₋₁₄ aryl is optionally substituted with 1, 2, 3, or 4 independently selected R^{E} substituents.

In some embodiments, Cy¹ is C₃₋₁₄ cycloalkyl, wherein the C₃₋₁₄ cycloalkyl is optionally substituted with 1, 2, 3, or 4 independently selected R^{E} substituents.

In some embodiments, Cy¹ is 5-14 membered heteroaryl, wherein the 5-14 membered heteroaryl is optionally substituted with 1, 2, 3, or 4 independently selected R^{E} substituents.

In some embodiments, Cy¹ is 4-14 membered heterocycloalkyl, wherein the 4-14 membered heterocycloalkyl is optionally substituted with 1, 2, 3, or 4 independently selected R^{E} substituents.

In some embodiments, Cy¹ is C₆₋₁₀ aryl, wherein the C₆₋₁₀ aryl is optionally substituted with 1, 2, 3, or 4 independently selected R^{E} substituents.

In some embodiments, Cy¹ is C₃₋₇ cycloalkyl, wherein the C₃₋₇ cycloalkyl is optionally substituted with 1, 2, 3, or 4 independently selected R^{E} substituents.

In some embodiments, Cy¹ is 5-10 membered heteroaryl, wherein the 5-10 membered heteroaryl is optionally substituted with 1, 2, 3, or 4 independently selected R^{E} substituents.

In some embodiments, Cy¹ is 4-10 membered heterocycloalkyl, wherein the 4-10 membered heterocycloalkyl is optionally substituted with 1, 2, 3, or 4 independently selected R^{E} substituents.

In some embodiments, Cy¹ is phenyl or 5-10 membered heteroaryl, wherein the phenyl or 5-10 membered heteroaryl is optionally substituted with 1, 2, 3, or 4 independently selected R^{E} substituents.

In some embodiments, Cy¹ is phenyl, optionally substituted with 1, 2, 3, or 4 independently selected R^{M} substituents, or C₇₋₁₄ aryl or 5-14 membered heteroaryl wherein the C₇₋₁₄ aryl and 5-14 membered heteroaryl are optionally substituted with 1, 2, 3, or 4 independently selected R^{E} substituents, and
wherein each R^{M} is independently selected from D, halo, oxo, C₁₋₆ alkyl, C₁₋₆ haloalkyl, C₁₋₆ alkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, 2-CN, 3-CN, NO₂, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₁₋₆ alkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl of R^{M} are each optionally substituted with 1, 2, 3, or 4 independently selected R^{E} substituents.

In some embodiments, Cy¹ is selected from phenyl, pyridinyl, furanyl, benzofuranyl, and pyrazolyl, each of which is optionally substituted with 1, 2, or 3 substituents selected from C₁₋₃ alkyl, halo, CN, and C₁₋₃ alkoxy.

In some embodiments, the optionally substituted Cy¹ is selected from cyanophenyl, cyanofluorophenyl, 2,3-dihydro-1H-pyrrolo[2,3,-b]pyridine, phenyl, methoxyphenyl, fluorophenyl, pyridinyl, methylfuranyl, benzofuranyl, and methyl-1H-pyrazolyl.

In some embodiments, the optionally substituted Cy¹ is selected from cyanophenyl, 2,3-dihydro-1H-pyrrolo[2,3,-b]pyridine, phenyl, methoxyphenyl, fluorophenyl, pyridinyl, methylfuranyl, benzofuranyl, and methyl-1H-pyrazolyl.

In some embodiments, Cy¹ is selected from 2-cyanophenyl, 3-cyanophenyl, 3-cyano-2-fluorophenyl, 2,3-dihydro-1H-pyrrolo[2,3,-b]pyridine, phenyl, 3-methoxyphenyl, 2-fluorophenyl, pyridine-4-yl, 2-methylfuran-3-yl, benzofuran-2-yl, and 1-methyl-1H-pyrazol-4-yl.

In some embodiments, the optionally substituted Cy¹ is selected from 2-cyanophenyl, 3-cyanophenyl, 2,3-dihydro-1H-pyrrolo[2,3,-b]pyridine, phenyl, 3-methoxyphenyl, 2-fluorophenyl, pyridine-4-yl, 2-methylfuran-3-yl, benzofuran-2-yl, and 1-methyl-1H-pyrazol-4-yl.

In some embodiments, the optionally substituted Cy¹ is selected from 3-cyanophenyl and phenyl.

In some embodiments, Cy¹ is 3-cyanophenyl.

In some embodiments, Cy² is C₆₋₁₄ aryl, C₄₋₁₄ cycloalkyl, 5-14 membered heteroaryl or 4-14 membered heterocycloalkyl wherein the C₆₋₁₄ aryl, C₄₋₁₄ cycloalkyl, 5-14 membered heteroaryl and 4-14 membered heterocycloalkyl are optionally substituted with 1, 2, 3, 4, 5, 6, 7, or 8 independently selected R^{F} substituents.

In some embodiments, Cy² is C₆₋₁₄ aryl, wherein the C₆₋₁₄ aryl is optionally substituted with 1, 2, 3, or 4 independently selected R^{F} substituents.

In some embodiments, Cy² is C₃₋₁₄ cycloalkyl, wherein the C₃₋₁₄ cycloalkyl is optionally substituted with 1, 2, 3, or 4 independently selected R^{F} substituents.

In some embodiments, Cy² is 5-14 membered heteroaryl, wherein the 5-14 membered heteroaryl is optionally substituted with 1, 2, 3, or 4 independently selected R^{F} substituents.

In some embodiments, Cy² is 4-14 membered heterocycloalkyl, wherein the 4-14 membered heterocycloalkyl is optionally substituted with 1, 2, 3, or 4 independently selected R^{F} substituents.

In some embodiments, Cy² is C₆₋₁₀ aryl, wherein the C₆₋₁₀ aryl is optionally substituted with 1, 2, 3, or 4 independently selected R^{F} substituents.

In some embodiments, Cy² is C₃₋₇ cycloalkyl, wherein the C₃₋₇ cycloalkyl is optionally substituted with 1, 2, 3, or 4 independently selected R^{F} substituents.

In some embodiments, Cy² is 5-10 membered heteroaryl, wherein the 5-10 membered heteroaryl is optionally substituted with 1, 2, 3, or 4 independently selected R^{F} substituents.

In some embodiments, Cy² is 4-10 membered heterocycloalkyl, wherein the 4-10 membered heterocycloalkyl is optionally substituted with 1, 2, 3, or 4 independently selected R^{F} substituents.

In some embodiments, Cy² is selected from C₃₋₆ cycloalkyl, phenyl, 5-10 membered heteroaryl, and 5-10 membered heterocycloalkyl;
wherein the 5-10 membered heteroaryl and 5-10 membered heterocycloalkyl each comprise one, two, or three nitrogen atoms as ring-forming heteroatoms, wherein one of the one or two nitrogen atoms is optionally an N-oxide, and wherein a ring-forming carbon atom is optionally substituted by oxo; and
wherein the C₃₋₆ cycloalkyl, phenyl, 5-10 membered heteroaryl, and 5-10 membered heterocycloalkyl are each optionally substituted with 1, 2, or 3 substituents selected from C₁₋₃ alkyl, C₁₋₃ alkyl-OH, halo, CN, C₁₋₃ alkoxy, and C(O)NH₂

In some embodiments, Cy² is selected from C₃₋₆ cycloalkyl, phenyl, 5-10 membered heteroaryl, and 5-10 membered heterocycloalkyl;
wherein the 5-10 membered heteroaryl and 5-10 membered heterocycloalkyl each comprise one or two nitrogen atoms as ring-forming heteroatoms, wherein one of the one or two nitrogen atoms is optionally an N-oxide, and wherein a ring-forming carbon atom is optionally substituted by oxo; and
wherein the C₃₋₆ cycloalkyl, C₆-aryl, 5-10 membered heteroaryl, and 5-10 membered heterocycloalkyl are each optionally substituted with 1, 2, or 3 substituents selected from C₁₋₃ alkyl, halo, CN, C₁₋₃ alkoxy, and C(O)NH₂.

In some embodiments, Cy² is selected from pyridinyl, tetrahydropyridinyl, piperidinyl, pyridine-N-oxide, oxo-dihydropyridinyl, phenyl, pyrazolo[1,5-a]pyridin-3-yl, pyrazolo[1,5-b]pyridazinyl, pyrazolyl, pyrimidinyl, quinolinyl, oxazolyl, 2,3-dihydro-[1,4]dioxino[2,3-b]pyridin-8-yl, and triazolyl each of which is optionally substituted with 1, 2, or 3 substituents selected from C₁₋₃ alkyl, C₁₋₃ alkyl-OH, halo, CN, C₁₋₃ alkoxy, and C(O)NH₂

In some embodiments, Cy² is selected from pyridinyl, tetrahydropyridinyl, piperidinyl, pyridine-N-oxide, oxo-dihydropyridinyl, phenyl, pyrazolo[1,5-a]pyridin-3-yl, pyrazolo[1,5-b]pyridazinyl, pyrazolyl, pyrimidinyl, quinolinyl, oxazolyl, and 2,3-dihydro-[1,4]dioxino[2,3-b]pyridin-8-yl, each of which is optionally substituted with 1, 2, or 3 substituents selected from C₁₋₃ alkyl, halo, CN, C₁₋₃ alkoxy, and C(O)NH₂.

In some embodiments, Cy² is selected from pyridinyl, tetrahydropyridinyl, piperidinyl, pyridine-N-oxide, oxo-dihydropyridinyl, phenyl, pyrazolo[1,5-a]pyridin-3-yl, pyrazolyl, pyrimidinyl, and 2,3-dihydro-[1,4]dioxino[2,3-b]pyridin-8-yl, each of which is optionally substituted with 1, 2, or 3 substituents selected from C₁₋₃ alkyl, halo, CN, C₁₋₃ alkoxy, and C(O)NH₂.

In some embodiments, Cy² is cyclopropyl optionally substituted with 1, 2, or 3 substituents selected from C₁₋₃ alkyl, halo, CN, C₁₋₃ alkoxy, and C(O)NH₂.

In some embodiments, the optionally substituted Cy² is selected from 2,6-dimethylpyridin-4-yl, pyridin-4-yl, 2-methylpyridin-4-yl, 1-carbamoyl-1,2,3,6-tetrahydropyridin-4-yl, 1-carbamoylpiperidin-4-yl, 2-methoxypyridin-4-yl, 2-methoxy-6-methylpyridin-4-yl, 2,6-dimethylpyridin-4-yl-1-oxide, 1-ethyl-6-oxo-1,6-dihydropyridin-3-yl, 3-methylpyridin-4-yl, 3-fluoropyridin-4-yl, 3-chloropyridin-4-yl, 3-methoxypyridin-4-yl, 3-cyanopyridin-4-yl, 4-carbamoylphenyl, pyrazolo[1,5-a]pyridin-3-yl, pyrazolo[1,5-b]pyridazin-3-yl, 5-methyl-1H-pyrazol-4-yl, 1-ethyl-1H-pyrazol-5-yl, 1-isopropyl-1H-pyrazol-5-yl, 1-propyl-1H-pyrazol-5-yl, pyrimidin-4-yl, 2,3-dihydro-[1,4]dioxino[2,3-b]pyridin-8-yl, quinolin-5-yl, 5-fluoropyrimidin-4-yl, oxazol-5-yl, 4-methyloxazol-5-yl, 4-ethyloxazol-5-yl, 4-(hydroxymethyl)-2-methyloxazol-5-yl, 4-(methoxymethyl)-2-methyloxazol-5-yl, 4-(hydroxymethyl)-2-methyloxazol-5-yl, 1-ethyl-1H-1,2,3-triazol-5-yl, and cyclopropyl

In some embodiments, the optionally substituted Cy² is selected from 2,6-dimethylpyridin-4-yl, pyridin-4-yl, 2-methylpyridin-4-yl, 1-carbamoyl-1,2,3,6-tetrahydropyridin-4-yl, 1-carbamoylpiperidin-4-yl, 2-methoxypyridin-4-yl, 2-methoxy-6-methylpyridin-4-yl, 2,6-dimethylpyridin-4-yl-1-oxide, 1-ethyl-6-oxo-1,6-dihydropyridin-3-yl, 3-methylpyridin-4-yl, 3-fluoropyridin-4-yl, 3-chloropyridin-4-yl, 3-methoxypyridin-4-yl, 3-cyanopyridin-4-yl, 4-carbamoylphenyl, pyrazolo[1,5-a]pyridin-3-yl, pyrazolo[1,5-b]pyridazin-3-yl, 5-methyl-1H-pyrazol-4-yl, 1-ethyl-1H-pyrazol-5-yl, 1-isopropyl-1H-pyrazol-5-yl, 1-propyl-1H-pyrazol-5-yl, pyrimidin-4-yl, 2,3-dihydro-[1,4]dioxino[2,3-b]pyridin-8-yl, quinolin-5-yl, 5-fluoropyrimidin-4-yl, 4-methyloxazol-5-yl, 4-(hydroxymethyl)-2-methyloxazol-5-yl, 4-(methoxymethyl)-2-methyloxazol-5-yl, and cyclopropyl.

In some embodiments, the optionally substituted Cy² is selected from 2,6-dimethylpyridin-4-yl, pyridin-4-yl, 2-methylpyridin-4-yl, 1-carbamoyl-1,2,3,6-tetrahydropyridin-4-yl, 1-carbamoylpiperidin-4-yl, 2-methoxypyridin-4-yl, 2,6-dimethylpyridin-4-yl-1-oxide, 1-ethyl-6-oxo-1,6-dihydropyridin-3-yl, 3-methylpyridin-4-yl, 3-fluoropyridin-4-yl, 3-chloropyridin-4-yl, 3-methoxypyridin-4-yl, 3-cyanopyridin-4-yl, 4-carbamoylphenyl, pyrazolo[1,5-a]pyridin-3-yl, 5-methyl-1H-pyrazol-4-yl, 1-ethyl-1H-pyrazol-5-yl, 1-isopropyl-1H-pyrazol-5-yl, 1-propyl-1H-pyrazol-5-yl, pyrimidin-4-yl, 2,3-dihydro-[1,4]dioxino[2,3-b]pyridin-8-yl, and cyclopropyl.

In some embodiments, R¹ is selected from H, D, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a1}, C(O)R^{b1}, C(O)NR^{c1}R^{d1}, C(O)OR^{a1}, C(═NR^{e1})R^{b1}, C(═NR^{e1})NR^{c1}R^{d1}, S(O)R^{b1}, S(O)NR^{c1}R^{d1}, S(O)₂R^{b1} and S(O)₂NR^{c1}R^{d1} wherein the C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R¹ are each optionally substituted with 1, 2, 3, 4, 5, 6, 7, or 8 independently selected R^{B} substituents.

In some embodiments, R¹ is selected from H, D, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-8 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-8 membered heteroaryl)-C₁₋₆ alkyl-, (4-14 membered heterocycloalkyl)-C₁₋₆ alkyl-, OR^{a1}, C(O)R^{b1}, C(O)NR^{c1}R^{d1}, C(O)OR^{a1}, C(═NR^{e1})R^{b1}, C(═NR^{e1})NR^{c1}R^{d1}, S(O)R^{b1}, S(O)NR^{c1}R^{d1}, S(O)₂R^{b1} and S(O)₂NR^{c1}R^{d1}, wherein the C₂₋₆ alkenyl, C₂₋₆ alkynyl, C₆₋₁₄ aryl, C₃₋₁₄ cycloalkyl, 5-8 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-8 membered heteroaryl)-C₁₋₆ alkyl-, and (4-14 membered heterocycloalkyl)-C₁₋₆ alkyl- of R¹ are each optionally substituted with 1, 2, 3, 4, 5, 6, 7, or 8 independently selected R^{B} substituents.

In some embodiments, R¹ is selected from H, C₁₋₆ alkyl, and 5-8 membered heteroaryl, wherein the 5-8 membered heteroaryl is optionally substituted by 1 or 2 independently selected R^{B} substituents.

In some embodiments, R¹ is H, C₁₋₆ alkyl, or a 5-8 membered heteroaryl

In some embodiments, R¹ is H or C₁₋₆ alkyl.

In some embodiments, R¹ is H or C₁₋₃ alkyl.

In some embodiments, R¹ is H, ethyl, or nicotinonitrile.

In some embodiments, R¹ is H or ethyl.

In some embodiments, R¹ is H.

In some embodiments, R¹ is 5-8 membered heteroaryl which is optionally substituted by 1 or 2 independently selected R^{B} substituents.

In some embodiments, R¹ is 5-8 membered heteroaryl.

In some embodiments, R¹ is pyridyl which is optionally substituted by 1 or 2 R^{B} substituents.

In some embodiments, R¹ is pyridyl which is optionally substituted by cyano.

In some embodiments, R¹ is nicotinonitrile.

In some embodiments, R¹ is 3-cyanopyridyl.

In some embodiments, R² is selected from halo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{b2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2} NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- are each optionally substituted with 1, 2, 3, 4, 5, 6, 7, or 8 independently selected R^{C} substituents; and
wherein the C₁₋₆ alkyl is substituted with 1, 2, 3, 4, 5, 6, 7, or 8 independently selected R^{C} substituents.

In some embodiments, R² is selected from C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, C(O)R^{b2}, C(O)NR^{c2}R^{d2}, C(O)OR^{a2}, and NR^{c2}R^{d2}, wherein the C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- are each optionally substituted with 1, 2, 3, 4, or 5 independently selected R^{C} substituents; and
wherein the C₁₋₆ alkyl is substituted with 1, 2, 3, 4, or 5 independently selected R^{C} substituents.

In some embodiments, R² is selected from C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, C(O)R^{b2}, C(O)NR^{c2}R^{d2}, C(O)OR^{a2}, and NR^{c2}R^{d2}, wherein the C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- are each optionally substituted with 1, 2, 3, 4, or 5 independently selected R^{C} substituents.

In some embodiments, R² is selected from C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- are each optionally substituted with 1, 2, 3, 4, or 5 independently selected R^{C} substituents.

In some embodiments, R² is selected from H, C₁₋₆ alkyl, C₃₋₁₄ cycloalkyl, C₆₋₁₄ aryl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, NR^{c2}R^{d2}, C(O)R^{b2}, C(O)NR^{c2}R^{d2}, and C(O)OR^{a2}, wherein the C₁₋₆ alkyl, C₃₋₁₄ cycloalkyl, C₆₋₁₄ aryl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- are each optionally substituted with 1, 2, or 3 independently selected R^{C} substituents.

In some embodiments, R² is selected from H, C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a2}, NR^{c2}R^{d2}, C(O)R^{b2}, C(O)NR^{c2}R^{d2}, and C(O)OR^{a2}, wherein the C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, are each optionally substituted with 1, 2, or 3 independently selected R^{C} substituents.

In some embodiments, R² is selected from H, C₁₋₆ alkyl, C₆₋₁₄ aryl, 5-14 membered heteroaryl, 4-14 membered heterocycloalkyl, C₆₋₁₄ aryl-C₁₋₆ alkyl-, (5-14 membered heteroaryl)-C₁₋₆ alkyl-, (4-14 membered heterocycloalkyl)-C₁₋₆ alkyl-, NR^{c2}R^{d2}, C(O)R^{b2}, C(O)NR^{c2}R^{d2}, and C(O)OR^{a2}, wherein the C₁₋₆ alkyl, C₆₋₁₄ aryl, 5-14 membered heteroaryl, 4-14 membered heterocycloalkyl, C₆₋₁₄ aryl-C₁₋₆ alkyl-, (5-14 membered heteroaryl)-C₁₋₆ alkyl-, and (4-14 membered heterocycloalkyl)-C₁₋₆ alkyl- are each optionally substituted with 1, 2, or 3 independently selected R^{C} substituents.

In some embodiments, R² is selected from H, C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a2}, NR^{c2}R^{d2}, C(O)R^{b2}, C(O)NR^{c2}R^{d2}, and C(O)OR^{a2}, wherein the C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, are each substituted with 1, 2, or 3 independently selected R^{C} substituents

In some embodiments, R² is selected from H, C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, NR^{c2}R^{d2}, C(O)R^{b2}, C(O)NR^{c2}R^{d2}, and C(O)OR^{a2}, wherein the C₁₋₆ alkyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- are each substituted with 1, 2, or 3 independently selected R^{C} substituents.

In some embodiments, R² is selected from H, C₆₋₁₄ aryl, 5-14 membered heteroaryl, C(O)R^{b2}, C(O)NR^{c2}R^{d2}, and C(O)OR^{a2}, wherein the C₆₋₁₄ aryl and 5-14 membered heteroaryl are each optionally substituted with 1, 2, or 3 independently selected R^{C} substituents.

In some embodiments, R² is selected from H, C₁₋₆ alkyl, phenyl, 5-6 membered heteroaryl, 4-10 membered heterocycloalkyl, C₆₋₁₀ aryl-C₁₋₆ alkyl-, (5-10 membered heteroaryl)-C₁₋₆ alkyl-, (4-10 membered heterocycloalkyl)-C₁₋₆ alkyl-, NR^{c2}R^{d2}, C(O)R^{b2}, C(O)NR^{c2}R^{d2}, and C(O)OR^{a2}, wherein the C₁₋₆ alkyl, phenyl, 5-6 membered heteroaryl, 4-10 membered heterocycloalkyl, C₆₋₁₀ aryl-C₁₋₆ alkyl-, (5-10 membered heteroaryl)-C₁₋₆ alkyl-, and (4-10 membered heterocycloalkyl)-C₁₋₆ alkyl- are each optionally substituted with 1 or 2 independently selected R^{C} substituents. In some embodiments, R² is selected from H, phenyl, 5-6 membered heteroaryl, C(O)R^{b2}, C(O)NR^{c2}R^{d2}, and C(O)OR^{a2}, wherein the phenyl and 5-6 membered heteroaryl are each optionally substituted with 1 or 2 independently selected R^{C} substituents.

In some embodiments, R² is selected from H, C(O)OEt, CONH₂, and C(O)NHEt.

In some embodiments, R² selected from phenyl and 5-6 membered heteroaryl, each of which is optionally substituted with C(O)OMe.

In some embodiments, the optionally substituted R² is selected from pyridinylmethyl, hydroxy(phenyl)methyl, hydroxyethylamino(phenyl)ethyl, cyclohexylmethyl, fluorobenzyl, hydroxy(fluorophenyl)methyl, (methylpyridinyl)methyl, (fluoropyridinyl)methyl, (trifluoromethylpyridinyl)methyl, ((hydroxymethyl)pyridinyl)methyl, (methoxypyridinyl)methyl, (methylpyrazolyl)benzyl, (methylpyrazolyl)methyl, benzoisoxazolylmethyl, (methylindazolyl)methyl, (hydroxyazetidinyl)methyl, benzoyl, phenylcyclopropyl, (cyan(phenyl)methyl)amino, tetrahydrofuranyl, phenyl(pyridinyloxy)methyl, fluoro ((fluorohydroxypyrrolidinyl)methyl)benzyl, ((carboxypiperidinyl)methyl)fluorobenzyl, fluoro((N-methylmethylsulfonamido)methyl)benzyl, ((dioxoimidazolidinyl)methyl)fluorobenzyl, (difluorophenyl)(hydroxy)methyl, (pyridinyl-1H-tetrazolyl)methyl, (pyrazolyl-1H-tetrazolyl)methyl, (thiazolyl-1H-tetrazolyl)methyl, (methyltrifluoromethylpyrazolyl)methyl, ((1,1-dioxidoisothiazolidinyl)methyl)fluorobenzyl, ((methyl-2,5-dioxoimidazolidinyl)methyl)benzyl, and (cyanophenoxy)methyl.

In some embodiments, the optionally substituted R² is selected from pyridinylmethyl, hydroxy(phenyl)methyl, hydroxyethylamino(phenyl)ethyl, cyclohexylmethyl, fluorobenzyl, hydroxy(fluorophenyl)methyl, (methylpyridinyl)methyl, (fluoropyridinyl)methyl, (methoxypyridinyl)methyl, (methylpyrazolyl)benzyl, benzoisoxazolylmethyl, (methylindazolyl)methyl, (hydroxyazetidinyl)methyl, benzoyl, phenylcyclopropyl, (cyano(phenyl)methyl)amino, tetrahydrofuranyl, phenyl(pyridinyloxy)methyl, fluoro ((fluorohydroxypyrrolidinyl)methyl)benzyl, ((carboxypiperidinyl)methyl)fluorobenzyl, fluoro((N-methylmethylsulfonamido)methyl)benzyl, ((dioxoimidazolidinyl)methyl)fluorobenzyl, and (difluorophenyl)(hydroxy)methyl.

In some embodiments, R² is selected from pyridinylmethyl, hydroxy(phenyl)methyl, hydroxyethylamino(phenyl)ethyl, cyclohexylmethyl, fluorobenzyl, hydroxy(fluorophenyl)methyl, (methylpyridinyl)methyl, (fluoropyridinyl)methyl, (methoxypyridinyl)methyl, (methylpyrazolyl)benzyl, benzoisoxazolylmethyl, (methylindazolyl)methyl, (hydroxyazetidinyl)methyl, benzoyl, phenylcyclopropyl, (cyano(phenyl)methyl)amino, tetrahydrofuranyl, and phenyl(pyridinyloxy)methyl.

In some embodiments, R² is

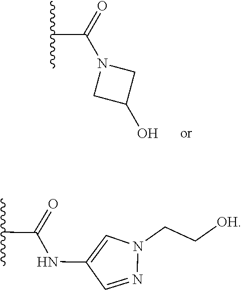 [SMILES: *C(=O)N1CC(O)C1 ; *C(=O)Nc1cnn(CCO)c1]

In some embodiments, R² is selected from pyridin-2-ylmethyl, hydroxy(phenyl)methyl, (2-hydroxyethylamino)(phenyl)methyl, cyclohexylmethyl, 2-fluorobenzyl, (2-fluorophenyl)(hydroxy)methyl, (6-methylpyridin-2-yl)methyl, (3-fluoropyridin-2-yl)methyl, (3-methoxypyridin-2-yl)methyl, 2-(1-methyl-1H-pyrazol-4-yl)benzyl, benzo[d]isoxazol-3-ylmethyl, (1-methyl-1H-indazol-3-yl)methyl, (3-hydroxyazetidin-1-yl)methyl, benzoyl, 1-phenylcyclopropyl, (cyano(phenyl)methyl)amino, tetrahydrofuran-3-yl, phenyl(pyridin-2-yloxy)methyl, 2-fluoro-6-(((3R,4R)-3-fluoro-4-hydroxypyrrolidin-1-yl)methyl)benzyl, 2-((4-carboxypiperidin-1-yl)methyl)-6-fluorobenzyl, 2-fluoro-6-((N-methylmethylsulfonamido)methyl)benzyl, 2-((2,5-dioxoimidazolidin-1-yl)methyl)-6-fluorobenzyl, (2,6-difluorophenyl)(hydroxy)methyl, (5-(pyridin-2-yl)-1H-tetrazol-1-yl)methyl, (5-(1H-pyrazol-1-yl)-1H-tetrazol-1-yl)methyl, (5-(thiazol-4-yl)-1H-tetrazol-1-yl)methyl, (5-methyl-3-(trifluoromethyl)-1H-pyrazol-1-yl)methyl, (3-methylpyridin-2-yl)methyl, 2-((1,1-dioxidoisothiazolidin-2-yl)methyl)-6-fluorobenzyl, 2-fluoro-6-((3-methyl-2,5-dioxoimidazolidin-1-yl)methyl)benzyl, (6-(trifluoromethyl)pyridin-2-yl)methyl, (3-(hydroxymethyl)pyridin-2-yl)methyl, (1-methyl-1H-pyrazol-3-yl)methyl, and (2-cyanophenoxy)methyl, (3-methylpyridin-2-yl)methoxy, (6-methylpyridin-2-yl)methoxy, and ((3-methylpyridin-2-yl)methyl)amino.

In some embodiments, R² is selected from pyridin-2-ylmethyl, hydroxy(phenyl)methyl, (2-hydroxyethylamino)(phenyl)methyl, cyclohexylmethyl, 2-fluorobenzyl, (2-fluorophenyl)(hydroxy)methyl, (6-methylpyridin-2-yl)methyl, (3-fluoropyridin-2-yl)methyl, (3-methoxypyridin-2-yl)methyl, 2-(1-methyl-1H-pyrazol-4-yl)benzyl, benzo[d]isoxazol-3-ylmethyl, (1-methyl-1H-indazol-3-yl)methyl, (3-hydroxyazetidin-1-yl)methyl, benzoyl, 1-phenylcyclopropyl, (cyano(phenyl)methyl)amino, tetrahydrofuran-3-yl, phenyl(pyridin-2-yloxy)methyl, 2-fluoro-6-(((3R,4R)-3-fluoro-4-hydroxypyrrolidin-1-yl)methyl)benzyl, 2-((4-carboxypiperidin-1-yl)methyl)-6-fluorobenzyl, 2-fluoro-6-((N-methylmethylsulfonamido)methyl)benzyl, 2-((2,5-dioxoimidazolidin-1-yl)methyl)-6-fluorobenzyl, and (2,6-difluorophenyl)(hydroxy)methyl.

In some embodiments, R² is selected from pyridin-2-ylmethyl, hydroxy(phenyl)methyl, 2-hydroxyethylamino)(phenyl)methyl, cyclohexylmethyl, 2-fluorobenzyl, 2-fluorophenyl)(hydroxy)methyl, 6-methylpyridin-2-yl)methyl, 3-fluoropyridin-2-yl)methyl, 3-methoxypyridin-2-yl)methyl, 2-(1-methyl-1H-pyrazol-4-yl)benzyl, benzo[d]isoxazol-3-ylmethyl, 1-methyl-1H-indazol-3-yl)methyl, 3-hydroxyazetidin-1-yl)methyl, benzoyl, 1-phenylcyclopropyl, (cyan(phenyl)methyl)amino, tetrahydrofuran-3-yl, and phenyl(pyridin-2-yloxy)methyl.

In some embodiments, R^{a2} is selected from H and C₁₋₆ alkyl, wherein the C₁₋₆ alkyl is optionally substituted 1, 2, or 3 independently selected R^{E} substituents.

In some embodiments, R^{b2} is selected from H, C₁₋₆ alkyl, C₆₋₁₄ aryl, and 4-14 membered heterocycloalkyl, wherein the C₁₋₆ alkyl, C₆₋₁₄ aryl, and 4-14 membered heterocycloalkyl are each optionally substituted with 1, 2, or 3 independently selected R^{G} substituents.

In some embodiments, R^{c2} and R^{d2} are each independently selected from H, C₁₋₆ alkyl, 5-14 membered heteroaryl, and C₆₋₁₄ aryl-C₁₋₆ alkyl-, wherein the C₁₋₆ alkyl, 5-14 membered heteroaryl, and C₆₋₁₄ aryl-C₁₋₆ alkyl- are each optionally substituted with 1, 2, or 3 independently selected R^{G} substituents.

In some embodiments, each R^{G} is independently selected from CN, OR^{a4}, and C₁₋₆ alkyl, wherein the C₁₋₆ alkyl is optionally substituted 1, 2, or 3 independently selected R^{E} substituents.

In some embodiments, each R^{C} is independently selected from halo, C₁₋₆ alkyl, C₆₋₁₄ aryl, 5-14 membered heteroaryl, (4-14 membered heterocycloalkyl)-C₁₋₆ alkyl-, OR^{a4}, C(O)OR^{a4}, and NR^{c4}R^{d4}, wherein the C₁₋₆ alkyl, 5-14 membered heteroaryl, and (4-14 membered heterocycloalkyl)-C₁₋₆ alkyl- are optionally substituted with 1, 2, or 3 independently selected R^{E} substituents.

In some embodiments, each R^{C} is independently selected from halo, C₁₋₆ alkyl, C₆₋₁₄ aryl, 5-14 membered heteroaryl, (4-14 membered heterocycloalkyl)-C₁₋₆ alkyl-, OR^{a4}, C(O)OR^{a4}, and NR^{c4}R^{d4}, wherein the C₁₋₆ alkyl, 5-14 membered heteroaryl, and (4-14 membered heterocycloalkyl)-C₁₋₆ alkyl- are substituted with 1, 2, or 3 independently selected R^{E} substituents.

In some embodiments, R^{a4} is selected from H, C₁₋₆ alkyl, C₆₋₁₄ aryl, and 5-14 membered heteroaryl.

In some embodiments, each R^{c4} and R^{d4}, are independently selected from H and C₁₋₆ alkyl, wherein the C₁₋₆ alkyl is optionally substituted by 1, 2, or 3 independently selected R^{E} substituents.

In some embodiments, each R^{H} is independently selected from halo, oxo, C₁₋₆ alkyl, C₁₋₆ haloalkyl, OR^{a5}, C(O)OR^{a5}, and NR^{c5}S(O)₂R^{b5}.

In some embodiments, each R^{a5}, and R^{cy} is selected from H and C₁₋₆ alkyl.

In some embodiments, R^{b5} is selected from H and C₁₋₆ alkyl.

In some embodiments, R³ is selected from H, D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}NR^{c3}R^{d3}, NR^{c3}C(O)R^{b3}, NR^{c3}C(O)OR^{d3}, NR^{c3}C(O)NR^{c3}R^{d3}, C(═NR^{e3})R^{b3}, C(═NR^{b3})NR^{c3}R^{d3}, NR^{c3}C(═NR^{e3})NR^{c3}R^{d3}, NR^{c3}(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3} S(O)R^{b3}, S(O)NR^{c3}R^{d3}, S(O)₂R^{b3}, OS(O)(═NR^{e3})R^{b3}, OS(O)₂R^{b3}, SF₅, P(O)R^{f3}R^{g3}, OP(O)(OR^{f3})(OR^{g3}), P(O)(OR^{f3})(OR^{g3}), B(OR^{h3})₂ and S(O)₂NR^{c3}R^{d3}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R³ are each optionally substituted with 1, 2, 3, 4, 5, 6, 7, or 8 independently selected R^{D} substituents.

In some embodiments, R³ is selected from H, halo, C₁₋₆ alkyl, C₁₋₆ haloalkyl, C₆₋₁₄ aryl, 5-14 membered heteroaryl, CN, and OR^{a3}, wherein the C₁₋₆ alkyl, C₆₋₁₄ aryl, and 5-14 membered heteroaryl are each optionally substituted with 1, 2, or 3 independently selected R^{D} substituents.

In some embodiments, R³ is selected from H, C₁₋₃ alkyl, halo, CN, morpholinomethyl, 4-ethoxyphenyl, 2-hydroxyethoxy, and pyridinyl.

In some embodiments, R³ is selected from H, methyl, bromo, CN, morpholinomethyl, 4-ethoxyphenyl, 2-hydroxyethoxy, and pyridinyl.

In some embodiments, X is CR³; and R¹ is H or C₁₋₆ alkyl.

In some embodiments, X is CR³; and R¹ is H or C₁₋₃ alkyl.

In some embodiments,
X is CR³;
R¹ is selected from H and C₁₋₆ alkyl;
R² is selected from H, D, C₆₋₁₄ aryl, 5-14 membered heteroaryl, C(O)R^{b2}, C(O)NR^{c2}R^{d2}, and C(O)OR^{a2}, wherein the C₆₋₁₄ aryl and 5-14 membered heteroaryl of R² are each optionally substituted with 1, 2, 3, or 4 independently selected R^{C} substituents;
R³ is selected from H, D, halo, C₁₋₆ alkyl, C₁₋₆ haloalkyl, C₆₋₁₄ aryl, 5-14 membered heteroaryl, CN, and OR^{a3}, wherein the C₁₋₆ alkyl, C₆₋₁₄ aryl, and 5-14 membered heteroaryl of R³ are each optionally substituted with 1, 2, 3, or 4 independently selected R^{D} substituents;
Cy¹ is phenyl optionally substituted with 1, 2, 3, or 4 independently selected R^{M} substituents, or C₁₀₋₁₄ aryl or 5-14 membered heteroaryl, wherein the C₁₀₋₁₄ aryl and 5-14 membered heteroaryl of Cy¹ is optionally substituted with 1, 2, 3, or 4 independently selected R^{E} substituents;
Cy² is C₆₋₁₄ aryl, C₃₋₁₄ cycloalkyl, 5-14 membered heteroaryl or 4-14 membered heterocycloalkyl, wherein the C₆₋₁₄ aryl, C₃₋₁₄ cycloalkyl, 5-14 membered heteroaryl and 4-14 membered heterocycloalkyl of Cy² are each optionally substituted with 1, 2, 3, or 4 independently selected R^{F} substituents;
each R^{a2}, R^{c2}, R^{d2}, and R^{a1} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a2}, R^{c2}, R^{d2}, and R^{a1} are each optionally substituted with 1, 2, 3, or 4 independently selected R^{G} substituents;
each R^{b2} is independently selected from C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{G} substituents;
each R^{C}, R^{D}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{c4}R^{d4}, C(O)OR^{a4} OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{c4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{C}, R^{D}, R^{E}, R^{F}, and R^{G} are each optionally substituted with 1, 2, 3, or 4 independently selected R^{E} substituents;
each R^{M} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, 2-CN, 3-CN, NO₂, OR^{a4}, SR^{a4}, NHOR^{a4}, C(O)R^{b4}, C(O)NR^{c4}R^{d4}, C(O)OR^{a4} OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{M} are each optionally substituted with 1, 2, 3, or 4 independently selected R^{E} substituents;
each R^{a4}, R^{c4}, and R^{d4} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4} are each optionally substituted with 1, 2, 3, or 4 independently selected R^{E} substituents;
each R^{b4} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{E} substituents;
each R^{e4} is independently selected from H, OH, CN, C₁₋₆ alkyl, C₁₋₆ alkoxy, C₁₋₆ haloalkyl, and C₁₋₆ haloalkoxy;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, CN, NO₂, OR^{a5}, SR^{a5}, NHOR^{a5}, C(O)R^{b5}, C(O)NR^{c5}R^{a5}, C(O)OR^{a5}, OC(O)R^{b5} OC(O)NR^{c5}R^{d5}, NR^{c5}R^{d5}, NR^{c5}C(O)R^{b5}NR^{c5}C(O)OR^{d5}, NR^{c5}C(O)NR^{c5}R^{d5}, C(═NR^{e5})R^{b5}, C(═NR^{e5})NR^{c5}R^{a5} NR^{c5}C(═NR^{e5})NR^{c5}R^{a5} NR^{c5}S(O)R^{b5}, NR^{c5}S(O)₂R^{b5}, and NR^{c5}S(O)₂NR^{c5}R^{a5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, of R^{E} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4}, are each optionally substituted with 1, 2, 3, or 4 independently selected R^{I} substituents;
each R^{b5} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, and C₁₋₆ haloalkoxy;
each R¹ is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, CN, NO₂, OR^{a6}, SR^{a6}, NHOR^{a6}, C(O)R^{b6}, C(O)NR^{c6}R^{d6}, C(O)OR^{a6}, OC(O)R^{b6} OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{e6})R^{b6}, C(═NR^{e6})NR^{c6}R^{d6}, NR^{c6}C(═NR^{e6})NR^{c6}R^{d6}, NR^{c6}S(O)R^{b6}, NR^{c6}S(O)₂R^{b6}, and NR^{c6}S(O)₂NR^{c6}R^{d6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, of R^{E} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a6}, R^{c6}, and R^{d6} are each optionally substituted with 1, 2, 3, or 4 independently selected R^{J} substituents;
each R^{b6} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, and C₁₋₆ haloalkoxy;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, CN, NO₂, OR^{a7}, SR^{a7}, NHOR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{e7})R^{b7}, C(═NR^{e7})NR^{c7}R^{d7}, NR^{c7}C(═NR^{e7})NR^{c7}R^{d7}, NR^{c7}S(O)R^{b7}, NR^{c7}S(O)₂R^{b7}, and NR^{c7}S(O)₂NR^{c7}R^{d7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, of R^{H} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a7}, R^{c7}, and R^{d7} are each optionally substituted with 1, 2, 3, or 4 independently selected R^{K} substituents;
each R^{b7} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{K} substituents;
each R^{e7} is independently selected from H, OH, CN, C₁₋₆ alkyl, C₁₋₆ alkoxy, C₁₋₆ haloalkyl, and C₁₋₆ haloalkoxy;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl; and
wherein any heteroaryl group of any of the above-recited substituents optionally comprises an N-oxide on any ring-forming nitrogen.

In some embodiments,
X is CR³;
R¹ is selected from H and C₁₋₆ alkyl;
R² is selected from H, D, C₆₋₁₄ aryl, 5-14 membered heteroaryl, C(O)R^{b2}, C(O)NR^{c2}R^{d2}, and C(O)OR^{a2}, wherein the C₆₋₁₄ aryl and 5-14 membered heteroaryl of R² are each optionally substituted with 1, 2, 3, or 4 independently selected R^{C} substituents;
R³ is selected from H, D, halo, C₁₋₆ alkyl, C₆₋₁₄ aryl, 5-14 membered heteroaryl, CN, and OR^{a3}, wherein the C₁₋₆ alkyl, C₆₋₁₄ aryl, and 5-14 membered heteroaryl of R³ are each optionally substituted with 1, 2, 3, or 4 independently selected R^{D} substituents;
Cy¹ is phenyl, optionally substituted with 1, 2, 3, or 4 independently selected R^{M} substituents; or Cy¹ is C₁₀₋₁₄ aryl or 5-14 membered heteroaryl, wherein the C₁₀₋₁₄ aryl and 5-14 membered heteroaryl of Cy¹ are each optionally substituted with 1, 2, 3, or 4 independently selected R^{E} substituents;
Cy² is C₆₋₁₄ aryl, C₃₋₁₄ cycloalkyl, 5-14 membered heteroaryl or 4-14 membered heterocycloalkyl, wherein the C₆₋₁₄ aryl, C₃₋₁₄ cycloalkyl, 5-14 membered heteroaryl and 4-14 membered heterocycloalkyl of Cy² are each optionally substituted with 1, 2, 3, or 4 independently selected R^{F} substituents;
each R^{a2}, R^{c2}, R^{d2}, and Rai is independently selected from H, C₁₋₆ alkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a2}, R^{c2}, R^{d2}, and Rai are each optionally substituted with 1, 2, 3, or 4 independently selected R^{G} substituents;
each R^{b2} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{G} substituents;
each R^{C}, R^{D}, R^{E}, R^{F}, and R^{G} is independently selected from D, halo, oxo, C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{c4}R^{d4}, C(O)OR^{a4}, OC(O)R^{b4} OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{C}, R^{D}, R^{E}, R^{F}, and R^{G} are each optionally substituted with 1, 2, 3, or 4 independently selected R^{E} substituents;
each R^{M} is independently selected from D, halo, oxo, C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, 2-CN, 3-CN, NO₂, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{M} are each optionally substituted with 1, 2, 3, or 4 independently selected R^{E} substituents;
each R^{a4}, R^{c4}, and R^{d4}, is independently selected from H, C₁₋₆ alkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4} are each optionally substituted with 1, 2, 3, or 4 independently selected R^{E} substituents;
each R^{b4} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{E} substituents;
each R^{e4} is independently selected from H, OH, CN, C₁₋₆ alkyl, C₁₋₆ alkoxy, C₁₋₆ haloalkyl, and C₁₋₆ haloalkoxy;
each R^{H} is independently selected from D, halo, oxo, C₁₋₆ alkyl, CN, NO₂, OR^{a5}, SR^{a5}, NHOR^{a5}, C(O)R^{b5}, C(O)NR^{c5}R^{a5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{b5})NR^{c5}R^{d5}, NR^{c5}S(O)R^{b5}, NR^{c5}S(O)₂R^{b5}, and NR^{c5}S(O)₂NR^{c5}R^{a5}; and
each R^{a5}, R^{c5}, and R^{d5} is independently selected from H, and C₁₋₆ alkyl;
each R^{b5} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl; and
each R^{e5} is independently selected from H and C₁₋₆ alkyl.

In some embodiments,
X is CR³;
R¹ is selected from H and C₁₋₆ alkyl;
R² is selected from H, D, C₆₋₁₀ aryl, 5-10 membered heteroaryl, C(O)R^{b2}, C(O)NR^{c2}R^{d2}, and C(O)OR^{a2}, wherein the C₆₋₁₀ aryl and 5-10 membered heteroaryl of R² are each optionally substituted with 1, 2, 3, or 4 independently selected R^{C} substituents;
R³ is selected from H, D, halo, C₁₋₆ alkyl, C₆₋₁₀ aryl, 5-10 membered heteroaryl, CN, and OR^{a3}, wherein the C₁₋₆ alkyl, C₆₋₁₀ aryl, and 5-10 membered heteroaryl of R³ are each optionally substituted with 1, 2, 3, or 4 independently selected R^{D} substituents;
Cy¹ is phenyl, optionally substituted with 1, 2, 3, or 4 independently selected R^{M} substituents; or Cy¹ is C₁₀ aryl, 4-10 membered heterocycloalkyl, or 5-10 membered heteroaryl, wherein the C₁₀ aryl, 4-10 membered heterocycloalkyl, and 5-10 membered heteroaryl of Cy¹ are each optionally substituted with 1, 2, 3, or 4 independently selected R^{E} substituents;
Cy² is C₆₋₁₀ aryl, C₃₋₇ cycloalkyl, 5-10 membered heteroaryl or 4-10 membered heterocycloalkyl, wherein the C₆₋₁₀ aryl, C₃₋₇ cycloalkyl, 5-10 membered heteroaryl and 4-10 membered heterocycloalkyl of Cy² are each optionally substituted with 1, 2, 3, or 4 independently selected R^{F} substituents;
each R^{a2}, R^{c2}, R^{d2}, and R^{a1} is independently selected from H, C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl of R^{a2}, R^{c2}, R^{d2}, and R^{a1} are each optionally substituted with 1, 2, 3, or 4 independently selected R^{G} substituents;
each R^{b2} is independently selected from C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl are each optionally substituted with 1, 2, 3, or 4 independently selected R^{G} substituents;
each R^{C}, R^{D}, R^{E}, R^{F}, and R^{G} is independently selected from D, halo, oxo, C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, CN, NO₂, OR^{a4}, SR^{a4}, NHOR^{a4}, C(O)R^{b4}, C(O)NR^{c4}R^{d4}, C(O)OR^{a4}, OC(O)R^{b4} OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl of R^{C}, R^{D}, R^{E}, R^{F}, and R^{G} are each optionally substituted with 1, 2, 3, or 4 independently selected R^{E} substituents;
each R^{M} is independently selected from D, halo, oxo, C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, 2-CN, 3-CN, NO₂, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl of R^{M} are each optionally substituted with 1, 2, 3, or 4 independently selected R^{H} substituents;
each R^{a4}, R^{c4}, and R^{d4}, is independently selected from H, C₁₋₆ alkyl, C₂₋₆ alkenyl, C₂₋₆ alkynyl, C₆₋₁₀ aryl, C₃₋₇cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₇cycloalkyl, 5-10 membered heteroaryl, and 4-10 membered heterocycloalkyl of R^{a4}, R^{c4}, and R^{d4}, are each optionally substituted with 1, 2, 3, or 4 independently selected R^{E} substituents;
each R^{b4} is independently selected from C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₇ cycloalkyl, 5-10 membered heteroaryl, and 4-10 membered heterocycloalkyl are each optionally substituted with 1, 2, 3, or 4 independently selected R^{E} substituents;
each R^{e4} is independently selected from H, OH, CN, C₁₋₆ alkyl, C₁₋₆ alkoxy, C₁₋₆ haloalkyl, and C₁₋₆ haloalkoxy;
each R^{H} is independently selected from D, halo, oxo, C₁₋₆ alkyl, CN, NO₂, OR^{a5}, SR^{a5}, NHOR^{a5}, C(O)R^{b5}, C(O)NR^{c5}R^{a5}, C(O)OR^{a5}, OC(O)R^{b5}, OC(O)NR^{c5}R^{d5} NR^{c5}R^{a5} NR^{c5}C(O)R^{b5} NR^{c5}C(O)OR^{a5}, NR^{c5}C(O)NR^{c5}R^{d5}, C(═NR^{e5})R^{b5}, C(═NR^{e5})NR^{c5}R^{d5} NR^{c5}C(═NR^{e5})NR^{c5}R^{a5}, NR^{c5}S(O)R^{b5}, NR^{c5}S(O)₂R^{b5}, and NR^{c5}S(O)₂NR^{c5}R^{d5}; and
each R^{a5}, R^{c5}, and R^{d5} is independently selected from H, and C₁₋₆ alkyl;
each R^{b5} is independently selected from C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl; and
each R^{e5} is independently selected from H and C₁₋₆ alkyl.

In some embodiments,
X is N;
R¹ is selected from H, C₁₋₆ alkyl, and a 5-14 membered heteroaryl, wherein the C₁₋₆ alkyl and a 5-14 membered heteroaryl are each optionally substituted with 1, 2, or 3 independently selected R^{B} substituents;
R² is selected from H, D, C₁₋₆ alkyl, C₆₋₁₄ aryl, 5-14 membered heteroaryl, 4-14 membered heterocycloalkyl, C₆₋₁₄ aryl-C₁₋₆ alkyl-, (5-14 membered heteroaryl)-C₁₋₆ alkyl-, (4-14 membered heterocycloalkyl)-C₁₋₆ alkyl-, NR^{c2}R^{d2}, C(O)R^{b2}, C(O)NR^{c2}R^{d2}, and C(O)OR^{a2}, wherein the C₁₋₆ alkyl, C₆₋₁₄ aryl, 5-14 membered heteroaryl, 4-14 membered heterocycloalkyl, C₆₋₁₄ aryl-C₁₋₆ alkyl-, (5-14 membered heteroaryl)-C₁₋₆ alkyl-, (4-14 membered heterocycloalkyl)-C₁₋₆ alkyl- of R² are each optionally substituted with 1, 2, 3, or 4 independently selected R^{C} substituents;
Cy¹ is phenyl, optionally substituted with 1, 2, 3, or 4 independently selected R^{M} substituents, or C₁₀₋₁₄ aryl or 5-14 membered heteroaryl, wherein the C₁₀₋₁₄ aryl and 5-14 membered heteroaryl of Cy¹ is optionally substituted with 1, 2, 3, or 4 independently selected R^{E} substituents;
Cy² is C₆₋₁₄ aryl, C₃₋₁₄ cycloalkyl, 5-14 membered heteroaryl or 4-14 membered heterocycloalkyl, wherein the C₆₋₁₄ aryl, C₃₋₁₄ cycloalkyl, 5-14 membered heteroaryl and 4-14 membered heterocycloalkyl of Cy² are each optionally substituted with 1, 2, 3, or 4 independently selected R^{F} substituents;
each R^{a2}, R^{c2}, and R^{d2} is independently selected from H, C₁₋₆ alkyl, C₂₋₆ alkenyl, C₂₋₆ alkynyl, C₆₋₁₄ aryl, C₆₋₁₄ aryl-C₁₋₆ alkyl-, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₆₋₁₄ aryl-C₁₋₆ alkyl-, C₃₋₁₄ cycloalkyl, 5-14 membered heteroaryl, and 4-14 membered heterocycloalkyl of R^{a2}, R^{c2}, and R^{d2} are each optionally substituted with 1, 2, 3, or 4 independently selected R^{G} substituents;
each R^{b2} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{G} substituents;
each R^{B}, R^{C}, R^{E}, R^{F}, and R^{G} is independently selected from D, halo, oxo, C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{c4}R^{d4}, C(O)OR^{a4}, OC(O)R^{b4} OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{B}, R^{C}, R^{E}, R^{F}, and R^{G} are each optionally substituted with 1, 2, 3, or 4 independently selected R^{E} substituents;
each R^{M} is independently selected from D, halo, oxo, C₁₋₆ alkyl, C₂₋₆ alkenyl, C₂₋₆ alkynyl, C₆₋₁₄ aryl, C₃₋₁₄cycloalkyl, 5-14 membered heteroaryl, 4-14 membered heterocycloalkyl, 2-CN, 3-CN, NO₂, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{M} are each optionally substituted with 1, 2, 3, or 4 independently selected R^{E} substituents;
each R^{a4}, R^{c4}, and R^{d4}, is independently selected from H, C₁₋₆ alkyl, C₂₋₆ alkenyl, C₂₋₆ alkynyl, C₆₋₁₄ aryl, C₃₋₁₄cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4}, are each optionally substituted with 1, 2, 3, or 4 independently selected R^{E} substituents;
each R^{b4} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{E} substituents;
each R^{e4} is independently selected from H, OH, CN, C₁₋₆ alkyl, C₁₋₆ alkoxy, C₁₋₆ haloalkyl, and C₁₋₆ haloalkoxy;
each R^{H} is independently selected from D, halo, oxo, C₁₋₆ alkyl, CN, NO₂, OR^{a5}, SR^{a5}, NHOR^{a5}, C(O)R^{b5}, C(O)NR^{c5}R^{a5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{e5})NR^{c5}R^{d5}, NR^{c5}C(═NR^{b5})NR^{c5}R^{d5}, NR^{c5}S(O)R^{b5}, NR^{c5}S(O)₂R^{b5}, and NR^{c5}S(O)₂NR^{c5}R^{d5}; and
each R^{a5}, R^{c5}, and R^{d5} is independently selected from H, and C₁₋₆ alkyl;
each R^{b5} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl; and
each R^{e5} is independently selected from H and C₁₋₆ alkyl.

In some embodiments,
X is N;
R¹ is H or a 5-14 membered heteroaryl optionally substituted with 1, 2, or 3 independently selected R^{B} substituents;
R² is selected C₁₋₆ alkyl, 4-14 membered heterocycloalkyl, C₆₋₁₄ aryl-C₁₋₆ alkyl-, (5-14 membered heteroaryl)-C₁₋₆ alkyl-, (4-14 membered heterocycloalkyl)-C₁₋₆ alkyl-, and NR^{c2}R^{d2}, wherein the C₁₋₆ alkyl, 4-14 membered heterocycloalkyl, C₆₋₁₄ aryl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, or 3 independently selected R^{C} substituents;
Cy¹ is phenyl optionally substituted with 1, 2, or 3 independently selected R^{E} substituents; and
Cy² is 5-14 membered heteroaryl optionally substituted with 1, 2, or 3 independently selected R^{F} substituents.

In some embodiments,
X is N;
R¹ is H or a 5-14 membered heteroaryl optionally substituted with 1, 2, or 3 independently selected R^{B} substituents;
each R^{B} is independently selected from D, halo, oxo, C₁₋₆ alkyl, C₁₋₆ haloalkyl, C₂₋₆ alkenyl, CN, NO₂ and OH;
R² is selected C₁₋₆ alkyl, 4-14 membered heterocycloalkyl, C₆₋₁₄ aryl-C₁₋₆ alkyl-, (5-14 membered heteroaryl)-C₁₋₆ alkyl-, (4-14 membered heterocycloalkyl)-C₁₋₆ alkyl-, and NR^{c2}R^{d2}, wherein the C₁₋₆ alkyl, 4-14 membered heterocycloalkyl, C₆₋₁₄ aryl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, or 3 independently selected R^{C} substituents;
each R^{c2} and R^{d2} are independently selected from H, C₁₋₆ alkyl, C₆₋₁₄ aryl-C₁₋₆ alkyl-, wherein the C₁₋₆ alkyl and C₆₋₁₄ aryl-C₁₋₆ alkyl- of R^{c2} and R^{d2} are each optionally substituted with 1, 2, or 3 independently selected R^{G} substituents;
each R^{C} is independently selected from D, halo, oxo, C₁₋₆ alkyl, C₆₋₁₄ aryl, C₃₋₁₄ cycloalkyl, 5-14 membered heteroaryl, 4-14 membered heterocycloalkyl, OR^{a4}, C(O)NR^{c4}R^{d4}, and NR^{c4}R^{d4}, wherein the C₁₋₆ alkyl, C₆₋₁₄ aryl, C₃₋₁₄ cycloalkyl, 5-14 membered heteroaryl, and 4-14 membered heterocycloalkyl, of R^{c} is optionally substituted with 1, 2, or 3 independently selected R^{H} substituents;
each R^{a4}, R^{c4}, and R^{d4}, is independently selected from H, C₁₋₆ alkyl, C₁₋₆ haloalkyl, C₂₋₆ alkenyl, and 5-14 membered heteroaryl, wherein the C₁₋₆ alkyl, C₂₋₆ alkenyl, and 5-14 membered heteroaryl, of R^{a4}, R^{c4}, and R^{d4}, are each optionally substituted with 1, 2, or 3 independently selected R^{E} substituents;
each R^{G} is independently selected from D, halo, oxo, C₁₋₆ alkyl, CN, NO₂, and OH;
Cy¹ is phenyl optionally substituted with 1, 2, or 3 independently selected R^{E} substituents;
each R^{E} is independently selected from D, halo, oxo, C₁₋₆ alkyl, CN, NO₂ and OH;
Cy² is 5-14 membered heteroaryl optionally substituted with 1, 2, or 3 independently selected R^{F} substituents;
each R^{F} is independently selected from D, halo, oxo, C₁₋₆ alkyl, C₂₋₆ alkenyl, CN, NO₂, and OR^{a4}, wherein the C₁₋₆ alkyl and C₂₋₆ alkenyl of R^{F} are each optionally substituted with 1, 2, or 3 independently selected R^{E} substituents;
each R^{a4} is independently selected from H, C₁₋₆ alkyl and C₁₋₆ alkoxy; and
each R^{H} is independently selected from D, halo, oxo, C₁₋₆ alkyl, CN, NO₂ and OH.

In some embodiments,
X is N;
R¹ is H or a 5-10 membered heteroaryl optionally substituted with 1, 2, or 3 independently selected R^{B} substituents;
each R^{B} is independently selected from D, halo, oxo, C₁₋₆ alkyl, C₁₋₆ haloalkyl, C₂₋₆ alkenyl, CN, NO₂ and OH;
R² is selected C₁₋₆ alkyl, 4-10 membered heterocycloalkyl, C₆₋₁₀ aryl-C₁₋₆ alkyl-, (5-10 membered heteroaryl)-C₁₋₆ alkyl-, (4-10 membered heterocycloalkyl)-C₁₋₆ alkyl-, and NR^{c2}R^{d2}, wherein the C₁₋₆ alkyl, 4-10 membered heterocycloalkyl, C₆₋₁₀ aryl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, or 3 independently selected R^{C} substituents;
each R^{c2} and R^{d2} are independently selected from H, C₁₋₆ alkyl, C₆₋₁₀ aryl-C₁₋₆ alkyl-, wherein the C₁₋₆ alkyl and C₆₋₁₀ aryl-C₁₋₆ alkyl- of R^{c2} and R^{d2} are each optionally substituted with 1, 2, or 3 independently selected R^{G} substituents;
each R^{C} is independently selected from D, halo, oxo, C₁₋₆ alkyl, C₆₋₁₀ aryl, C₃₋₇ cycloalkyl, 5-10 membered heteroaryl, 4-10 membered heterocycloalkyl, OR^{a4}, C(O)NR^{c4}R^{d4}, and NR^{c4}R^{d4}, wherein the C₁₋₆ alkyl, C₆₋₁₀ aryl, C₃₋₇ cycloalkyl, 5-10 membered heteroaryl, and 4-10 membered heterocycloalkyl, of R^{c} is optionally substituted with 1, 2, or 3 independently selected R^{E} substituents;
each R^{a4}, R^{c4}, and R^{d4}, is independently selected from H, C₁₋₆ alkyl, C₁₋₆ haloalkyl, C₂₋₆ alkenyl, and 5-10 membered heteroaryl, wherein the C₁₋₆ alkyl, C₂₋₆ alkenyl, and 5-10 membered heteroaryl, of R^{a4}, R^{c4}, and R^{d4}, are each optionally substituted with 1, 2, or 3 independently selected R^{E} substituents;
each R^{G} is independently selected from D, halo, oxo, C₁₋₆ alkyl, CN, NO₂, and OH;
Cy¹ is phenyl optionally substituted with 1, 2, or 3 independently selected R^{E} substituents;
each R^{E} is independently selected from D, halo, oxo, C₁₋₆ alkyl, CN, NO₂ and OH;
Cy² is 5-10 membered heteroaryl optionally substituted with 1, 2, or 3 independently selected R^{F} substituents;
each R^{F} is independently selected from D, halo, oxo, C₁₋₆ alkyl, C₂₋₆ alkenyl, CN, NO₂, and OR^{a4}, wherein the C₁₋₆ alkyl and C₂₋₆ alkenyl of R^{F} are each optionally substituted with 1, 2, or 3 independently selected R^{E} substituents;
each R^{a4} is independently selected from H, C₁₋₆ alkyl and C₁₋₆ alkoxy; and
each R^{H} is independently selected from D, halo, oxo, C₁₋₆ alkyl, CN, NO₂ and OH.

In some embodiments,
X is N;
R¹ is H or nicotinonitrile;
R² is pyridinylmethyl, hydroxy(phenyl)methyl, hydroxyethylamino(phenyl)ethyl, cyclohexylmethyl, fluorobenzyl, hydroxy(fluorophenyl)methyl, methylpyridinylmethyl, fluoropyridinylmethyl, methoxypyridinylmethyl, methylpyrazolylbenzyl-benzoisoxazolylmethyl, methylindazolylmethyl, hydroxyazetidinylmethyl, benzoyl, phenylcyclopropyl, cyano(phenyl)methylamino, tetrahydrofuranyl, or phenyl(pyridin-2-yloxy)methyl;
Cy¹ is cyanophenyl; and
Cy² is pyrimidinyl, ethylpyrazolyl propylpyrazolyl, quinolinyl, fluoropyrimidinyl, pyridinyl, methylpyridinyl, methoxy-methylpyridinyl, pyrazolopyridazinyl, methyloxazolyl, hydroxymethyl-methyloxazolyl, or methoxymethyl-methyloxazolyl.

In some embodiments,
X is N;
R¹ is H;
R² is selected from pyridin-2-ylmethyl, hydroxy(phenyl)methyl, (2-hydroxyethylamino)(phenyl)methyl, cyclohexylmethyl, 2-fluorobenzyl, (2-fluorophenyl)(hydroxy)methyl, (6-methylpyridin-2-yl)methyl, (3-fluoropyridin-2-yl)methyl, (3-methoxypyridin-2-yl)methyl, 2-(1-methyl-1H-pyrazol-4-yl)benzyl, benzo[d]isoxazol-3-ylmethyl, (1-methyl-1H-indazol-3-yl)methyl, (3-hydroxyazetidin-1-yl)methyl, benzoyl, 1-phenylcyclopropyl, (cyano(phenyl)methyl)amino, tetrahydrofuran-3-yl, phenyl(pyridin-2-yloxy)methyl, 2-fluoro-6-(((3R,4R)-3-fluoro-4-hydroxypyrrolidin-1-yl)methyl)benzyl, 2-((4-carboxypiperidin-1-yl)methyl)-6-fluorobenzyl, 2-fluoro-6-((N-methylmethylsulfonamido)methyl)benzyl, 2-((2,5-dioxoimidazolidin-1-yl)methyl)-6-fluorobenzyl, (2,6-difluorophenyl)(hydroxy)methyl, (5-(pyridin-2-yl)-1H-tetrazol-1-yl)methyl, (5-(1H-pyrazol-1-yl)-1H-tetrazol-1-yl)methyl, (5-(thiazol-4-yl)-1H-tetrazol-1-yl)methyl, (5-methyl-3-(trifluoromethyl)-1H-pyrazol-1-yl)methyl, (3-methylpyridin-2-yl)methyl, 2-((1,1-dioxidoisothiazolidin-2-yl)methyl)-6-fluorobenzyl, 2-fluoro-6-((3-methyl-2,5-dioxoimidazolidin-1-yl)methyl)benzyl, (6-(trifluoromethyl)pyridin-2-yl)methyl, (3-(hydroxymethyl)pyridin-2-yl)methyl, (1-methyl-1H-pyrazol-3-yl)methyl, and (2-cyanophenoxy)methyl, (3-methylpyridin-2-yl)methoxy, (6-methylpyridin-2-yl)methoxy, and ((3-methylpyridin-2-yl)methyl)amino;
Cy¹ is cyanophenyl; and
Cy² is selected from 2,6-dimethylpyridin-4-yl, pyridin-4-yl, 2-methylpyridin-4-yl, 1-carbamoyl-1,2,3,6-tetrahydropyridin-4-yl, 1-carbamoylpiperidin-4-yl, 2-methoxypyridin-4-yl, 2-methoxy-6-methylpyridin-4-yl, 2,6-dimethylpyridin-4-yl-1-oxide, 1-ethyl-6-oxo-1,6-dihydropyridin-3-yl, 3-methylpyridin-4-yl, 3-fluoropyridin-4-yl, 3-chloropyridin-4-yl, 3-methoxypyridin-4-yl, 3-cyanopyridin-4-yl, 4-carbamoylphenyl, pyrazolo[1,5-a]pyridin-3-yl, pyrazolo[1,5-b]pyridazin-3-yl, 5-methyl-1H-pyrazol-4-yl, 1-ethyl-1H-pyrazol-5-yl, 1-isopropyl-1H-pyrazol-5-yl, 1-propyl-1H-pyrazol-5-yl, pyrimidin-4-yl, 2,3-dihydro-[1,4]dioxino[2,3-b]pyridin-8-yl, quinolin-5-yl, 5-fluoropyrimidin-4-yl, oxazol-5-yl, 4-methyloxazol-5-yl, 4-ethyloxazol-5-yl, 4-(hydroxymethyl)-2-methyloxazol-5-yl, 4-(methoxymethyl)-2-methyloxazol-5-yl, 4-(hydroxymethyl)-2-methyloxazol-5-yl, 1-ethyl-1H-1,2,3-triazol-5-yl, and cyclopropyl.

In some embodiments, the compound is the (S)-enantiomer of one of the preceding compounds, or a pharmaceutically acceptable salt thereof. In some embodiments, the compound is the (R)-enantiomer of one of the preceding compounds, or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl”, employed alone or in combination with other tens, refers to an aromatic hydrocarbon group, which may be monocyclic or polycyclic (e.g., having 2, 3, or 4 fused rings). The term “Cₙ₋ₘ aryl” refers to an aryl group having from n tom ring carbon atoms. Aryl groups include, e.g., phenyl, naphthyl, anthracenyl, phenanthrenyl, indanyl, indenyl, and the like. In some embodiments, aryl groups have from 5 to 14 carbon atoms. In some embodiments, the aryl group has from 5 to 10 carbon atoms. In some embodiments, the aryl group is phenyl or naphthyl. In some embodiments, the aryl group is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCF₂. An example haloalkoxy group is OCHF₂. In some embodiments, the haloalkoxy group is fluorinated only. In some embodiments, the alkyl group has 1 to 6, 1 to 4, or 1 to 3 carbon atoms.

As used herein, the teen “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CCl₃, CHCl₂, C₂Cl₅ (and the like.

As used herein, the term “thio” refers to a group of formula —SH.

As used herein, the term “carbamyl” to a group of formula —C(O)NH₂.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3, or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, 12, 13, or 14 ring-forming carbons (i.e., C₃₋₁₄). In some embodiments, the cycloalkyl is a monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, S, and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-6 monocyclic heteroaryl having 1, 2, or 3 heteroatom ring members independently selected from N, O, S, and B. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, S, and B. In some embodiments, the heteroaryl group contains 3 to 14, 4 to 14, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridine, pyrimidine, pyrazine, pyridazine, pyrrole, pyrazole, azolyl, oxazole, isoxazole, thiazole, isothiazole, imidazole, furan, thiophene, triazole, tetrazole, thiadiazole, quinoline, isoquinoline, indole, benzothiophene, benzofuran, benzisoxazole, imidazo[1,2-b]thiazole, purine, triazine, thieno[3,2-b]pyridine, imidazo[1,2-a]pyridine, 1,5-naphthyridine, 1H-pyrazolo[4,3-b]pyridine, and the like.

A five-membered heteroaryl is a heteroaryl group having five ring-forming atoms wherein one or more (e.g., 1, 2, or 3) of the ring-forming atoms are independently selected from N, O, B, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine.

A six-membered heteroaryl ring is a heteroaryl with a ring having six ring-forming atoms wherein one or more (e.g., 1, 2, or 3) ring atoms are independently selected from N, O, S, and B. Exemplary six-membered ring heteroaryls are pyridyl, pyrazinyl, pyrimidinyl, triazinyl and pyridazinyl.

As used herein, “heterocycloalkyl” refers to monocyclic or polycyclic heterocycles having at least one non-aromatic ring (saturated or partially saturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfide (e.g., C(O), S(O), C(S), or S(O)₂, etc). Heterocycloalkyl groups include monocyclic and polycyclic (e.g., having 2, 3, or 4 fused rings) systems. Included in heterocycloalkyl are monocyclic and polycyclic 3-14-, 4-14-, 3-10-, 4-10-, 5-10-, 4-7-, 5-7-, 5-6-, 5- or 6-membered heterocycloalkyl groups. Heterocycloalkyl groups can also include spirocycles and bridged rings (e.g., a 5-14 membered bridged biheterocycloalkyl ring having one or more ring-forming carbon atoms replaced by a heteroatom independently selected from N, O, S. and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Example heterocycloalkyl groups include pyrrolidonyl, pyrrolidin-2-one, 1,3-isoxazolidin-2-one, pyranyl, tetrahydropyran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl and the like. In some embodiments, the heterocycloalkyl group is pyrrolidonyl, pyrrolidin-2-one, 1,3-isoxazolidin-2-one, pyranyl, tetrahydropuran, oxetanyl, azetidinyl, morpholinyl, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, or azepanyl.

In some embodiments, the heterocycloalkyl group contains 3 to 14 ring-forming atoms, 4 to 14 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S, and B and having one or more oxidized ring members. In some embodiments, the heterocycloalkyl is a monocyclic or bicyclic 4-10 membered heterocycloalkyl having 1, 2, 3, or 4 heteroatoms independently selected from N, O, S, and B and having one or more oxidized ring members.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

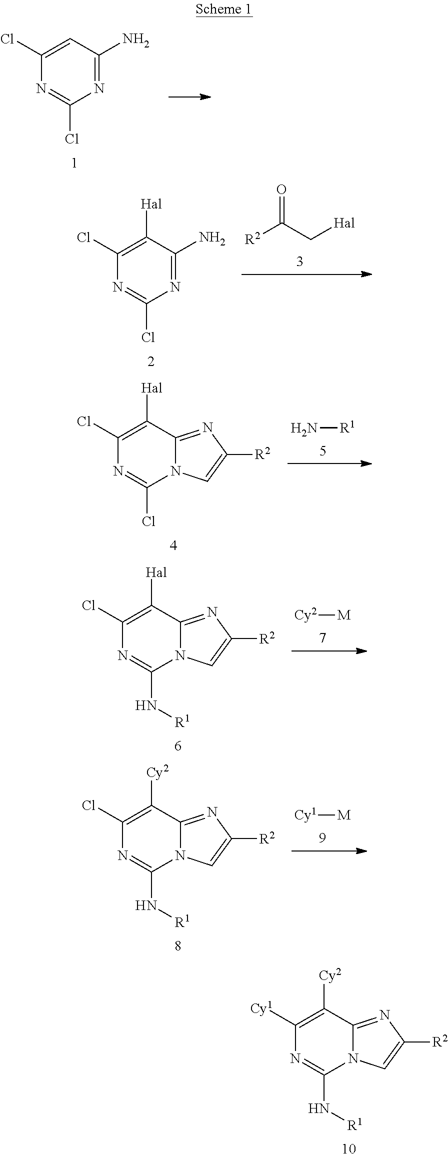

Compound of formula 10 can be prepared via the synthetic route as outlined in Scheme 1. The commercially available starting material 1 can undergo a halogenation reaction, such as electrophilic aromatic substitution (S_{E}Ar), with an appropriate reagent, such as N-bromosuccinimide (NBS), to afford compound 2 (Hal is a halide, such as F, Cl, Br, or I). Condensation of compound 2 with a carbonyl adduct of formula 3 at elevated temperature can generate the bicyclic compound 4. Selective chloride displacement of compound 4 via either nucleophilic substitution, or a coupling reaction, with compound 5 can deliver compound 6. Compound 6 can then be selectively coupled to an adduct of formula 7, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative of formula 8. Introduction of Cy¹ can then be achieved by the coupling of compound 8 with an adduct of formula 9, using similar conditions as described for the preparation of compound 8 from compound 6, to afford compound 10.

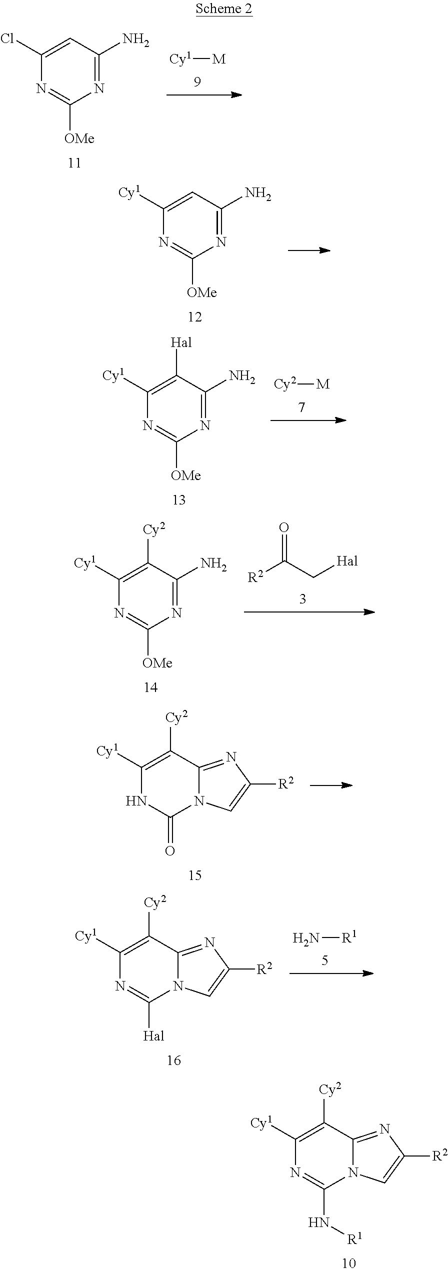

Alternatively, compound of formula 10 can be prepared via the synthetic route as outlined in Scheme 2. The commercially available starting material 11 can undergo a coupling reaction with an adduct of formula 9, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative of formula 12. Compound 12 can undergo a halogenation reaction, such as electrophilic aromatic substitution (S_{E}Ar), with an appropriate reagent, such as N-bromosuccinimide (NBS), to afford compound 13 (Hal is a halide, such as F, Cl, Br, or I). Coupling of compound 13 with an adduct of formula 7, using similar conditions as described for the preparation of compound 12 from compound 11, can afford compound 14. Condensation of compound 14 with a carbonyl adduct of formula 3 at elevated temperature can generate the bicyclic compound 15. Treatment of compound 15 with an appropriate reagent, such as phosphoryl chloride (POCl₃), at elevated temperature can afford the halide adduct 16.

Displacement of the halogen in compound 16 via nucleophilic substitution, or a coupling reaction, with adduct 5 can then afford compound 10.

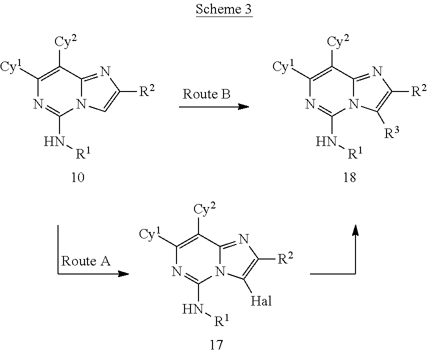

Compound 18 can be prepared via the synthetic route (Route A) as outlined in Scheme 3. Compound 10 can first undergo a halogenation reaction, such as electrophilic aromatic substitution (S_{E}Ar), with an appropriate reagent, such as N-bromosuccinimide (NBS), to afford compound 17 (Hal is a halide, such as F, Cl, Br, or I). R³ can then be introduced either via nucleophilic substitution, or a coupling reaction, to afford compound 18. Alternatively, compound 10 can undergo a direct chemical transformation, such as electrophilic substitution, to generate compound 18 (Route B).

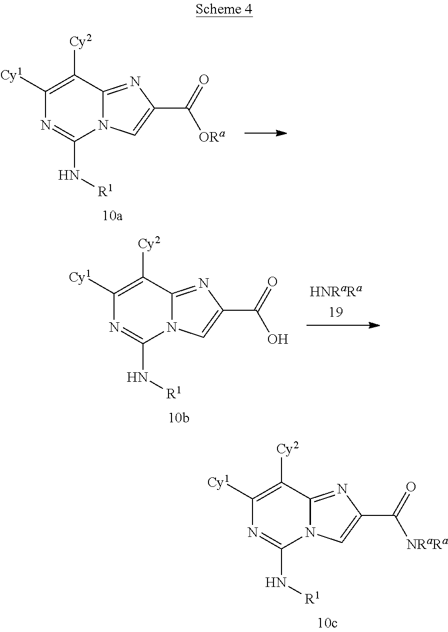

Compound 10c can be prepared via the synthetic route as outlined in Scheme 4 starting from compound 10a, which can be prepared as described in either Scheme 1 or Scheme 2. Ester hydrolysis of compound 10a using an appropriate reagent, such as lithium hydroxide (LiOH), can deliver carboxylic acid 10b, which can then be coupled with amine 19 using an appropriate coupling reagent (such as HATU, BOP, or PyBOP) to afford compound 10c.

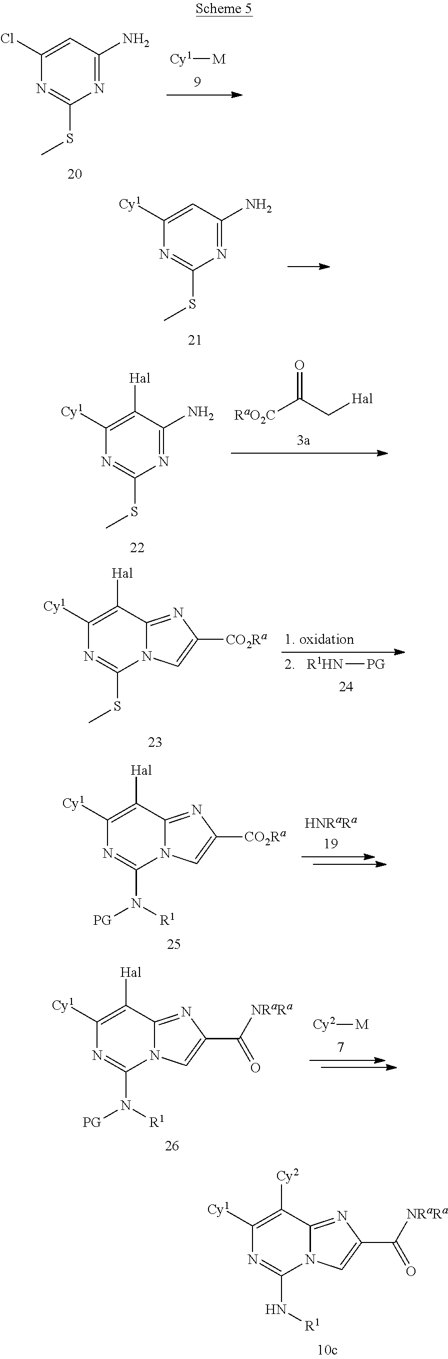

Alternatively, compound 10c can be prepared using the synthetic route as outlined in Scheme 5. The commercially available starting material 20 can undergo a coupling reaction with an adduct of formula 9, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative of formula 21. Compound 21 can then be subjected to a halogenation reaction, such as electrophilic aromatic substitution (S_{E}Ar), with an appropriate reagent, such as N-bromosuccinimide (NBS), to afford compound 22 (Hal is a halide, such as F, Cl, Br, or I). Condensation of compound 22 with a carbonyl adduct of formula 3a at elevated temperature can generate the bicyclic compound of formula 23. Oxidation of compound 23 with an appropriate oxidant, such as mCPBA, followed by nucleophilic substitution with a protected amine adduct 24 can deliver compound 25. Ester hydrolysis of compound 25 in the presence of an appropriate reagent, such as lithium hydroxide (LiOH), can generate the corresponding carboxylic acid, which can then be coupled with an amine adduct of formula 19, using an appropriate coupling reagent (such as HATU, BOP, or PyBOP), to afford compound 26. Alternatively, compound 26 can be accessed by reacting compound 25 directly with amine adduct 19 at elevated temperature. Finally, introduction of Cy² can be achieved by the coupling of compound 26 with an adduct of formula 7, using similar conditions as described for the preparation of compound 21 from compound 20. The protecting group (PG) can then be removed under appropriate conditions to afford compound 10c. Alternatively, compound 10c can also be prepared by first removal of the protecting group (PG) in compound 26, followed by the coupling reaction with adduct 7.

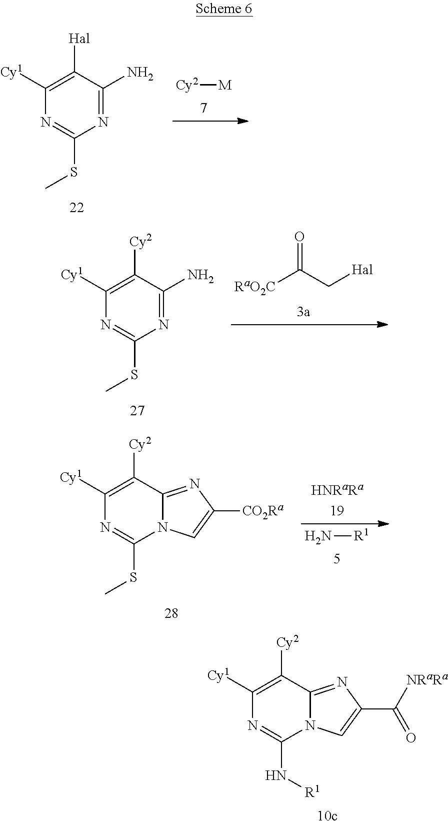

Compound 10c can also be prepared using the synthetic route as outlined in Scheme 6. Compound 22 (prepared as described in Scheme 5) can first undergo a coupling reaction with an adduct of formula 7, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative of formula 27. Condensation of compound 27 with a carbonyl adduct of formula 3a at elevated temperature can generate the bicyclic compound of formula 28. Compound 28 can then react with amine 19 and amine 5, in either order, to afford Compound 10c.

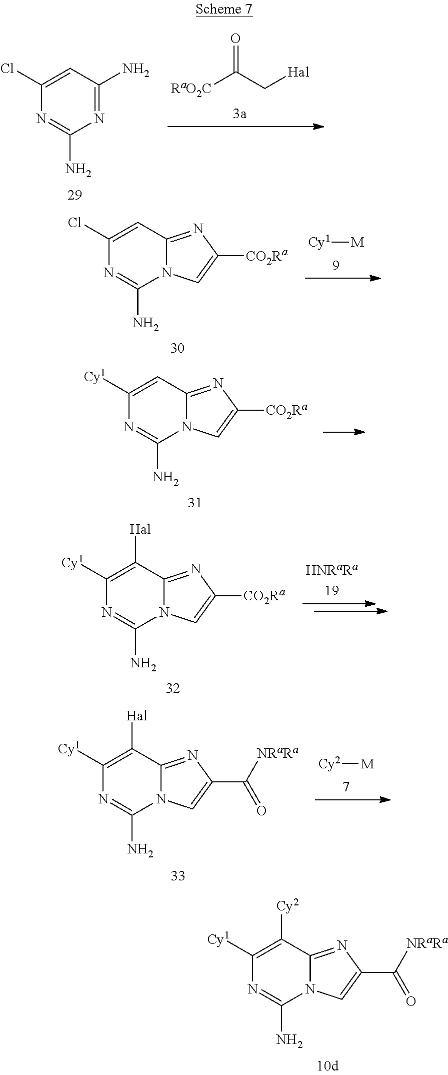

Compounds 10d can be prepared using the synthetic route as outlined in Scheme 7. Condensation of commercially available starting material 29 with a carbonyl adduct of formula 3a at elevated temperature can generate the bicyclic compound of formula 30. Compound 30 can then undergo a coupling reaction with an adduct of formula 9, in which M is a boronic acid, a boronic ester or an appropriately substituted metal [e.g., M is B(OR)₂, Sn(Alkyl)₃, Zn-Hal, etc.], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to give a derivative of formula 31. Compound 31 can then be subjected to a halogenation reaction, such as electrophilic aromatic substitution (S_{E}Ar), with an appropriate reagent, such as N-bromosuccinimide (NBS), to afford compound 32 (Hal is a halide, such as F, Cl, Br, or I). Ester hydrolysis of compound 32 in the presence of an appropriate reagent, such as lithium hydroxide (LiOH), can generate the corresponding carboxylic acid, which can then be coupled with an amine adduct of formula 19, using an appropriate coupling reagent (such as HATU, BOP, or PyBOP), to afford compound 33. Alternatively, compound 33 can be accessed by reacting compound 32 directly with amine adduct 19 at elevated temperature. Introduction of Cy² can then be achieved by the coupling of compound 33 with an adduct of formula 7, using similar conditions as described for the preparation of compound 31 from compound 30, to afford compound 10d.

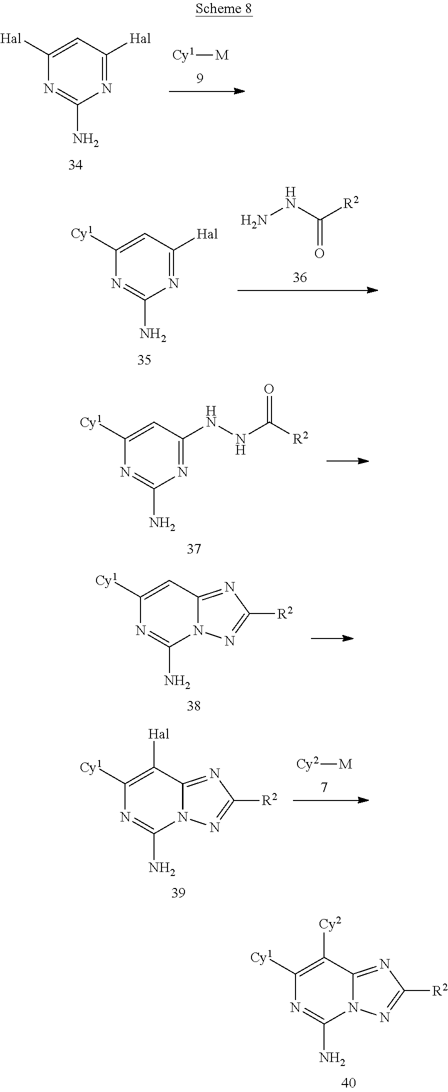

Compounds of formula 40 can be synthesized via the synthetic route outlined in Scheme 8. Starting material 34 first undergoes a cross-coupling reaction with reagent 9 to generate compound 35, in which M is a boronic acid, boronic ester or an appropriately substituted metal [e.g., M is B(OR)₂, Sn(Alkyl)₃, or Zn-Hal], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst). A nucleophilic aromatic substitution (S_{N}Ar) reaction of compound 35 with hydrazide 36 then affords compound 37, which undergoes a cyclization reaction at elevated temperature in the presence of a suitable reagent, such as N,O-bis(trimethylsilyl)acetamide, to produce bicycle 38. Halogenation of 38 with an appropriate reagent, such as N-bromosuccinimide (NBS), affords compound 39. The final product 40 can be prepared by a cross-coupling reaction between compound 39 and a derivative of formula 7, using similar procedures as described for the preparation of compound 35 from starting material 34. At various stages during this synthetic sequence, the R² group can be further functionalized as seen appropriate.

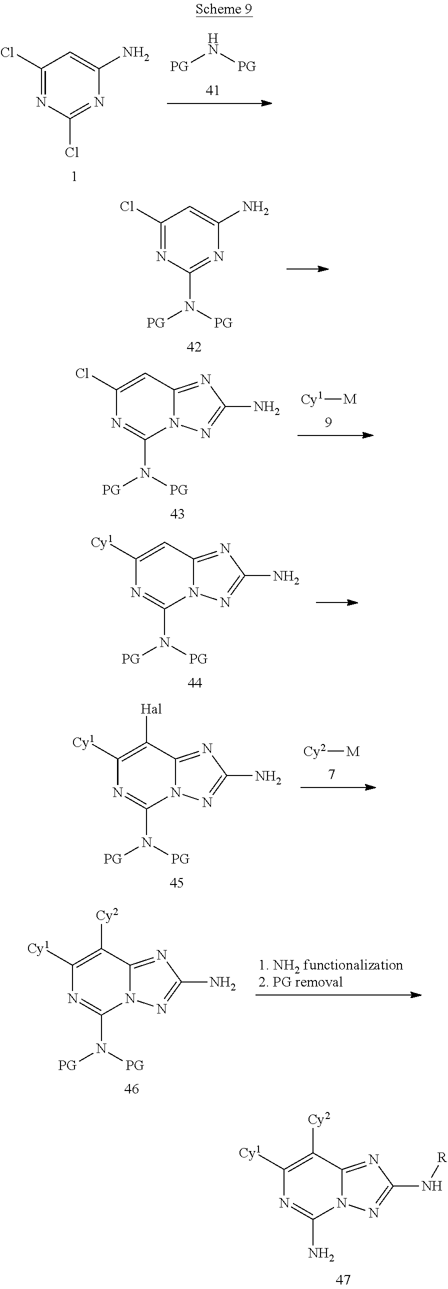

Compounds of formula 47 can be synthesized via the synthetic route outlined in Scheme 9. Selective nucleophilic aromatic substitution (S_{N}Ar) reaction of starting material 1 with amine 41 (PG represents a suitable protecting group, such as 4-methoxybenzyl) affords compound 42. Compound 42 can then be cyclized to intermediate 43 via appropriate chemical transformations, such as a two-step sequence using O-ethyl carbonisothiocyanatidate and hydroxylamine hydrochoride. A cross-coupling reaction between 43 and a reagent of formula 9, in which M is a boronic acid, boronic ester or an appropriately substituted metal [e.g., M is B(OR)₂, Sn(Alkyl)₃, or Zn-Hal], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), will generate intermediate 44. Halogenation of 44 using a suitable reagent, such as N-bromosuccinimide (NBS), gives compound 45. A cross-coupling reaction between 45 and a derivative of formula 7, using similar procedures as described for the preparation of compound 44 from compound 43, generates intermediate 46. The amino group of 46 can then be functionalized using suitable chemical transformations, such as Buchwald-Hartwig coupling conditions in the presence of a palladium catalyst (e.g., chloro(2-dicyclohexylphosphino-2′,4′,6′-triisopropyl-1,1′-biphenyl)[2-(2′-amino-1,1′-biphenyl)]palladium(II)) and a base (e.g., sodium tert-butoxide), or reductive amination conditions (e.g., in the presence of a suitable hydride source), or Strecker reaction conditions (e.g., in the presence of a suitable cyanide source), followed by protecting group removal to afford product 47.

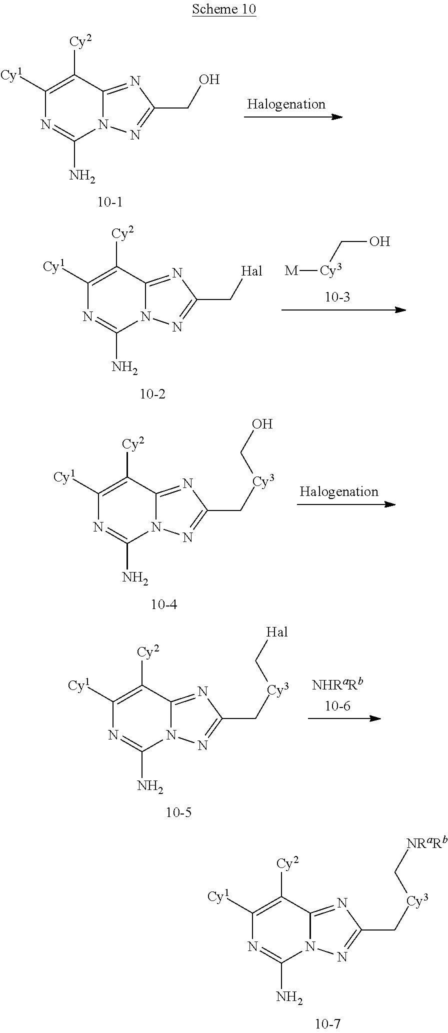

Compounds of formula 10-7 can be synthesized via the synthetic route outlined in Scheme 10. Advanced intermediate 10-1 (which can be prepared using synthetic procedures as outlined in Scheme 8) first undergoes a halogenation reaction (using an suitable reagent, such as thionyl chloride) to generate compound 10-2 (Hal is a halide, such as F, Cl, Br, or I). Compound 10-2 can then be subjected to a cross-coupling reaction with reagents of formula 10-3, in which M is a boronic acid, boronic ester or an appropriately substituted metal [e.g., M is B(OR)₂, Sn(Alkyl)₃, or Zn-Hal], under standard Suzuki cross-coupling conditions (e.g., in the presence of a palladium catalyst and a suitable base), or standard Stille cross-coupling conditions (e.g., in the presence of a palladium catalyst), or standard Negishi cross-coupling conditions (e.g., in the presence of a palladium catalyst), to afford compound 10-4. The hydroxyl group in 10-4 can then be converted to a halogen to provide compound 10-5 (using similar procedures as described for the conversion of 10-1 to 10-2). Product 10-7 can then be prepared from intermediate 10-5 and reagents of formula 10-6, using an appropriate transformation, such as a nucleophilic substitution (S_{N}2) reaction.

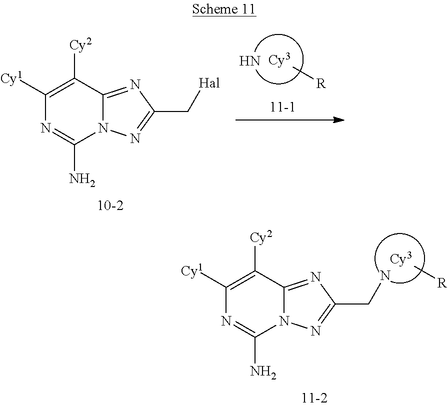

Compounds of formula 11-2 can be synthesized via the synthetic route outlined in Scheme 11. Intermediate 10-2 (which can be prepared using synthetic procedures as outlined in Scheme 10, Hal is a halide, such as F, Cl, Br, or I) can undergo a nucleophic substitution reaction (S_{N}2) with reagent of formula 11-1, to afford compound 11-2.

### Methods of Use

The compounds of the present disclosure can modulate the activity of adenosine receptors, such as subtypes A2A and A2B receptors. Accordingly, the compounds, salts or stereoisomers described herein can be used in methods of inhibiting adenosine receptors (e.g., A2A and/or A2B receptors) by contacting the receptor with any one or more of the compounds, salts, or compositions described herein. In some embodiments, the compounds or salts can be used in methods of inhibiting activity of an adenosine receptor in an individual/patient in need of the inhibition by administering an effective amount of a compound or salt of described herein. In some embodiments, modulating is inhibiting. In some embodiments, the contacting is in vivo. In some embodiments, the contacting is ex vivo or in vitro.

The compounds or salts described herein can be selective. By “selective,” it is meant that the compound binds to or inhibits an adenosine receptor with greater affinity or potency, respectively, compared to at least one other receptor, kinase, etc. The compounds of the present disclosure can also be dual antagonists (i.e., inhibitors) of adenosine receptors, e.g., A2A and A2B adenosine receptors.

Another aspect of the present disclosure pertains to methods of treating an adenosine receptor associated disease or disorder in an individual (e.g., patient) by administering to the individual in need of such treatment a therapeutically effective amount or dose of one or more compounds of the present disclosure or a pharmaceutical composition thereof. An adenosine receptor associated disease or disorder can include any disease, disorder or condition that is directly or indirectly linked to expression or activity of the adenosine receptor, including overexpression and/or abnormal activity levels.

The compounds of the present disclosure are useful in the treatment of diseases related to the activity of adenosine receptors including, for example, cancer, inflammatory diseases, cardiovascular diseases, neurodegenerative diseases, immunomodulatory disorders, central nerve system diseases, and diabetes.

Based on the compelling roles of adenosine, e.g., A2A, A2B, receptors in multiple immunosuppressive mechanisms, developing inhibitors can boost the immune system to suppress tumor progression. Adenosine receptor inhibitors can be used to treat, alone or in combination with other therapies, bladder cancer, lung cancer (e.g., non-small cell lung cancer (NSCLC), lung metastasis), melanoma (e.g., metastatic melanoma), breast cancer, cervical cancer, ovarian cancer, colorectal cancer, pancreatic cancer, esophageal cancer, prostate cancer, kidney cancer, skin cancer, thyroid cancer, liver cancer, uterine cancer, head and neck cancer, and renal cell carcinoma (Antonioli, L. et al., Nature Reviews Cancer, 2013, 13, 842-857). See also, https://globenewswire.com/news-release/2017/04/04/954192/0/en/Corvus-Pharmaceuticals-Announces-Interim-Results-from-Ongoing-Phase-1-1b-Study-Demonstrating-Safety-and-Clinical-Activity-of-Lead-Checkpoint-Inhibitor-CPI-444-in-Patients-with-Adva.html; Cekic C. et al., J Immunol, 2012, 188:198-205; Iannone, R. et al., Am. J. Cancer Res. 2014, 4:172-181 (study shows that both A2A and CD73 blockade enhance the antitumor activity of anti-CTLA-4 mAb therapy in a B16F10 murine melanoma model); Iannone, R. et al., Neoplasia, 2013, 15:1400-1410 and Beavis P A., et al., Proc Natl Acad Sci. USA, 2013, 110:14711-14716 (study shows that A2A and CD73 blockade decreased metastasis in 4T1 breast tumor model with has high CD73 expression). In some embodiments, the prostate cancer is metastatic castrate-resistant prostate carcinoma (mCRPC). In some embodiments, the colorectal cancer is colorectal carcinoma (CRC).

In some embodiments, the disease or disorder is lung cancer (e.g., non-small cell lung cancer), melanoma, pancreatic cancer, breast cancer, head and neck squamous cell carcinoma, prostate cancer, liver cancer, color cancer, endometrial cancer, bladder cancer, skin cancer, cancer of the uterus, renal cancer, gastric cancer, or sarcoma. In some embodiments, the sarcoma is Askin's tumor, sarcoma botryoides, chondrosarcoma, Ewing's sarcoma, malignant hemangioendothelioma, malignant schwannoma, osteosarcoma, alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, rhabdomyosarcoma, synovial sarcoma, or undifferentiated pleomorphic sarcoma.

In some embodiments, the disease or disorder is mesothelioma or adrenocarcinoma. In some embodiments, the disease or disorder is mesothelioma. In some embodiments, the disease or disorder is adrenocarcinoma.

MDSC (myeloid-derived suppressor cells) are a heterogenous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions. In some embodiments, the compounds, etc. described herein can be used in methods related to cancer tissue (e.g., tumors) with high infiltration of MDSCs, including solid tumors with high basal level of macrophage and/or MDSC infiltration.

In some embodiments, the compounds of the disclosure can be used in treating pulmonary inflammation, including bleomycin-induced pulmonary fibrosis and injury related to adenosine deaminase deficiency (Baraldi, et al., Chem. Rev., 2008, 108, 238-263).

In some embodiments, the compounds of the disclosure can be used as a treatment for inflammatory disease such as allergic reactions (e.g., A2B adenosine receptor dependent allergic reactions) and other adenosine receptor dependent immune reactions. Further inflammatory diseases that can be treated by compounds of the disclosure include respiratory disorders, sepsis, reperfusion injury, and thrombosis.

In some embodiments, the compounds of the disclosure can be used as a treatment for cardiovascular disease such as coronary artery disease (myocardial infarction, angina pectoris, heart failure), cerebrovascular disease (stroke, transient ischemic attack), peripheral artery disease, and aortic atherosclerosis and aneurysm. Atherosclerosis is an underlying etiologic factor in many types of cardiovascular disease. Atherosclerosis begins in adolescence with fatty streaks, which progress to plaques in adulthood and finally results in thrombotic events that cause occlusion of vessels leading to clinically significant morbidity and mortality. Antagonists to the A2B adenosine receptor and A2A adenosine receptor may be beneficial in preventing atherosclerotic plaque formation (Eisenstein, A. et al., J. Cell Physiol., 2015, 230(12), 2891-2897).

In some embodiments, the compounds of the disclosure can be used as a treatment for disorders in motor activity; deficiency caused by degeneration of the striatonigral dopamine system; and Parkinson's disease; some of the motivational symptoms of depression (Collins, L. E. et al. Pharmacol. Biochem. Behav., 2012, 100, 498-505).

In some embodiments, the compounds of the disclosure can be used as a treatment for diabetes and related disorders, such as insulin resistance. Diabetes affects the production of adenosine and the expression of A2B adenosine receptors (A2BRs) that stimulate IL-6 and CRP production, insulin resistance, and the association between A_{2B}R gene single-nucleotide polymorphisms (ADORA2B SNPs) and inflammatory markers. The increased A2BR signaling in diabetes may increase insulin resistance in part by elevating pro-inflammatory mediators. Selective A2BR blockers may be useful to treat insulin resistance (Figler, R. A. et al. Diabetes, 2011, 60 (2), 669-679).

It is believed that compounds provided herein, e.g., compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual” or “patient”, used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Immune-Checkpoint Therapies

In some embodiments, A2A and A2B dual inhibitors provided herein can be used in combination with one or more immune checkpoint inhibitors for the treatment of cancer as described herein. In one embodiment, the combination with one or more immune checkpoint inhibitors as described herein can be used for the treatment of melanoma. Compounds of the present disclosure can be used in combination with one or more immune checkpoint inhibitors. Exemplary immune checkpoint inhibitors include inhibitors against immune checkpoint molecules such as CD20, CD28, CD40, CD122, CD96, CD73, CD47, GITR, CSF1R, JAK, PI3K delta, PI3K gamma, TAM, arginase, HPK1, CD137 (also known as 4-1BB), ICOS, B7-H3, B7-H4, BTLA, CTLA-4, LAG3, TIM3, VISTA, TIGIT, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT and VISTA. In some embodiments, the compounds of the disclosure provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the A2A and A2B dual inhibitors provided herein can be used in combination with one or more agonists of immune checkpoint molecules, e.g., OX40, CD27, OX40, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), durvalumab (Imfinzi®), pidilizumab, SHR-1210, PDR001, MGA012, PDR001, AB122 or AMP-224. In some embodiments, the anti-PD-1 monoclonal antibody is nivolumab or pembrolizumab. In some embodiments, the anti-PD1 antibody is pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g. urelumab, utomilumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of PD-1 and PD-L1, e.g., an anti-PD-1/PD-L1 monoclonal antibody. In some embodiments, the anti-PD-1/PD-L1 is MCLA-136.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3, tumor specific antigens (e.g., CD70), or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### II. Cancer Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the present disclosure can be used in combination with one or more other enzyme/protein/receptor inhibitors or one or more therapies for the treatment of diseases, such as cancer. Examples of diseases and indications treatable with combination therapies include those as described herein.

The compounds of the present disclosure can be used in combination with one or more additional pharmaceutical agents such as, for example, chemotherapeutics, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF and FAK kinase inhibitors. The one or more additional pharmaceutical agents can be administered to a patient simultaneously or sequentially.

For example, the compounds as disclosed herein can be combined with one or more inhibitors of the following kinases for the treatment of cancer and other diseases or disorders described herein: Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Non-limiting examples of inhibitors that can be combined with the compounds of the present disclosure for treatment of cancer and other diseases and disorders described herein include an FGFR inhibitor (FGFR1, FGFR2, FGFR3 or FGFR4, e.g., INCB54828, INCB62079 and INCB63904), a JAK inhibitor (JAK1 and/or JAK2, e.g., ruxolitinib, baricitinib or INCB39110), an IDO inhibitor (e.g., epacadostat, NLG919, or BMS-986205), an LSD1 inhibitor (e.g., INCB59872 and INCB60003), a TDO inhibitor, a PI3K-delta inhibitor (e.g., INCB50797 and INCB50465), a Pim inhibitor, a CSF1R inhibitor, a TAM receptor tyrosine kinases (Tyro-3, Axl, and Mer), a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643) and an adenosine receptor antagonist or combinations thereof.

Example antibodies for use in combination therapy include but are not limited to Trastuzumab (e.g. anti-HER2), Ranibizumab (e.g. anti-VEGF-A), Bevacizumab (trade name Avastin, e.g. anti-VEGF, Panitumumab (e.g. anti-EGFR), Cetuximab (e.g. anti-EGFR), Rituxan (anti-CD20) and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, IRESSA™, (gefitinib), TARCEVA™, (erlotinib), antibodies to EGFR, GLEEVEC™, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™,™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17 alpha-.-ethinylestradiol, diethylstilbestrol, testosterone, prednisone, fluoxymesterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™, (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101, 731.

The compounds of the present disclosure can further be used in combination with other methods of treating cancers, for example by chemotherapy, irradiation therapy, tumortargeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, Toll receptor agonists, STING agonists, oncolytic virotherapy and immunomodulating small molecules, including thalidomide or JAK1/2 inhibitor and the like. The compounds can be administered in combination with one or more anti cancer drugs, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, olaparib, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, rucaparib, streptozocin, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, niraparib, veliparib, talazoparib, and zoledronate.

Additional examples of chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a PI3K inhibitor of the present disclosure with an additional agent.

In some embodiments, the compounds of the disclosure can be used in combination with an inhibitor of JAK or PI3Kδ.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp 100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV).

In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum. Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating A2A and/or A2B receptors in tissue samples, including human, and for identifying A2A and/or A2B antagonists by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes adenosine receptor (e.g., A2A and/or A2B) assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in any of the disclosed Formulas, e.g., Formula (I), can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of any “alkyl”, “alkenyl”, “alkynyl”, “aryl”, “phenyl”, “cycloalkyl”, “heterocycloalkyl”, or “heteroaryl” substituents or “—C₁₋₆ alkyl-”, “alkylene”, “alkenylene” and “alkynylene” linking groups, as described herein, are each optionally replaced by a deuterium atom.

Synthetic methods for including isotopes into organic compounds are known in the art (Deuterium Labeling in Organic Chemistry by Alan F. Thomas (New York, N.Y., Appleton-Century-Crofts, 1971; The Renaissance of HID Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind an adenosine receptor by monitoring its concentration variation when contacting with the adenosine receptor, through tracking of the labeling. For example, a test compound (labeled) can be evaluated for its ability to reduce binding of another compound which is known to bind to an adenosine receptor (i.e., standard compound). Accordingly, the ability of a test compound to compete with the standard compound for binding to the adenosine receptor directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of adenosine receptor-associated diseases or disorders (such as, e.g., cancer, an inflammatory disease, a cardiovascular disease, or a neurodegenerative disease) which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of an adenosine receptor (e.g., A2A and/or A2B) according to at least one assay described herein.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:
pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)).
pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)).

Separation of some of the racemic compounds into enantiopure samples were prepared on preparative scale by chiral-phase high performance liquid chromatography under the following conditions: Instrument: Agilent 1100 Prep HPLC; Column: Phenomenex Lux Cellulose-4, 21.2×250 mm, 5 μm; eluting with isocratic mobile phase 45% EtOH in hexanes with a flow rate of 20 mL/minute.

### Example 1. 3-(5-Amino-8-(2,6-dimethylpyridin-4-yl)imidazo[1,2-c]pyrimidin-7-yl)benzonitrile

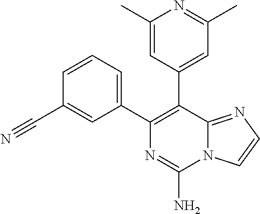 [SMILES: Cc1cc(-c2c(-c3cccc(C#N)c3)nc(N)n3ccnc23)cc(C)n1]

### Step 1: 5-Bromo-2,6-dichloropyrimidin-4-amine

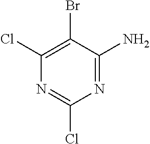

To a solution of 2,6-dichloropyrimidin-4-amine (Combi-Blocks, cat #OR-0412) (10 g, 61 mmol) in DMF (50 mL) at 0° C. was added N-bromosuccinimide (11 g, 61 mmol). The reaction mixture was stirred for 16 h at room temperature before water (100 mL) was added. The resulting precipitate was collected by filtration, and then dried to give the desired product (13.1 g, 88%), which was used in the next step without further purification. LC-MS calculated for C₄H₃BrCl₂N₃ (M+H)⁺: m/z=241.9. found 241.8.

### Step 2: 8-Bromo-5,7-dichloroimidazo[1,2-c]pyrimidine

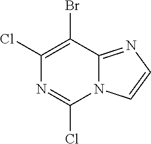 [SMILES: Clc1nc(Cl)n2ccnc2c1Br]

2-Bromo-1,1-diethoxyethane (11 mL, 70 mmol) was added to a mixture of 5-bromo-2,6-dichloropyrimidin-4-amine (2.0 g, 8.2 mmol) in MeCN (25 mL). The resulting mixture was stirred at 120° C. for 1 h then cooled to room temperature and concentrated under reduced pressure. The residue was triturated with EtOAc to give the desired product as the HBr salt (2.4 g, 84%), which was used in the next step without further purification. LC-MS calculated for C₆H₃BrCl₂N₃ (M+H)⁺: m/z=265.9. found 265.8.

### Step 3: 8-Bromo-7-chloroimidazo[1,2-c]pyrimidin-5-amine

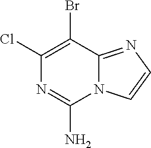 [SMILES: Nc1nc(Cl)c(Br)c2nccn12]

To a solution of 8-bromo-5,7-dichloroimidazo[1,2-c]pyrimidine hydrobromide (2.2 g, 6.3 mmol) in THE (30 mL) was added concentrated ammonium hydroxide (57 mL, 14 M). The reaction mixture was stirred at room temperature for 16 h before the volatiles were removed under reduced pressure. The resulting solid was collected by filtration, washed with water (100 mL) and then dried to give the desired product (0.75 g, 48%), which was used in the next step without further purification. LC-MS calculated for C₆H₅BrClN₄ (M+H)⁺: m/z=246.9. found 247.0.

### Step 4: 7-Chloro-8-(2,6-dimethylpyridin-4-yl)imidazo[1,2-c]pyrimidin-5-amine

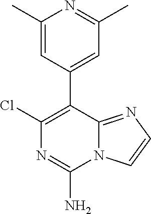 [SMILES: Cc1cc(-c2c(Cl)nc(N)n3ccnc23)cc(C)n1]

[1,1′-Bis(diphenylphosphino)ferrocene]dichloropalladium(II) (7.3 mg, 10 mol %) was added to a mixture of 8-bromo-7-chloroimidazo[1,2-c]pyrimidin-5-amine (25 mg, 0.10 mmol), 2,6-dimethyl-4-(4,4,5,5-tetramethyl-1,3,2-dioxaborolan-2-yl)pyridine (35 mg, 0.15 mmol), and sodium carbonate (32 mg, 0.30 mmol) in THE (0.36 mL) and water (0.07 mL). The mixture was purged with nitrogen, and then stirred at 70° C. for 16 h. The reaction mixture was cooled to room temperature and concentrated under reduced pressure. The crude product was purified by flash chromatography on a silica gel column eluting with 0 to 15% MeOH/DCM to give the desired product, which was used in the next step without further purification. LC-MS calculated for C₁₃H₁₃ClN₅ (M+H)⁺: m/z=274.1. found 274.1.

### Step 5: 3-(5-Amino-8-(2,6-dimethylpyridin-4-yl)imidazo[1,2-c]pyrimidin-7-yl)benzonitrile

To a microwave vial was added 7-chloro-8-(2,6-dimethylpyridin-4-yl)imidazo[1,2-c]pyrimidin-5-amine (40 mg, 0.15 mmol), (3-cyanophenyl)boronic acid (64 mg, 0.44 mmol), tripotassium phosphate (120 mg, 0.59 mmol), DMF (2.4 mL), water (0.60 mL) and [1,1′-bis(diphenylphosphino)ferrocene]dichloropalladium(II) (11 mg, 10 mol %). The reaction solution was purged with nitrogen, and then the microwave vial was sealed and heated in a microwave reactor at 120° C. for 20 min. The reaction mixture was cooled to room temperature, filtered through a Celite plug with 50% EtOAc/DCM, and then concentrated under reduced pressure. The crude product was purified by prep-LCMS (pH=2, MeCN/water with TFA) to give the desired product as the TFA salt. LC-MS calculated for C₂₀H₁₇N₆ (M+H)⁺: m/z=341.2. found 341.1.

### Example 2. 7-(2,3-Dihydro-1H-pyrrolo[2,3-b]pyridin-5-yl)-8-(2,6-dimethylpyridin-4-yl)imidazo[1,2-c]pyrimidin-5-amine

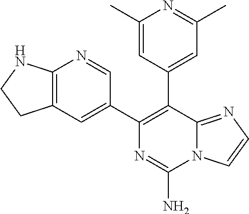 [SMILES: Cc1cc(-c2c(-c3cnc4c(c3)CCN4)nc(N)n3ccnc23)cc(C)n1]

This compound was prepared using similar procedures as described for Example 1, with (2,3-dihydro-1H-pyrrolo[2,3-b]pyridin-5-yl)boronic acid replacing (3-cyanophenyl)boronic acid in Step 5. The product was purified by prep-LCMS (pH=10, MeCN/water with NH₄OH) to give the desired product as the free base. LC-MS calculated for C₂₀H₂₀N₇ (M+H)⁺: m/z=358.2. found 358.2.

### Example 3. 8-(2,6-Dimethylpyridin-4-yl)-3-(morpholinomethyl)-7-phenylimidazo[1,2-c]pyrimidin-5-amine

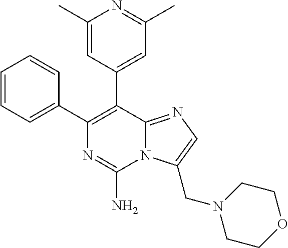 [SMILES: Cc1cc(-c2c(-c3ccccc3)nc(N)n3c(CN4CCOCC4)cnc23)cc(C)n1]

To a mixture of 8-(2,6-dimethylpyridin-4-yl)-7-phenylimidazo[1,2-c]pyrimidin-5-amine (prepared using similar procedures as described in Example 1, with phenylboronic acid replacing (3-cyanophenyl)boronic acid in Step 5) (25 mg, 0.08 mmol) and morpholine (9.5 μL, 0.16 mmol) was added (diacetoxyiodo)benzene (51 mg, 0.16 mmol). The reaction mixture was stirred at 50° C. for 2 h, and the product was purified by prep-LCMS (pH=2, MeCN/water with TFA) to give the desired product as the TFA salt. LC-MS calculated for C₂₄H₂₇N₆O (M+H)⁺: m/z=415.2. found 415.2.

### Example 4. 7-(3-Methoxyphenyl)-8-(pyridin-4-yl)imidazo[1,2-c]pyrimidin-5-amine

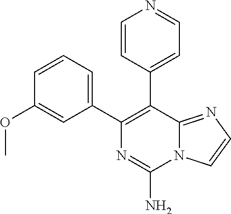 [SMILES: COc1cccc(-c2nc(N)n3ccnc3c2-c2ccncc2)c1]

[1,1′-bis(dicyclohexylphosphino)ferrocene]dichloropalladium(II) (6.1 mg, 10 mol %) was added to a mixture of 8-bromo-7-chloroimidazo[1,2-c]pyrimidin-5-amine (prepared in Example 1, Step 3) (20 mg, 0.08 mmol), pyridin-4-ylboronic acid (12 mg, 0.10 mmol), and cesium carbonate (79 mg, 0.24 mmol) in tert-butanol (0.45 mL) and water (0.09 mL). The solution was purged with nitrogen, stirred at 75° C. for 2 h, and cooled to room temperature. Cesium carbonate (53 mg, 0.24 mmol), [1,1′-bis(dicyclohexylphosphino)ferrocene]dichloropalladium(II) (3.1 mg, 5 mol %), and (3-methoxyphenyl)boronic acid (18 mg, 0.12 mmol) were then added. The reaction mixture was stirred at 105° C. for 4 h, and cooled to room temperature. The mixture was filtered through a Celite plug with 50% EtOAc/DCM and concentrated under reduced pressure. The product was purified by prep-LCMS (pH=2, MeCN/water with TFA) to give the desired product as the TFA salt. LC-MS calculated for C₁₈H₁₆N₅O (M+H)⁺: m/z=318.1. found 318.2.

### Example 5. 7,8-Di(pyridin-4-yl)imidazo[1,2-c]pyrimidin-5-amine

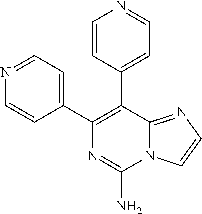 [SMILES: Nc1nc(-c2ccncc2)c(-c2ccncc2)c2nccn12]

This compound was prepared using similar procedures as described for Example 4, with pyridin-4-ylboronic acid replacing (3-methoxyphenyl)boronic acid. The product was purified by prep-LCMS (pH=2, MeCN/water with TFA) to give the desired product as the TFA salt. LC-MS calculated for C₁₆H₁₃N₆ (M+H)⁺: m/z=289.1. found 289.1.

### Example 6. 7-(2-Methylfuran-3-yl)-8-(pyridin-4-yl)imidazo[1,2-c]pyrimidin-5-amine

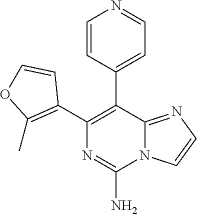 [SMILES: Cc1occc1-c1nc(N)n2ccnc2c1-c1ccncc1]

This compound was prepared using similar procedures as described for Example 4, with 4,4,5,5-tetramethyl-2-(2-methylfuran-3-yl)-1,3,2-dioxaborolane replacing (3-methoxyphenyl)boronic acid. In addition, the reaction mixture was stirred at 105° C. for 20 h. The product was purified by prep-LCMS (pH=2, MeCN/water with TFA) to give the desired product as the TFA salt. LC-MS calculated for C₁₆H₁₄N₅O (M+H)⁺: m/z=292.1. found 292.1.

### Example 7. 7-(2-Fluorophenyl)-8-(pyridin-4-yl)imidazo[1,2-c]pyrimidin-5-amine

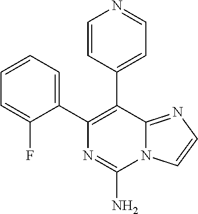 [SMILES: Nc1nc(-c2ccccc2F)c(-c2ccncc2)c2nccn12]

This compound was prepared using similar procedures as described for Example 4, with (2-fluorophenyl)boronic acid replacing (3-methoxyphenyl)boronic acid. The product was purified by prep-LCMS (pH=2, MeCN/water with TFA) to give the desired product as the TFA salt. LC-MS calculated for C₁₇H₁₃FN₅ (M+H)⁺: m/z=306.1. found 306.1.

### Example 8. 7-(Benzofuran-2-yl)-8-(pyridin-4-yl)imidazo[1,2-c]pyrimidin-5-amine

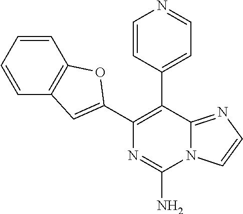 [SMILES: Nc1nc(-c2cc3ccccc3o2)c(-c2ccncc2)c2nccn12]

This compound was prepared using similar procedures as described for Example 4, with benzofuran-2-ylboronic acid replacing (3-methoxyphenyl)boronic acid. The product was purified by prep-LCMS (pH=2, MeCN/water with TFA) to give the desired product as the TFA salt. LC-MS calculated for C₁₉H₁₄N₅O (M+H)⁺: m/z=328.1. found 328.1.

### Example 9. 7-(1-Methyl-1H-pyrazol-4-yl)-8-(2-methylpyridin-4-yl)imidazo[1,2-c]pyrimidin-5-amine

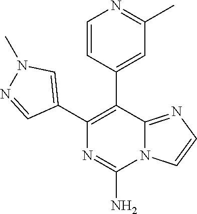 [SMILES: Cc1cc(-c2c(-c3cnn(C)c3)nc(N)n3ccnc23)ccn1]

### Step 1: 7-Chloro-8-(2-methylpyridin-4-yl)imidazo[1,2-c]pyrimidin-5-amine

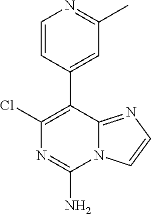 [SMILES: Cc1cc(-c2c(Cl)nc(N)n3ccnc23)ccn1]

[1,1′-bis(dicyclohexylphosphino)ferrocene]dichloropalladium(II) (110 mg, 10 mol %) was added to a mixture of 8-bromo-7-chloroimidazo[1,2-c]pyrimidin-5-amine (prepared in Example 1, Step 3) (370 mg, 1.5 mmol), 2-methyl-4-(4,4,5,5-tetramethyl-1,3,2-dioxaborolan-2-yl)pyridine (490 mg, 2.2 mmol), and cesium carbonate (1.5 g, 4.5 mmol) in tert-butanol (8.3 mL) and water (1.7 mL). The solution was purged with nitrogen, and then stirred at 75° C. for 5 h. The reaction mixture was cooled to room temperature and filtered through a Celite plug with EtOAc. The filtrate was concentrated under reduced pressure. The crude product was purified by flash chromatography on a silica gel column eluting with 0-10% MeOH/DCM to give the desired product, which was used in the next step without further purification. LC-MS calculated for C₁₂H₁₄ClN₅ (M+H)⁺: m/z=260.1. found 260.0.

### Step 2: 7-(1 Methyl-1H pyrazol-4-yl)-8-(2-methylpyridin-4-yl)imidazo[1,2-c]pyrimidin-5-amine

To a microwave vial was added 7-chloro-8-(2-methylpyridin-4-yl)imidazo[1,2-c]pyrimidin-5-amine (43 mg, 0.17 mmol), 1-methyl-4-(4,4,5,5-tetramethyl-1,3,2-dioxaborolan-2-yl)-1H-pyrazole (100 mg, 0.50 mmol), tripotassium phosphate (140 mg, 0.66 mmol), DMF (1.3 mL), water (0.33 mL) and [1,1′-bis(diphenylphosphino)ferrocene]dichloropalladium(II) (12 mg, 10 mol %). The reaction solution was purged with nitrogen, and then the microwave vial was sealed and heated in a microwave reactor at 120° C. for 20 min. The reaction mixture was cooled to room temperature and filtered through a Celite plug with 50% EtOAc/DCM, then concentrated under reduced pressure. The product was purified by prep-LCMS (pH=10, MeCN/water with NH₄OH) to give the desired product as the free base. LC-MS calculated for C₁₆H₁₆N₇ (M+H)⁺: m/z=306.1. found 306.1.

### Example 10. 8-(2-Methylpyridin-4-yl)-2,7-diphenylimidazo[1,2-c]pyrimidin-5-amine

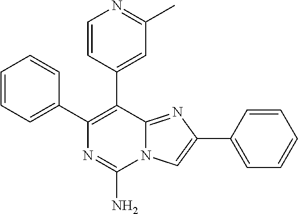 [SMILES: Cc1cc(-c2c(-c3ccccc3)nc(N)n3cc(-c4ccccc4)nc23)ccn1]

### Step 1: 2 Methoxy-6 phenylpyrimidin-4-amine

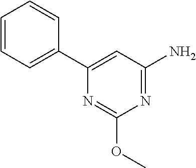

Bis(di-tert-butyl(4-dimethylaminophenyl)phosphine)dichloropalladium(II) (0.89 g, 5 mol %) was added to a mixture of 6-chloro-2-methoxypyrimidin-4-amine (Ark Pharm, Inc, cat #AK-25131) (4.0 g, 25 mmol), phenylboronic acid (4.6 g, 38 mmol) and cesium carbonate (16 g, 50 mmol) in toluene (130 mL) and water (13 mL). The solution was purged with nitrogen, and then stirred at 115° C. for 16 h. The reaction mixture was cooled to room temperature, filtered through a Celite plug with DCM and concentrated under reduced pressure. Water (100 mL) was added to the residue and the resulting solid was collected by filtration, and then dried to give the desired product (5.0 g, 99%), which was used in the next step without further purification. LC-MS calculated for C₁₁H₁₂N₃O (M+H)⁺: m/z=202.1. found 202.0.

### Step 2: 5-Bromo-2-methoxy-6-phenylpyrimidin-4-amine

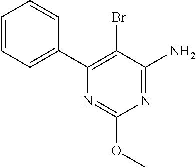 [SMILES: COc1nc(N)c(Br)c(-c2ccccc2)n1]

To a solution of 2-methoxy-6-phenylpyrimidin-4-amine (5.1 g, 25 mmol) in DMSO (51 mL), MeCN (27 mL) and water (1.7 mL) at 0° C. was added N-bromosuccinimide (4.5 g, 25 mmol). The reaction mixture was stirred for 2 h at room temperature before water (100 mL) was added. The resulting precipitate was collected by filtration then dried to give the desired product (5.2 g, 73%), which was used in the next step without further purification. LC-MS calculated for C₁₁H₁₁BrN₃O (M+H)⁺: m/z=280.0. found 280.0.

### Step 3: 2-Methoxy-5-(2-methylpyridin-4-yl)-6-phenylpyrimidin-4-amine

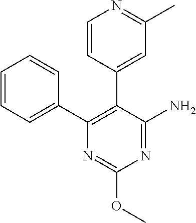 [SMILES: COc1nc(N)c(-c2ccnc(C)c2)c(-c2ccccc2)n1]

[1,1′-bis(dicyclohexylphosphino)ferrocene]dichloropalladium(II) (0.41 g, 10 mol %) was added to a mixture of 5-bromo-2-methoxy-6-phenylpyrimidin-4-amine (1.5 g, 5.4 mmol), 2-methyl-4-(4,4,5,5-tetramethyl-1,3,2-dioxaborolan-2-yl)pyridine (1.8 g, 8.0 mmol), and cesium carbonate (3.5 g, 11 mmol) in tert-butanol (20 mL) and water (3.9 mL). The solution was purged with nitrogen, and then stirred at 120° C. for 1.5 h. The reaction mixture was cooled to room temperature, filtered through a Celite plug with DCM and concentrated under reduced pressure. Water (100 mL) was added to the residue and the resulting solid was collected by filtration then dried to give the desired product (1.02 g, 65%), which was used in the next step without further purification. LC-MS calculated for C₁₇H₁₇N₄O (M+H)⁺: m/z=293.1. found 293.1.

### Step 4: 8-(2 Methylpyridin-4-yl)-2,7-diphenylimidazo[1,2-c]pyrimidin-5(6H)-one

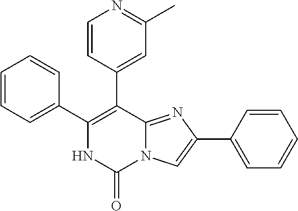 [SMILES: Cc1cc(-c2c(-c3ccccc3)[nH]c(=O)n3cc(-c4ccccc4)nc23)ccn1]

To 2-methoxy-5-(2-methylpyridin-4-yl)-6-phenylpyrimidin-4-amine (100 mg, 0.34 mmol) in acetic acid (1.4 mL) was added 2-bromo-1-phenylethan-1-one (170 mg, 0.86 mmol). The reaction mixture was stirred at 120° C. for 20 h. After cooling to room temperature the volatiles were removed under reduced pressure. The resulting solid was washed with Et₂O, collected by filtration and then dried to give the crude product, which was used in the next step without further purification. LC-MS calculated for C₂₄H₁₉N₄O (M+H)⁺: m/z=379.2. found 379.1.

### Step 5: 5-Chloro-8-(2-methylpyridin-4-yl)-2,7-diphenylimidazo[1,2-c]pyrimidine

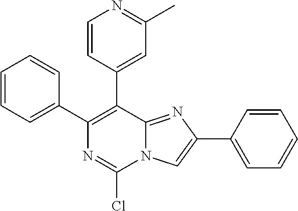 [SMILES: Cc1cc(-c2c(-c3ccccc3)nc(Cl)n3cc(-c4ccccc4)nc23)ccn1]

To a stirred solution of 8-(2-methylpyridin-4-yl)-2,7-diphenylimidazo[1,2-c]pyrimidin-5(6H)-one acetate (150 mg, 0.34 mmol) and POCl₃ (0.25 mL, 2.6 mmol) in toluene (1.3 mL) at 0° C. was added dropwise N,N-diisopropylethylamine (0.26 mL, 1.5 mmol). The resulting solution was slowly heated to 120° C. over 1 h, and then stirred at 120° C. for 16 h. After cooling to room temperature the volatiles were removed under reduced pressure. The resulting residue was diluted with 1 N HCl (2.0 mL) and water (20 mL), and extracted with DCM (5×20 mL). The organic layers were combined, dried over Na₂SO₄, filtered and concentrated under reduced pressure. The resulting crude product mixture was used in the next step without further purification. LC-MS calculated for C₂₄H₁₈ClN₄ (M+H)⁺: m/z=397.1. found 397.1.

### Step 6: 8-(2 Methylpyridin-4-yl)-2,7-diphenylimidazo[1,2-c]pyrimidin-5-amine

This compound was prepared using a similar procedure as described for Example 1, Step 3, with 5-chloro-8-(2-methylpyridin-4-yl)-2,7-diphenylimidazo[1,2-c]pyrimidine replacing 8-bromo-5,7-dichloroimidazo[1,2-c]pyrimidine hydrobromide. The product was purified by prep-LCMS (pH=2, MeCN/water with TFA) to give the desired product as the TFA salt. LC-MS calculated for C₂₄H₂₀N₅ (M+H)⁺: m/z=378.2. found 378.1.

### Example 11. Ethyl 5-amino-8-(2-methylpyridin-4-yl)-7-phenylimidazo[1,2-c]pyrimidine-2-carboxylate

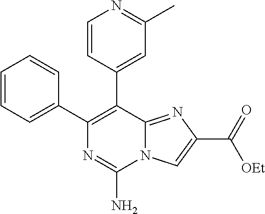 [SMILES: CCOC(=O)c1cn2c(N)nc(-c3ccccc3)c(-c3ccnc(C)c3)c2n1]

This compound was prepared using similar procedures as described for Example 10, with ethyl 3-bromo-2-oxopropanoate replacing 2-bromo-1-phenylethan-1-one in Step 4. The product was purified by prep-LCMS (pH=10, MeCN/water with NH₄OH) to give the desired product as the free base. LC-MS calculated for C₂₁H₂₀N₅O₂ (M+H)⁺: m/z=374.2. found 374.0.

### Example 12. Methyl 5-(5-amino-7-phenyl-8-(pyridin-4-yl)imidazo[1,2-c]pyrimidin-2-yl)isoxazole-3-carboxylate

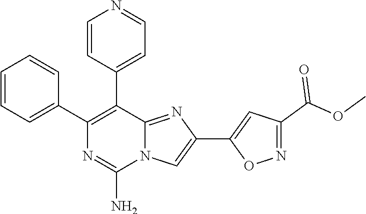 [SMILES: COC(=O)c1cc(-c2cn3c(N)nc(-c4ccccc4)c(-c4ccncc4)c3n2)on1]

### Step 1: Ethyl 5-(5-oxo-7 phenyl-8-(pyridin-4-yl)-5,6-dihydroimidazo[1,2-c]pyrimidin-2-yl)isoxazole-3-carboxylate

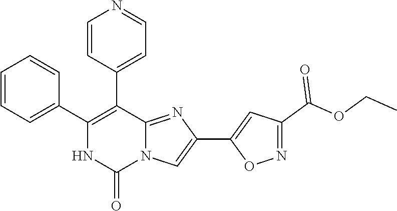 [SMILES: CCOC(=O)c1cc(-c2cn3c(=O)[nH]c(-c4ccccc4)c(-c4ccncc4)c3n2)on1]

To a mixture of 2-methoxy-6-phenyl-5-(pyridin-4-yl)pyrimidin-4-amine (prepared using similar procedures as described in Example 10, Step 1-3, with pyridin-4-ylboronic acid replacing 2-methyl-4-(4,4,5,5-tetramethyl-1,3,2-dioxaborolan-2-yl)pyridine in Step 3) (50 mg, 0.18 mmol) in 2-propanol (0.54 mL) was added ethyl 5-(2-bromoacetyl)isoxazole-3-carboxylate (Combi-Blocks, cat #SS-6738) (71 mg, 0.27 mmol). The reaction mixture was stirred at 110° C. for 4 h. After cooling to room temperature the volatiles were removed under reduced pressure. The resulting solid was washed with EtOAc, collected by filtration, and then dried to give the crude product as the HBr salt, which was used in the next step without further purification. LC-MS calculated for C₂₃H₁₈N₅O₄ (M+H)⁺: m/z=428.1. found 428.0.

### Step 2: Ethyl 5-(5-chloro-7 phenyl-8-(pyridin-4-yl)imidazo[1,2-c]pyrimidin-2 yl)isoxazole-3-carboxylate

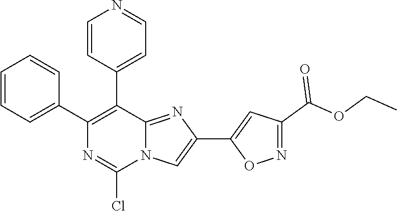 [SMILES: CCOC(=O)c1cc(-c2cn3c(Cl)nc(-c4ccccc4)c(-c4ccncc4)c3n2)on1]

To a stirred solution of ethyl 5-(5-oxo-7-phenyl-8-(pyridin-4-yl)-5,6-dihydroimidazo[1,2-c]pyrimidin-2-yl)isoxazole-3-carboxylate hydrobromide (72 mg, 0.14 mmol) in MeCN (0.53 mL) was added POCl₃ (0.54 mL, 5.8 mmol). The resulting solution was stirred at 120° C. for 16 h. After cooling to room temperature the volatiles were removed under reduced pressure. To the residue was slowly added ice water (20 mL) and the slurry was stirred for 20 min. The resulting solid was collected by filtration then dried to give the crude product, which was used in the next step without further purification. LC-MS calculated for C₂₃H₁₇ClN₅O₃ (M+H)⁺: m/z=446.1. found 446.0.

### Step 3: Methyl 5-(5-amino-7 phenyl-8-(pyridin-4-yl)imidazo[1,2-c]pyrimidin-2 yl)isoxazole-3-carboxylate

To a solution of ethyl 5-(5-chloro-7-phenyl-8-(pyridin-4-yl)imidazo[1,2-c]pyrimidin-2-yl)isoxazole-3-carboxylate (70 mg, 0.16 mmol) in THE (0.70 mL) and MeOH (0.10 mL) was added concentrated ammonium hydroxide (1.4 mL, 14 M). The reaction mixture was stirred at room temperature for 16 h before the volatiles were removed under reduced pressure. The product was purified by prep-LCMS (pH=2, MeCN/water with TFA) to give the desired product as the TFA salt. LC-MS calculated for C₂₂H₁₇N₆O₃ (M+H)⁺: m/z=413.1. found 413.1.

### Example 13. 3-(4-Ethoxyphenyl)-8-(2-methylpyridin-4-yl)-7-phenylimidazo[1,2-c]pyrimidin-5-amine

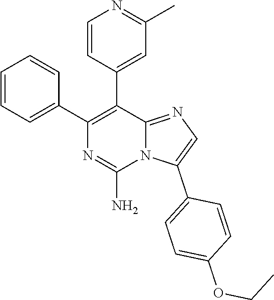 [SMILES: CCOc1ccc(-c2cnc3c(-c4ccnc(C)c4)c(-c4ccccc4)nc(N)n23)cc1]

### Step 1: 8-(2 Methylpyridin-4-yl)-7 phenylimidazo[1,2-c]pyrimidin-5(6H)-one

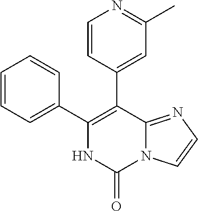 [SMILES: Cc1cc(-c2c(-c3ccccc3)[nH]c(=O)n3ccnc23)ccn1]

To a mixture of 2-methoxy-5-(2-methylpyridin-4-yl)-6-phenylpyrimidin-4-amine (from Example 10, Step 3) (400 mg, 1.4 mmol) in 2-propanol (4.2 mL) was added 2-chloroacetaldehyde (2.0 mL, 7 M in water). The reaction mixture was stirred at 110° C. for 4 h. After cooling to room temperature the volatiles were removed under reduced pressure. The resulting residue was triturated with EtOAc to give the title compound as the HCl salt (395 mg, 85%), which was used in the next step without further purification. LC-MS calculated for C₁₈H₁₅N₄O (M+H)⁺: m/z=303.1. found 303.1.

### Step 2: 8-(2 Methylpyridin-4-yl)-7 phenylimidazo[1,2-c]pyrimidin-5-amine

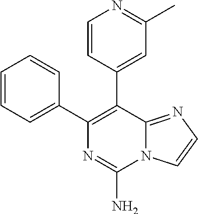 [SMILES: Cc1cc(-c2c(-c3ccccc3)nc(N)n3ccnc23)ccn1]

This compound was prepared using similar procedures as described for Example 10, Step 5-6, with 8-(2-methylpyridin-4-yl)-7-phenylimidazo[1,2-c]pyrimidin-5(6H)-one replacing 8-(2-methylpyridin-4-yl)-2,7-diphenylimidazo[1,2-c]pyrimidin-5(6H)-one in Step 5. The product was purified by prep-LCMS (pH=2, MeCN/water with TFA) to give the desired product as the TFA salt. LC-MS calculated for C₁₈H₁₆N₅ (M+H)⁺: m/z=302.1. found 302.1.

### Step 3: 3-Bromo-8-(2-methylpyridin-4-yl)-7 phenylimidazo[1,2-c]pyrimidin-5-amine

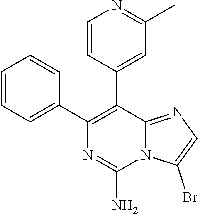 [SMILES: Cc1cc(-c2c(-c3ccccc3)nc(N)n3c(Br)cnc23)ccn1]

To a mixture of 8-(2-methylpyridin-4-yl)-7-phenylimidazo[1,2-c]pyrimidin-5-amine 2,2,2-trifluoroacetate (31 mg, 0.08 mmol) and sodium bicarbonate (14 mg, 0.17 mmol) in MeOH (0.31 mL) and water (0.31 mL) was added bromine (5.8 μL, 0.11 mmol) dropwise at 0° C. The reaction mixture was stirred at room temperature for 16 h. The resulting solution was concentrated under reduced pressure to give the crude product, which was used in the next step without further purification. LC-MS calculated for C₁₈H₁₅BrN₅ (M+H)⁺: m/z=380.1. found 380.1.

### Step 4: 3-(4-Ethoxyphenyl)-8-(2-methylpyridin-4-yl)-7 phenylimidazo[1,2-c]pyrimidin-5-amine

To a microwave vial was added 3-Bromo-8-(2-methylpyridin-4-yl)-7-phenylimidazo[1,2-c]pyrimidin-5-amine (3 mg, 0.01 mmol), (4-ethoxyphenyl)boronic acid (4 mg, 0.02 mmol), tripotassium phosphate (7 mg, 0.03 mmol), DMF (2.4 mL), water (0.60 mL) and [1,1′-bis(diphenylphosphino)ferrocene]dichloropalladium(II) (2 mg, 25 mol %). The reaction solution was purged with nitrogen, and then the microwave vial was sealed and heated in a microwave reactor at 120° C. for 20 min. The reaction mixture was cooled to room temperature and filtered through a Celite plug with 50% EtOAc/DCM, then concentrated under reduced pressure. The crude product was purified by prep-LCMS (pH=2, MeCN/water with TFA) to give the desired product as the TFA salt. LC-MS calculated for C₂₆H₂₄N₅O (M+H)⁺: m/z=422.2. found 422.2.

### Example 14. 5-Amino-8-(2-methylpyridin-4-yl)-7-phenylimidazo[1,2-c]pyrimidine-2-carboxamide

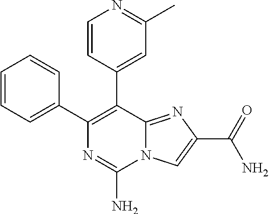 [SMILES: Cc1cc(-c2c(-c3ccccc3)nc(N)n3cc(C(N)=O)nc23)ccn1]

### Step 1: 5-Amino-8-(2-methylpyridin-4-yl)-7 phenylimidazo[1,2-c]pyrimidine-2-carboxylic acid

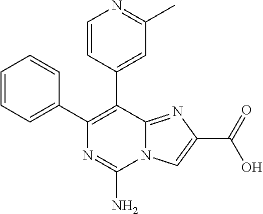 [SMILES: Cc1cc(-c2c(-c3ccccc3)nc(N)n3cc(C(=O)O)nc23)ccn1]

To a stirred solution of ethyl 5-amino-8-(2-methylpyridin-4-yl)-7-phenylimidazo[1,2-c]pyrimidine-2-carboxylate (prepared in Example 11) (66 mg, 0.18 mmol) in THE (0.90 mL) was added lithium hydroxide monohydrate (15 mg, 0.36 mmol) and water (0.90 mL). The resulting mixture was stirred at 50° C. for 16 h. After cooling to room temperature, 1 N HCl (0.38 mL) and water (0.9 mL) were added to give a precipitate which was removed by filtration. The filtrate was concentrated to give the crude product, which was used in the next step without further purification. LC-MS calculated for C₁₉H₁₆N₅O₂ (M+H)⁺: m/z=346.1. found 346.1.

### Step 2: 5-Amino-8-(2-methylpyridin-4-yl)-7 phenylimidazo[1,2-c]pyrimidine-2-carboxamide

To a solution of 5-amino-8-(2-methylpyridin-4-yl)-7-phenylimidazo[1,2-c]pyrimidine-2-carboxylic acid (48 mg, 0.14 mmol) in DMF (0.87 mL) was added HATU (63 mg, 0.17 mmol) and triethylamine (39 μL, 0.28 mmol). The reaction mixture was stirred at room temperature for 30 min before ammonium chloride (8.0 mg, 0.15 mmol) was added and the solution continued stirring at room temperature for 1.5 h. The mixture was diluted with EtOAc (5.0 mL) and washed with saturated aqueous sodium bicarbonate solution (5.0 mL) and brine (5.0 mL). The organic layer were dried over Na₂SO₄, filtered and concentrated under reduced pressure. The product was purified by prep-LCMS (pH=2, MeCN/water with TFA) to give the desired product as the TFA salt. LC-MS calculated for C₁₉H₁₇N₆O (M+H)⁺: m/z=345.1. found 345.1.

### Example 15. 5-Amino-8-(1-carbamoyl-1,2,3,6-tetrahydropyridin-4-yl)-N-ethyl-7-phenylimidazo[1,2-c]pyrimidine-2-carboxamide

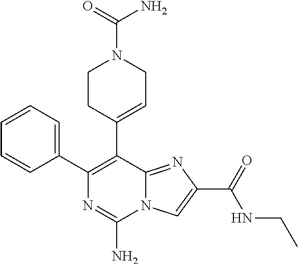 [SMILES: CCNC(=O)c1cn2c(N)nc(-c3ccccc3)c(C3=CCN(C(N)=O)CC3)c2n1]

### Step 1: 2-(Methylthio)-6-phenylpyrimidin-4-amine

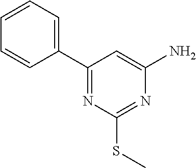

A mixture of 6-chloro-2-(methylthio)pyrimidin-4-amine (Combi-Blocks Catalog, #ST-1384) (10.0 g, 56.9 mmol), phenylboronic acid (8.33 g, 68.3 mmol), and bis(di-tert-butyl(4-dimethylaminophenyl)phosphine)dichloropalladium(II) (1.01 g, 1.42 mmol) in 1,4-dioxane (50 mL) and water (10 mL) was added cesium carbonate (37.1 g, 114.0 mmol). The reaction mixture was purged with nitrogen and then stirred at 100° C. for 12 h. After being cooled to room temperature, the reaction mixture was diluted with EtOAc, washed with brine, dried over Na₂SO₄, filtered and concentrated under reduced pressure. Light yellow solid precipitated from the solution, which was filtered and dried to obtain the desired product. LC-MS calculated for C₁₁H₁₂N₃S (M+H)⁺: m/z=218.1. found 218.1.

### Step 2: 5-Bromo-2-(methylthio)-6-phenylpyrimidin-4-amine

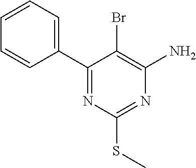 [SMILES: CSc1nc(N)c(Br)c(-c2ccccc2)n1]

To a stirred solution of 2-(methylthio)-6-phenylpyrimidin-4-amine (2.5 g, 11.5 mmol) in DMF (50 mL) was added N-bromosuccinimide (2.05 g, 11.5 mmol). The resulting mixture was stirred at room temperature for 2 h before water (200 mL) was added. Light yellow solid precipitated from the solution, which was filtered and dried to obtain the desired product. LC-MS calculated for C₁₁H₁₁BrN₃S (M+H)⁺: m/z=296.0. found 296.0.

### Step 3: Ethyl 8-bromo-5-(methylthio)-7-phenylimidazo[1,2-c]pyrimidine-2-carboxylate

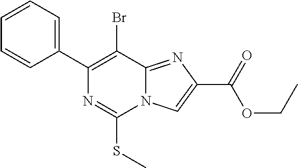 [SMILES: CCOC(=O)c1cn2c(SC)nc(-c3ccccc3)c(Br)c2n1]

To a solution of 5-bromo-2-(methylthio)-6-phenylpyrimidin-4-amine (3.69 g, 12.5 mmol) in 1,2-dimethoxyethane (40 mL) was added ethyl 3-bromo-2-oxopropanoate (4.69 mL, 37.4 mmol). The mixture was heated to 110° C. for 12 h. After being cooled to room temperature, the reaction mixture was washed with saturated NaHCO₃ solution and brine, dried over Na₂SO₄, filtered, and concentrated under reduced pressure to afford light brown solid as the desired product. LC-MS calculated for C₁₆H₁₅BrN₃O₂S (M+H)⁺: m/z=392.0. found 392.0.

### Step 4: Ethyl 8-bromo-5-(2,4-dimethoxybenzylamino)-7 phenylimidazo[1,2-c]pyrimidine-2-carboxylate

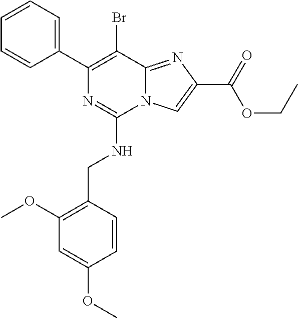 [SMILES: CCOC(=O)c1cn2c(NCc3ccc(OC)cc3OC)nc(-c3ccccc3)c(Br)c2n1]

In a 500 mL round bottom flask, ethyl 8-bromo-5-(methylthio)-7-phenylimidazo[1,2-c]pyrimidine-2-carboxylate (2.1 g, 5.35 mmol) was dissolved in 100 mL of DCM. To this solution, 3-chloroperbenzoic acid (mCPBA) (2.28 g, 10.2 mmol) in DCM (30 mL) was added dropwise through an addition funnel at 0° C. After addition, the reaction mixture was allowed to warm to room temperature and stirred for 4 h. The reaction was then quenched by adding saturated NaHCO₃ solution and the resulting two layers were separated. The organic layer was washed with brine, dried over Na₂SO₄, and filtrated. To this filtrate, (2,4-dimethoxyphenyl)methanamine (1.61 mL, 10.7 mmol) was added dropwise at room temperature. The resulting mixture was stirred for 2 h, and concentrated under reduced pressure. The resulting residue was purified by flash chromatography on a silica gel column eluting with 0 to 100% EtOAc in hexanes to afford the desired product. LC-MS calculated for C₂₄H₂₄BrN₄O₄ (M+H)⁺: m/z=511.1. found 511.1.

### Step 5: 8-Bromo-5-(2,4-dimethoxybenzylamino)-N-ethyl-7-phenylimidazo[1,2-c]pyrimidine-2-carboxamide

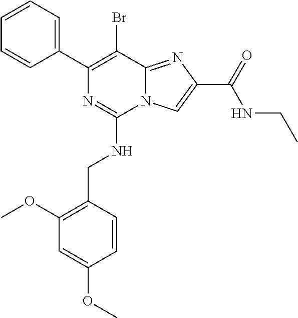 [SMILES: CCNC(=O)c1cn2c(NCc3ccc(OC)cc3OC)nc(-c3ccccc3)c(Br)c2n1]

Ethyl 8-bromo-5-(2,4-dimethoxybenzylamino)-7-phenylimidazo[1,2-c]pyrimidine-2-carboxylate (1.80 g, 3.5 mmol) in MeOH (20 mL), THF (20 mL), and water (10 mL) was added LiOH (0.34 g, 14.1 mmol) in one portion. The reaction mixture was stirred at 45° C. for 2 h, cooled to room temperature, and concentrated under reduced pressure. The resulting residue was dissolved in DMF (30 mL), followed by addition of ethanamine (2 M solution in THF, 3.52 mL, 7.04 mmol), N-ethyl-N-isopropylpropan-2-amine (1.84 mL, 10.6 mmol), and (benzotriazol-1-yloxy)tripyrrolidinophosphonium hexafluorophosphate (PyBOP) (3.66 g, 7.04 mmol). The reaction mixture was stirred at room temperature overnight before 100 mL of water was added. The resulting solid was collected and dried to afford the desired product as a yellow solid. LC-MS calculated for C₂₄H₂₅BrN₅O₃ (M+H)⁺: m/z=510.1. found 510.1.

### Step 6: tert-Butyl 4-(5-(2,4-dimethoxybenzylamino)-2-(ethylcarbamoyl)-7-phenylimidazo[1,2-c]pyrimidin-8 yl)-5,6-dihydropyridine-1(2H)-carboxylate

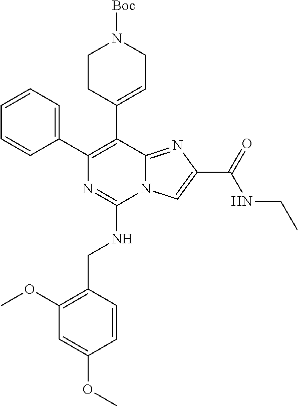 [SMILES: CCNC(=O)c1cn2c(NCc3ccc(OC)cc3OC)nc(-c3ccccc3)c(C3=CCN(C(=O)OC(C)(C)C)CC3)c2n1]

A mixture of 8-bromo-5-(2,4-dimethoxybenzylamino)-N-ethyl-7-phenylimidazo[1,2-c]pyrimidine-2-carboxamide (20.0 mg, 0.039 mmol), tert-butyl 4-(4,4,5,5-tetramethyl-1,3,2-dioxaborolan-2-yl)-3,6-dihydropyridine-1(2H)-carboxylate (14.5 mg, 0.047 mmol), XPhos Pd G2 (2.0 mg, 2.5 μmol), and Cs₂CO₃ (38.2 mg, 0.12 mmol) in 1,4-dioxane (1 mL) and water (0.2 mL) was degassed and stirred at 90° C. for 2 h. The reaction mixture was then cooled to room temperature, and concentrated under reduced pressure. The resulting residue was purified by flash chromatography on a silica gel column eluting with 0 to 100% EtOAc in hexanes to afford the desired product. LC-MS calculated for C₃₄H₄₁N₆O₅ (M+H)⁺: m/z=613.3. found 613.3.

### Step 7: 5-Amino-8-(1-carbamoyl-1,2,3,6-tetrahydropyridin-4-yl)-N-ethyl-7 phenylimidazo[1,2-c]pyrimidine-2-carboxamide

In a 10 mL reaction vial, tert-butyl 4-(5-(2,4-dimethoxybenzylamino)-2-(ethylcarbamoyl)-7-phenylimidazo[1,2-c]pyrimidin-8-yl)-5,6-dihydropyridine-1(2H)-carboxylate (20.0 mg, 0.033 mmol) was dissolved in 1 mL of TFA. The reaction mixture was stirred at 70° C. for 10 min, cooled to room temperature, concentrated and quenched with saturated NaHCO₃ solution. The resulting mixture was extracted with 3:1 DCM/IPA, and the combined organic layers were washed with brine, dried over Na₂SO₄, filtered and concentrated under reduced pressure. The resulting residue was dissolved in DCM (1 mL), and isocyanatotrimethylsilane (7.5 mg, 0.065 mmol) was added. The resulting mixture was stirred for 4 h, concentrated and purified by prep-LC-MS (pH=2, MeCN/water with TFA) to give the desired product as the TFA salt. LC-MS calculated for C₂₁H₂₄N₇O₂ (M+H)⁺: m/z=406.2. found 406.2.

### Example 16. 5-Amino-8-(1-carbamoylpiperidin-4-yl)-N-ethyl-7-phenylimidazo[1,2-c]pyrimidine-2-carboxamide

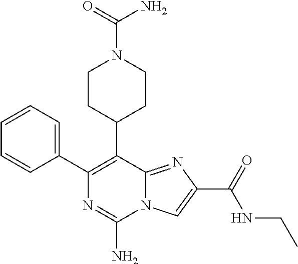 [SMILES: CCNC(=O)c1cn2c(N)nc(-c3ccccc3)c(C3CCN(C(N)=O)CC3)c2n1]

In a 10 mL reaction vial, 5-amino-8-(1-carbamoyl-1,2,3,6-tetrahydropyridin-4-yl)-N-ethyl-7-phenylimidazo[1,2-c]pyrimidine-2-carboxamide TFA salt (prepared in Example 15) (10.0 mg, 0.020 mmol) and palladium hydroxide on carbon (10 wt %, 3.7 mg, 2.3 μmol) were dissolved in 1 mL of MeOH. The reaction mixture was then stirred at 50° C. for 5 h under 1 atm of H₂. After completion, the reaction mixture was filtered and purified by prep-LC-MS (pH=2, MeCN/water with TFA) to give the desired product as the TFA salt. LC-MS calculated for C₂₁H₂₆N₇O₂ (M+H)⁺: m/z=408.2. found 408.2.

### Example 17. 5-Amino-7-(3-cyanophenyl)-N-ethyl-3-(2-hydroxyethoxy)-8-(2-methoxypyridin-4-yl)imidazo[1,2-c]pyrimidine-2-carboxamide

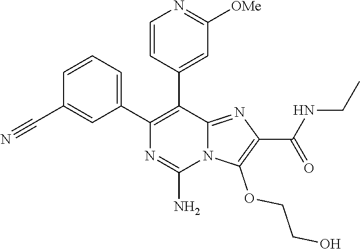 [SMILES: CCNC(=O)c1nc2c(-c3ccnc(OC)c3)c(-c3cccc(C#N)c3)nc(N)n2c1OCCO]

### Step 1: 3-(6 Amino-2-(methylthio)pyrimidin-4-yl)benzonitrile

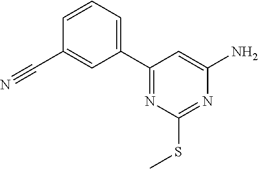 [SMILES: CSc1nc(N)cc(-c2cccc(C#N)c2)n1]

Bis(di-tert-butyl(4-dimethylaminophenyl)phosphine)dichloropalladium(II) (1.0 g, 5 mol %) was added to a mixture of 6-chloro-2-(methylthio)pyrimidin-4-amine (Combi-Blocks, cat #ST-1384) (5.0 g, 29 mmol), (3-cyanophenyl)boronic acid (8.4 g, 57 mmol) and cesium carbonate (37 g, 114 mmol) in toluene (100 mL) and water (10 mL). The mixture was purged with nitrogen, and then stirred at 115° C. for 16 h. The reaction mixture was cooled to room temperature, filtered through a Celite plug with DCM and concentrated under reduced pressure. Water (200 mL) was added to the residue and the resulting solid was collected by filtration, and then dried to give the desired product (6.5 g, 94%), which was used in the next step without further purification. LC-MS calculated for C₁₂H₁₁N₄S (M+H)⁺: m/z=243.1. found 243.2.

### Step 2: 3-(6 Amino-5-bromo-2-(methylthio)pyrimidin-4-yl)benzonitrile

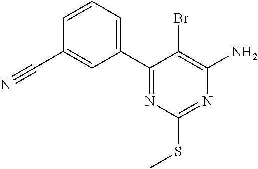 [SMILES: CSc1nc(N)c(Br)c(-c2cccc(C#N)c2)n1]

To a solution of 3-(6-amino-2-(methylthio)pyrimidin-4-yl)benzonitrile (6.5 g, 27 mmol) in DMSO (55 mL), MeCN (30 mL) and water (1.8 mL) at 0° C. was added N-bromosuccinimide (4.8 g, 27 mmol). The reaction mixture was stirred for 2 h at room temperature before water (200 mL) was added. The resulting precipitate was collected by filtration, and then dried to give the desired product (8.6 g, 99%), which was used in the next step without further purification. LC-MS calculated for C₁₂H₁₀BrN₄S (M+H)⁺: m/z=321.0. found 321.1.

### Step 3: Ethyl 8-bromo-7-(3-cyanophenyl)-5-(methylthio)imidazo[1,2-c]pyrimidine-2-carboxylate

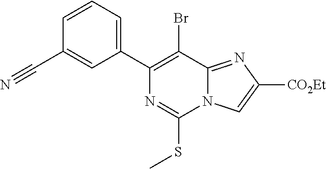 [SMILES: CCOC(=O)c1cn2c(SC)nc(-c3cccc(C#N)c3)c(Br)c2n1]

To a solution of 3-(6-amino-5-bromo-2-(methylthio)pyrimidin-4-yl)benzonitrile (2.0 g, 6.2 mmol) in DME (26 mL) was added ethyl 3-bromo-2-oxopropanoate (2.3 mL, 19 mmol). The reaction mixture was stirred at 110° C. for 3 h before the volatiles were removed under reduced pressure. The resulting solid was collected by filtration, washed with Et₂O (100 mL), and dried to give the desired product as HBr salt (1.4 g, 54%). LC-MS calculated for C₁₇H₁₄BrN₄O₂S (M+H)⁺: m/z=417.0. found 417.0.

### Step 4: Ethyl 8-bromo-7-(3-cyanophenyl)-5-(2,4-dimethoxybenzylamino)imidazo[1,2-c]pyrimidine-2-carboxylate

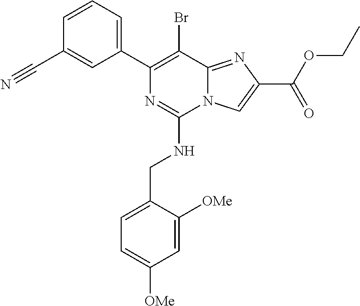 [SMILES: CCOC(=O)c1cn2c(NCc3ccc(OC)cc3OC)nc(-c3cccc(C#N)c3)c(Br)c2n1]

To a solution of ethyl 8-bromo-7-(3-cyanophenyl)-5-(methylthio)imidazo[1,2-c]pyrimidine-2-carboxylate hydrobromide (1.4 g, 3.4 mmol) in DCM (170 ml) at 0° C. was added a solution of mCPBA (1.6 g, 6.4 mmol, 70%) in DCM (15 mL) dropwise. The solution was stirred at room temperature for 2 h. Following complete consumption of starting material, (2,4-dimethoxyphenyl)methanamine (1.02 ml, 6.8 mmol) in DCM (15 mL) was added and the suspension was stirred for 2 h. The reaction mixture was then washed with saturated NaHCO₃ solution (100 mL), water (100 mL), and brine (50 mL). The organic layer was dried over MgSO₄, filtered and concentrated under reduced pressure. The resulting material was purified by column chromatography eluting with 0-100% EtOAc/hexanes to give the desired product (1.4 g, 54%). LC-MS calculated for C₂₅H₂₃BrN₅O₄ (M+H)⁺: m/z=536.1. found 536.2.

### Step 5: Ethyl 7-(3-cyanophenyl)-5-(2,4-dimethoxybenzylamino)-8-(2-methoxypyridin-4-yl)imidazo[1,2-c]pyrimidine-2-carboxylate

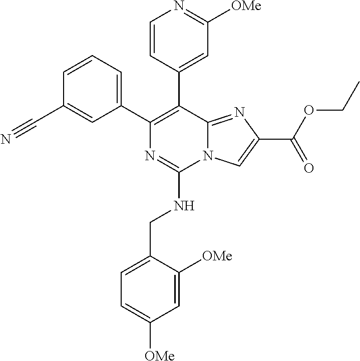 [SMILES: CCOC(=O)c1cn2c(NCc3ccc(OC)cc3OC)nc(-c3cccc(C#N)c3)c(-c3ccnc(OC)c3)c2n1]

[1,1′-bis(dicyclohexylphosphino)ferrocene]dichloropalladium(II) (230 mg, 10 mol %) was added to a mixture of ethyl 8-bromo-7-(3-cyanophenyl)-5-(2,4-dimethoxybenzylamino)imidazo[1,2-c]pyrimidine-2-carboxylate (1.7 g, 3.1 mmol), 2-methoxypyridin-4-ylboronic acid (570 mg, 3.7 mmol), and cesium carbonate (1.7 g, 5.2 mmol) in tert-butanol (13 mL) and water (2.6 mL). The reaction mixture was purged with nitrogen, and stirred at 120° C. for 3 h. The reaction mixture was then cooled to room temperature, filtered through a Celite plug with DCM and concentrated under reduced pressure. The resulting material was purified by column chromatography eluting with 0-100% EtOAc/hexanes to give the desired product (370 mg). LC-MS calculated for C₃₁H₂₉N₆O₅ (M+H)⁺: m/z=565.2. found 565.4.

### Step 6: 5-Amino-7-(3-cyanophenyl)-N-ethyl-8-(2-methoxypyridin-4-yl)imidazo[1,2-c]pyrimidine-2-carboxamide

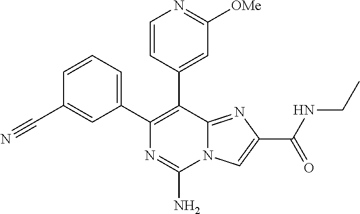 [SMILES: CCNC(=O)c1cn2c(N)nc(-c3cccc(C#N)c3)c(-c3ccnc(OC)c3)c2n1]

To ethyl 7-(3-cyanophenyl)-NH_{Z}-methoxypyridin-4-yl)imidazo[1,2-c]pyrimidine-2-carboxylate (370 mg, 0.65 mmol) was added ethanamine (3.3 mL, 2 M in MeOH). The solution was stirred at 85° C. in a sealed vial for 16 h, cooled to room temperature and the volatiles were removed under reduced pressure. TFA (2.0 mL) was added to the residue and the mixture was stirred at 100° C. for 10 min in a sealed vial. The reaction mixture was then cooled to room temperature, and the volatiles were removed under reduced pressure. The product was purified by column chromatography eluting with 0-10% MeOH/DCM containing 0.5% triethylamine. LC-MS calculated for C₂₂H₂₀N₇O₂ (M+H)⁺: m/z=414.2. found 414.3.

### Step 7: 5-Amino-3-bromo-7-(3-cyanophenyl)-N-ethyl-8-(2-methoxypyridin-4-yl)imidazo[1,2-c]pyrimidine-2-carboxamide

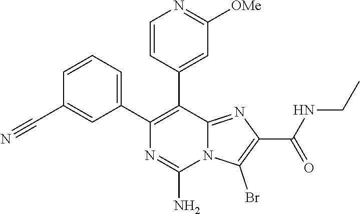 [SMILES: CCNC(=O)c1nc2c(-c3ccnc(OC)c3)c(-c3cccc(C#N)c3)nc(N)n2c1Br]

To a solution of 5-amino-7-(3-cyanophenyl)-N-ethyl-8-(2-methoxypyridin-4-yl)imidazo[1,2-c]pyrimidine-2-carboxamide (110 mg, 0.27 mmol) in DMF (0.5 mL) at 0° C. was added N-bromosuccinimide (47 mg, 0.27 mmol). The reaction mixture was stirred for 2 h at room temperature before water (1.0 mL) was added. The resulting precipitate was collected by filtration, and dried to give the desired product, which was used in the next step without further purification. LC-MS calculated for C₂₂H₁₉BrN₇O₂ (M+H)⁺: m/z=492.1. found 492.1.

### Step 8: 5-Amino-7-(3-cyanophenyl)-N-ethyl-3-(2-hydroxyethoxy)-8-(2-methoxypyridin-4-yl)imidazo[1,2-c]pyrimidine-2-carboxamide

To a reaction vial was added copper(I) iodide (1.2 mg, 6.1 μmol), 3,4,7,8-tetramethyl-1,10-phenanthroline (2.9 mg, 0.01 mmol), 5-amino-3-bromo-7-(3-cyanophenyl)-N-ethyl-8-(2-methoxypyridin-4-yl)imidazo[1,2-c]pyrimidine-2-carboxamide (30 mg, 0.06 mmol), and cesium carbonate (30 mg, 0.09 mmol). The reaction vial was flushed with nitrogen and fitted with a rubber septum. Toluene (0.2 mL) and ethane-1,2-diol (0.17 mL, 3.1 mmol) were added and the rubber septum was replaced with a Teflon-lined cap. The reaction mixture was stirred at 110° C. for 24 h, cooled to room temperature, diluted with ethyl acetate (2 mL), and filtered through a plug of silica gel. The filtrate was concentrated and purified by prep-LCMS (pH=2, MeCN/water with TFA) to give the desired product as the TFA salt. LC-MS calculated for C₂₄H₂₄N₇O₄ (M+H)⁺: m/z=474.2. found 474.2.

### Example 18. 5-Amino-7-(3-cyanophenyl)-N-ethyl-8-(2-methoxypyridin-4-yl)-3-methylimidazo[1,2-c]pyrimidine-2-carboxamide

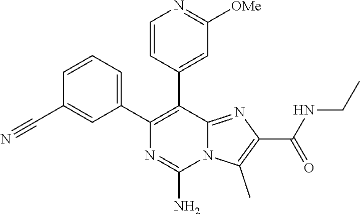 [SMILES: CCNC(=O)c1nc2c(-c3ccnc(OC)c3)c(-c3cccc(C#N)c3)nc(N)n2c1C]

To a mixture of 5-amino-3-bromo-7-(3-cyanophenyl)-N-ethyl-8-(2-methoxypyridin-4-yl)imidazo[1,2-c]pyrimidine-2-carboxamide (prepared in Example 17, Step 7) (40 mg, 0.08 mmol) and [1,1′-Bis(diphenylphosphino)ferrocene]dichloropalladium(II), complex with dichloromethane (3.3 mg, 4.1 μcool) in 1,4-dioxane (0.50 mL) under nitrogen atmosphere was added dimethylzinc in toluene (0.27 mL, 1.2 M) dropwise. The resulting mixture was stirred at 90° C. overnight, cooled to room temperature, diluted with DCM (5 mL) and filtered through a Celite plug. The filtrate was concentrated under reduced pressure and the crude material was purified by prep-LCMS (pH=2, MeCN/water with TFA) to give the desired product as the TFA salt. LC-MS calculated for C₂₃H₂₂N₇O₂ (M+H)⁺: m/z=428.2. found 428.2.

### Example 19. 5-Amino-7-(3-cyanophenyl)-N-ethyl-8-(2-methoxypyridin-4-yl)-3-(pyridin-2-yl)imidazo[1,2-c]pyrimidine-2-carboxamide

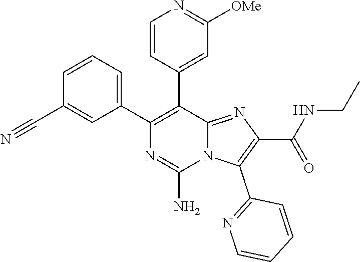 [SMILES: CCNC(=O)c1nc2c(-c3ccnc(OC)c3)c(-c3cccc(C#N)c3)nc(N)n2c1-c1ccccn1]

Bis(triphenylphosphine)palladium(II) chloride (4.3 mg, 6.1 μmol) was added to a mixture of 5-amino-3-bromo-7-(3-cyanophenyl)-N-ethyl-8-(2-methoxypyridin-4-yl)imidazo[1,2-c]pyrimidine-2-carboxamide (prepared in Example 17, Step 7) (30 mg, 0.06 mmol) and 2-(tributylstannyl)pyridine (0.05 mL, 0.12 mmol) in DMF (0.5 mL). The reaction mixture was purged with nitrogen and then stirred at 100° C. for 5 h. After cooling to room temperature, the mixture was diluted with DCM (5 mL) and filtered through a Celite plug. The filtrate was concentrated under reduced pressure and the crude material was purified by prep-LCMS (pH=2, MeCN/water with TFA) to give the desired product as the TFA salt. LC-MS calculated for C₂₇H₂₃N₈O₂ (M+H)⁺: m/z=491.2. found 491.2.

### Example 20. 5-Amino-3-bromo-8-(2,6-dimethylpyridin-4-yl)-N-ethyl-7-phenylimidazo[1,2-c]pyrimidine-2-carboxamide

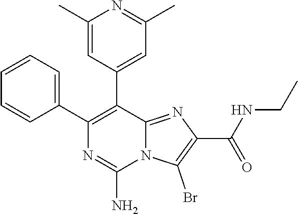 [SMILES: CCNC(=O)c1nc2c(-c3cc(C)nc(C)c3)c(-c3ccccc3)nc(N)n2c1Br]

### Step 1: Ethyl 5-(2,4-dimethoxybenzylamino)-8-(2,6-dimethylpyridin-4-yl)-7 phenylimidazo[1,2-c]pyrimidine-2-carboxylate

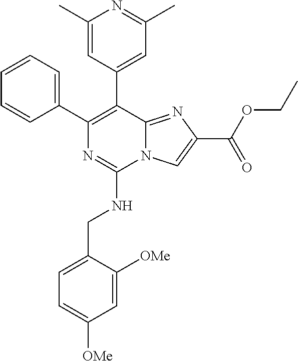 [SMILES: CCOC(=O)c1cn2c(NCc3ccc(OC)cc3OC)nc(-c3ccccc3)c(-c3cc(C)nc(C)c3)c2n1]

[1,1′-bis(dicyclohexylphosphino)ferrocene]dichloropalladium(II) (400 mg, 10 mol %) was added to a mixture of ethyl 8-bromo-5-((2,4-dimethoxybenzyl)amino)-7-phenylimidazo[1,2-c]pyrimidine-2-carboxylate (prepared in Example 15, Step 4) (2.7 g, 5.3 mmol), (2,6-dimethylpyridin-4-yl)boronic acid (1.2 g, 7.9 mmol), and cesium carbonate (3.4 g, 11 mmol) in tert-butanol (20 mL) and water (3.8 mL). The mixture was purged with nitrogen, and then stirred at 120° C. for 1.5 h. The reaction mixture was cooled to room temperature, filtered through a Celite plug with DCM and concentrated under reduced pressure. The resulting material was purified by column chromatography eluting with 0-20% MeOH/DCM to give the desired product (2.8 g, 99%). LC-MS calculated for C₃₁H₃₂N₅O₄ (M+H)⁺: m/z=538.2. found 538.3.

### Step 2: 5-(2,4-Dimethoxybenzylamino)-8-(2,6-dimethylpyridin-4-yl)-7 phenylimidazo[1,2-c]pyrimidine-2-carboxylic acid

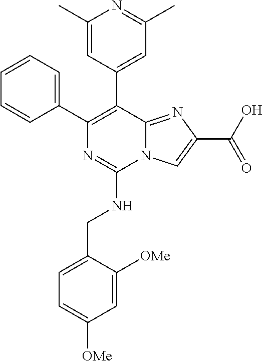 [SMILES: COc1ccc(CNc2nc(-c3ccccc3)c(-c3cc(C)nc(C)c3)c3nc(C(=O)O)cn23)c(OC)c1]

To a solution of ethyl 5-(2,4-dimethoxybenzylamino)-8-(2,6-dimethylpyridin-4-yl)-7-phenylimidazo[1,2-c]pyrimidine-2-carboxylate (1.0 g, 1.9 mmol) in THF (3.9 mL) was added lithium hydroxide (0.18 g, 7.4 mmol) and water (3.9 mL). The resulting mixture was stirred at 50° C. for 16 h. After cooling to room temperature, water (2.0 mL) was added and the pH was adjusted using 1 N HCl to pH 2. The resulting precipitate was collected by filtration, washed with water and dried to afford the crude product, which was used in the next step without further purification. LC-MS calculated for C₂₉H₂₈N₅O₄ (M+H)⁺: m/z=510.2. found 510.2.

### Step 3: 5-Amino-8-(2,6-dimethylpyridin-4-yl)-N-ethyl-7 phenylimidazo[1,2-c]pyrimidine-2-carboxamide

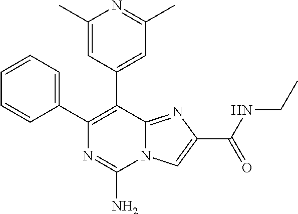 [SMILES: CCNC(=O)c1cn2c(N)nc(-c3ccccc3)c(-c3cc(C)nc(C)c3)c2n1]

To a solution of 5-(2,4-dimethoxybenzylamino)-8-(2,6-dimethylpyridin-4-yl)-7-phenylimidazo[1,2-c]pyrimidine-2-carboxylic acid (0.95 g, 1.9 mmol) in DMF (17 mL) was added triethylamine (0.78 mL, 5.6 mmol). The solution was stirred for 5 min before the addition of BOP (1.2 g, 2.8 mmol) and ethanamine (9.3 mL, 2 M in THF). The reaction mixture was then stirred at room temperature for 30 min, quenched with water (20 mL), and extracted with EtOAc (5×30 mL). The combined organic layers were washed with water (50 mL) and brine (30 mL), dried with MgSO₄, and concentrated under reduced pressure. To the resulting residue was added TFA (3.0 mL), and the reaction mixture was stirred at 100° C. for 10 min. After cooling to room temperature, the volatiles were removed under reduced pressure to afford the crude product, which was used in the next step without further purification. LC-MS calculated for C₂₂H₂₃N₆O (M+H)⁺: m/z=387.2. found 387.3.

### Step 4: 5-Amino-3-bromo-8-(2,6-dimethylpyridin-4-yl)-N-ethyl-7 phenylimidazo[1,2-c]pyrimidine-2-carboxamide

To a solution of 5-amino-8-(2,6-dimethylpyridin-4-yl)-N-ethyl-7-phenylimidazo[1,2-c]pyrimidine-2-carboxamide (700 mg, 1.8 mmol) in DMSO (3.6 mL)/MeCN (1.9 mL)/water (0.12 mL) at 0° C. was added N-bromosuccinimide (320 mg, 1.8 mmol). The reaction mixture was stirred for 2 h at room temperature, at which point water (20 mL) was added and the desired product was collected by filtration. The product was purified by prep-LCMS (pH=2, MeCN/water with TFA) to give the desired product as the TFA salt. LC-MS calculated for C₂₂H₂₂BrN₆O (M+H)⁺: m/z=465.1. found 465.1.

### Example 21. 5-Amino-3-cyano-8-(2,6-dimethylpyridin-4-yl)-N-ethyl-7-phenylimidazo[1,2-c]pyrimidine-2-carboxamide

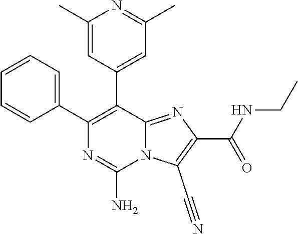 [SMILES: CCNC(=O)c1nc2c(-c3cc(C)nc(C)c3)c(-c3ccccc3)nc(N)n2c1C#N]

To a microwave vial was added copper(I) cyanide (8.7 mg, 0.10 mmol) and 5-amino-3-bromo-8-(2,6-dimethylpyridin-4-yl)-N-ethyl-7-phenylimidazo[1,2-c]pyrimidine-2-carboxamide (prepared in Example 20) (30 mg, 0.06 mmol) in DMF (0.30 mL). The vial was flushed with nitrogen and sealed before being heated in a microwave reactor at 180° C. for 10 min. After cooling to room temperature, the reaction mixture was diluted with NH₄OH (1 mL) and H₂O (1 mL), and extracted with EtOAc (3×5 mL). The combined organic layers were dried over MgSO₄, filtered, and concentrated under reduced pressure. The resulting material was purified by prep-LCMS (pH=2, MeCN/water with TFA) to give the desired product as the TFA salt. LC-MS calculated for C₂₃H₂₂N₇O (M+H)⁺: m/z=412.2. found 412.2.

### Example 22. 8-(2,6-Dimethylpyridin-4-yl)-N-ethyl-5-(ethylamino)-7-phenylimidazo[1,2-c]pyrimidine-2-carboxamide

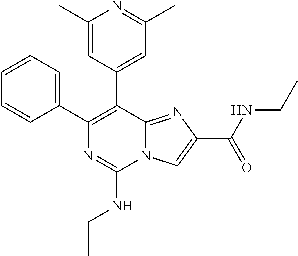 [SMILES: CCNC(=O)c1cn2c(NCC)nc(-c3ccccc3)c(-c3cc(C)nc(C)c3)c2n1]

### Step 1: 5-(2,6-Dimethylpyridin-4-yl)-2-(methylthio)-6 phenylpyrimidin-4-amine

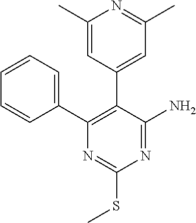 [SMILES: CSc1nc(N)c(-c2cc(C)nc(C)c2)c(-c2ccccc2)n1]

[1,1′-bis(dicyclohexylphosphino)ferrocene]dichloropalladium(II) (510 mg, 10 mol %) was added to a mixture of 5-bromo-2-(methylthio)-6-phenylpyrimidin-4-amine (prepared in Example 15, Step 2) (2.0 g, 6.8 mmol), (2,6-dimethylpyridin-4-yl)boronic acid (1.5 g, 10 mmol), and cesium carbonate (4.4 g, 14 mmol) in tert-butanol (25 mL) and water (5.0 mL). The mixture was purged with nitrogen, and then stirred at 120° C. for 2 h. The reaction mixture was cooled to room temperature and filtered through a Celite plug. The filtrate was concentrated under reduced pressure and purified by flash chromatography on a silica gel column eluting with 0-100% EtOAc/hexanes to give the desired product (440 mg, 20%). LC-MS calculated for C₁₈H₁₉N₄S (M+H)⁺: m/z=323.1. found 323.1.

### Step 2: Ethyl 8-(2,6-dimethylpyridin-4-yl)-5-(methylthio)-7 phenylimidazo[1,2-c]pyrimidine-2-carboxylate

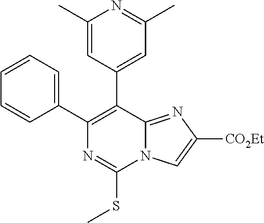 [SMILES: CCOC(=O)c1cn2c(SC)nc(-c3ccccc3)c(-c3cc(C)nc(C)c3)c2n1]

To a solution of 5-(2,6-dimethylpyridin-4-yl)-2-(methylthio)-6-phenylpyrimidin-4-amine (800 mg, 2.5 mmol) in DME (10 mL) was added ethyl 3-bromo-2-oxopropanoate (0.93 mL, 7.4 mmol). The reaction mixture was stirred at 110° C. for 2 h before the volatiles were removed under reduced pressure. The resulting residue was diluted with DCM (20 mL), washed with saturated NaHCO₃ solution (20 mL), water (20 mL) and brine (10 mL), dried over MgSO₄, filtered, and concentrated under reduced pressure. The resulting material was purified by column chromatography eluting with 0-20% MeOH/DCM to give the desired product (1.0 g, 99%). LC-MS calculated for C₂₃H₂₃N₄O₂S (M+H)⁺: m/z=419.2. found 419.1.

### Step 3: 8-(2,6-Dimethylpyridin-4-yl)-N-ethyl-5-(ethylamino)-7 phenylimidazo[1,2-c]pyrimidine-2-carboxamide

Ethyl 8-(2,6-dimethylpyridin-4-yl)-5-(methylthio)-7-phenylimidazo[1,2-c]pyrimidine-2-carboxylate (100 mg, 0.24 mmol) was suspended in a solution of ethanamine (1.2 mL, 2 M in MeOH) and heated at reflux for 16 h. After cooling to room temperature, the solvent was removed under reduced pressure and the resulting material was purified by prep-LCMS (pH=2, MeCN/water with TFA) to give the desired product as the TFA salt. LC-MS calculated for C₂₄H₂₇N₆O (M+H)⁺: m/z=415.2. found 415.1.

### Example 23. 4-(5-Amino-2-(ethylcarbamoyl)-7-phenylimidazo[1,2-c]pyrimidin-8-yl)-2,6-dimethylpyridine 1-oxide

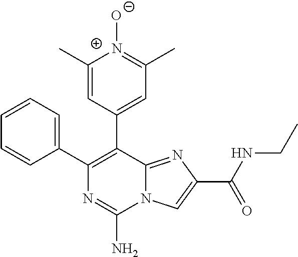 [SMILES: CCNC(=O)c1cn2c(N)nc(-c3ccccc3)c(-c3cc(C)[n+]([O-])c(C)c3)c2n1]

### Step 1: 4-(2-(Ethoxycarbonyl)-5-(methylsulfonyl)-7-phenylimidazo[1,2-c]pyrimidin-8 yl)-2,6-dimethylpyridine 1-oxide

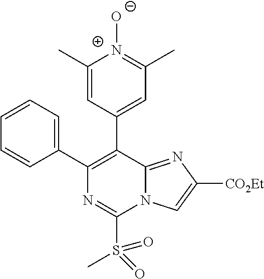 [SMILES: CCOC(=O)c1cn2c(S(C)(=O)=O)nc(-c3ccccc3)c(-c3cc(C)[n+]([O-])c(C)c3)c2n1]

To a mixture of ethyl 8-(2,6-dimethylpyridin-4-yl)-5-(methylthio)-7-phenylimidazo[1,2-c]pyrimidine-2-carboxylate (prepared in Example 22, Step 2) (50 mg, 0.12 mmol) in DCM (10 mL) at 0° C. was added mCPBA (88 mg, 0.36 mmol). The resulting solution was stirred at room temperature for 3 h, and then the volatiles were removed under reduced pressure. The resulting residue was taken up in EtOAc (20 mL), washed with a mixture of saturated Na₂S₂O₃ solution (10 mL) and saturated NaHCO₃ solution (10 mL), and then brine (10 mL). The organic layer was dried over MgSO₄ and concentrated under reduced pressure to afford the crude product, which was used in the next step without further purification. LC-MS calculated for C₂₃H₂₃N₄O₅S (M+H)⁺: m/z=467.1. found 467.1.

### Step 2: 4-(5 Amino-2-(ethoxycarbonyl)-7 phenylimidazo[1,2-c]pyrimidin-8 yl)-2,6-dimethylpyridine 1-oxide

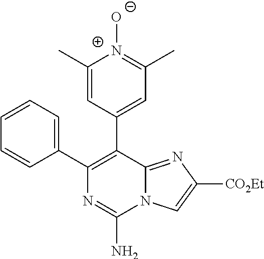 [SMILES: CCOC(=O)c1cn2c(N)nc(-c3ccccc3)c(-c3cc(C)[n+]([O-])c(C)c3)c2n1]

To a solution of 4-(2-(ethoxycarbonyl)-5-(methylsulfonyl)-7-phenylimidazo[1,2-c]pyrimidin-8-yl)-2,6-dimethylpyridine 1-oxide (54 mg, 0.12 mmol) in acetonitrile (1.0 mL) was added concentrated ammonium hydroxide (0.09 mL, 14 M). The reaction mixture was stirred at room temperature for 30 min before the volatiles were removed under reduced pressure. The resulting material was purified by prep-LCMS (pH=10, MeCN/water with NH₄OH) to give the desired product as the free base. LC-MS calculated for C₂₂H₂₂N₅O₃ (M+H)⁺: m/z=404.2. found 404.2.

### Step 3: 4-(5 Amino-2-(ethylcarbamoyl)-7 phenylimidazo[1,2-c]pyrimidin-8 yl)-2,6-dimethylpyridine 1-oxide

4-(5-Amino-2-(ethoxycarbonyl)-7-phenylimidazo[1,2-c]pyrimidin-8-yl)-2,6-dimethylpyridine 1-oxide (15 mg, 0.03 mmol) was suspended in a solution of ethanamine (1.0 mL, 2 M in methanol) and heated at reflux for 1 h. After cooling to room temperature, the solvent was removed under reduced pressure and the product was purified by prep-LCMS (pH=2, MeCN/water with TFA) to give the desired product as the TFA salt. LC-MS calculated for C₂₂H₂₃N₆O₂ (M+H)⁺: m/z=403.2. found 403.1.

### Example 24. 3-(5-Amino-8-(1-ethyl-6-oxo-1,6-dihydropyridin-3-yl)-2-(3-hydroxyazetidine-1-carbonyl)imidazo[1,2-c]pyrimidin-7-yl)benzonitrile

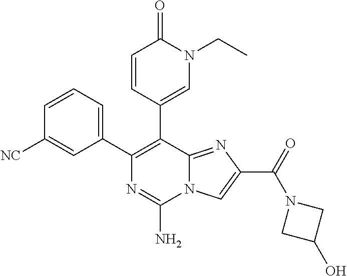 [SMILES: CCn1cc(-c2c(-c3cccc(C#N)c3)nc(N)n3cc(C(=O)N4CC(O)C4)nc23)ccc1=O]

### Step 1: 3-(8-Bromo-5-(2,4-dimethoxybenzylamino)-2-(3-hydroxyazetidine-1-carbonyl)imidazo[1,2-c]pyrimidin-7-yl)benzonitrile

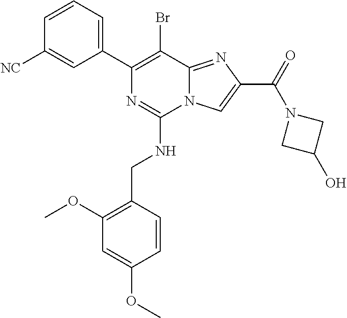 [SMILES: COc1ccc(CNc2nc(-c3cccc(C#N)c3)c(Br)c3nc(C(=O)N4CC(O)C4)cn23)c(OC)c1]

Ethyl 8-bromo-7-(3-cyanophenyl)-5-(2,4-dimethoxybenzylamino)imidazo[1,2-c]pyrimidine-2-carboxylate (prepared in Example 17, Step 4) (100 mg, 0.186 mmol) in MeOH (2 mL), THE (2 mL), and water (1 mL) was added LiOH (17.9 mg, 0.75 mmol). The reaction mixture was stirred at 45° C. for 2 h, and the solvent was removed under reduced pressure. The resulting residue was dissolved in DMF (3 mL), followed by the addition of azetidin-3-ol (27.3 mg, 0.37 mmol), N-ethyl-N-isopropylpropan-2-amine (98 μL, 0.56 mmol), and HATU (142 mg, 0.37 mmol). The reaction mixture was stirred at room temperature overnight before 10 mL of water was added. The precipitated solid was collected and dried to afford the desired product as a yellow solid. LC-MS calculated for C₂₆H₂₄BrN₆O₄ (M+H)⁺: m/z=563.1. found 563.1.

### Step 2: 3-(5-Amino-8-(1-ethyl-6-oxo-1,6-dihydropyridin-3 yl)-2-(3-hydroxyazetidine-1-carbonyl)imidazo[1,2-c]pyrimidin-7-yl)benzonitrile

A mixture of 3-(8-bromo-5-(2,4-dimethoxybenzylamino)-2-(3-hydroxyazetidine-1-carbonyl)imidazo[1,2-c]pyrimidin-7-yl)benzonitrile (20 mg, 0.048 mmol), 1-ethyl-5-(4,4,5,5-tetramethyl-1,3,2-dioxaborolan-2-yl)pyridin-2(1H)-one (14.5 mg, 0.058 mmol), XPhos Pd G2 (2.0 mg, 2.5 μmol), and Cs₂CO₃ (47 mg, 0.15 mmol) in 1,4-dioxane (1 mL) and water (0.2 mL) was degassed and sealed. The reaction was stirred at 90° C. for 2 h, cooled to room temperature, and concentrated. To the resulting residue, 1 mL of TFA was added, and the resulting mixture was stirred at 70° C. for 30 min. After completion, the reaction mixture was concentrated, diluted with methanol, and purified with prep-LC-MS (pH=2, MeCN/water with TFA) to give the desired product as a TFA salt. LC-MS calculated for C₂₄H₂₂N₇O₃ (M+H)⁺: m/z=456.2. found 456.2.

### Example 25. 5-Amino-7-(3-cyanophenyl)-8-(1-ethyl-6-oxo-1,6-dihydropyridin-3-yl)-N-(1-(2-hydroxyethyl)-1H-pyrazol-4-yl)imidazo[1,2-c]pyrimidine-2-carboxamide

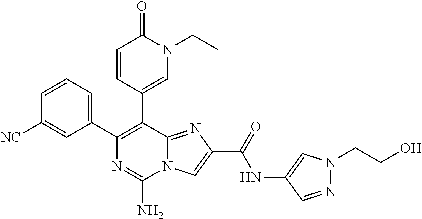 [SMILES: CCn1cc(-c2c(-c3cccc(C#N)c3)nc(N)n3cc(C(=O)Nc4cnn(CCO)c4)nc23)ccc1=O]

This compound was prepared using similar procedures as described for Example 24 with 2-(4-amino-1H-pyrazol-1-yl)ethanol (AstaTech Product List #50515) replacing azetidin-3-ol in Step 1. The product was purified by prep-LC-MS (pH=2, MeCN/water with TFA) to give the desired product as a TFA salt. LC-MS calculated for C₂₆H₂₄N₉O₃ (M+H)⁺: m/z=510.2. found 510.2.

### Example 26. 5-Amino-7-(3-cyanophenyl)-N-ethyl-8-(pyridin-4-yl)imidazo[1,2-c]pyrimidine-2-carboxamide

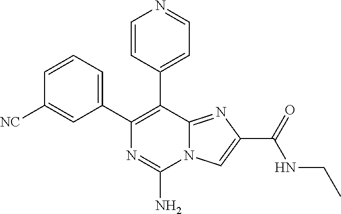 [SMILES: CCNC(=O)c1cn2c(N)nc(-c3cccc(C#N)c3)c(-c3ccncc3)c2n1]

### Step 1: Ethyl 5-amino-7-chloroimidazo[1,2-c]pyrimidine-2-carboxylate

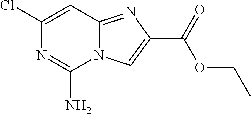 [SMILES: CCOC(=O)c1cn2c(N)nc(Cl)cc2n1]

A solution of 6-chloropyrimidine-2,4-diamine (2 g, 13.8 mmol) and ethyl 3-bromo-2-oxopropanoate (2.6 ml, 20.8 mmol) in DME (50 ml) was stirred at 70° C. overnight. After completion, the reaction was cooled to room temperature and the solid was collected by filtration. The crude solid was dissolved in hot methanol (25 mL) and the desired product was recrystallized by slowly cooling the solution to room temperature. The product was filtered, washed with EtOAc, and dried to give the desired product. LC-MS calculated for C₉H₁₀ClN₄O₂ (M+H)⁺: m/z=241.0. found 241.1.

### Step 2: Ethyl 5-amino-7-(3-cyanophenyl)imidazo[1,2-c]pyrimidine-2-carboxylate

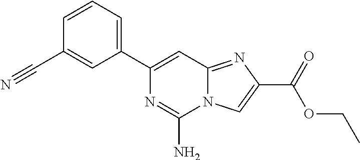 [SMILES: CCOC(=O)c1cn2c(N)nc(-c3cccc(C#N)c3)cc2n1]

A mixture of ethyl 5-amino-7-chloroimidazo[1,2-c]pyrimidine-2-carboxylate (0.60 g, 2.49 mmol), (3-cyanophenyl)boronic acid (0.44 g, 2.99 mmol), XPhos Pd G2 (0.098 g, 0.125 mmol) and sodium carbonate (0.53 g, 4.99 mmol) in 1,4-dioxane (50 mL) and water (5.0 mL) was purged with nitrogen and then stirred at 100° C. for 1 h. After being cooled to room temperature, the reaction mixture diluted with EtOAc, washed with brine, dried over Na₂SO₄, filtered, and concentrated under reduced pressure. Light yellow solid precipitated from the solution, which was filtered and dried to afford the desired product. LC-MS calculated for C₁₆H₁₄N₅O₂ (M+H)⁺: m/z=308.1. found 308.1.

### Step 3: Ethyl 5-amino-8-bromo-7-(3-cyanophenyl)imidazo[1,2-c]pyrimidine-2-carboxylate

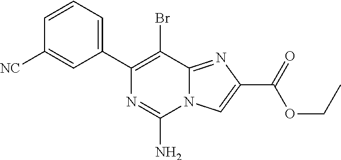 [SMILES: CCOC(=O)c1cn2c(N)nc(-c3cccc(C#N)c3)c(Br)c2n1]

To a solution of ethyl 5-amino-7-(3-cyanophenyl)imidazo[1,2-c]pyrimidine-2-carboxylate (2.3 g, 7.48 mmol) in DMF (50 ml) was slowly added a solution of N-bromosuccinimide (1.33 g, 7.48 mmol) in DMF (5.0 mL) at 0° C. The reaction mixture was then stirred at room temperature for 2 h before water (100 mL) was added. The resulting light yellow solid was collected by filtration and dried to obtain the desired product (2.3 g, 80%). LC-MS calculated for C₁₆H₁₃BrN₅O₂ (M+H)⁺: m/z=386.0. found 386.0.

Alternatively, this compound can be prepared using the following procedure: to a solution of ethyl 8-bromo-7-(3-cyanophenyl)-5-(methylthio)imidazo[1,2-c]pyrimidine-2-carboxylate (prepared in Example 17, Step 3) (2.40 g, 5.75 mmol) in DCM (100 mL) was added a solution mCPBA (77%, 1.93 g, 8.63 mmol) in DCM (30 mL) this solution was dried over anhydrous magnesium sulfate through an addition funnel at room temperature for 30 minutes. The reaction mixture was then stirred for 4 h, and quenched by bubbling NH₃ gas via cannula for 1 h. The reaction mixture was then concentrated under reduced pressure to give a crude mixture, which was poured into a saturated NaHCO₃ solution (150 mL). The resulting solid was collected by filtration, washed with water and hexanes, and dried to afford the desired product as a brown solid (1.9 g, 86%). LC-MS calculated for C₁₆H₁₃BrN₅O₂ (M+H)⁺: m/z=386.0. found 386.0.

### Step 4: 5-Amino-8-bromo-7-(3-cyanophenyl)-N-ethylimidazo[1,2-c]pyrimidine-2-carboxamide

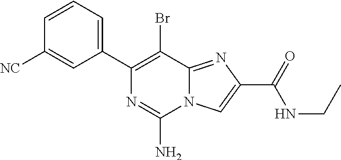 [SMILES: CCNC(=O)c1cn2c(N)nc(-c3cccc(C#N)c3)c(Br)c2n1]

To a mixture of ethyl 5-amino-8-bromo-7-(3-cyanophenyl)imidazo[1,2-c]pyrimidine-2-carboxylate (1.00 g, 2.59 mmol) in MeOH (20 mL), THE (20 mL), and water (10 mL) was added LiOH (124 mg, 5.18 mmol). The reaction mixture was stirred at room temperature for 2 h, and the solvent was removed under reduced pressure. The resulting residue was dissolved in DMF (30 mL), followed by the addition of ethylamine solution (2.1 mL, 25.9 mmol, 70% in water), triethyl amine (1.08 mL, 7.77 mmol), and BOP (2.29 g, 5.18 mmol). The reaction mixture was stirred at room temperature overnight before 100 mL of water was added. The resulting solid was collected by filtration and dried to afford the desired product as a yellow solid (0.77 g). LC-MS calculated for C₁₆H₁₄BrN₆O (M+H)⁺: m/z=385.0. found 385.1.

### Step 5: 5-Amino-7-(3-cyanophenyl)-N-ethyl-8-(pyridin-4-yl)imidazo[1,2-c]pyrimidine-2-carboxamide

A mixture of 5-amino-8-bromo-7-(3-cyanophenyl)-N-ethylimidazo[1,2-c]pyrimidine-2-carboxamide (10.0 mg, 0.026 mmol), pyridin-4-ylboronic acid (4.8 mg, 0.039 mmol), XPhos Pd G2 (2.0 mg, 2.51 μmol), and Na₂CO₃ (8.3 mg, 0.078 mmol) in 1,4-dioxane (1.5 mL) and water (0.15 mL) was degassed and sealed. The reaction mixture was stirred at 110° C. for 1 h, cooled to room temperature, diluted with MeOH, and purified with prep-LC-MS (pH=2, MeCN/water with TFA) to give the desired product as a TFA salt. LC-MS calculated for C₂₁H₁₈N₇O (M+H)⁺: m/z=384.2. found 384.2.

### Example 27. 5-Amino-7-(3-cyanophenyl)-N-ethyl-8-(3-methylpyridin-4-yl)imidazo[1,2-c]pyrimidine-2-carboxamide

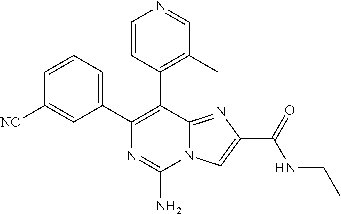 [SMILES: CCNC(=O)c1cn2c(N)nc(-c3cccc(C#N)c3)c(-c3ccncc3C)c2n1]

This compound was prepared using similar procedures as described for Example 26 with 3-methylpyridin-4-ylboronic acid replacing pyridin-4-ylboronic acid in Step 5. The product was purified by prep-LC-MS (pH=2, MeCN/water with TFA) to give the desired product as a TFA salt. LC-MS calculated for C₂₂H₂₀N₇O (M+H)⁺: m/z=398.2. found 398.2.

### Example 28. 5-Amino-7-(3-cyanophenyl)-N-ethyl-8-(3-fluoropyridin-4-yl)imidazo[1,2-c]pyrimidine-2-carboxamide

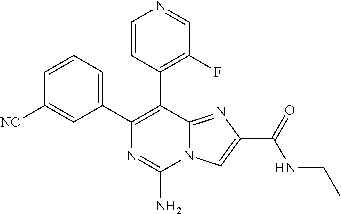 [SMILES: CCNC(=O)c1cn2c(N)nc(-c3cccc(C#N)c3)c(-c3ccncc3F)c2n1]

This compound was prepared using similar procedures as described for Example 26 with 3-fluoropyridin-4-ylboronic acid replacing pyridin-4-ylboronic acid in Step 5. The product was purified by prep-LC-MS (pH=2, MeCN/water with TFA) to give the desired product as a TFA salt. LC-MS calculated for C₂₁H₁₇FN₇O (M+H)⁺: m/z=402.1. found 402.2.

### Example 29. 5-Amino-7-(3-cyanophenyl)-N-ethyl-8-(3-chloropyridin-4-yl)imidazo[1,2-c]pyrimidine-2-carboxamide

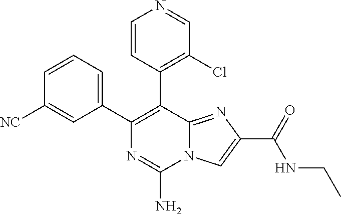 [SMILES: CCNC(=O)c1cn2c(N)nc(-c3cccc(C#N)c3)c(-c3ccncc3Cl)c2n1]

This compound was prepared using similar procedures as described for Example 26 with 3-chloropyridin-4-ylboronic acid replacing pyridin-4-ylboronic acid in Step 5. The product was purified by prep-LC-MS (pH=2, MeCN/water with TFA) to give the desired product as a TFA salt. LC-MS calculated for C₂₁H₁₇ClN₇O (M+H)⁺: m/z=418.1. found 418.2.

### Example 30. 5-Amino-7-(3-cyanophenyl)-N-ethyl-8-(3-methoxypyridin-4-yl)imidazo[1,2-c]pyrimidine-2-carboxamide

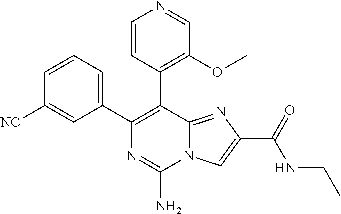 [SMILES: CCNC(=O)c1cn2c(N)nc(-c3cccc(C#N)c3)c(-c3ccncc3OC)c2n1]

This compound was prepared using similar procedures as described for Example 26 with 3-methoxypyridin-4-ylboronic acid replacing pyridin-4-ylboronic acid in Step 5. The product was purified by prep-LC-MS (pH=2, MeCN/water with TFA) to give the desired product as a TFA salt. LC-MS calculated for C₂₂H₂₀N₇O₂ (M+H)⁺: m/z=414.2. found 414.2.

### Example 31. 5-Amino-7-(3-cyanophenyl)-N-ethyl-8-(3-cyanopyridin-4-yl)imidazo[1,2-c]pyrimidine-2-carboxamide

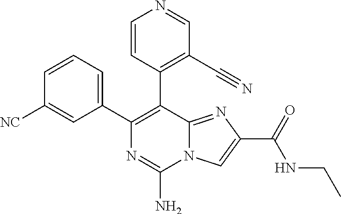 [SMILES: CCNC(=O)c1cn2c(N)nc(-c3cccc(C#N)c3)c(-c3ccncc3C#N)c2n1]

This compound was prepared using similar procedures as described for Example 26 with 4-(4,4,5,5-tetramethyl-1,3,2-dioxaborolan-2-yl)nicotinonitrile replacing pyridin-4-ylboronic acid in Step 5. The product was purified by prep-LC-MS (pH=2, MeCN/water with TFA) to give the desired product as a TFA salt. LC-MS calculated for C₂₂H₁₇N₈O (M+H)⁺: m/z=409.2. found 409.2.

### Example 32. 5-Amino-8-(4-carbamoylphenyl)-7-(3-cyanophenyl)-N-ethylimidazo[1,2-c]pyrimidine-2-carboxamide

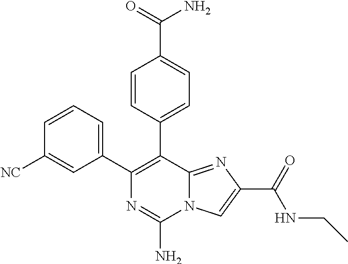 [SMILES: CCNC(=O)c1cn2c(N)nc(-c3cccc(C#N)c3)c(-c3ccc(C(N)=O)cc3)c2n1]

This compound was prepared using similar procedures as described for Example 26 with (4-carbamoylphenyl)boronic acid replacing pyridin-4-ylboronic acid in Step 5. The product was purified by prep-LC-MS (pH=2, MeCN/water with TFA) to give the desired product as a TFA salt. LC-MS calculated for C₂₃H₂₀N₇O₂ (M+H)⁺: m/z=426.2. found 426.2.

### Example 33. 5-Amino-7-(3-cyanophenyl)-N-ethyl-8-(pyrazolo[1,5-a]pyridin-3-yl)imidazo[1,2-c]pyrimidine-2-carboxamide

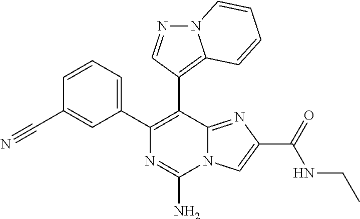 [SMILES: CCNC(=O)c1cn2c(N)nc(-c3cccc(C#N)c3)c(-c3cnn4ccccc34)c2n1]

This compound was prepared using similar procedures as described for Example 26 with 3-(4,4,5,5-tetramethyl-1,3,2-dioxaborolan-2-yl)pyrazolo[1,5-a]pyridine replacing pyridin-4-ylboronic acid in Step 5. The product was purified by prep-LC-MS (pH=2, MeCN/water with TFA) to give the desired product as a TFA salt. LC-MS calculated for C₂₃H₁₉N₈O (M+H)⁺: m/z=423.2. found 423.2.

### Example 34. 5-Amino-7-(3-cyanophenyl)-N-ethyl-8-(5-methyl-1H-pyrazol-4-yl)imidazo[1,2-c]pyrimidine-2-carboxamide

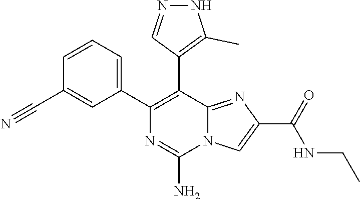 [SMILES: CCNC(=O)c1cn2c(N)nc(-c3cccc(C#N)c3)c(-c3cn[nH]c3C)c2n1]

This compound was prepared using similar procedures as described for Example 26 with 5-methyl-4-(4,4,5,5-tetramethyl-1,3,2-dioxaborolan-2-yl)-1H-pyrazole replacing pyridin-4-ylboronic acid in Step 5. The product was purified by prep-LC-MS (pH=2, MeCN/water with TFA) to give the desired product as a TFA salt. LC-MS calculated for C₂H₁₉N₈O (M+H)⁺: m/z=387.2. found 387.2.

### Example 35. 5-Amino-7-(3-cyanophenyl)-N-ethyl-8-(1-ethyl-1H-pyrazol-5-yl)imidazo[1,2-c]pyrimidine-2-carboxamide

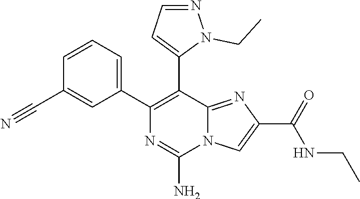 [SMILES: CCNC(=O)c1cn2c(N)nc(-c3cccc(C#N)c3)c(-c3ccnn3CC)c2n1]

This compound was prepared using similar procedures as described for Example 26 with 1-ethyl-5-(4,4,5,5-tetramethyl-1,3,2-dioxaborolan-2-yl)-1H-pyrazole replacing pyridin-4-ylboronic acid in Step 5. The product was purified by prep-LC-MS (pH=2, MeCN/water with TFA) to give the desired product as a TFA salt. LC-MS calculated for C₂₁H₂₁N₈O (M+H)⁺: m/z=401.2. found 401.2.

### Example 36. 5-Amino-7-(3-cyanophenyl)-N-ethyl-8-(1-isopropyl-1H-pyrazol-5-yl)imidazo[1,2-c]pyrimidine-2-carboxamide

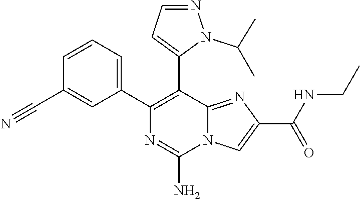 [SMILES: CCNC(=O)c1cn2c(N)nc(-c3cccc(C#N)c3)c(-c3ccnn3C(C)C)c2n1]

This compound was prepared using similar procedures as described for Example 26 with 1-isopropyl-5-(4,4,5,5-tetramethyl-1,3,2-dioxaborolan-2-yl)-1H-pyrazole replacing pyridin-4-ylboronic acid in Step 5. The product was purified by prep-LC-MS (pH=2, MeCN/water with TFA) to give the desired product as a TFA salt. LC-MS calculated for C₂₂H₂₃N₈O (M+H)⁺: m/z=415.2. found 415.2.

### Example 37. 5-Amino-7-(3-cyanophenyl)-N-ethyl-8-(1-propyl-1H-pyrazol-5-yl)imidazo[1,2-c]pyrimidine-2-carboxamide

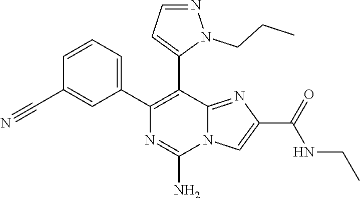 [SMILES: CCCn1nccc1-c1c(-c2cccc(C#N)c2)nc(N)n2cc(C(=O)NCC)nc12]

This compound was prepared using similar procedures as described for Example 26 with 1-propyl-5-(4,4,5,5-tetramethyl-1,3,2-dioxaborolan-2-yl)-1H-pyrazole replacing pyridin-4-ylboronic acid in Step 5. The product was purified by prep-LC-MS (pH=2, MeCN/water with TFA) to give the desired product as a TFA salt. LC-MS calculated for C₂₂H₂₃N₈O (M+H)⁺: m/z=415.2. found 415.2.

### Example 38. 5-Amino-7-(3-cyanophenyl)-N-ethyl-8-(pyrimidin-4-yl)imidazo[1,2-c]pyrimidine-2-carboxamide

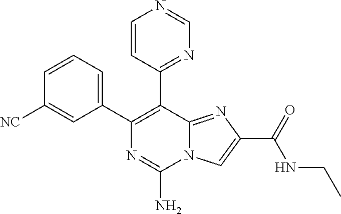 [SMILES: CCNC(=O)c1cn2c(N)nc(-c3cccc(C#N)c3)c(-c3ccncn3)c2n1]

A mixture of 5-amino-8-bromo-7-(3-cyanophenyl)-N-ethylimidazo[1,2-c]pyrimidine-2-carboxamide (prepared in Example 26, Step 4) (50 mg, 0.13 mmol), 4-(tributylstannyl)pyrimidine (96 mg, 0.260 mmol), tetrakis(triphenylphosphine)palladium(0) (15.0 mg, 0.013 mmol), copper(I) chloride (15.4 mg, 0.156 mmol) and lithium chloride (6.6 mg, 0.156 mmol) in THE (2 mL) was degassed and sealed. The reaction mixture was stirred at 80° C. for 12 h, cooled to room temperature, and concentrated under reduced pressure. The resulting residue was purified by prep-LC-MS (pH=2, MeCN/water with TFA) to give the desired product as a TFA salt. LC-MS calculated for C₂₀H₁₇N₈O (M+H)⁺: m/z=385.2. found 385.2.

### Example 39. 5-Amino-7-(3-cyanophenyl)-8-(2,3-dihydro-[1,4]dioxino[2,3-b]pyridin-8-yl)-N-ethylimidazo[1,2-c]pyrimidine-2-carboxamide

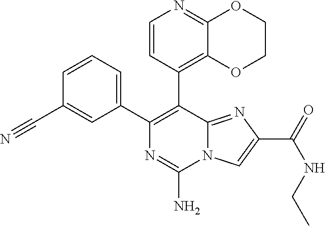 [SMILES: CCNC(=O)c1cn2c(N)nc(-c3cccc(C#N)c3)c(-c3ccnc4c3OCCO4)c2n1]

A mixture of 5-amino-8-bromo-7-(3-cyanophenyl)-N-ethylimidazo[1,2-c]pyrimidine-2-carboxamide (prepared in Example 26, Step 4) (72 mg, 0.19 mmol), (2,3-dihydro-[1,4]dioxino[2,3-b]pyridin-8-yl)boronic acid (33.8 mg, 0.19 mmol), cesium carbonate (122 mg, 0.37 mmol) and [1,1′-bis(dicyclohexylphosphino)ferrocene]dichloropalladium (II) (14.1 mg, 0.019 mmol) in dioxane (2.0 mL) and water (0.2 mL) was stirred at 120° C. for 1 h under microwave irradiation. The reaction mixture was then cooled to room temperature, and directly purified by prep-LC-MS (pH=2, MeCN/water with TFA) to give the desired product as a TFA salt. LC-MS calculated for C₂₃H₂₀N₇O₃ (M+H)⁺: m/z=442.2. found 442.3.

### Example 40. 5-Amino-7-(3-cyanophenyl)-8-cyclopropyl-N-ethylimidazo[1,2-c]pyrimidine-2-carboxamide

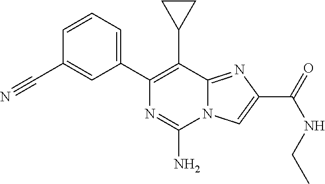 [SMILES: CCNC(=O)c1cn2c(N)nc(-c3cccc(C#N)c3)c(C3CC3)c2n1]

A mixture of 5-amino-8-bromo-7-(3-cyanophenyl)-N-ethylimidazo[1,2-c]pyrimidine-2-carboxamide (prepared in Example 26, Step 4) (72 mg, 0.19 mmol), cyclopropylboronic acid (32.1 mg, 0.37 mmol), cesium carbonate (122 mg, 0.37 mmol) and [1,1′-bis(dicyclohexylphosphino)ferrocene]dichloropalladium (II) (14.1 mg, 0.019 mmol) in dioxane (2.0 mL) and water (0.2 mL) was stirred at 80° C. for 1 h under microwave irradiation. The reaction mixture was then cooled to room temperature, and directly purified by prep-LC-MS (pH=2, MeCN/water with TFA) to give the desired product as a TFA salt. LC-MS calculated for C₁₉H₁₉N₆O (M+H)⁺: m/z=347.2. found 347.3.

### Example 41. 3-(5-Amino-2-(pyridin-2-ylmethyl)-8-(pyrimidin-4-yl)-[1,2,4]triazolo[1,5-c]pyrimidin-7-yl)benzonitrile

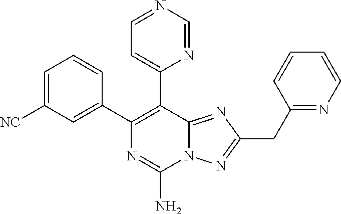 [SMILES: N#Cc1cccc(-c2nc(N)n3nc(Cc4ccccn4)nc3c2-c2ccncn2)c1]

### Step 1: 3-(2-Amino-6-chloropyrimidin-4-yl)benzonitrile

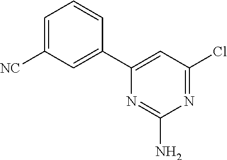 [SMILES: N#Cc1cccc(-c2cc(Cl)nc(N)n2)c1]

A mixture of 4,6-dichloropyrimidin-2-amine (2.5 g, 15.2 mmol), (3-cyanophenyl)boronic acid (2.02 g, 13.7 mmol), tetrakis(triphenylphosphine)palladium(0) (1.06 g, 0.92 mmol) and sodium carbonate (3.23 g, 30.5 mmol) in 1,4-dioxane (60 mL), and water (5 mL) was degassed with nitrogen, then the resulting mixture was heated and stirred at 60° C. for two days. After cooled to room temperature (r.t.), the mixture was concentrated, diluted with water, and extracted with DCM (30 mL×3). The combined organic layers were dried over MgSO₄, filtered, and concentrated. The resulting residue was purified by flash chromatography on a silica gel column eluting with 8% EtOAc in dichloromethane to afford the desired product. LCMS calculated for C₁₁H₈ClN₄ (M+H)⁺: 231.0. Found: 231.0.

### Step 2: 2-(Pyridin-2 yl)acetohydrazide

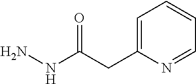 [SMILES: NNC(=O)Cc1ccccn1]

Hydrazine (4.15 mL, 132 mmol) was added to a ethanol (66 mL) solution of methyl 2-(pyridin-2-yl)acetate (10 g, 66.2 mmol) at r.t. The mixture was heated and stirred at 85° C. for 4 h, and then cooled to r.t. White solid was formed upon standing, which was collected via filtration and used in next step without further purification. LCMS calculated for C₇H₁₀N₃O (M+H)⁺: 152.1. Found: 152.0.

### Step 3: 3-(5 Amino-2-(pyridin-2 ylmethyl)-[1,2,4]triazolo[1,5-c]pyrimidin-7-yl)benzonitrile

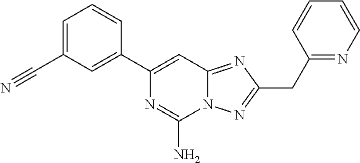 [SMILES: N#Cc1cccc(-c2cc3nc(Cc4ccccn4)nn3c(N)n2)c1]

2-(pyridin-2-yl)acetohydrazide (2.62 g, 17.34 mmol) was added to a ethanol (35 mL) solution of 3-(2-amino-6-chloropyrimidin-4-yl)benzonitrile (4.00 g, 17.34 mmol) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₈H₁₄N₇ (M+H)⁺: 328.1. found 328.1.

### Step 4: 3-(5 Amino-8-bromo-2-(pyridin-2 ylmethyl)-[1,2,4]triazolo[1,5-c]pyrimidin-7-yl)benzonitrile

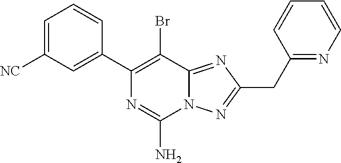 [SMILES: N#Cc1cccc(-c2nc(N)n3nc(Cc4ccccn4)nc3c2Br)c1]

To a mixture of 3-(5-amino-2-(pyridin-2-ylmethyl)-[1,2,4]triazolo[1,5-c]pyrimidin-7-yl)benzonitrile (2 g, 6.11 mmol) in DMF (12 mL) at −30° C. was added NBS (1.09 g, 6.11 mmol) portion-wise. The reaction mixture was allowed to slowly warm to 0° C., resulting a homogenous solution. After stirring at 0° C. for 1 h, the reaction mixture was diluted with saturated NaHCO₃ solution and the resulting solid was collected by filtration. The solid was then purified by flash chromatography on a silica gel column eluting with 0 to 10% MeOH in DCM to afford the desired product. LCMS calculated for C₁₈H₁₃BrN₇ (M+H)⁺: 406.0. found 406.0.

### Step 5: 3-(5 Amino-2-(pyridin-2 ylmethyl)-8-(pyrimidin-4-yl)-[1,2,4]triazolo[1,5-c]pyrimidin-7-yl)benzonitrile

Pd(Ph₃P)₄ (284 mg, 0.246 mmol) was added to a mixture of 4-(tributylstannyl)pyrimidine (1090 mg, 2.95 mmol), 3-(5-amino-8-bromo-2-(pyridin-2-ylmethyl)-[1,2,4]triazolo[1,5-c]pyrimidin-7-yl)benzonitrile (1000 mg, 2.46 mmol), and copper(I) chloride (244 mg, 2.46 mmol) in 1,4-dioxane (12 mL). The reaction mixture was purged with N₂ and stirred at 80° C. for 7 h.

The resulting mixture was cooled to r.t., concentrated, diluted with DCM (50 mL) and washed with saturated NH₄OH solution. The organic layer was dried over Na₂SO₄, concentrated, and purified by preparative LC-MS (pH 2, acetonitrile/water with TFA) to afford the product as a TFA salt. LCMS calculated for C₂₂H₁₆N₉ (M+H)⁺: 406.2. found 406.2. ¹H NMR (500 MHz, DMSO) δ 8.95 (s, 1H), 8.83 (d, J=5.3 Hz, 1H), 8.59 (d, J=5.1 Hz, 1H), 7.96 (m, 1H), 7.88 (d, J=5.1 Hz, 1H), 7.82 (d, J=7.6 Hz, 1H), 7.76 (s, 1H), 7.60-7.53 (m, 2H), 7.53-7.48 (m, 1H), 7.48-7.42 (m, 1H), 4.49 (s, 2H).

### Example 42. 3-(5-Amino-8-(1-ethyl-1H-pyrazol-5-yl)-2-(pyridin-2-ylmethyl)-[1,2,4]triazolo[1,5-c]pyrimidin-7-yl)benzonitrile

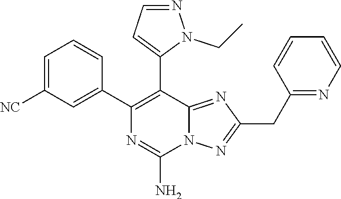 [SMILES: CCn1nccc1-c1c(-c2cccc(C#N)c2)nc(N)n2nc(Cc3ccccn3)nc12]

A mixture of 3-(5-amino-8-bromo-2-(pyridin-2-ylmethyl)-[1,2,4]triazolo[1,5-c]pyrimidin-7-yl)benzonitrile (from Example 41, Step 4) (50 mg, 0.123 mmol), 1-ethyl-5-(4,4,5,5-tetramethyl-1,3,2-dioxaborolan-2-yl)-1H-pyrazole (54.7 mg, 0.246 mmol), chloro(2-dicyclohexylphosphino-2′,4′,6′-triisopropyl-1,1′-biphenyl) [2-(2′-amino-1,1′-biphenyl)]palladium(II) (9.68 mg, 0.012 mmol), and sodium carbonate (13.0 mg, 0.123 mmol) in 1,4-dioxane (1119 μL) and water (112 μL) was stirred at 100° C. for 1 h. The resulting mixture was cooled to r.t., concentrated, and purified by preparative LC-MS (pH 2, acetonitrile/water with TFA) to afford the product as a TFA salt. LCMS calculated for C₂₃H₂₀N₉ (M+H)⁺: 422.2. found 422.2.

### Example 43. 3-(5-Amino-8-(1-propyl-1H-pyrazol-5-yl)-2-(pyridin-2-ylmethyl)-[1,2,4]triazolo[1,5-c]pyrimidin-7-yl)benzonitrile

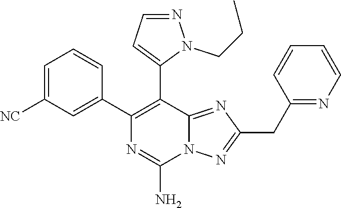 [SMILES: CCCn1nccc1-c1c(-c2cccc(C#N)c2)nc(N)n2nc(Cc3ccccn3)nc12]

This compound was prepared using similar procedures as described for Example 42 with 1-propyl-5-(4,4,5,5-tetramethyl-1,3,2-dioxaborolan-2-yl)-1H-pyrazole replacing 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₄H₂₂N₉ (M+H)⁺: 436.2. found 436.2.

### Example 44. 3-(5-Amino-2-(pyridin-2-ylmethyl)-8-(quinolin-5-yl)-[1,2,4]triazolo[1,5-c]pyrimidin-7-yl)benzonitrile

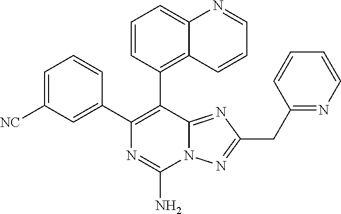 [SMILES: N#Cc1cccc(-c2nc(N)n3nc(Cc4ccccn4)nc3c2-c2cccc3ncccc23)c1]

This compound was prepared using similar procedures as described for Example 42 with -(4,4,5,5-tetramethyl-1,3,2-dioxaborolan-2-yl)quinoline replacing 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₇H₁₉N₈ (M+H)⁺: 455.2. found 455.2.

### Example 45. 3-(5-Amino-8-(5-fluoropyrimidin-4-yl)-2-(hydroxy(phenyl)methyl)-[1,2,4]triazolo[1,5-c]pyrimidin-7-yl)benzonitrile

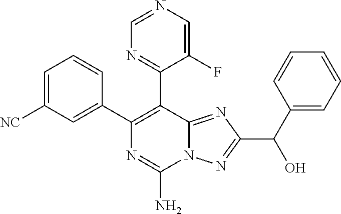 [SMILES: N#Cc1cccc(-c2nc(N)n3nc(C(O)c4ccccc4)nc3c2-c2ncncc2F)c1]

### Step 1: 3-(5 Amino-8-bromo-2-(hydroxy(phenyl)methyl)-[1,2,4]triazolo[1,5-c]pyrimidin-7-yl)benzonitrile

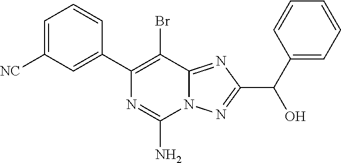 [SMILES: N#Cc1cccc(-c2nc(N)n3nc(C(O)c4ccccc4)nc3c2Br)c1]

This compound was prepared using similar procedures as described for Example 41, Step 1 to Step 4, with 2-hydroxy-2-phenylacetohydrazide replacing 2-(pyridin-2-yl)acetohydrazide in Step 3. LCMS calculated for C₁₉H₁₄BrN₆O (M+H)⁺: 421.0. found 421.0.

### Step 2: 5-Fluoro-4-(trimethylstannyl)pyrimidine

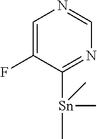

Pd(Ph₃P)₄ (43.6 mg, 0.038 mmol) and 1,1,1,2,2,2-hexamethyldistannane (124 mg, 0.377 mmol) were successively added to a mixture of 4-chloro-5-fluoropyrimidine (50 mg, 0.377 mmol) in 1,4-dioxane (1886 μL) under a nitrogen atmosphere. The reaction mixture was then stirred at reflux overnight, cooled to r.t., and filtered. The filtrate was used in next step without further purification.

### Step 3: 3-(5-Amino-8-(S fluoropyrimidin-4-yl)-2-(hydroxy(phenyl)methyl)-[1,2,4]triazolo[1,5-c]pyrimidin-7-yl)benzonitrile

This compound was prepared using similar procedures as described for Example 41, Step 5, with 5-fluoro-4-(trimethylstannyl)pyrimidine replacing 4-(tributylstannyl)pyrimidine, and with 3-(5-amino-8-bromo-2-(hydroxy(phenyl)methyl)-[1,2,4]triazolo[1,5-c]pyrimidin-7-yl)benzonitrile replacing 3-(5-amino-8-bromo-2-(pyridin-2-ylmethyl)-[1,2,4]triazolo[1,5-c]pyrimidin-7-yl)benzonitrile. The final material was purified by preparative LC-MS (pH 2, acetonitrile/water with TFA) to afford the product as a TFA salt. LCMS calculated for C₂₃H₁₆FN₈₀ (M+H)⁺: 439.1. found 439.1.

### Example 46. 3-(5-Amino-8-(5-fluoropyrimidin-4-yl)-2-(pyridin-2-ylmethyl)-[1,2,4]triazolo[1,5-c]pyrimidin-7-yl)benzonitrile

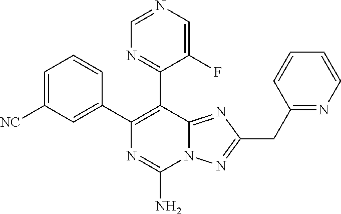 [SMILES: N#Cc1cccc(-c2nc(N)n3nc(Cc4ccccn4)nc3c2-c2ncncc2F)c1]

This compound was prepared using similar procedures as described for Example 41, with 5-fluoro-4-(trimethylstannyl)pyrimidine (from Example 45, Step 2) replacing 4-(tributylstannyl)pyrimidine. The final material was purified by preparative HPLC (pH 2, acetonitrile/water with TFA) to afford the product as a TFA salt. LCMS calculated for C₂₂H₁₅FN₉ (M+H)⁺: 424.1. found 424.2.

### Example 47. 3-(5-Amino-2-((2-hydroxyethylamino)(phenyl)methyl)-8-(pyridin-4-yl)-[1,2,4]triazolo[1,5-c]pyrimidin-7-yl)benzonitrile

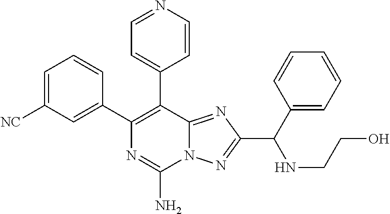 [SMILES: N#Cc1cccc(-c2nc(N)n3nc(C(NCCO)c4ccccc4)nc3c2-c2ccncc2)c1]

### Step 1: 3-(5 Amino-8-bromo-2-(chloro(phenyl)methyl)-[1,2,4]triazolo[1,5-c]pyrimidin-7-yl)benzonitrile

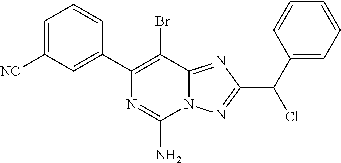 [SMILES: N#Cc1cccc(-c2nc(N)n3nc(C(Cl)c4ccccc4)nc3c2Br)c1]

Thionyl chloride (87 μL, 1.19 mmol) was added to 3-(5-amino-8-bromo-2-(hydroxy(phenyl)methyl)-[1,2,4]triazolo[1,5-c]pyrimidin-7-yl)benzonitrile (from Example 45, Step 1) (10 mg, 0.024 mmol) at r.t. After stirring for 30 min, the reaction mixture was concentrated, and the resulting residue was used in the next step without further purification. LCMS calculated for C₁₉H₁₃BrClN₆ (M+H)⁺: 441.0. found 441.0.

### Step 2: 3-(5 Amino-8-bromo-2-((2-hydroxyethylamino)(phenyl)methyl)-[1,2,4]triazolo[1,5-c]pyrimidin-7-yl)benzonitrile

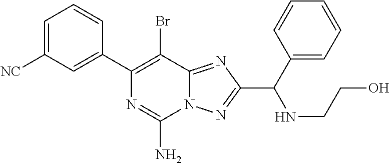 [SMILES: N#Cc1cccc(-c2nc(N)n3nc(C(NCCO)c4ccccc4)nc3c2Br)c1]

2-aminoethan-1-ol (4.35 mg, 0.071 mmol) was added to a DMF (237 μL) solution of 3-(5-amino-8-bromo-2-(chloro(phenyl)methyl)-[1,2,4]triazolo[1,5-c]pyrimidin-7-yl)benzonitrile (10 mg, 0.024 mmol). The reaction mixture was stirred at r.t. overnight, concentrated, and used in the next step without further purification. LCMS calculated for C₂₁H₁₉BrN₇O (M+H)⁺: 464.1. found 464.2.

### Step 3: 3-(5 Amino-2-((2-hydroxyethylamino)(phenyl)methyl)-8-(pyridin-4-yl)-[1,2,4]triazolo[1,5-c]pyrimidin-7-yl)benzonitrile

A mixture of 3-(5-amino-8-bromo-2-(((2-hydroxyethyl)amino)(phenyl)methyl)-[1,2,4]triazolo[1,5-c]pyrimidin-7-yl)benzonitrile (10 mg, 0.022 mmol), pyridin-4-ylboronic acid (6.0 mg, 0.043 mmol), chloro(2-dicyclohexylphosphino-2′,4′,6′-triisopropyl-1,1′-biphenyl)[2-(2′-amino-1,1′-biphenyl)]palladium(II) (1.70 mg, 2.15 μmol), and sodium carbonate (2.3 mg, 0.022 mmol) in 1,4-dioxane (196 μL) and water (19.6 μL) was heated and stirred at 100° C. for 1 h. The reaction mixture was then cooled to r.t., concentrated, purified by preparative LC-MS (pH 2, acetonitrile/water with TFA) to afford the product as a TFA salt. LCMS calculated for C₂₆H₂₃N₈O (M+H)⁺: 463.2. found 463.2.

### Example 48. 3-(5-Amino-2-(cyclohexylmethyl)-8-(1-ethyl-1H-pyrazol-5-yl)-[1,2,4]triazolo[1,5-c]pyrimidin-7-yl)benzonitrile

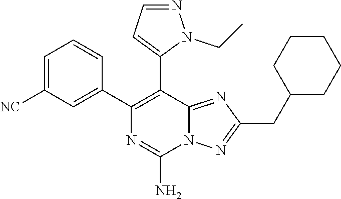 [SMILES: CCn1nccc1-c1c(-c2cccc(C#N)c2)nc(N)n2nc(CC3CCCCC3)nc12]

### Step 1: 3-(5 Amino-8-bromo-2-(cyclohexylmethyl)-[1,2,4]triazolo[1,5-c]pyrimidin-7-yl)benzonitrile

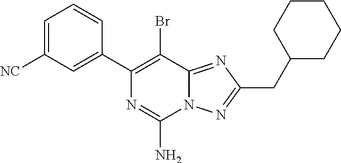 [SMILES: N#Cc1cccc(-c2nc(N)n3nc(CC4CCCCC4)nc3c2Br)c1]

This compound was prepared using similar procedures as described for Example 41, Step 1 to Step 4, with 2-cyclohexylacetohydrazide replacing 2-(pyridin-2-yl)acetohydrazide in Step 3. LCMS calculated for C₁₉H₂₀BrN₆ (M+H)⁺: 411.1. found 411.1.

### Step 2: 3-(5-Amino-2-(cyclohexylmethyl)-8-(1-ethyl-1H-pyrazol-5-yl)-[1,2,4]triazolo[1,5-c]pyrimidin-7-yl)benzonitrile

This compound was prepared using similar procedures as described for Example 42, with 3-(5-amino-8-bromo-2-(cyclohexylmethyl)-[1,2,4]triazolo[1,5-c]pyrimidin-7-yl)benzonitrile replacing 3-(5-amino-8-bromo-2-(pyridin-2-ylmethyl)-[1,2,4]triazolo[1,5-c]pyrimidin-7-yl)benzonitrile. The final material was purified by preparative LC-MS (pH 2, acetonitrile/water with TFA) to afford the product as a TFA salt. LCMS calculated for C₂₄H₂₇N₈ (M+H)⁺: 427.2. found 427.2.

### Example 49. 3-(5-Amino-2-(2-fluorobenzyl)-8-(pyrimidin-4-yl)-[1,2,4]triazolo[1,5-c]pyrimidin-7-yl)benzonitrile

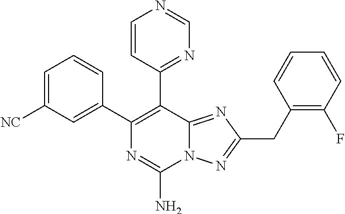 [SMILES: N#Cc1cccc(-c2nc(N)n3nc(Cc4ccccc4F)nc3c2-c2ccncn2)c1]

This compound was prepared using similar procedures as described for Example 41, with 2-(2-fluorophenyl)acetohydrazide replacing 2-(pyridin-2-yl)acetohydrazide in Step 3. The final material was purified by preparative LC-MS (pH 2, acetonitrile/water with TFA) to afford the product as a TFA salt. LCMS calculated for C₂₃H₁₆FN₈ (M+H)⁺: 423.2. found 423.2.

### Example 50. 3-(5-Amino-2-((2-fluorophenyl)(hydroxy)methyl)-8-(pyrimidin-4-yl)-[1,2,4]triazolo[1,5-c]pyrimidin-7-yl)benzonitrile

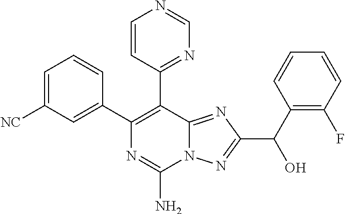 [SMILES: N#Cc1cccc(-c2nc(N)n3nc(C(O)c4ccccc4F)nc3c2-c2ccncn2)c1]

This compound was prepared using similar procedures as described for Example 41, with 2-(2-fluorophenyl)-2-hydroxyacetohydrazide replacing 2-(pyridin-2-yl)acetohydrazide in Step 3. The final material was purified by preparative LC-MS (pH 2, acetonitrile/water with TFA) to afford the product as a TFA salt. LCMS calculated for C₂₃H₁₆FN₈₀ (M+H)⁺: 439.1. found 439.1.

### Example 51. 3-(5-Amino-2-((6-methylpyridin-2-yl)methyl)-8-(pyrimidin-4-yl)-[1,2,4]triazolo[1,5-c]pyrimidin-7-yl)benzonitrile

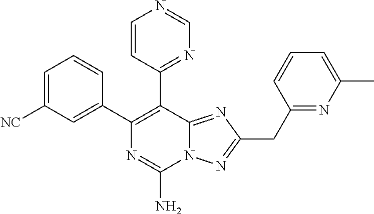 [SMILES: Cc1cccc(Cc2nc3c(-c4ccncn4)c(-c4cccc(C#N)c4)nc(N)n3n2)n1]

### Step 1: 2-(6 Methylpyridin-2 yl)acetohydrazide

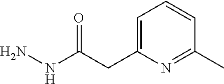

This compound was prepared using similar procedures as described for Example 41, Step 2, with ethyl 2-(6-methylpyridin-2-yl)acetate replacing methyl 2-(pyridin-2-yl)acetate. LCMS calculated for C₈H₁₂N₃₀ (M+H)⁺: 166.1. found 166.1.

### Step 2: 3-(5-Amino-2-((6-methylpyridin-2 yl)methyl)-8-(pyrimidin-4-yl)-[1,2,4]triazolo[1,5-c]pyrimidin-7-yl)benzonitrile

This compound was prepared using similar procedures as described for Example 41, with 2-(6-methylpyridin-2-yl)acetohydrazide replacing 2-(pyridin-2-yl)acetohydrazide in Step 3. The final material was purified by preparative LC-MS (pH 2, acetonitrile/water with TFA) to afford the product as a TFA salt. LCMS calculated for C₂₃H₁₈N₉ (M+H)⁺: 420.2. found 420.2.

### Example 52. 3-(5-Amino-8-(1-ethyl-1H-pyrazol-5-yl)-2-((3-fluoropyridin-2-yl)methyl)-[1,2,4]triazolo[1,5-c]pyrimidin-7-yl)benzonitrile

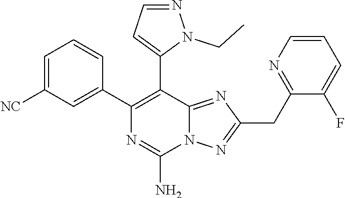 [SMILES: CCn1nccc1-c1c(-c2cccc(C#N)c2)nc(N)n2nc(Cc3ncccc3F)nc12]

### Step 1: 2-(3-Fluoropyridin-2-yl)acetohydrazide

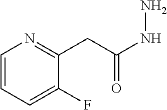 [SMILES: NNC(=O)Cc1ncccc1F]

Hunig's base (169 μL, 0.967 mmol) was added to a DMF (2149 μL) solution of 2-(3-fluoropyridin-2-yl)acetic acid (100 mg, 0.645 mmol), tert-butyl hydrazinecarboxylate (102 mg, 0.774 mmol), and BOP (428 mg, 0.967 mmol) at r.t. The reaction mixture was stirred at r.t. for 2 h, concentrated, and purified by flash chromatography on a silica gel column eluting with 0 to 10% MeOH in DCM. The purified intermediate tert-butyl 2-(2-(3-fluoropyridin-2-yl)acetyl)hydrazinecarboxylate was then treated with TFA (0.5 mL), stirred at r.t. overnight, concentrated, and diluted with ether. The resulting white precipitate was collected by filtration, and used in next step without further purification. LCMS calculated for C₇H₉FN₃₀ (M+H)⁺: 170.1. found 170.1.

### Step 2: 3-(5-Amino-8-bromo-2-((3 fluoropyridin-2 yl)methyl)-[1,2,4]triazolo[1,5-c]pyrimidin-7-yl)benzonitrile

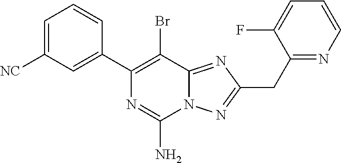 [SMILES: N#Cc1cccc(-c2nc(N)n3nc(Cc4ncccc4F)nc3c2Br)c1]

This compound was prepared using similar procedures as described for Example 41, Step 1 to Step 4, with 2-(3-fluoropyridin-2-yl)acetohydrazide replacing 2-(pyridin-2-yl)acetohydrazide in Step 3. LCMS calculated for C₁₈H₁₂BrFN₇ (M+H)⁺: 424.0. found 424.0.

### Step 3: 3-(5-Amino-8-(1-ethyl-1H-pyrazol-5-yl)-2-((3 fluoropyridin-2 yl)methyl)-[1,2,4]triazolo[1,5-c]pyrimidin-7-yl)benzonitrile

This compound was prepared using similar procedures as described for Example 42 with 3-(5-amino-8-bromo-2-((3-fluoropyridin-2-yl)methyl)-[1,2,4]triazolo[1,5-c]pyrimidin-7-yl)benzonitrile replacing 3-(5-amino-8-bromo-2-(pyridin-2-ylmethyl)-[1,2,4]triazolo[1,5-c]pyrimidin-7-yl)benzonitrile. The final material was purified by preparative LC-MS (pH 2, acetonitrile/water with TFA) to afford the product as a TFA salt. LCMS calculated for C₂₃H₁₉FN₉ (M+H)⁺: 440.2. found 440.2.

### Example 53. 3-(5-Amino-8-(1-ethyl-1H-pyrazol-5-yl)-2-((3-methoxypyridin-2-yl)methyl)-[1,2,4]triazolo[1,5-c]pyrimidin-7-yl)benzonitrile

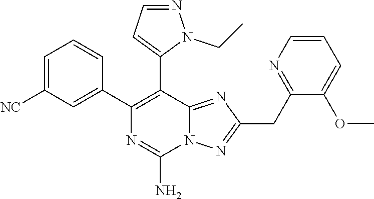 [SMILES: CCn1nccc1-c1c(-c2cccc(C#N)c2)nc(N)n2nc(Cc3ncccc3OC)nc12]

This compound was prepared using similar procedures as described for Example 52, with 2-(3-methoxypyridin-2-yl)acetic acid replacing 2-(3-fluoropyridin-2-yl)acetic acid in Step 1. The final material was purified by preparative LC-MS (pH 2, acetonitrile/water with TFA) to afford the product as a TFA salt. LCMS calculated for C₂₄H₂₂N₉O (M+H)⁺: 452.2. found 452.2.

### Example 54. 3-(5-Amino-2-(2-(1-methyl-1H-pyrazol-4-yl)benzyl)-8-(pyrimidin-4-yl)-[1,2,4]triazolo[1,5-c]pyrimidin-7-yl)benzonitrile

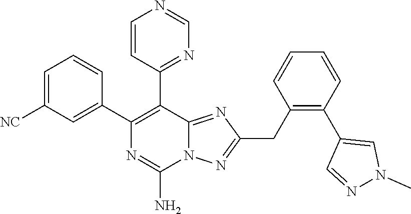 [SMILES: Cn1cc(-c2ccccc2Cc2nc3c(-c4ccncn4)c(-c4cccc(C#N)c4)nc(N)n3n2)cn1]

### Step 1: 3-(5 Amino-2-(2-bromobenzyl)-[1,2,4]triazolo[1,5-c]pyrimidin-7-yl)benzonitrile

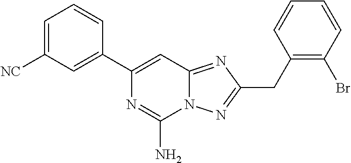 [SMILES: N#Cc1cccc(-c2cc3nc(Cc4ccccc4Br)nn3c(N)n2)c1]

This compound was prepared using similar procedures as described for Example 41, Step 1 to Step 3, with 2-(2-bromophenyl)acetohydrazide replacing 2-(pyridin-2-yl)acetohydrazide in Step 3. LCMS calculated for C₁₉H₁₄BrN₆ (M+H)⁺: 405.0. found 405.0.

### Step 2: 3-(5-Amino-2-(2-bromobenzyl)-8-iodo-[1,2,4]triazolo[1,5-c]pyrimidin-7-yl)benzonitrile

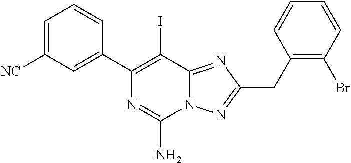 [SMILES: N#Cc1cccc(-c2nc(N)n3nc(Cc4ccccc4Br)nc3c2I)c1]

NIS (153 mg, 0.679 mmol) was added to a DMF (3084 μL) solution of 3-(5-amino-2-(2-bromobenzyl)-[1,2,4]triazolo[1,5-c]pyrimidin-7-yl)benzonitrile (250 mg, 0.617 mmol) at r.t. After stirring at 50° C. for 1 h, the reaction mixture was cooled to r.t., diluted with water and the resulting precipitate was collected by filtration. The brown solid was dissolved in DCM and purified by flash chromatography on a silica gel column eluting with 0 to 50% EtOAc in DCM to afford the desired product. LCMS calculated for C₁₉H₁₃BrIN₆ (M+H)⁺: 531.0. found 531.0.

### Step 3: 3-(5 Amino-2-(2-bromobenzyl)-8-(pyrimidin-4-yl)-[1,2,4]triazolo[1,5-c]pyrimidin-7-yl)benzonitrile

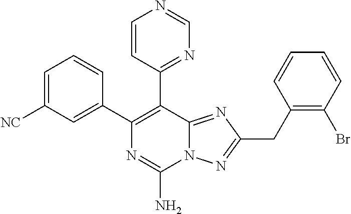 [SMILES: N#Cc1cccc(-c2nc(N)n3nc(Cc4ccccc4Br)nc3c2-c2ccncn2)c1]

This compound was prepared using similar procedures as described for Example 41, Step 5, with 3-(5-amino-2-(2-bromobenzyl)-8-iodo-[1,2,4]triazolo[1,5-c]pyrimidin-7-yl)benzonitrile replacing 3-(5-amino-8-bromo-2-(pyridin-2-ylmethyl)-[1,2,4]triazolo[1,5-c]pyrimidin-7-yl)benzonitrile. The final material was purified by flash chromatography on a silica gel column eluting with 0 to 15% MeOH in DCM to afford the desired product. LCMS calculated for C₂₃H₁₆BrN₈ (M+H)⁺: 483.1. found 483.1.

### Step 4: 3-(5-Amino-2-(2-(1-methyl-1H-pyrazol-4-yl)benzyl)-8-(pyrimidin-4-yl)-[1,2,4]triazolo[1,5-c]pyrimidin-7-yl)benzonitrile

A mixture of 3-(5-amino-2-(2-bromobenzyl)-8-(pyrimidin-4-yl)-[1,2,4]triazolo[1,5-c]pyrimidin-7-yl)benzonitrile (10 mg, 0.021 mmol), (1-methyl-1H-pyrazol-4-yl)boronic acid (5.2 mg, 0.041 mmol), PdCl₂(dppf)-CH₂Cl₂ adduct (1.7 mg, 2.07 μmol), and sodium carbonate (2.2 mg, 0.021 mmol) in 1,4-dioxane (172 μL) and water (35 μL) was purged with N₂ and stirred at 90° C. for 1 h. The reaction mixture was cooled to r.t., concentrated, and purified by preparative LC-MS (pH 2, acetonitrile/water with TFA) to afford the product as a TFA salt. LCMS calculated for C₂₇H₂₁N₁₀ (M+H)⁺: 485.2. found 485.1.

### Example 55. 3-(5-Amino-2-(benzo[d]isoxazol-3-ylmethyl)-8-(1-ethyl-1H-pyrazol-5-yl)-[1,2,4]triazolo[1,5-c]pyrimidin-7-yl)benzonitrile

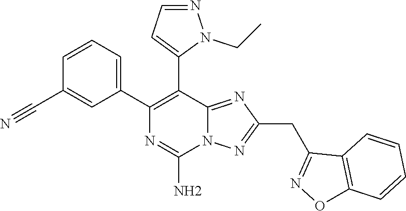 [SMILES: CCn1nccc1-c1c(-c2cccc(C#N)c2)nc(N)n2nc(Cc3noc4ccccc34)nc12]

### Step 1: 3-(5 Amino-2-(benzo[d]isoxazol-3 ylmethyl)-8-bromo-[1,2,4]triazolo[1,5-c]pyrimidin-7-yl)benzonitrile

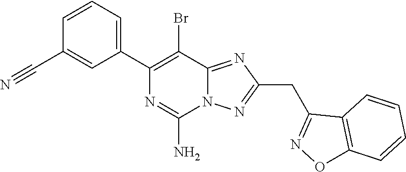 [SMILES: N#Cc1cccc(-c2nc(N)n3nc(Cc4noc5ccccc45)nc3c2Br)c1]

This compound was prepared using similar procedures as described in Example 41, Step 1 to Step 4, with 2-(benzo[d]isoxazol-3-yl)acetohydrazide in place of 2-(pyridin-2-yl)acetohydrazide in Step 3. LCMS calculated for C₂₀H₁₃BrN₇O (M+H)⁺: 446.3. Found: 446.1.

### Step 2: 3-(5-Amino-2-(benzo[d]isoxazol-3 ylmethyl)-8-(1-ethyl-1H-pyrazol-5-yl)-[1,2,4]triazolo[1,5-c]pyrimidin-7-yl)benzonitrile

A vial was charged with 3-(5-amino-2-(benzo[d]isoxazol-3-ylmethyl)-8-bromo-[1,2,4]triazolo[1,5-c]pyrimidin-7-yl)benzonitrile (24 mg, 0.054 mmol), (1-ethyl-1H-pyrazol-5-yl)boronic acid (17 mg, 0.12 mmol), XPhos Pd G2 (4.3 mg, 0.0054 mmol), K₃PO₄ (23 mg, 0.11 mmol), dioxane (1 mL) and water (0.2 mL). The reaction mixture was then heated and stirred at 80° C. for 1 h, cooled to r.t., diluted with saturated NH₄Cl solution (1 mL), and extracted with EtOAc (5 mL). The organic phase was separated, dried over Na₂SO₄, concentrated, and purified by preparative LC-MS (pH 2, acetonitrile/water with TFA) to afford the product as a TFA salt. LCMS calculated for C₂₅H₂₀N₉O (M+H)⁺: 462.2. Found: 462.2.

### Example 56. 3-(5-Amino-8-(1-ethyl-1H-pyrazol-5-yl)-2-((l-methyl-1H-indazol-3-yl)methyl)-[1,2,4]triazolo[1,5-c]pyrimidin-7-yl)benzonitrile

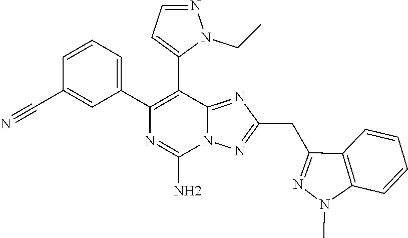 [SMILES: CCn1nccc1-c1c(-c2cccc(C#N)c2)nc(N)n2nc(Cc3nn(C)c4ccccc34)nc12]

### Step 1: Ethyl 5-amino-8-bromo-7-(3-cyanophenyl)-[1,2,4]triazolo[1,5-c]pyrimidine-2-carboxylate

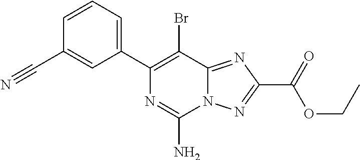 [SMILES: CCOC(=O)c1nc2c(Br)c(-c3cccc(C#N)c3)nc(N)n2n1]

This compound was prepared using the same procedures as described in Example 41, Step 1 to Step 4, with ethyl 2-hydrazinyl-2-oxoacetate in place of 2-(pyridin-2-yl)acetohydrazide in Step 3. LCMS calculated for C₁₅H₁₂BrN₆O₂ (M+H)⁺: 387.2. Found: 387.0.

### Step 2: 3-(5-Amino-8-bromo-2-(hydroxymethyl)-[1,2,4]triazolo[1,5-c]pyrimidin-7-yl)benzonitrile

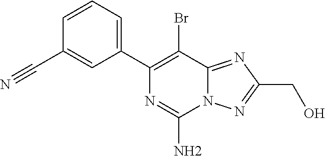 [SMILES: N#Cc1cccc(-c2nc(N)n3nc(CO)nc3c2Br)c1]

To a solution of ethyl 5-amino-8-bromo-7-(3-cyanophenyl)-[1,2,4]triazolo[1,5-c]pyrimidine-2-carboxylate (77 mg, 0.20 mmol) in THF (5 mL) at 0° C. was added LiBH₄ solution (0.2 mL, 2.0 M in THF) dropwise. The reaction mixture was stirred at r.t. for 10 min, then quenched by adding water (1 mL) and saturated Rochelle salt solution (5 mL). After stirring for another 2 h, the organic layer was separated, and the aqueous layer was extracted with EtOAc (3×5 mL). The combined organic layers were washed with brine, dried over Na₂SO₄, and concentrated to afford the crude product, which was used in next step without further purification.

### Step 3: 3-(5-Amino-8-(1-ethyl-1H-pyrazol-5-yl)-2-(hydroxymethyl)-[1,2,4]triazolo[1,5-c]pyrimidin-7-yl)benzonitrile

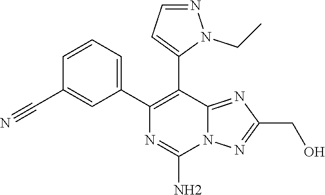 [SMILES: CCn1nccc1-c1c(-c2cccc(C#N)c2)nc(N)n2nc(CO)nc12]

To the crude 3-(5-amino-8-bromo-2-(hydroxymethyl)-[1,2,4]triazolo[1,5-c]pyrimidin-7-yl)benzonitrile from previous step was added (1-ethyl-1H-pyrazol-5-yl)boronic acid (47 mg, 0.34 mmol), XPhos Pd G2 (13 mg, 0.017 mmol), K₃PO₄ (71 mg, 0.34 mmol), dioxane (3 mL) and water (0.6 mL). The reaction mixture was heated and stirred at 90° C. for 1 h, cooled to r.t., diluted with saturated NH₄Cl solution (3 mL), and extracted with EtOAc (15 mL). The organic phase was separated, dried over Na₂SO₄, concentrated, and purified by flash chromatography (0 to 70% EtOAc in dichloromethane) to give the desired product (40 mg, 55%). LCMS calculated for C₁₈H₁₇N₈O (M+H)⁺: 361.1. Found: 361.1.

### Step 4: 3-(5-Amino-2-(chloromethyl)-8-(1-ethyl-1H-pyrazol-5-yl)-[1,2,4]triazolo[1,5-c]pyrimidin-7-yl)benzonitrile

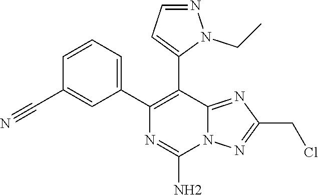 [SMILES: CCn1nccc1-c1c(-c2cccc(C#N)c2)nc(N)n2nc(CCl)nc12]

3-(5-Amino-8-(1-ethyl-1H-pyrazol-5-yl)-2-(hydroxymethyl)-[1,2,4]triazolo[1,5-c]pyrimidin-7-yl)benzonitrile (40 mg, 0.11 mmol) was dissolved in MeCN (2 mL). SOCl₂ (0.02 mL, 0.27 mmol) was added to the solution dropwise at r.t. The reaction mixture was stirred at r.t. for 30 min, quenched with saturated NaHCO₃ solution, and extracted with EtOAc (5×5 mL). the combined organic layers were dried over Na₂SO₄, and concentrated to afford the crude product, which was used in the next step without further purification.

### Step 5: 3-(5-Amino-8-(1-ethyl-1H-pyrazol-5-yl)-2-((1-methyl-1H-indazol-3 yl)methyl)-[1,2,4]triazolo[1,5-c]pyrimidin-7-yl)benzonitrile

A vial was charged with 3-(5-amino-2-(chloromethyl)-8-(1-ethyl-1H-pyrazol-5-yl)-[1,2,4]triazolo[1,5-c]pyrimidin-7-yl)benzonitrile (16 mg, 0.042 mmol), 1-methyl-3-(4,4,5,5-tetramethyl-1,3,2-dioxaborolan-2-yl)-1H-indazole (32 mg, 0.12 mmol), XPhos Pd G2 (3.3 mg, 0.0042 mmol), Cs₂CO₃ (41 mg, 0.13 mmol), and dioxane (1 mL). The reaction mixture was heated and stirred at 90° C. for 1 h, cooled to r.t., diluted with saturated NH₄Cl solution, and extracted with EtOAc (5 mL). The organic phase was separated, dried over Na₂SO₄, concentrated, and purified by preparative LC-MS (pH 2, acetonitrile/water with TFA) to afford the product as a TFA salt (2 mg, 10%). LCMS calculated for C₂₆H₂₃N₁O (M+H)⁺: 475.2. Found: 475.1.

### Example 57. 3-(5-Amino-2-((3-hydroxyazetidin-1-yl)methyl)-8-(pyrimidin-4-yl)-[1,2,4]triazolo[1,5-c]pyrimidin-7-yl)benzonitrile

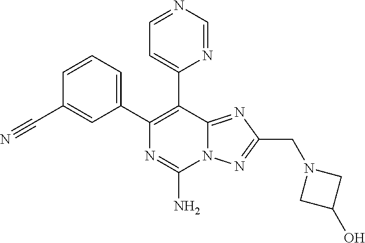 [SMILES: N#Cc1cccc(-c2nc(N)n3nc(CN4CC(O)C4)nc3c2-c2ccncn2)c1]

### Step 1: 3-(5 Amino-2-(hydroxymethyl)-8-(pyrimidin-4-yl)-[1,2,4]triazolo[1,5-c]pyrimidin-7-yl)benzonitrile

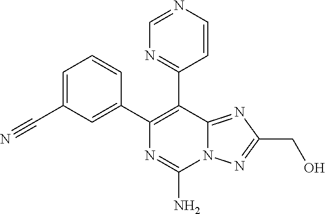 [SMILES: N#Cc1cccc(-c2nc(N)n3nc(CO)nc3c2-c2ccncn2)c1]

This compound was prepared using similar procedures as described for Example 41 Step 5 with 3-(5-amino-8-bromo-2-(hydroxymethyl)-[1,2,4]triazolo[1,5-c]pyrimidin-7-yl)benzonitrile (Example 56, Step 2) replacing 3-(5-amino-8-bromo-2-(pyridin-2-ylmethyl)-[1,2,4]triazolo[1,5-c]pyrimidin-7-yl)benzonitrile. LCMS calculated for C₁₇H₁₃N₈O (M+H)⁺: 345.1. found 345.1.

### Step 2: (5-Amino-7-(3-cyanophenyl)-8-(pyrimidin-4-yl)-[1,2,4]triazolo[1,5-c]pyrimidin-2-yl)methyl methanesulfonate

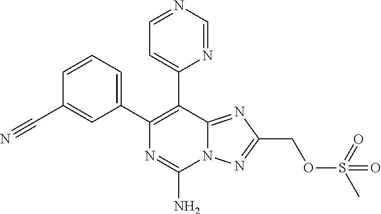 [SMILES: CS(=O)(=O)OCc1nc2c(-c3ccncn3)c(-c3cccc(C#N)c3)nc(N)n2n1]

Methanesulfonyl chloride (11.3 μL, 0.145 mmol) was added to a mixture of 3-(5-amino-2-(hydroxymethyl)-8-(pyrimidin-4-yl)-[1,2,4]triazolo[1,5-c]pyrimidin-7-yl)benzonitrile (50 mg, 0.145 mmol) and pyridine (23.5 μL, 0.290 mmol) in DCM (4.0 mL) at 0° C. The reaction mixture was stirred at r.t. for 30 min, quenched with saturated NaHCO₃ solution, and extracted with EtOAc (3×10 mL). The combined organic layers were dried over MgSO₄, and concentrated to afford the desired product. LCMS calculated for C₁₈H₁₅N₈O₃S (M+H)⁺: 423.1. found 423.1.

### Step 3: 3-(5-Amino-2-((3-hydroxyazetidin-1 yl)methyl)-8-(pyrimidin-4-yl)-[1,2,4]triazolo[1,5-c]pyrimidin-7-yl)benzonitrile

A mixture of (5-amino-7-(3-cyanophenyl)-8-(pyrimidin-4-yl)-[1,2,4]triazolo[1,5-c]pyrimidin-2-yl)methyl methanesulfonate (10.0 mg, 0.024 mmol), azetidin-3-ol hydrochloride (3.9 mg, 0.036 mmol), and DIPEA (8.3 μL, 0.047 mmol) in DMF (0.5 mL) was stirred at 90° C. until completion. The reaction mixture was then cooled to r.t., and directly purified by preparative LC-MS (pH 10, acetonitrile/water with NH₄OH) to give the desired product. LCMS calculated for C₂₀H₁₈N₉O (M+H)⁺: 400.2. found 400.2.

### Example 58. 3-(5-Amino-8-(3-methylpyridin-4-yl)-2-(pyridin-2-ylmethyl)-[1,2,4]triazolo[1,5-c]pyrimidin-7-yl)benzonitrile

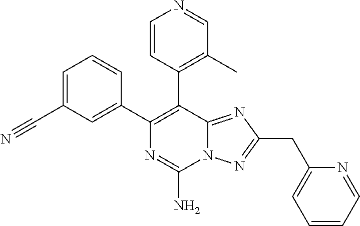 [SMILES: Cc1cnccc1-c1c(-c2cccc(C#N)c2)nc(N)n2nc(Cc3ccccn3)nc12]

This compound was prepared using similar procedures as described in Example 42 using 3-methylpyridin-4-ylboronic acid in place of 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₄H₁₉N₈ (M+H)⁺: 419.2. Found: 419.3.

### Example 59. 3-(5-Amino-8-(2-methoxy-6-methylpyridin-4-yl)-2-(pyridin-2-ylmethyl)-[1,2,4]triazolo[1,5-c]pyrimidin-7-yl)benzonitrile

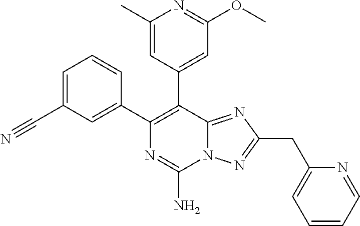 [SMILES: COc1cc(-c2c(-c3cccc(C#N)c3)nc(N)n3nc(Cc4ccccn4)nc23)cc(C)n1]

This compound was prepared using similar procedures as described in Example 42 using 2-methoxy-6-methylpyridin-4-ylboronic acid in place of 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₅H₂₁N₈O (M+H)⁺: 449.2. Found: 449.3.

### Example 60. 3-(5-Amino-8-(pyrazolo[1,5-b]pyridazin-3-yl)-2-(pyridin-2-ylmethyl)-[1,2,4]triazolo[1,5-c]pyrimidin-7-yl)benzonitrile

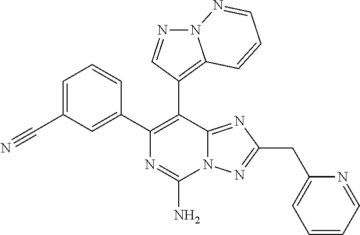 [SMILES: N#Cc1cccc(-c2nc(N)n3nc(Cc4ccccn4)nc3c2-c2cnn3ncccc23)c1]

This compound was prepared using similar procedures as described in Example 42 using 3-(4,4,5,5-tetramethyl-1,3,2-dioxaborolan-2-yl)pyrazolo[1,5-b]pyridazine in place of 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₄H₁₇N₁₀ (M+H)⁺: 445.2. Found: 445.3.

### Example 61. 3-(5-Amino-8-(4-methyloxazol-5-yl)-2-(pyridin-2-ylmethyl)-[1,2,4]triazolo[1,5-c]pyrimidin-7-yl)benzonitrile

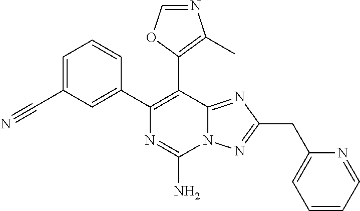 [SMILES: Cc1ncoc1-c1c(-c2cccc(C#N)c2)nc(N)n2nc(Cc3ccccn3)nc12]

### Step 1: 4-Methyl-5-(4,4,5,5-tetramethyl-1,3,2-dioxaborolan-2 yl)oxazole

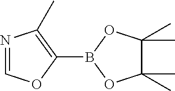

To a mixture of (1,5-cyclooctadiene)(methoxy)iridium(I) dimer (20 mg, 0.030 mmol) in pentane (2 mL) under N₂ gas was added 4,4,5,5-tetramethyl-1,3,2-dioxaborolane (0.22 mL, 1.5 mmol) at r.t. After stirring for 15 min, 4,4′-di-tert-butyl-2,2′-dipyridyl was added and the resulting mixture was stirred for another 15 min before a solution of 3-methyloxazole (83 mg, 1.0 mmol) in Et₂O (2 mL) was added. The reaction mixture was then stirred at r.t. for 2 h, and concentrated to afford the crude product, which was used in the next step without further purification. LC-MS calculated for C₁₀H₁₇BNO₃ (M+H)⁺: m/z=210.1. found 210.1.

### Step 2: 3-(5-Amino-8-(4-methyloxazol-5 yl)-2-(pyridin-2 ylmethyl)-[1,2,4]triazolo[1,5-c]pyrimidin-7-yl)benzonitrile

This compound was prepared using similar procedures as described in Example 42 using 4-methyl-5-(4,4,5,5-tetramethyl-1,3,2-dioxaborolan-2-yl)oxazole in place of 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₂H₁₇N₈O (M+H)⁺: 409.1. Found: 409.2.

### Example 62. 3-(5-Amino-8-(4-(hydroxymethyl)-2-methyloxazol-5-yl)-2-(pyridin-2-ylmethyl)-[1,2,4]triazolo[1,5-c]pyrimidin-7-yl)benzonitrile

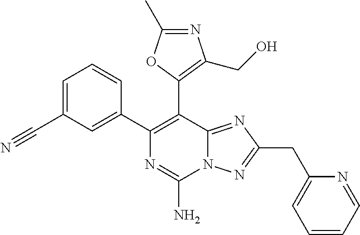 [SMILES: Cc1nc(CO)c(-c2c(-c3cccc(C#N)c3)nc(N)n3nc(Cc4ccccn4)nc23)o1]

### Step 1: 4-((tert-Butyldimethylsilyloxy)methyl)-2-methyloxazole

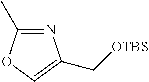

To a solution of (2-methyloxazol-4-yl)methanol (1.0 g, 8.84 mmol) and imidazole (0.90 g, 13.3 mmol) in DCM (20 ml) was added TBSCl (1.5 g, 9.7 mmol). The reaction mixture was stirred at r.t. for 2 h, and concentrated. The resulting residue was then diluted with Et₂O (20 mL), washed with saturated NH₄Cl solution and brine, dried over MgSO₄, and concentrated to afford the crude product which was used in the next step without further purification. LC-MS calculated for C₁₁H₂₂NO₂Si (M+H)⁺: m/z=228.1. found 228.1.

### Step 2: 4-((tert-Butyldimethylsilyloxy)methyl)-2-methyl-5-(4,4,5,5-tetramethyl-1,3,2-dioxaborolan-2 yl)oxazole

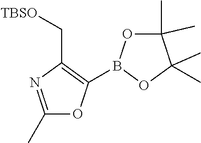

This compound was prepared using similar procedures as described in Example 61, Step 1, using 4-((tert-butyldimethylsilyloxy)methyl)-2-methyloxazole in place of 3-methyloxazole. LCMS calculated for C₁₇H₃₃BNO₄Si (M+H)⁺: 354.2. Found: 354.2.

### Step 3: 3-(5-Amino-8-(4-((tert-butyldimethylsilyloxy)methyl)-2-methyloxazol-5-yl)-2-(pyridin-2-ylmethyl)-[1,2,4]triazolo[1,5-c]pyrimidin-7-yl)benzonitrile

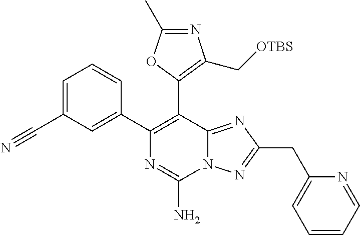 [SMILES: Cc1nc(CO[Si](C)(C)C(C)(C)C)c(-c2c(-c3cccc(C#N)c3)nc(N)n3nc(Cc4ccccn4)nc23)o1]

This compound was prepared using similar procedures as described of Example 42 using 4-((tert-butyldimethylsilyloxy)methyl)-2-methyl-5-(4,4,5,5-tetramethyl-1,3,2-dioxaborolan-2-yl)oxazole in place of 1-ethyl-5-(4,4,5,5-tetramethyl-1,3,2-dioxaborolan-2-yl)-1H-pyrazole. The crude material from this step was used in the next step without further purification. LCMS calculated for C₂₉H₃₃N₈O₂Si (M+H)⁺: 553.2. Found: 553.2.

### Step 4: 3-(5-Amino-8-(4-(hydroxymethyl)-2-methyloxazol-5-yl)-2-(pyridin-2 ylmethyl)-[1,2,4]triazolo[1,5-c]pyrimidin-7-yl)benzonitrile

The crude material from previous step was treated with TFA (0.5 mL) and stirred for 0.5 h at 100° C. The reaction mixture was cooled to r.t., diluted with MeOH, and purified by preparative LC-MS (pH 2, acetonitrile/water with TFA) to afford the product as a TFA salt. LCMS calculated for C₂₃H₁₉N₈O₂ (M+H)⁺: 439.2. Found: 439.3.

### Example 63. 3-(5-Amino-8-(4-(methoxymethyl)-2-methyloxazol-5-yl)-2-(pyridin-2-ylmethyl)-[1,2,4]triazolo[1,5-c]pyrimidin-7-yl)benzonitrile

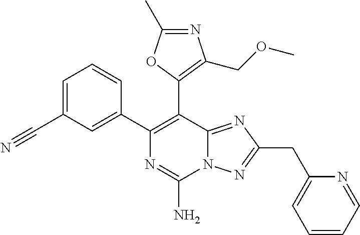 [SMILES: COCc1nc(C)oc1-c1c(-c2cccc(C#N)c2)nc(N)n2nc(Cc3ccccn3)nc12]

### Step 1: 4-(Methoxymethyl)-2-methyloxazole

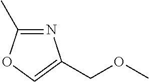

To a solution of (2-methyloxazol-4-yl)methanol (113 mg, 1.0 mmol) in THE (10 ml) was added NaH (48 mg, 60 wt %, 1.2 mmol) at 0° C. After stirring for 0.5 h, iodomethane (170 mg, 1.2 mmol) was added. The reaction mixture was stirred at r.t. for 2 h, diluted with Et₂O (20 mL), washed with saturated NH₄Cl solution and brine, dried over MgSO₄, and concentrated to give the crude product which was used in the next step without further purification. LC-MS calculated for C₆H₁₀NO₂ (M+H)⁺: m/z=128.1. found 128.1.

### Step 2: 4-(Methoxymethyl)-2-methyl-5-(4,4,5,5-tetramethyl-1,3,2-dioxaborolan-2 yl)oxazole

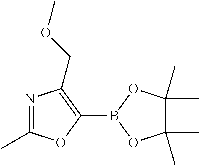

This compound was prepared using similar procedures as described of Example 61, Step 1, using 4-(methoxymethyl)-2-methyloxazole in place of 3-methyloxazole. LCMS calculated for C₁₂H₂₁BNO₄ (M+H)⁺: 254.2. Found: 254.2.

### Step 3: 3-(5-Amino-8-(4-(methoxymethyl)-2-methyloxazol-5-yl)-2-(pyridin-2 ylmethyl)-[1,2,4]triazolo[1,5-c]pyrimidin-7-yl)benzonitrile

This compound was prepared using similar procedures as described in Example 42 using 4-(methoxymethyl)-2-methyl-5-(4,4,5,5-tetramethyl-1,3,2-dioxaborolan-2-yl)oxazole in place of 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₄H₂₁N₈O₂ (M+H)⁺: 453.2. Found: 453.2.

### Examples 64-65. (S)-3-(5-amino-2-(hydroxy(phenyl)methyl)-8-(pyrimidin-4-yl)-[1,2,4]triazolo[1,5-c]pyrimidin-7-yl)benzonitrile (Example 64) & (R)-3-(5-amino-2-(hydroxy(phenyl)methyl)-8-(pyrimidin-4-yl)-[1,2,4]triazolo[1,5-c]pyrimidin-7-yl)benzonitrile (Example 65)

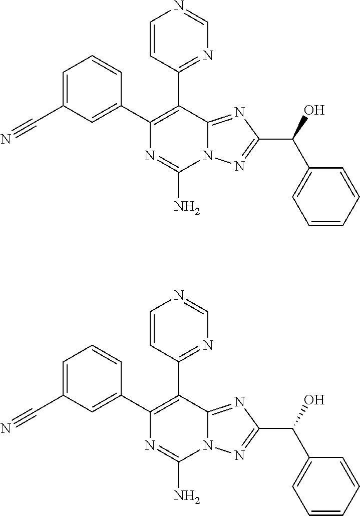 [SMILES: N#Cc1cccc(-c2nc(N)n3nc([C@@H](O)c4ccccc4)nc3c2-c2ccncn2)c1 ; N#Cc1cccc(-c2nc(N)n3nc([C@H](O)c4ccccc4)nc3c2-c2ccncn2)c1]

These compounds were prepared using similar procedures as described for Example 41, with 2-hydroxy-2-phenylacetohydrazide (Alfa Aesar-L11653) replacing 2-(pyridin-2-yl)acetohydrazide in Step 3. The two enantiomers were first separated by chiral HPLC using a Phenomenex Lux Cellulose-4 column (21.2×250 mm, 5 μm particle size) eluting with an isocratic mobile phase 45% EtOH in hexanes with a flow rate of 20 mL/minute. The retention times of Peak 1 (Example 64) and Peak 2 (Example 65) were 9.47 min and 14.42 min, respectively. Following their separation, the enantiomers were individually purified by prep-LCMS (pH=2, MeCN/water with TFA) to give the desired products both as TFA salt. For both products, LC-MS calculated for C₂₃H₁₇N₈O (M+H)⁺: m/z=421.2. found 421.3.

Alternatively, Example 64 could be prepared using similar procedures as described for Example 41, with methyl (S)-(+)-mandelate (Sigma-Aldrich-251542) replacing methyl 2-(pyridin-2-yl)acetate in Step 2. The crude material was purified by prep-LCMS (pH=2, MeCN/water with TFA) to give the desired product as a TFA salt. LC-MS calculated for C₂₃H₁₇N₈O (M+H)⁺: m/z=421.2. found 421.3.

Example 65 could be prepared using similar procedures as described for Example 41, with methyl (R)-(−)-mandelate (Sigma-Aldrich-251550) replacing methyl 2-(pyridin-2-yl)acetate in Step 2. The crude material was purified by prep-LCMS (pH=2, MeCN/water with TFA) to give the desired product as a TFA salt. LC-MS calculated for C₂₃H₁₇N₈O (M+H)⁺: m/z=421.2. found 421.3.

### Example 66. 3-(5-Amino-2-benzoyl-8-(pyrimidin-4-yl)-[1,2,4]triazolo[1,5-c]pyrimidin-7-yl)benzonitrile

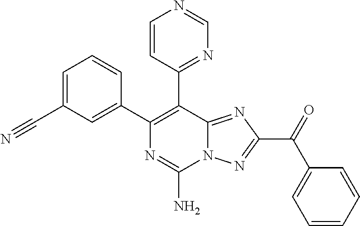 [SMILES: N#Cc1cccc(-c2nc(N)n3nc(C(=O)c4ccccc4)nc3c2-c2ccncn2)c1]

To solution of 3-(5-amino-2-(hydroxy(phenyl)methyl)-8-(pyrimidin-4-yl)-[1,2,4]triazolo[1,5-c]pyrimidin-7-yl)benzonitrile (from Example 64) (370 mg, 0.87 mmol) in acetonitrile (7.2 mL) and DMF (1.4 mL) was added tetrakisacetonitrile copper(I) trifilate (65 mg, 0.17 mmol), 4,4′-dimethoxy-2,2′-bipyridine (38 mg, 0.17 mmol), 9-azabicyclo[3.3.1]nonane N-oxyl (5.8 mg, 0.04 mmol), and 1-methyl-1H-imidazole (28 μL, 0.35 mmol). The reaction mixture was stirred for 30 min open to air at r.t., and then volatiles were removed under reduced pressure. The crude material was purified by prep-LCMS (pH=2, MeCN/water with TFA) to give the desired product as a TFA salt. LC-MS calculated for C₂₃H₁₅N₈O (M+H)⁺: m/z=419.1. found 419.3.

### Example 67. 3-(5-Amino-8-(1-ethyl-1H-pyrazol-5-yl)-2-(1-phenylcyclopropyl)-[1,2,4]triazolo[1,5-c]pyrimidin-7-yl)benzonitrile

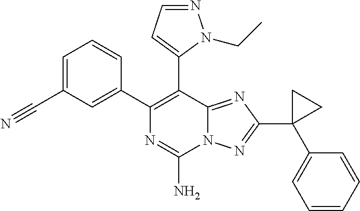 [SMILES: CCn1nccc1-c1c(-c2cccc(C#N)c2)nc(N)n2nc(C3(c4ccccc4)CC3)nc12]

### Step 1: 3-(5 Amino-8-bromo-2-(1 phenylcyclopropyl)-[1,2,4]triazolo[1,5-c]pyrimidin-7-yl)benzonitrile

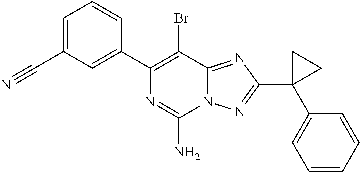 [SMILES: N#Cc1cccc(-c2nc(N)n3nc(C4(c5ccccc5)CC4)nc3c2Br)c1]

To a solution of 3-(5-amino-2-(1-phenylcyclopropyl)-[1,2,4]triazolo[1,5-c]pyrimidin-7-yl)benzonitrile (prepared using similar procedures as described for Example 41, Step 1 to Step 4, with 1-phenylcyclopropane-1-carbohydrazide replacing 2-(pyridin-2-yl)acetohydrazide in Step 3) (380 mg, 1.1 mmol) in DMF (2.1 mL) was slowly added NBS (190 mg, 1.1 mmol) at 0° C. The reaction mixture was then stirred at r.t. for 30 min before water (10 mL) was added. The resulting solid was collected by filtration, and dried to obtain the desired product. LC-MS calculated for C₂₁H₁₆BrN₆ (M+H)⁺: m/z=431.0. found 431.2.

### Step 2: 3-(5-Amino-8-(1-ethyl-1H-pyrazol-5-yl)-2-(1 phenylcyclopropyl)-[1,2,4]triazolo[1,5-c]pyrimidin-7-yl)benzonitrile

A mixture of 3-(5-amino-8-bromo-2-(1-phenylcyclopropyl)-[1,2,4]triazolo[1,5-c]pyrimidin-7-yl)benzonitrile (20 mg, 0.046 mmol), 1-ethyl-5-(4,4,5,5-tetramethyl-1,3,2-dioxaborolan-2-yl)-1H-pyrazole (10 mg, 0.046 mmol), XPhos Pd G2 (7.0 mg, 9.3 μmol), and Na₂CO₃ (20 mg, 0.19 mmol) in 1,4-dioxane (0.50 mL) and water (0.05 mL) was flushed with nitrogen and sealed. The reaction mixture was stirred at 110° C. for 1 h, cooled to room temperature, diluted with methanol, and purified with prep-LC-MS (pH=2, MeCN/water with TFA) to give the desired product as a TFA salt. LC-MS calculated for C₂₆H₂₃N₈ (M+H)⁺: m/z=447.2. found 447.3.

### Example 68. 2-((7-(3-Cyanophenyl)-2-(hydroxy(phenyl)methyl)-8-(pyrimidin-4-yl)-[1,2,4]triazolo[1,5-c]pyrimidin-5-yl)amino)nicotinonitrile

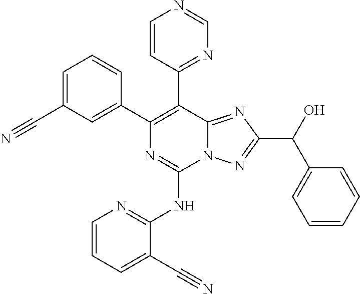 [SMILES: N#Cc1cccc(-c2nc(Nc3ncccc3C#N)n3nc(C(O)c4ccccc4)nc3c2-c2ccncn2)c1]

### Step 1: 2-((8-Bromo-7-(3-cyanophenyl)-2-(hydroxy(phenyl)methyl)-[1,2,4]triazolo[1,5-c]pyrimidin-5 yl)amino)nicotinonitrile

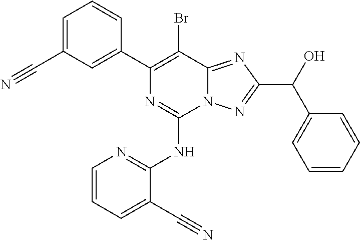 [SMILES: N#Cc1cccc(-c2nc(Nc3ncccc3C#N)n3nc(C(O)c4ccccc4)nc3c2Br)c1]

To a solution of 3-(5-amino-8-bromo-2-(hydroxy(phenyl)methyl)-[1,2,4]triazolo[1,5-c]pyrimidin-7-yl)benzonitrile (from Example 45, Step 1) (50 mg, 0.12 mmol) in THE (0.60 mL) was slowly added sodium hydride (7.1 mg, 0.18 mmol, 60 wt %) at 0° C. The reaction mixture was then stirred at r.t. for 30 min before 2-fluoronicotinonitrile (23 mg, 0.19 mmol) was added. The reaction mixture was then stirred at reflux for 2 h, quenched with water (5 mL), and extracted with EtOAc (5×5 mL). The combined organic layers were washed with water and brine, dried with MgSO₄, and concentrated. The resulting material was purified by column chromatography eluting with 0-20% MeOH/DCM to give the desired product. LC-MS calculated for C₂₅H₁₆BrN₈O (M+H)⁺: m/z=523.0. found 523.0.

### Step 2: 2-((7-(3-Cyanophenyl)-2-(hydroxy(phenyl)methyl)-8-(pyrimidin-4-yl)-[1,2,4]triazolo[1,5-c]pyrimidin-5-yl)amino)nicotinonitrile

A mixture of 2-((8-bromo-7-(3-cyanophenyl)-2-(hydroxy(phenyl)methyl)-[1,2,4]triazolo[1,5-c]pyrimidin-5-yl)amino)nicotinonitrile (25 mg, 0.05 mmol), 4-(tributylstannyl)pyrimidine (17 μL, 0.05 mmol), tetrakis(triphenylphosphine)palladium(0) (5.6 mg, 4.9 μmol), copper(I) iodide (1.9 mg, 9.8 μmol) and cesium fluoride (15 mg, 0.10 mmol) in dioxane (0.50 mL) was heated and stirred at 140° C. for 60 min in a microwave reactor. The reaction mixture was then cooled to r.t., filtered through a Celite plug (washed with DCM), and concentrated. The resulting residue was purified by prep-LC-MS (pH=2, MeCN/water with TFA) to give the desired product as a TFA salt. LC-MS calculated for C₂₉H₁₉N₁₀O (M+H)⁺: m/z=523.2. found 523.2.

### Example 69. 3-(5-Amino-2-((cyano(phenyl)methyl)amino)-8-(pyrimidin-4-yl)-[1,2,4]triazolo[1,5-c]pyrimidin-7-yl)benzonitrile

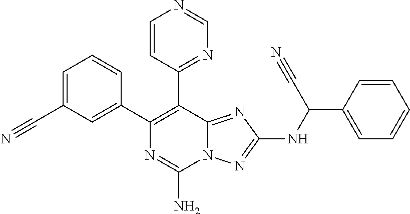 [SMILES: N#Cc1cccc(-c2nc(N)n3nc(NC(C#N)c4ccccc4)nc3c2-c2ccncn2)c1]

### Step 1: 6-Chloro-N²,N²-bis(4-methoxybenzyl)pyrimidine-2,4-diamine

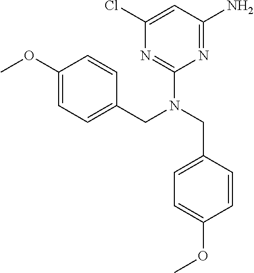 [SMILES: COc1ccc(CN(Cc2ccc(OC)cc2)c2nc(N)cc(Cl)n2)cc1]

To a solution of 2,6-dichloropyrimidin-4-amine (5.0 g, 31 mmol) in 2-propanol (31 mL) was added N,N-diisopropylethylamine (6.4 ml, 37 mmol) and bis(4-methoxybenzyl)amine (7.9 g, 31 mmol). The resulting solution was stirred at 100° C. for 16 h, cooled to r.t., diluted with water (100 mL), and extracted with EtOAc (100 mL). The organic layer was washed with water and brine, dried over anhydrous sodium sulfate, and concentrated to yield the crude product, which was used in the next step without further purification. LC-MS calculated for C₂₀H₂₂ClN₄O₂ (M+H)⁺: 385.1. found 385.1.

### Step 2: 7-Chloro-N⁵,N⁵-bis(4-methoxybenzyl)-[1,2,4]triazolo[1,5-c]pyrimidine-2,5-diamine

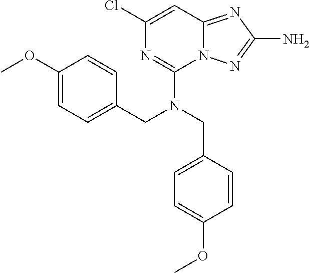 [SMILES: COc1ccc(CN(Cc2ccc(OC)cc2)c2nc(Cl)cc3nc(N)nn23)cc1]

O-ethyl carbonisothiocyanatidate (3.1 mL, 26 mmol) was added to a 1,4-dioxane (5.0 mL) solution of 6-chloro-N²,N²-bis(4-methoxybenzyl)pyrimidine-2,4-diamine (1.0 g, 2.6 mmol) at r.t. The reaction mixture was then stirred at 90° C. overnight, cooled to r.t., and concentrated. The resulting material was dissolved in methanol (12 mL) and ethanol (12 mL), and N,N-diisopropylethylamine (0.91 mL, 5.2 mmol) was added, followed by hydroxylamine hydrochoride (0.54 g, 7.8 mmol). The reaction mixture was stirred at 45° C. for 2 h, cooled to r.t., and concentrated. The resulting material was taken into EtOAc, washed with water, dried over anhydrous sodium sulfate, and concentrated. The crude material was then purified by silica gel chromatography eluting with 0% to 50% EtOAc in hexanes to afford the product. LC-MS calculated for C₂₁H₂₂ClN₆O₂ (M+H)⁺: 425.1. found 425.2.

Step 3: 3-(2-Amino-5-(bis(4-methoxybenzyl)amino)-[1,2,4]triazolo[1,5-c]pyrimidin-7-yl)benzonitrile

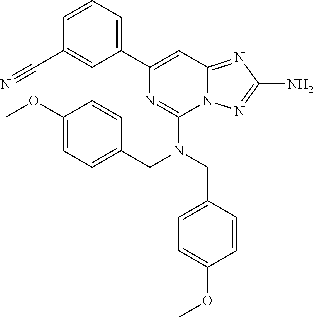 [SMILES: COc1ccc(CN(Cc2ccc(OC)cc2)c2nc(-c3cccc(C#N)c3)cc3nc(N)nn23)cc1]

Chloro(2-dicyclohexylphosphino-2′,4′,6′-tri-i-propyl-1,1′-biphenyl)(2′-amino-1,1′-biphenyl-2-yl) palladium(II) (330 mg, 0.42 mmol) was added to a mixture of (3-cyanophenyl)boronic acid (460 mg, 3.2 mmol), 7-chloro-N⁵,N⁵-bis(4-methoxybenzyl)-[1,2,4]triazolo[1,5-c]pyrimidine-2,5-diamine (890 mg, 2.1 mmol), and sodium carbonate (890 mg, 8.4 mmol) in 1,4-dioxane (8.8 mL) and water (1.8 mL). The mixture was purged with N₂ and stirred at 95° C. overnight. The reaction mixture was then cooled to r.t., concentrated, and purified by silica gel chromatography eluting with 0% to 50% EtOAc in DCM to afford the desired product. LC-MS calculated for C₂₈H₂₆N₇O₂ (M+H)⁺: 492.2. found 492.2.

Step 4: 3-(2 Amino-5-(bis(4-methoxybenzyl)amino)-8-bromo-[1,2,4]triazolo[1,5-c]pyrimidin-7-yl)benzonitrile

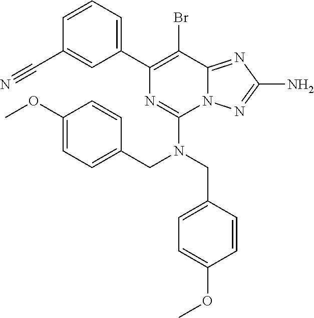 [SMILES: COc1ccc(CN(Cc2ccc(OC)cc2)c2nc(-c3cccc(C#N)c3)c(Br)c3nc(N)nn23)cc1]

To a solution of 3-(2-amino-5-(bis(4-methoxybenzyl)amino)-[1,2,4]triazolo[1,5-c]pyrimidin-7-yl)benzonitrile (330 mg, 0.66 mmol) in DMF (1.4 mL) was slowly added NBS (120 mg, 0.66 mmol) at 0° C. The reaction mixture was then stirred at r.t. for 30 min before water (10 mL) was added. The resulting solid was collected by filtration, and dried to obtain the desired product. LC-MS calculated for C₂₈H₂₅BrN₇O₂ (M+H)⁺: m/z=570.1. found 570.2.

### Step 5: 3-(2 Amino-5-(bis(4-methoxybenzyl)amino)-8-(pyrimidin-4-yl)-[1,2,4]triazolo[1,5-c]pyrimidin-7-yl)benzonitrile

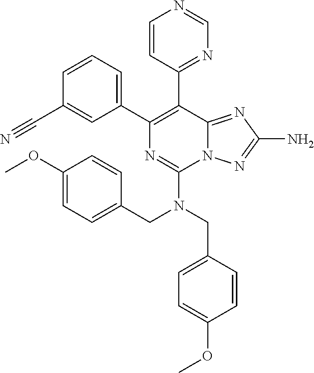 [SMILES: COc1ccc(CN(Cc2ccc(OC)cc2)c2nc(-c3cccc(C#N)c3)c(-c3ccncn3)c3nc(N)nn23)cc1]

A mixture of 3-(2-amino-5-(bis(4-methoxybenzyl)amino)-8-bromo-[1,2,4]triazolo[1,5-c]pyrimidin-7-yl)benzonitrile (350 mg, 0.61 mmol), 4-(tributylstannyl)pyrimidine (210 μL, 0.67 mmol), tetrakis(triphenylphosphine)palladium(0) (70 mg, 0.060 mmol), copper(I) iodide (23 mg, 0.12 mmol) and cesium fluoride (180 mg, 1.2 mmol) in dioxane (4.7 mL) was heated and stirred at 140° C. for 30 min in a microwave reactor. The reaction mixture was then cooled to r.t., filtered through a Celite plug (washed with DCM), and concentrated. The resulting material was purified by silica gel column chromatography eluting with 0-20% MeOH/DCM to give the desired product. LC-MS calculated for C₃₂H₂₈N₉O₂ (M+H)⁺: m/z=570.2. found 570.3.

Step 6: 3-(5-Amino-2-((cyano(phenyl)methyl)amino)-8-(pyrimidin-4-yl)-[1,2,4]triazolo[1,5-c]pyrimidin-7-yl)benzonitrile

A mixture of 3-(2-amino-5-(bis(4-methoxybenzyl)amino)-8-(pyrimidin-4-yl)-[1,2,4]triazolo[1,5-c]pyrimidin-7-yl)benzonitrile (20 mg, 0.04 mmol) and benzaldehyde (7.2 μL, 0.070 mmol) in DCM (0.20 mL) and methanol (0.20 mL) were heated and stirred at 100° C. for 5 min. The reaction mixture was cooled to r.t. and trimethylsilyl cyanide (19 μL, 0.14 mmol) was added. After stirring at 100° C. for 30 min, the reaction mixture was cooled to r.t., diluted with EtOAc, washed with water and brine, dried over anhydrous sodium sulfate, and concentrated. Trifluoroacetic acid (1.0 mL) was then added to the resulting residue and the mixture was stirred at 100° C. for 30 min, cooled to r.t., and concentrated. The resulting residue was purified by prep-LC-MS (pH=2, MeCN/water with TFA) to give the desired product as a TFA salt. LC-MS calculated for C₂₄H₁₇N₁O (M+H)⁺: m/z=445.2. found 445.2.

### Example 70. 3-(5-Amino-8-(pyridin-4-yl)-2-(tetrahydrofuran-3-yl)-[1,2,4]triazolo[1,5-c]pyrimidin-7-yl)benzonitrile

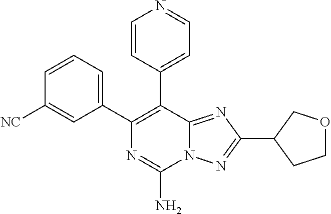 [SMILES: N#Cc1cccc(-c2nc(N)n3nc(C4CCOC4)nc3c2-c2ccncc2)c1]

### Step 1: 3-(5 Amino-8-bromo-2-(tetrahydrofuran-3 yl)-[1,2,4]triazolo[1,5-c]pyrimidin-7-yl)benzonitrile

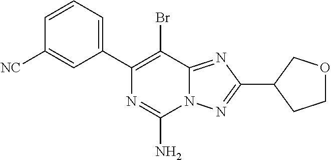 [SMILES: N#Cc1cccc(-c2nc(N)n3nc(C4CCOC4)nc3c2Br)c1]

This compound was prepared using similar procedures as described for Example 41, Step 1 to Step 4, with tetrahydrofuran-3-carbohydrazide replacing 2-(pyridin-2-yl)acetohydrazide in Step 3. LCMS calculated for C₁₆H₁₄BrN₆₀ (M+H)⁺: 385.0. found 385.0.

### Step 2: 3-(5 Amino-8-(pyridin-4-yl)-2-(tetrahydrofuran-3 yl)-[1,2,4]triazolo[1,5-c]pyrimidin-7-yl)benzonitrile

A mixture of 3-(5-amino-8-bromo-2-(tetrahydrofuran-3-yl)-[1,2,4]triazolo[1,5-c]pyrimidin-7-yl)benzonitrile (15 mg, 0.039 mmol), pyridin-4-ylboronic acid (10 mg, 0.078 mmol), chloro(2-dicyclohexylphosphino-2′,4′,6′-triisopropyl-1,1′-biphenyl)[2-(2′-amino-1,1′-biphenyl)]palladium(II) (3.1 mg, 3.89 μmol), and sodium carbonate (12.4 mg, 0.117 mmol) in 1,4-dioxane (1.0 mL) and water (0.2 mL) was purged with N₂ and stirred at 90° C. overnight. The reaction mixture was then cooled to r.t., concentrated, and purified by prep-LC-MS (pH=2, MeCN/water with TFA) to give the desired product as a TFA salt. LC-MS calculated for C₂₁H₁₈N₇O (M+H)⁺: m/z=384.1. found 384.1.

### Example 71. 3-(5-Amino-2-(phenyl(pyridin-2-yloxy)methyl)-8-(pyrimidin-4-yl)-[1,2,4]triazolo[1,5-c]pyrimidin-7-yl)benzonitrile

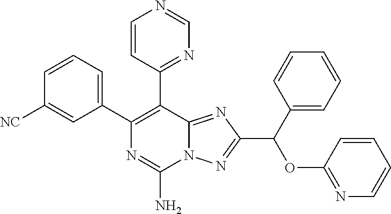 [SMILES: N#Cc1cccc(-c2nc(N)n3nc(C(Oc4ccccn4)c4ccccc4)nc3c2-c2ccncn2)c1]

### Step 1: 3-(5-Amino-8-bromo-2-(phenyl(pyridin-2 yloxy)methyl)-[1,2,4]triazolo[1,5-c]pyrimidin-7-yl)benzonitrile

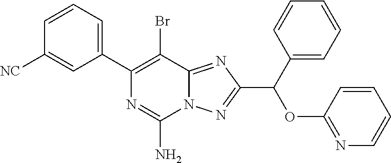 [SMILES: N#Cc1cccc(-c2nc(N)n3nc(C(Oc4ccccn4)c4ccccc4)nc3c2Br)c1]

To a solution of 3-(5-amino-8-bromo-2-(hydroxy(phenyl)methyl)-[1,2,4]triazolo[1,5-c]pyrimidin-7-yl)benzonitrile (from Example 45, Step 1) (110 mg, 0.261 mmol) in DCM (2 mL) was added thionyl chloride (0.057 mL, 0.783 mmol). The reaction solution was stirred at r.t. for 1 h, and concentrated. The resulting residue was dissolved in DMF (1 mL), and added potassium carbonate (108 mg, 0.783 mmol) and pyridin-2-ol (49.7 mg, 0.522 mmol). The reaction mixture was then stirred at 90° C. for 2 h, cooled to r.t., quenched with water, and extracted with DCM.

The combined organic layers were dried over MgSO₄, concentrated, and purified by flash chromatography on a silica gel column eluting with 50% EtOAc in dichloromethane to afford the desired product. LCMS calculated for C₂₄H₁₇BrN₇O (M+H)⁺: 498.1. Found: 498.1.

### Step 2: 3-(5-Amino-2-(phenyl(pyridin-2 yloxy)methyl)-8-(pyrimidin-4-yl)-[1,2,4]triazolo[1,5-c]pyrimidin-7-yl)benzonitrile

To a mixture of 3-(5-amino-8-bromo-2-(phenyl(pyridin-2-yloxy)methyl)-[1,2,4]triazolo[1,5-c]pyrimidin-7-yl)benzonitrile (15 mg, 0.030 mmol), 4-(tributylstannyl)pyrimidine (22.2 mg, 0.060 mmol), and lithium chloride (5.1 mg, 0.120 mmol) in 1,4-dioxane (100 μL) was added dichlorobis(triphenylphosphine)-palladium(II) (2.1 mg, 3.01 μmol) and copper(I) chloride (11.9 mg, 0.120 mmol). The resulting mixture was purged with N₂ and stirred at 100° C. overnight. The reaction mixture was then cooled to r.t., concentrated, and purified by prep-LC-MS (pH=2, MeCN/water with TFA) to give the desired product as a TFA salt. LC-MS calculated for C₂₈H₂₀N₉O (M+H)⁺: m/z=498.2. found 498.1.

### Example 72. 3-(5-Amino-2-(2-fluoro-6-(((3R,4R)-3-fluoro-4-hydroxypyrrolidin-1-yl)methyl)benzyl)-8-(pyrimidin-4-yl)-[1,2,4]triazolo[1,5-c]pyrimidin-7-yl)benzonitrile

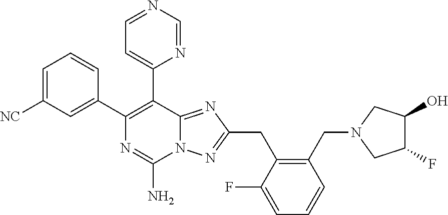 [SMILES: N#Cc1cccc(-c2nc(N)n3nc(Cc4c(F)cccc4CN4C[C@@H](O)[C@H](F)C4)nc3c2-c2ccncn2)c1]

### Step 1: 3-(5 Amino-2-(hydroxymethyl)-[1,2,4]triazolo[1,5-c]pyrimidin-7-yl)benzonitrile

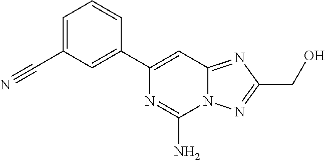 [SMILES: N#Cc1cccc(-c2cc3nc(CO)nn3c(N)n2)c1]

2-Hydroxyacetohydrazide (2.34 g, 26.01 mmol) was added to a ethanol (35 mL) solution of 3-(2-amino-6-chloropyrimidin-4-yl)benzonitrile (4.00 g, 17.34 mmol) (Example 41, Step 1) at r.t. After being heated and stirred at reflux for 2 h, the reaction mixture was cooled to r.t., and concentrated. The resulting residue was taken into N,O-bis(trimethylsilyl)acetamide (20 mL) and stirred at 120° C. for 7 h. The mixture was then cooled to r.t., poured onto ice, and allowed to stir at r.t. for 1 h. The resulting solid was collected by filtration, and taken into 20 mL of 1 N HCl solution. The resulting mixture was stirred at r.t. for 1 h, filtered, and the aqueous layer was neutralized by addition of saturated NaHCO₃ solution. The resulting precipitate was collected by filtration, and dried to obtain the desired product as a brown solid. LCMS calculated for C₁₃H₁₁N₆O (M+H)⁺: 267.1. found 267.1.

### Step 2: 3-(5-Amino-8-bromo-2-(hydroxymethyl)-[1,2,4]triazolo[1,5-c]pyrimidin-7-yl)benzonitrile

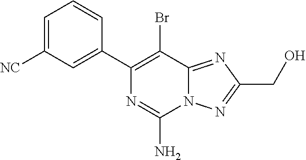 [SMILES: N#Cc1cccc(-c2nc(N)n3nc(CO)nc3c2Br)c1]

To a mixture of 3-(5-amino-2-(hydroxymethyl)-[1,2,4]triazolo[1,5-c]pyrimidin-7-yl)benzonitrile (1.0 g, 3.76 mmol) in DMF (12 mL) at −30° C. was added NBS (0.67 g, 3.76 mmol) portion-wise. The reaction mixture was allowed to slowly warm to 0° C., resulting a homogenous solution. After stirring at 0° C. for 1 h, the reaction mixture was diluted with saturated NaHCO₃ solution and the desired product was collected by filtration and dried. LCMS calculated for C₁₃H₁₀BrN₆O (M+H)⁺: 345.0. found 345.0.

### Step 3: 3-(5 Amino-2-(hydroxymethyl)-8-(pyrimidin-4-yl)-[1,2,4]triazolo[1,5-c]pyrimidin-7-yl)benzonitrile

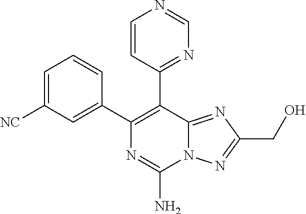 [SMILES: N#Cc1cccc(-c2nc(N)n3nc(CO)nc3c2-c2ccncn2)c1]

Tetrakis(triphenylphosphine)palladium(0) (0.067 g, 0.058 mmol) was added to a mixture of 4-(tributylstannyl)pyrimidine (0.321 g, 0.869 mmol), 3-(5-amino-8-bromo-2-(hydroxymethyl)-[1,2,4]triazolo[1,5-c]pyrimidin-7-yl)benzonitrile (0.20 g, 0.579 mmol), CsF (0.176 g, 1.159 mmol), and copper(I)iodide (0.022 g, 0.116 mmol) in 1,4-dioxane (5.0 mL). The reaction mixture was purged with N₂ and stirred at 80° C. for 7 h. The resulting mixture was cooled to r.t., concentrated and purified by flash column chromatography eluting with 0% to 10% methanol in DCM to afford the product. LC-MS calculated for C₁₇H₁₃N₈O (M+H)⁺: 345.1. found 345.1.

### Step 4: 3-(5-Amino-2-(chloromethyl)-8-(pyrimidin-4-yl)-[1,2,4]triazolo[1,5-c]pyrimidin-7-yl)benzonitrile

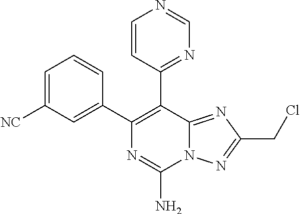 [SMILES: N#Cc1cccc(-c2nc(N)n3nc(CCl)nc3c2-c2ccncn2)c1]

To a mixture of 3-(5-amino-2-(hydroxymethyl)-8-(pyrimidin-4-yl)-[1,2,4]triazolo[1,5-c]pyrimidin-7-yl)benzonitrile (0.1 g, 0.290 mmol) in Acetonitrile (10 ml) was added thionyl chloride (0.212 ml, 2.90 mmol) at r.t. The reaction mixture was stirred at r.t. for 5 h, concentrated, and purified by flash chromatography eluting with 0% to 5% methanol in DCM to afford the product. LC-MS calculated for C₁₇H₁₂ClN₈ (M+H)⁺: 363.1. found 363.1.

### Step 5: 3-(5-Amino-2-(2 fluoro-6-(hydroxymethyl)benzyl)-8-(pyrimidin-4-yl)-[1,2,4]triazolo[1,5-c]pyrimidin-7-yl)benzonitrile

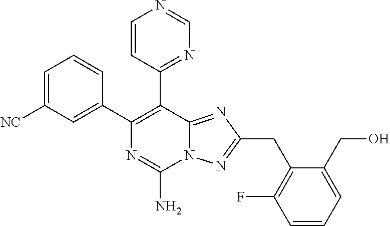 [SMILES: N#Cc1cccc(-c2nc(N)n3nc(Cc4c(F)cccc4CO)nc3c2-c2ccncn2)c1]

A mixture of 3-(5-amino-2-(chloromethyl)-8-(pyrimidin-4-yl)-[1,2,4]triazolo[1,5-c]pyrimidin-7-yl)benzonitrile (0.2 g, 0.55 mmol), (2-fluoro-6-(hydroxymethyl)phenyl)boronic acid (0.141 g, 0.827 mmol), chloro(2-dicyclohexylphosphino-2′,4′,6′-tri-i-propyl-1,1′-biphenyl)(2′-amino-1,1′-biphenyl-2-yl) palladium(II) (0.551 mmol) and Na₂CO₃ (0.117 g, 1.10 mmol) in Dioxane (30 mL) and water (3.0 mL) was stirred at 110° C. for 2 h. The resulting mixture was cooled to r.t., and diluted with water (20 mL). The resulting solid was collected by filtration, and dried to afford the product. LCMS calculated for C₂₄H₁₈FN₈O (M+H)⁺: 453.2. found 453.2.

### Step 6: 3-(5-Amino-2-(2-(chloromethyl)-6 fluorobenzyl)-8-(pyrimidin-4-yl)-[1,2,4]triazolo[1,5-c]pyrimidin-7-yl)benzonitrile

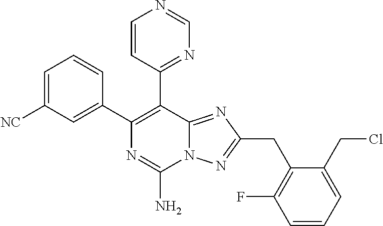 [SMILES: N#Cc1cccc(-c2nc(N)n3nc(Cc4c(F)cccc4CCl)nc3c2-c2ccncn2)c1]

To a mixture of 3-(5-Amino-2-(2-fluoro-6-(hydroxymethyl)benzyl)-8-(pyrimidin-4-yl)-[1,2,4]triazolo[1,5-c]pyrimidin-7-yl)benzonitrile (0.2 g, 0.290 mmol) in acetonitrile (10 ml) was added thionyl chloride (0.212 ml, 2.90 mmol) at r.t. The reaction mixture was stirred at r.t. for 5 h, concentrated, and purified by flash chromatography eluting with 0% to 5% methanol in DCM to afford the product. LC-MS calculated for C₂₄H₁₇ClFN₈ (M+H)⁺: 471.1. found 471.1.

### Step 7: 3-(5-Amino-2-(2 fluoro-6-(((3R,4R)-3 fluoro-4-hydroxypyrrolidin-1 yl)methyl)benzyl)-8-(pyrimidin-4-yl)-[1,2,4]triazolo[1,5-c]pyrimidin-7-yl)benzonitrile

A mixture of 3-(5-amino-2-(2-(chloromethyl)-6-fluorobenzyl)-8-(pyrimidin-4-yl)-[1,2,4]triazolo[1,5-c]pyrimidin-7-yl)benzonitrile (10 mg, 0.021 mmol), (3R,4R)-4-fluoropyrrolidin-3-ol hydrochloride (4.51 mg, 0.032 mmol) and Cs₂CO₃ (20.7 mg, 0.064 mmol) in DMF (1 mL) was stirred at 100° C. for 10 min. The reaction mixture was then cooled to r.t., diluted with methanol (4 mL), and purified by preparative LC-MS (pH 2, acetonitrile/water with TFA) to afford the product as a TFA salt. LCMS calculated for C₂₈H₂₄F₂N₉₀ (M+H)⁺: 540.2. found 540.2.

### Example 73. 1-(2-((5-Amino-7-(3-cyanophenyl)-8-(pyrimidin-4-yl)-[1,2,4]triazolo[1,5-c]pyrimidin-2-yl)methyl)-3-fluorobenzyl)piperidine-4-carboxylic acid

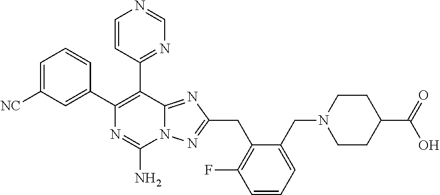 [SMILES: N#Cc1cccc(-c2nc(N)n3nc(Cc4c(F)cccc4CN4CCC(C(=O)O)CC4)nc3c2-c2ccncn2)c1]

### Step 1: Methyl J-(2-((5-amino-7-(3-cyanophenyl)-8-(pyrimidin-4-yl)-[1,2,4]triazolo[1,5-c]pyrimidin-2 yl)methyl)-3 fluorobenzyl)piperidine-4-carboxylate

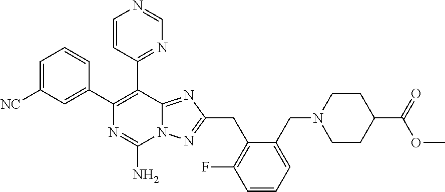 [SMILES: COC(=O)C1CCN(Cc2cccc(F)c2Cc2nc3c(-c4ccncn4)c(-c4cccc(C#N)c4)nc(N)n3n2)CC1]

This compound was prepared using similar procedures, on the same scale, as described for Example 72, with methyl piperidine-4-carboxylate replacing (3R,4R)-4-fluoropyrrolidin-3-ol hydrochloride in Step 7. LCMS calculated for C₃₁H₂₉FN₉O₂ (M+H)⁺: 578.2. found 578.2.

### Step 2: 1-(2-((5-Amino-7-(3-cyanophenyl)-8-(pyrimidin-4-yl)-[1,2,4]triazolo[1,5-c]pyrimidin-2-yl)methyl)-3 fluorobenzyl)piperidine-4-carboxylic acid

The product from the previous step was treated with LiOH (2.5 mg, 0.106 mmol) in water (2.0 mL), and stirred at r.t. for 1.5 h. The mixture was diluted with methanol (5 mL) and purified by preparative LC-MS (pH 2, acetonitrile/water with TFA) to afford the product as a TFA salt. LCMS calculated for C₃₀H₂₇FN₉O₂ (M+H)⁺: 564.2. found 564.2.

### Example 74. N-(2-((5-Amino-7-(3-cyanophenyl)-8-(pyrimidin-4-yl)-[1,2,4]triazolo[1,5-c]pyrimidin-2-yl)methyl)-3-fluorobenzyl)-N-methylmethanesulfonamide

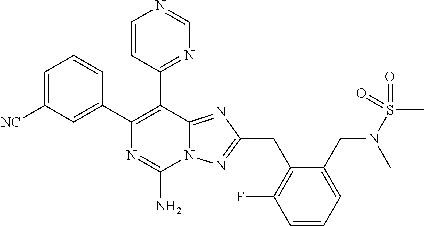 [SMILES: CN(Cc1cccc(F)c1Cc1nc2c(-c3ccncn3)c(-c3cccc(C#N)c3)nc(N)n2n1)S(C)(=O)=O]

This compound was prepared using similar procedures as described for Example 72, with N-methylmethanesulfonamide replacing (3R,4R)-4-fluoropyrrolidin-3-ol hydrochloride in Step 7. The product was purified by preparative LC-MS (pH 2, acetonitrile/water with TFA) to afford the product as a TFA salt. LCMS calculated for C₂₆H₂₃FN₉O₂S (M+H)⁺: 544.2. found 544.2.

### Example 75. 3-(5-Amino-2-(2-((2,5-dioxoimidazolidin-1-yl)methyl)-6-fluorobenzyl)-8-(pyrimidin-4-yl)-[1,2,4]triazolo[1,5-c]pyrimidin-7-yl)benzonitrile

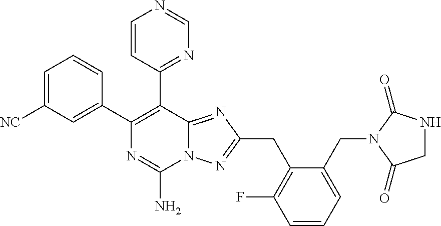 [SMILES: N#Cc1cccc(-c2nc(N)n3nc(Cc4c(F)cccc4CN4C(=O)CNC4=O)nc3c2-c2ccncn2)c1]

This compound was prepared using similar procedures as described for Example 72, with hydantoin replacing (3R,4R)-4-fluoropyrrolidin-3-ol hydrochloride in Step 7. The product was purified by preparative LC-MS (pH 2, acetonitrile/water with TFA) to afford the product as a TFA salt. LCMS calculated for C₂₇H₂₀FN₁₀O₂ (M+H)⁺: 535.2. found 535.2.

### Example 76. 3-(5-Amino-2-((2,6-difluorophenyl)(hydroxy)methyl)-8-(pyrimidin-4-yl)-[1,2,4]triazolo[1,5-c]pyrimidin-7-yl)benzonitrile (Peak 1)

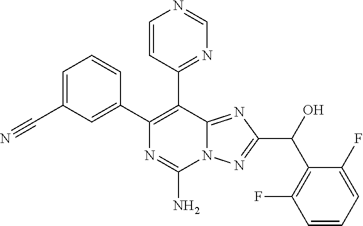 [SMILES: N#Cc1cccc(-c2nc(N)n3nc(C(O)c4c(F)cccc4F)nc3c2-c2ccncn2)c1]

### Step 1: Methyl 2-(2,6-difluorophenyl)-2-hydroxyacetate

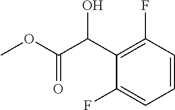

Concentrated sulfuric acid (1.42 mL, 27 mmol) was added to a methanol (45 mL) solution of 2,6-difluoromandelic acid (5 g, 27 mmol) at 0° C. The mixture was stirred at r.t. for 4 h before being concentrated. To the resulting slurry was added saturated NaHCO₃ solution (30 mL). The resulting mixture was extracted with DCM (3×20 mL). The combined organic layers were washed with water, dried over Mg₂SO₄, filtered, and concentrated to afford the crude product, which was used in the next step without further purification. LC-MS calculated for C₁₁H₁₂F₂NO₃ (M+H+MeCN)⁺: m/z=244.1. found 244.2.

### Step 2: 3-(5-Amino-2-((2,6-difluorophenyl) (hydroxy)methyl)-8-(pyrimidin-4-yl)-[1,2,4]triazolo[1,5-c]pyrimidin-7-yl)benzonitrile

This compound was prepared using similar procedures as described for Example 41, with methyl 2-(2,6-difluorophenyl)-2-hydroxyacetate replacing methyl 2-(pyridin-2-yl)acetate in Step 2. The two enantiomers were separated by chiral SFC using a Phenomenex Lux Cellulose-1 column (21.2×250 mm, 5 μm particle size) eluting with an isocratic mobile phase 25% MeOH in CO₂ with a flow rate of 80 mL/minute. Peak 1 was isolated, and further purified by prep-LCMS (pH=2, MeCN/water with TFA) to give the desired product as a TFA salt. LC-MS calculated for C₂₃H₁₅F₂N₈O (M+H)⁺: m/z=457.1. found 457.1. ¹H NMR (500 MHz, DMSO) δ 8.94 (d, J=1.3 Hz, 1H), 8.81 (d, J=5.2 Hz, 1H), 7.85 (dd, J=5.3, 1.4 Hz, 1H), 7.81 (dt, J=7.4, 1.5 Hz, 1H), 7.76 (t, J=1.7 Hz, 1H), 7.55 (dt, J=7.8, 1.5 Hz, 1H), 7.49 (t, J=7.8 Hz, 1H), 7.44 (tt, J=8.4, 6.4 Hz, 1H), 7.09 (t, J=8.3 Hz, 2H), 6.27 (s, 1H).

### Example 77. 3-(5-Amino-2-((2,6-difluorophenyl)(hydroxy)methyl)-8-(pyrimidin-4-yl)-[1,2,4]triazolo[1,5-c]pyrimidin-7-yl)benzonitrile (Peak 2)

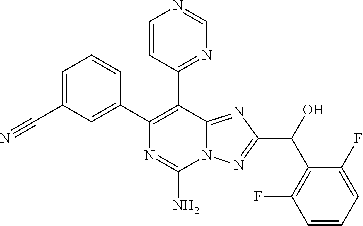 [SMILES: N#Cc1cccc(-c2nc(N)n3nc(C(O)c4c(F)cccc4F)nc3c2-c2ccncn2)c1]

This compound was prepared using the same procedures as described for Example 76. The two enantiomers were separated by chiral SFC using a Phenomenex Lux Cellulose-1 column (21.2×250 mm, 5 μm particle size) eluting with an isocratic mobile phase 25% MeOH in CO₂ with a flow rate of 80 mL/minute. Peak 2 was isolated, and further purified by prep-LCMS (pH=2, MeCN/water with TFA) to give the desired product as a TFA salt. LC-MS calculated for C₂₃H₁₅F₂N₈O (M+H)⁺: m/z=457.1. found 457.1. ¹H NMR (500 MHz, DMSO) δ 8.94 (d, J=1.3 Hz, 1H), 8.81 (d, J=5.2 Hz, 1H), 7.85 (dd, J 5.3, 1.4 Hz, 1H), 7.81 (dt, J=7.4, 1.5 Hz, 1H), 7.76 (t, J 1.7 Hz, 1H), 7.55 (dt, J 7.8, 1.5 Hz, 1H), 7.49 (t, J=7.8 Hz, 1H), 7.44 (tt, J=8.4, 6.4 Hz, 1H), 7.09 (t, J=8.3 Hz, 2H), 6.27 (s, 1H).

### Examples 78A-78B. 3-(5-Amino-2-((5-(pyridin-2-yl)-2H-tetrazol-2-yl)methyl)-8-(pyrimidin-4-yl)-[1,2,4]triazolo[1,5-c]pyrimidin-7-yl)benzonitrile (Example 78A) and 3-(5-Amino-2-((5-(pyridin-2-yl)-1H-tetrazol-1-yl)methyl)-8-(pyrimidin-4-yl)-[1,2,4]triazolo[1,5-c]pyrimidin-7-yl)benzonitrile (Example 78B)

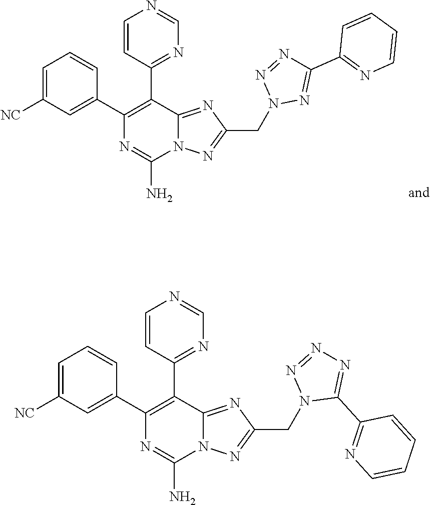 [SMILES: N#Cc1cccc(-c2nc(N)n3nc(Cn4nnc(-c5ccccn5)n4)nc3c2-c2ccncn2)c1 ; N#Cc1cccc(-c2nc(N)n3nc(Cn4nnnc4-c4ccccn4)nc3c2-c2ccncn2)c1]

A mixture of 3-(5-amino-2-(chloromethyl)-8-(pyrimidin-4-yl)-[1,2,4]triazolo[1,5-c]pyrimidin-7-yl)benzonitrile (10 mg, 0.028 mmol) (From Example 72, Step 4), 2-(1H-tetrazol-5-yl)pyridine (8.1 mg, 0.055 mmol) and Cs₂CO₃ (20.7 mg, 0.064 mmol) in DMF (1 mL) was stirred at 100° C. for 10 min. The reaction mixture was then cooled to r.t., diluted with methanol (4 mL), and purified by preparative LC-MS (pH 2, acetonitrile/water with TFA) to afford the mixture of products (1.5:1 ratio of Example 78A:Example 78B) as TFA salts. LCMS calculated for C₂₃H₁₆N₁₃ (M+H)⁺: 474.2. found 474.2. Example 78A: ¹H NMR (500 MHz, DMSO) δ 8.99 (d, J=1.4 Hz, 1H), 8.85 (d, J=5.3 Hz, 1H), 8.80-8.71 (m, 1H), 8.71-8.39 (b, 2H), 8.18 (d, J=7.7, 1.1 Hz, 1H), 8.04 (t, J=7.8, 1.8 Hz, 1H), 7.85 (m, 2H), 7.80-7.76 (m, 1H), 7.62-7.55 (m, 2H), 7.53 (t, J=7.8 Hz, 1H), 6.39 (s, 2H).

### Examples 79A-79B. 3-(2-((5-(1H-Pyrazol-1-yl)-2H-tetrazol-2-yl)methyl)-5-amino-8-(pyrimidin-4-yl)-[1,2,4]triazolo[1,5-c]pyrimidin-7-yl)benzonitrile (Example 79A) and 3-(2-((5-(1H-Pyrazol-1-yl)-1H-tetrazol-1-yl)methyl)-5-amino-8-(pyrimidin-4-yl)-[1,2,4]triazolo[1,5-c]pyrimidin-7-yl)benzonitrile (Example 79B)

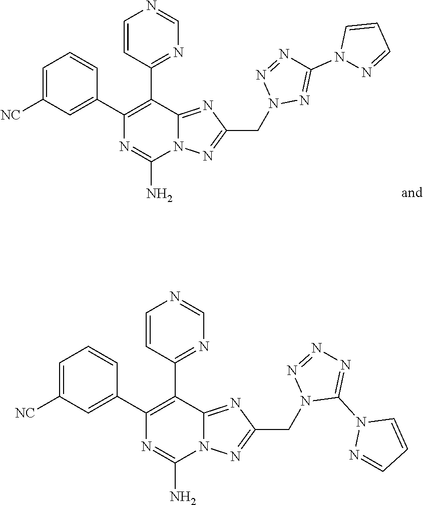 [SMILES: N#Cc1cccc(-c2nc(N)n3nc(Cn4nnc(-n5cccn5)n4)nc3c2-c2ccncn2)c1 ; N#Cc1cccc(-c2nc(N)n3nc(Cn4nnnc4-n4cccn4)nc3c2-c2ccncn2)c1]

This title mixture of compounds was prepared using similar procedures as described for Example 78, with 5-(1H-pyrazol-1-yl)-1H-tetrazole replacing 2-(1H-tetrazol-5-yl)pyridine. The products were purified by preparative LC-MS (pH 2, acetonitrile/water with TFA) to afford the products as TFA salts. LCMS calculated for C₂₁H₁₅N₁₄ (M+H)⁺: 463.2. found 463.2.

### Examples 80A-80B. 3-(5-Amino-8-(pyrimidin-4-yl)-2-((5-(thiazol-4-yl)-2H-tetrazol-2-yl)methyl)-[1,2,4]triazolo[1,5-a]pyridin-7-yl)benzonitrile (Example 80A) and 3-(5-Amino-8-(pyrimidin-4-yl)-2-((5-(thiazol-4-yl)-1H-tetrazol-1-yl)methyl)-[1,2,4]triazolo[1,5-c]pyrimidin-7-yl)benzonitrile (Example 80B)

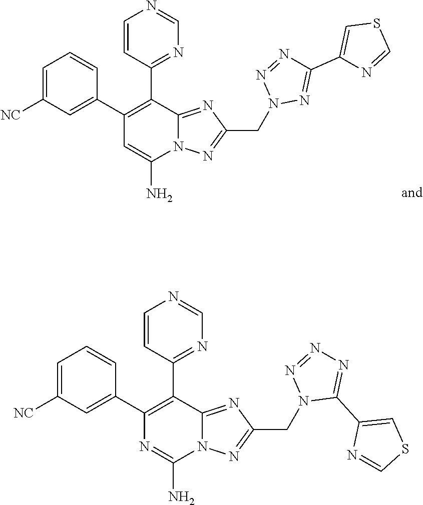 [SMILES: N#Cc1cccc(-c2cc(N)n3nc(Cn4nnc(-c5cscn5)n4)nc3c2-c2ccncn2)c1 ; N#Cc1cccc(-c2nc(N)n3nc(Cn4nnnc4-c4cscn4)nc3c2-c2ccncn2)c1]

The title mixture of compounds was prepared using similar procedures as described for Example 78, with 4-(1H-tetrazol-5-yl)thiazole replacing 2-(1H-tetrazol-5-yl)pyridine. The products were purified by preparative LC-MS (pH 2, acetonitrile/water with TFA) to afford the products as TFA salts. LCMS calculated for C₂₁H₁₄N₁₃S (M+H)⁺: 480.1. found 480.1.

### Example 81. 3-(5-Amino-2-((5-methyl-3-(trifluoromethyl)-1H-pyrazol-1-yl)methyl)-8-(pyrimidin-4-yl)-[1,2,4]triazolo[1,5-c]pyrimidin-7-yl)benzonitrile

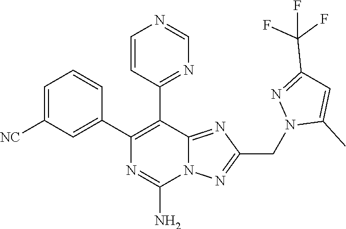 [SMILES: Cc1cc(C(F)(F)F)nn1Cc1nc2c(-c3ccncn3)c(-c3cccc(C#N)c3)nc(N)n2n1]

This compound was prepared using similar procedures as described for Example 78, with 5-methyl-3-(trifluoromethyl)-1H-pyrazole replacing 2-(1H-tetrazol-5-yl)pyridine. The product was purified by preparative LC-MS (pH 2, acetonitrile/water with TFA) to afford the product as a TFA salt. LCMS calculated for C₂₂H₁₆F₃N₁₀ (M+H)⁺: 477.2. found 477.1.

### Example 82. 3-(5-Amino-8-(4-ethyloxazol-5-yl)-2-(pyridin-2-ylmethyl)-[1,2,4]triazolo[1,5-c]pyrimidin-7-yl)benzonitrile

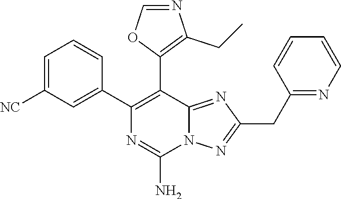 [SMILES: CCc1ncoc1-c1c(-c2cccc(C#N)c2)nc(N)n2nc(Cc3ccccn3)nc12]

This compound was prepared using similar procedures as described for Example 42 with 4-ethyl-5-(4,4,5,5-tetramethyl-1,3,2-dioxaborolan-2-yl)oxazole replacing 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₃H₁₉N₈O (M+H)⁺: 423.2. found 423.2.

### Example 83. 3-(5-Amino-8-(1-ethyl-1H-1,2,3-triazol-5-yl)-2-(pyridin-2-ylmethyl)-[1,2,4]triazolo[1,5-c]pyrimidin-7-yl)benzonitrile

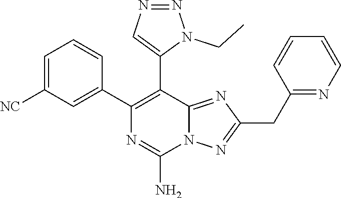 [SMILES: CCn1nncc1-c1c(-c2cccc(C#N)c2)nc(N)n2nc(Cc3ccccn3)nc12]

This compound was prepared using similar procedures as described for Example 41, Step 5, with 1-ethyl-5-(trimethylstannyl)-1H-1,2,3-triazole replacing 4-(tributylstannyl)pyrimidine. The final material was purified by preparative LC-MS (pH 2, acetonitrile/water with TFA) to afford the product as a TFA salt. LCMS calculated for C₂₂H₁₉N₁₀ (M+H)⁺: 423.2. found 423.2

### Example 84. 3-(5-Amino-8-(4-methyloxazol-5-yl)-2-((3-methylpyridin-2-yl)methyl)-[1,2,4]triazolo[1,5-c]pyrimidin-7-yl)benzonitrile

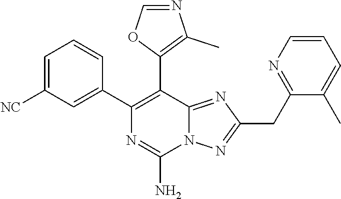 [SMILES: Cc1cccnc1Cc1nc2c(-c3ocnc3C)c(-c3cccc(C#N)c3)nc(N)n2n1]

### Step 1: 3-(5-Amino-8-bromo-2-((3-methylpyridin-2 yl)methyl)-[1,2,4]triazolo[1,5-c]pyrimidin-7-yl)benzonitrile

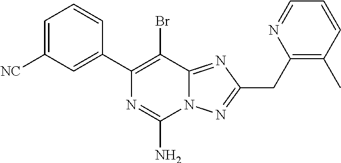 [SMILES: Cc1cccnc1Cc1nc2c(Br)c(-c3cccc(C#N)c3)nc(N)n2n1]

This compound was prepared using similar procedures as described for Example 41, Step 3 to Step 4, with 2-(3-methylpyridin-2-yl)acetohydrazide replacing 2-(pyridin-2-yl)acetohydrazide in Step 3. LCMS calculated for C₁₉H₁₅BrN₇ (M+H)⁺: 420.1. found 420.1.

### Step 2: 3-(5-Amino-8-(4-methyloxazol-5-yl)-2-((3-methylpyridin-2 yl)methyl)-[1,2,4]triazolo[1,5-c]pyrimidin-7-yl)benzonitrile

This compound was prepared using similar procedures as described for Example 61, Step 2, with 3-(5-amino-8-bromo-2-((3-methylpyridin-2-yl)methyl)-[1,2,4]triazolo[1,5-c]pyrimidin-7-yl)benzonitrile replacing 3-(5-Amino-8-bromo-2-(pyridin-2-ylmethyl)-[1,2,4]triazolo[1,5-c]pyrimidin-7-yl)benzonitrile. The final material was purified by preparative LC-MS (pH 2, acetonitrile/water with TFA) to afford the product as a TFA salt. LCMS calculated for C₂₃H₁₉N₈O (M+H)⁺: 423.2. found 423.2.

### Example 85. 3-(5-Amino-2-((3-fluoropyridin-2-yl)methyl)-8-(4-(hydroxymethyl)-2-methyloxazol-5-yl)-[1,2,4]triazolo[1,5-c]pyrimidin-7-yl)benzonitrile

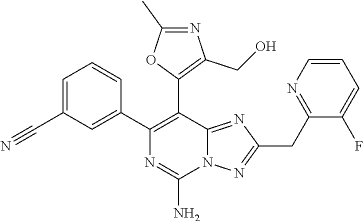 [SMILES: Cc1nc(CO)c(-c2c(-c3cccc(C#N)c3)nc(N)n3nc(Cc4ncccc4F)nc23)o1]

This compound was prepared using similar procedures as described for Example 62, Step 3 to Step 4, with 3-(5-Amino-8-bromo-2-((3-fluoropyridin-2-yl)methyl)-[1,2,4]triazolo[1,5-c]pyrimidin-7-yl)benzonitrile replacing 3-(5-Amino-8-bromo-2-(pyridin-2-ylmethyl)-[1,2,4]triazolo[1,5-c]pyrimidin-7-yl)benzonitrile in Step 3. The final material was purified by preparative LC-MS (pH 2, acetonitrile/water with TFA) to afford the product as a TFA salt. LCMS calculated for C₂₃H₁₈FN₈O₂ (M+H)⁺: 457.2. found 457.2.

### Example 86. 3-(5-Amino-2-(2-((1,1-dioxidoisothiazolidin-2-yl)methyl)-6-fluorobenzyl)-8-(pyrimidin-4-yl)-[1,2,4]triazolo[1,5-c]pyrimidin-7-yl)benzonitrile

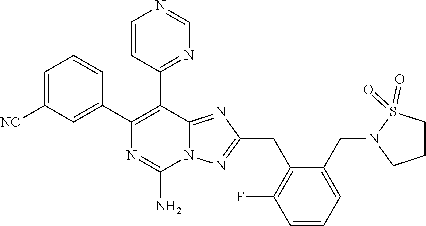 [SMILES: N#Cc1cccc(-c2nc(N)n3nc(Cc4c(F)cccc4CN4CCCS4(=O)=O)nc3c2-c2ccncn2)c1]

This compound was prepared using similar procedures as described for Example 72, with 1,2-thiazolidine 1,1-dioxide replacing (3R,4R)-4-fluoropyrrolidin-3-ol hydrochloride in Step 7. The product was purified by preparative LC-MS (pH 2, acetonitrile/water with TFA) to afford the product as a TFA salt. LCMS calculated for C₂₇H₂₃FN₉O₂S (M+H)⁺: 556.2. found 556.2.

### Example 87. 3-(5-Amino-2-(2-fluoro-6-((3-methyl-2,5-dioxoimidazolidin-1-yl)methyl)benzyl)-8-(pyrimidin-4-yl)-[1,2,4]triazolo[1,5-c]pyrimidin-7-yl)benzonitrile

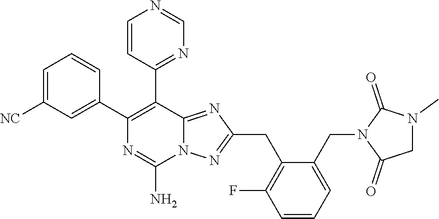 [SMILES: CN1CC(=O)N(Cc2cccc(F)c2Cc2nc3c(-c4ccncn4)c(-c4cccc(C#N)c4)nc(N)n3n2)C1=O]

This compound was prepared using similar procedures as described for Example 72, with 1-methylimidazolidine-2,4-dione replacing (3R,4R)-4-fluoropyrrolidin-3-ol hydrochloride in Step 7. The product was purified by preparative LC-MS (pH 2, acetonitrile/water with TFA) to afford the product as a TFA salt. LCMS calculated for C₂₈H₂₂FN₁₀O₂ (M+H)⁺: 549.2. found 549.2.

### Example 88. 3-(5-Amino-2-((3-fluoropyridin-2-yl)methyl)-8-(pyrimidin-4-yl)-[1,2,4]triazolo[1,5-c]pyrimidin-7-yl)benzonitrile

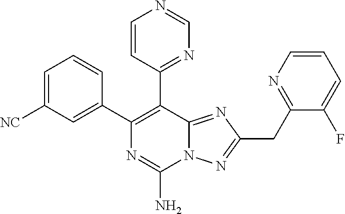 [SMILES: N#Cc1cccc(-c2nc(N)n3nc(Cc4ncccc4F)nc3c2-c2ccncn2)c1]

This compound was prepared using similar procedures as described for Example 41, Step 5, with 3-(5-amino-8-bromo-2-((3-fluoropyridin-2-yl)methyl)-[1,2,4]triazolo[1,5-c]pyrimidin-7-yl)benzonitrile (Example 52, Step 2) replacing 3-(5-amino-8-bromo-2-(pyridin-2-ylmethyl)-[1,2,4]triazolo[1,5-c]pyrimidin-7-yl)benzonitrile. The final material was purified by preparative LC-MS (pH 2, acetonitrile/water with TFA) to afford the product as a TFA salt. LCMS calculated for C₂₂H₁₅FN₉ (M+H)⁺: 424.1. found 424.1.

### Example 89. 3-(5-Amino-8-(4-methyloxazol-5-yl)-2-((6-(trifluoromethyl)pyridin-2-yl)methyl)-[1,2,4]triazolo[1,5-c]pyrimidin-7-yl)benzonitrile

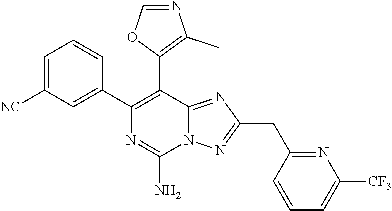 [SMILES: Cc1ncoc1-c1c(-c2cccc(C#N)c2)nc(N)n2nc(Cc3cccc(C(F)(F)F)n3)nc12]

### Step 1: Ethyl 2-(6-(trifluoromethyl)pyridin-2-yl)acetate

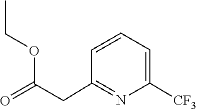 [SMILES: CCOC(=O)Cc1cccc(C(F)(F)F)n1]

A THF (10.3 ml) solution of 2-methyl-6-(trifluoromethyl)pyridine (500 mg, 3.10 mmol) was cooled to −78° C., followed by addition of n-butyllithium (1.49 ml, 3.72 mmol) dropwise. After 10 min, diethyl carbonate (0.564 ml, 4.65 mmol) was added in one portion. The mixture was allowed to stir at −78° C. for 30 min. The mixture was then diluted with aq. NH₄Cl and extracted with DCM. The combined organic layers were dried over MgSO₄, filtered, and concentrated. The resulting residue was purified by flash chromatography on a silica gel column eluting with 0 to 50% EtOAc in Hexanes to afford the desired product. LCMS calculated for C₁₀H₁₁F₃NO₂ (M+H)⁺: 234.1. Found: 234.1.

### Step 2: 3-(5-Amino-8-bromo-2-((6-(trifluoromethyl)pyridin-2 yl)methyl)-[1,2,4]triazolo[1,5-c]pyrimidin-7-yl)benzonitrile

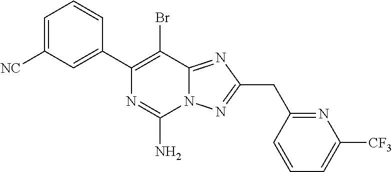 [SMILES: N#Cc1cccc(-c2nc(N)n3nc(Cc4cccc(C(F)(F)F)n4)nc3c2Br)c1]

This compound was prepared using similar procedures as described for Example 41, Step 1 to Step 4, with ethyl 2-(6-(trifluoromethyl)pyridin-2-yl)acetate replacing methyl 2-(pyridin-2-yl)acetate in Step 2. LCMS calculated for C₁₉H₁₂BrF₃N₇ (M+H)⁺: 474.0. found 474.0.

### Step 3: 3-(5-Amino-8-(4-methyloxazol-5-yl)-2-((6-(trifluoromethyl)pyridin-2 yl)methyl)-[1,2,4]triazolo[1,5-c]pyrimidin-7-yl)benzonitrile

This compound was prepared using similar procedures as described for Example 42 with 3-(5-amino-8-bromo-2-((6-(trifluoromethyl)pyridin-2-yl)methyl)-[1,2,4]triazolo[1,5-c]pyrimidin-7-yl)benzonitrile replacing 3-(5-amino-8-bromo-2-(pyridin-2-ylmethyl)-[1,2,4]triazolo[1,5-c]pyrimidin-7-yl)benzonitrile, and with 4-methyl-5-(4,4,5,5-tetramethyl-1,3,2-dioxaborolan-2-yl)oxazole (Example 61, Step 1) replacing 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₃H₁₆F₃N₈O (M+H)⁺: 477.1. found 477.2.

### Example 90. 3-(5-Amino-8-(4-methyloxazol-5-yl)-2-(pyridin-2-ylmethyl)-[1,2,4]triazolo[1,5-c]pyrimidin-7-yl)-2-fluorobenzonitrile

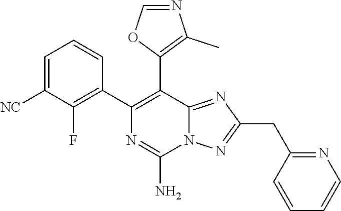 [SMILES: Cc1ncoc1-c1c(-c2cccc(C#N)c2F)nc(N)n2nc(Cc3ccccn3)nc12]

### Step 1: 3-(5 Amino-8-bromo-2-(pyridin-2 ylmethyl)-[1,2,4]triazolo[1,5-c]pyrimidin-7-yl)-2-fluorobenzonitrile

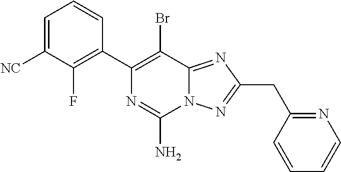 [SMILES: N#Cc1cccc(-c2nc(N)n3nc(Cc4ccccn4)nc3c2Br)c1F]

This compound was prepared using similar procedures as described for Example 41, Step 1 to Step 4, with (3-cyano-2-fluorophenyl)boronic acid replacing (3-cyanophenyl)boronic acid in Step 1. LCMS calculated for C₁₈H₁₂BrFN₇ (M+H)⁺: 424.0. found 424.0.

### Step 2: 3-(5 Amino-8-(4-methyloxazol-5-yl)-2-(pyridin-2 ylmethyl)-[1,2,4]triazolo[1,5-c]pyrimidin-7-yl)-2 fluorobenzonitrile

This compound was prepared using similar procedures as described for Example 42 with 3-(5-amino-8-bromo-2-(pyridin-2-ylmethyl)-[1,2,4]triazolo[1,5-c]pyrimidin-7-yl)-2-fluorobenzonitrile replacing 3-(5-amino-8-bromo-2-(pyridin-2-ylmethyl)-[1,2,4]triazolo[1,5-c]pyrimidin-7-yl)benzonitrile, and with 4-methyl-5-(4,4,5,5-tetramethyl-1,3,2-dioxaborolan-2-yl)oxazole (Example 61, Step 1) replacing 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₂H₁₆FN₈O (M+H)⁺: 427.1. found 427.2.

### Example 91. 3-(5-Amino-2-((3-(hydroxymethyl)pyridin-2-yl)methyl)-8-(4-methyloxazol-5-yl)-[1,2,4]triazolo[1,5-c]pyrimidin-7-yl)benzonitrile

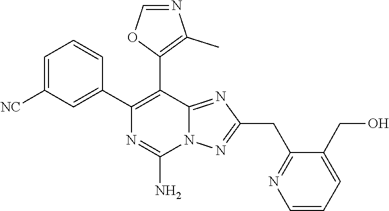 [SMILES: Cc1ncoc1-c1c(-c2cccc(C#N)c2)nc(N)n2nc(Cc3ncccc3CO)nc12]

### Step 1: 3-(((tert-Butyldimethylsilyl)oxy)methyl)-2-methylpyridine

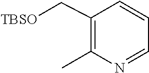 [SMILES: Cc1ncccc1CO[Si](C)(C)C(C)(C)C]

TBS-Cl (918 mg, 6.09 mmol) was added to a CH₂Cl₂ (20 ml) solution of (2-methylpyridin-3-yl)methanol (750 mg, 6.09 mmol) and imidazole (415 mg, 6.09 mmol) at rt. After stirring for 30 min, the mixture was diluted with water and extracted with DCM (×3). The combined organic layers were dried over MgSO₄, filtered, and concentrated. The resulting residue was purified by flash chromatography on a silica gel column eluting with 0 to 50% EtOAc in Hexanes to afford the desired product. LCMS calculated for C₁₃H₂₄NOSi (M+H)⁺: 238.2. Found: 238.2.

### Step 2: Ethyl 2-(3-(((tert-butyldimethylsilyl)oxy)methyl)pyridin-2 yl)acetate

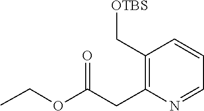 [SMILES: CCOC(=O)Cc1ncccc1CO[Si](C)(C)C(C)(C)C]

A THF (7.0 ml) solution 3-(((tert-butyldimethylsilyl)oxy)methyl)-2-methylpyridine (500 mg, 2.106 mmol) was cooled to −78° C., followed by addition of n-butyllithium (1095 μl, 2.74 mmol). After stirring for 1 h at −78° C., diethyl carbonate (765 μl, 6.32 mmol) was added in one portion. Then the mixture was allowed to slowly warm to room temperature and stirred overnight. The mixture was diluted with aq. NH₄Cl and extracted with DCM (×3). The combined organic layers were dried over MgSO₄, filtered, and concentrated. The resulting residue was purified by flash chromatography on a silica gel column eluting with 0 to 50% EtOAc in Hexanes to afford the desired product. LCMS calculated for C₁₆H₂₈NO₃Si (M+H)⁺: 310.2. Found: 310.2.

### Step 3: 3-(5-Amino-2-((3-(hydroxymethyl)pyridin-2 yl)methyl)-[1,2,4]triazolo[1,5-c]pyrimidin-7-yl)benzonitrile

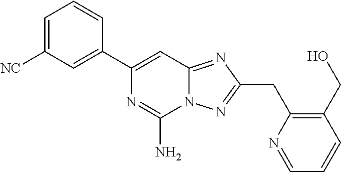 [SMILES: N#Cc1cccc(-c2cc3nc(Cc4ncccc4CO)nn3c(N)n2)c1]

This compound was prepared using similar procedures as described for Example 41, Step 1 to Step 3, with ethyl 2-(3-(((tert-butyldimethylsilyl)oxy)methyl)pyridin-2-yl)acetate replacing methyl 2-(pyridin-2-yl)acetate in Step 2. Then the corresponding product was taken into 3 mL of THE and treated with 3 mL of 6 N HCl solution. The mixture was allowed to stir at room temperature for 3 h. The mixture was diluted with water and extracted with DCM (×3). The combined organic layers were dried over MgSO₄, filtered, and concentrated. The resulting residue was purified by flash chromatography on a silica gel column eluting with 0 to 20% MeOH in DCM to afford the desired product. LCMS calculated for C₁₉H₁₆N₇O (M+H)⁺: 358.1. Found: 358.2.

### Step 4: 3-(5-Amino-8-bromo-2-((3-(hydroxymethyl)pyridin-2 yl)methyl)-[1,2,4]triazolo[1,5-c]pyrimidin-7-yl)benzonitrile

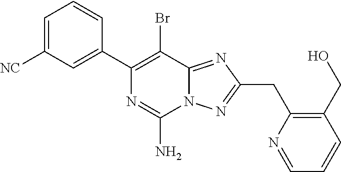 [SMILES: N#Cc1cccc(-c2nc(N)n3nc(Cc4ncccc4CO)nc3c2Br)c1]

This compound was prepared using similar procedures as described for Example 41, Step 4, with 3-(5-amino-2-((3-(hydroxymethyl)pyridin-2-yl)methyl)-[1,2,4]triazolo[1,5-c]pyrimidin-7-yl)benzonitrile replacing 3-(5-amino-2-(pyridin-2-ylmethyl)-[1,2,4]triazolo[1,5-c]pyrimidin-7-yl)benzonitrile. The resulting crude mixture was diluted with water and extracted with DCM (×3). The combined organic layers were dried over MgSO₄, filtered, and concentrated. The resulting residue was purified by flash chromatography on a silica gel column eluting with 0 to 15% MeOH in DCM to afford the desired product. LCMS calculated for C₁₉H₁₅BrN₇O (M+H)⁺: 436.1. Found: 436.0.

### Step 5: 3-(5-Amino-2-((3-(hydroxymethyl)pyridin-2 yl)methyl)-8-(4-methyloxazol-5-yl)-[1,2,4]triazolo[1,5-c]pyrimidin-7-yl)benzonitrile

This compound was prepared using similar procedures as described for Example 42, with 3-(5-amino-8-bromo-2-((3-(hydroxymethyl)pyridin-2-yl)methyl)-[1,2,4]triazolo[1,5-c]pyrimidin-7-yl)benzonitrile replacing 3-(5-amino-8-bromo-2-(pyridin-2-ylmethyl)-[1,2,4]triazolo[1,5-c]pyrimidin-7-yl)benzonitrile, and with 4-methyl-5-(4,4,5,5-tetramethyl-1,3,2-dioxaborolan-2-yl)oxazole (Example 61, Step 1) replacing 1-ethyl-5-(4,4,5,5-tetramethyl-1,3,2-dioxaborolan-2-yl)-1H-pyrazole. The final material was purified by preparative LC-MS (pH 2, acetonitrile/water with TFA) to afford the product as a TFA salt. LCMS calculated for C₂₃H₁₉N₈O₂ (M+H)⁺: 439.2. found 439.2.

### Example 92. 3-(5-Amino-2-((1-methyl-1H-pyrazol-3-yl)methyl)-8-(4-methyloxazol-5-yl)-[1,2,4]triazolo[1,5-c]pyrimidin-7-yl)benzonitrile

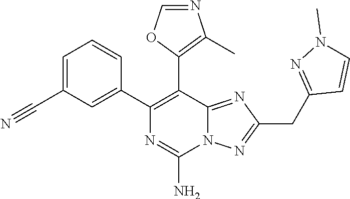 [SMILES: Cc1ncoc1-c1c(-c2cccc(C#N)c2)nc(N)n2nc(Cc3ccn(C)n3)nc12]

### Step 1: Methyl 2-(1-methyl-1H-pyrazol-3 yl)acetate

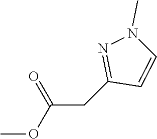

Acetyl chloride (0.355 ml, 4.99 mmol) was added to MeOH (3 ml) at 0° C. and then stirred for 20 min. To this mixture was added 2-(1-methyl-1H-pyrazol-3-yl)acetic acid (140 mg, 0.999 mmol) and then the mixture was stirred at rt overnight. The solvent was removed and the residue was used in the next step directly. LCMS calculated for C₇H₁₁N₂O₂ (M+H)⁺: 155.1. found 155.1.

### Step 2: 2-(1-Methyl-1H pyrazol-3-yl)acetohydrazide

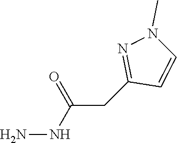

Hydrazine (0.061 ml, 1.946 mmol) was added to a solution of methyl 2-(1-methyl-1H-pyrazol-3-yl)acetate (0.15 g, 0.973 mmol) in ethanol (1.5 mL) at rt and then the reaction mixture was heated and stirred at 100° C. for 2 h. After cooling to rt, the resulting mixture was partially concentrated until solid presented. To this mixture diethyl ether (1.0 mL) was added and the resulting solid was collected by filtration, rinsed with ether, and dried to afford the desired product as a white solid, which was used in the next step without further purification. LCMS calculated for C₆H₁₁N₄O (M+H)⁺: 155.1. found 155.2.

### Step 3: 3-(5-Amino-2-((1-methyl-1H-pyrazol-3 yl)methyl)-[1,2,4]triazolo[1,5-c]pyrimidin-7-yl)benzonitrile

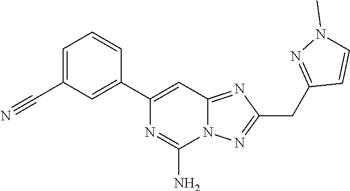 [SMILES: Cn1ccc(Cc2nc3cc(-c4cccc(C#N)c4)nc(N)n3n2)n1]

This compound was prepared using similar procedure as described for Example 41, Step 3, replacing 2-(pyridin-2-yl)acetohydrazide with 2-(1-methyl-1H-pyrazol-3-yl)acetohydrazide. LCMS calculated for C₁₇H₁₅N₈ (M+H)⁺: 331.1. found 331.1.

### Step 4: 3-(5-Amino-8-bromo-2-((1-methyl-1H pyrazol-3 yl)methyl)-[1,2,4]triazolo[1,5-c]pyrimidin-7-yl)benzonitrile

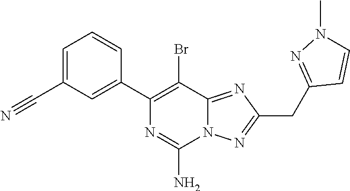 [SMILES: Cn1ccc(Cc2nc3c(Br)c(-c4cccc(C#N)c4)nc(N)n3n2)n1]

NBS (35.6 mg, 0.200 mmol) was added to a mixture of 3-(5-amino-2-((1-methyl-1H-pyrazol-3-yl)methyl)-[1,2,4]triazolo[1,5-c]pyrimidin-7-yl)benzonitrile (66.0 mg, 0.200 mmol) in DMSO (0.4 ml)/CH₂Cl₂ (0.4 ml) at −30° C. and the reaction mixture was stirred for 1 h. The low boiling point solvent was removed and to the residue was added water. The resulting solid was then collected by filtration, and dried to provide the product, which was used in the next step directly. LCMS calculated for C₁₇H₁₄BrN₈ (M+H)⁺: 409.1. found 409.1.

### Step 5: 3-(5-Amino-2-((1-methyl-1H-pyrazol-3 yl)methyl)-8-(4-methyloxazol-5-yl)-[1,2,4]triazolo[1,5-c]pyrimidin-7-yl)benzonitrile

A mixture of 3-(5-amino-8-bromo-2-((1-methyl-1H-pyrazol-3-yl)methyl)-[1,2,4]triazolo[1,5-c]pyrimidin-7-yl)benzonitrile (10.0 mg, 0.024 mmol), 4-methyl-5-(4,4,5,5-tetramethyl-1,3,2-dioxaborolan-2-yl)oxazole (10.22 mg, 0.049 mmol), CsF (18.56 mg, 0.122 mmol) and chloro(1-t-butylindenyl)[2-(dicyclohexylphosphino)-2′,4′,6′-tri-i-propyl-1,1′-biphenyl]palladium(II) (1.355 mg, 2.443 μmol) in dioxane (0.5 mL)/water (0.1 mL) was first purged with nitrogen, and then heated and stirred at 105° C. for 2 h. The reaction mixture was then cooled to rt, and concentrated. The resulting mixture was diluted with acetonitrile/water and purified using prep-LCMS (pH 2, acetonitrile/water with TFA) to afford the desired product as its TFA salt. LC-MS calculated for C₂₁H₁₈N₉O (M+H)⁺: m/z=412.2. found 412.2.

### Example 93. 3-(5-Amino-2-((1-methyl-1H-pyrazol-3-yl)methyl)-8-(oxazol-5-yl)-[1,2,4]triazolo[1,5-c]pyrimidin-7-yl)benzonitrile

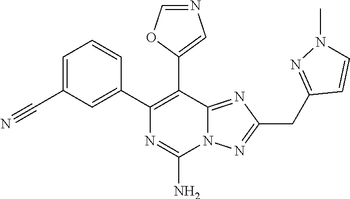 [SMILES: Cn1ccc(Cc2nc3c(-c4cnco4)c(-c4cccc(C#N)c4)nc(N)n3n2)n1]

This compound was prepared using similar procedures as described in Example 92, Step 5, replacing 4-methyl-5-(4,4,5,5-tetramethyl-1,3,2-dioxaborolan-2-yl)oxazole with 5-(4,4,5,5-tetramethyl-1,3,2-dioxaborolan-2-yl)oxazole. LC-MS calculated for C₂₀H₁₆N₉O (M+H)⁺: m/z=398.1. found 398.1.

### Example 94. 3-(5-Amino-2-((3-methylpyridin-2-yl)methoxy)-8-(pyrimidin-4-yl)-[1,2,4]triazolo[1,5-c]pyrimidin-7-yl)benzonitrile

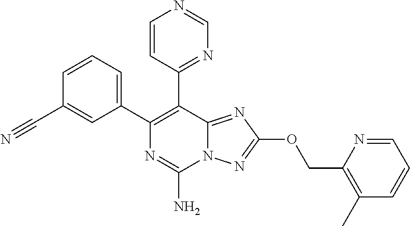 [SMILES: Cc1cccnc1COc1nc2c(-c3ccncn3)c(-c3cccc(C#N)c3)nc(N)n2n1]

### Step 1: 3-(5-(Bis(4-methoxybenzyl)amino)-2-bromo-8-(pyrimidin-4-yl)-[1,2,4]triazolo[1,5-c]pyrimidin-7-yl)benzonitrile

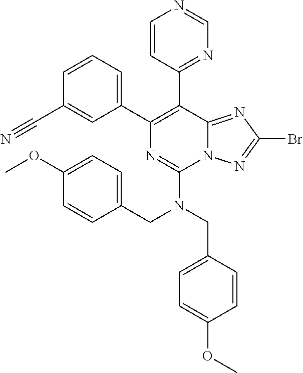 [SMILES: COc1ccc(CN(Cc2ccc(OC)cc2)c2nc(-c3cccc(C#N)c3)c(-c3ccncn3)c3nc(Br)nn23)cc1]

To a mixture of copper(II) bromide (91 mg, 0.407 mmol) and tert-butyl nitrite (0.054 ml, 0.407 mmol) in acetonitrile (3 mL) under nitrogen at 50° C. was added dropwise 3-(2-amino-5-(bis(4-methoxybenzyl)amino)-8-(pyrimidin-4-yl)-[1,2,4]triazolo[1,5-c]pyrimidin-7-yl)benzonitrile (100 mg, 0.203 mmol) (from Example 69, step 5) in acetonitrile (3 mL). The mixture was stirred at 50° C. for 2 hours. After cooling to room temperature, 1 N aqueous NH₄OH solution (20 mL) was added and the mixture was extracted three times with CH₂Cl₂ (20 mL). The combined organic layers were dried over sodium sulfate, filtered and concentrated. The crude material was purified by silica gel column chromatography eluting with 50-100% ethyl acetate/hexane to give the desired product. LC-MS calculated for C₃₂H₂₆BrN₈O₂ (M+H)⁺: m/z=633.1. found 633.2.

### Step 2: 3-(5-Amino-2-((3-methylpyridin-2 yl)methoxy)-8-(pyrimidin-4-yl)-[1,2,4]triazolo[1,5-c]pyrimidin-7-yl)benzonitrile

A suspension of sodium hydride (60% in mineral oil, 3.8 mg, 0.095 mmol), 3-(5-(bis(4-methoxybenzyl)amino)-2-bromo-8-(pyrimidin-4-yl)-[1,2,4]triazolo[1,5-c]pyrimidin-7-yl)benzonitrile (20 mg, 0.032 mmol) and (3-methylpyridin-2-yl)methanol (9.1 μL, 0.095 mmol) in 1,4-dioxane (1 mL) was heated and stirred at 110° C. under nitrogen overnight. The reaction mixture was then cooled to rt, concentrated, and added TFA (1.0 mL). The resulting mixture was then stirred at 110° C. for 30 min, cooled to rt, diluted with acetonitrile, filtered and purified by preparative LC-MS (pH 2, acetonitrile/water with TFA) to give desired product as a TFA salt. LC-MS calculated for C₂₃H₁₈N₉O (M+H)⁺: m/z=436.2. found 436.2. ¹H NMR (600 MHz, DMSO) δ 8.97 (d, J=1.4 Hz, 1H), 8.88 (d, J=5.2 Hz, 1H), 8.58-8.52 (m, 1H), 7.97 (d, J=7.8 Hz, 1H), 7.88 (dd, J=5.4, 1.4 Hz, 1H), 7.85 (dt, J 7.5, 1.5 Hz, 1H), 7.78 (t, J 1.8 Hz, 1H), 7.60-7.54 (m, 2H), 7.53 (t, J=7.8 Hz, 1H), 5.69 (s, 2H), 2.48 (s, 3H).

### Example 95. 3-(5-Amino-2-((6-methylpyridin-2-yl)methoxy)-8-(pyrimidin-4-yl)-[1,2,4]triazolo[1,5-c]pyrimidin-7-yl)benzonitrile

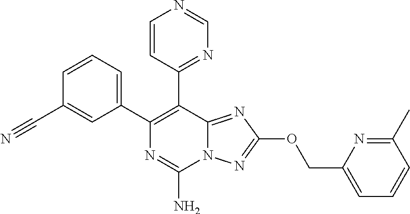 [SMILES: Cc1cccc(COc2nc3c(-c4ccncn4)c(-c4cccc(C#N)c4)nc(N)n3n2)n1]

This compound was prepared using similar procedures as described for Example 94, with (6-methylpyridin-2-yl)methanol replacing (3-methylpyridin-2-yl)methanol in Step 2. The crude was purified by preparative LC-MS (pH 2, acetonitrile/water with TFA) to afford the product as a TFA salt. LCMS calculated for C₂₃H₁₈N₉O (M+H)⁺: m/z=436.2. found 436.2.

### Example 96. 2-((5-Amino-7-(3-cyanophenyl)-8-(pyrimidin-4-yl)-[1,2,4]triazolo[1,5-c]pyrimidin-2-yl)methoxy)benzonitrile

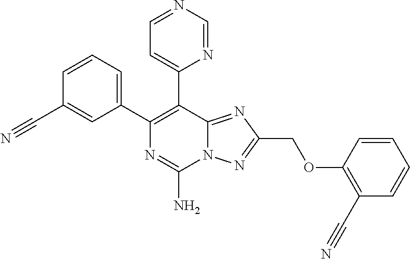 [SMILES: N#Cc1cccc(-c2nc(N)n3nc(COc4ccccc4C#N)nc3c2-c2ccncn2)c1]

A mixture of 3-(5-amino-2-(chloromethyl)-8-(pyrimidin-4-yl)-[1,2,4]triazolo[1,5-c]pyrimidin-7-yl)benzonitrile (20 mg, 0.055 mmol) (from Example 72, step 4), Cs₂CO₃ (35.9 mg, 0.110 mmol) and 2-hydroxybenzonitrile (13.1 mg, 0.110 mmol) in acetonitrile (1.0 mL) was heated and stirred at 70° C. for 1 h. The resulting mixture was then cooled to room temperature, diluted with methanol, filtered and purified by preparative LC-MS (pH 2, acetonitrile/water with TFA) to give the desired product as a TFA salt. LCMS calculated for C₂₄H₁₆N₉O (M+H)⁺: m/z=446.1. found 446.2.

### Example 97. 3-(5-Amino-2-(((3-methylpyridin-2-yl)methyl)amino)-8-(pyrimidin-4-yl)-[1,2,4]triazolo[1,5-c]pyrimidin-7-yl)benzonitrile

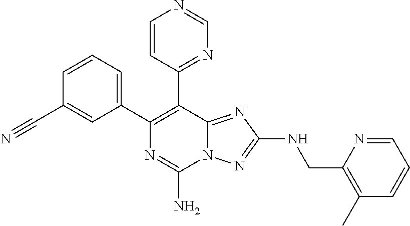 [SMILES: Cc1cccnc1CNc1nc2c(-c3ccncn3)c(-c3cccc(C#N)c3)nc(N)n2n1]

A mixture of Triethyl orthoformate (0.029 mL, 0.176 mmol), 3-(2-amino-5-(bis(4-methoxybenzyl)amino)-8-(pyrimidin-4-yl)-[1,2,4]triazolo[1,5-c]pyrimidin-7-yl)benzonitrile (20 mg, 0.035 mmol) (from Example 69, step 5), and 3-methylpicolinaldehyde (12.8 mg, 0.105 mmol) in EtOH (1 mL) was stirred at 120° C. overnight. The reaction mixture was then cooled to 0° C., and NaBH₄ (4.0 mg, 0.105 mmol) was added. After stirring at 0° C. for 1 h, the reaction mixture was quenched with a few drops of TFA and diluted with MeOH. The crude mixture was then purified by preparative LC-MS (pH 2, acetonitrile/water with TFA) to give the intermediate, which was then dissolved in TFA (1.0 mL). The resulting mixture was stirred at 120° C. for 25 min, cooled to rt, diluted with acetonitrile, and purified by preparative LC-MS (pH 2, acetonitrile/water with TFA) to give the desired product as a TFA salt. LCMS calculated for C₂₃H₁₉N₁₀ (M+H)⁺: m/z=435.2. found 435.2.

### Example a. Adenosine A2A Receptor Cyclic AMP GS Assay

Stably transfected HEK-293 cells expressing the human adenosine A2A receptor (Perkin Elmer) are maintained in MEM culture medium with 10% FBS and 400 μg/ml Geneticin (Life Technologies). 18 to 24 hours prior to assay, geneticin is removed from culture. The cisbio cAMP-GS Dynamic kit utilizing the FRET (Fluorescence Resonance Energy Transfer) technology is used to measure cAMP accumulation in the cells. Compounds of the present disclosure at an appropriate concentration are mixed with 10000 cells/well in white 96 well half area plates (Perkin Elmer) for 30 min at room temperature (RT) gently shaking. Agonist, CGS21680 (R&D Technologies) at 4 nM is added to each well for 60 min at RT gently shaking. Detection reagents, d2-labeled cAMP (acceptor) and anti-cAMP cryptate (donor) are added to each well for 60 min at RT gently shaking. Plates are read on Pherastar (BMG Labtech), fluorescence ratio 665/620 is calculated and EC₅₀ determination is performed by fitting the curve of percent of control versus the log of the compound concentration using GraphPad Prism.

### Example B. Adenosine A2B Receptor Cyclic AMP GS Assay

Stably transfected HEK-293 cells expressing the human adenosine A2B receptor (Perkin Elmer) were maintained in MEM culture medium with 10% FBS and 100 μg/ml Geneticin (Life Technologies). 18 to 24 hours prior to assay, geneticin was removed from culture. The cisbio cAMP-GS Dynamic kit utilizing the FRET (Fluorescence Resonance Energy Transfer) technology was used to measure cAMP accumulation in the cells. Compounds of the present disclosure at an appropriate concentration were mixed with 10000 cells/well in white 96 well half area plates (Perkin Elmer) for 30 min at RT gently shaking. Agonist, NECA (R&D Technologies) at 12 nM was added to each well for 60 min at RT gently shaking. Detection reagents, d2-labeled cAMP (acceptor) and anti-cAMP cryptate (donor) were added to each well for 60 min at RT gently shaking. Plates were read on Pherastar (BMG Labtech), fluorescence ratio 665/620 was calculated and EC₅₀ determination was performed by fitting the curve of percent of control versus the log of the compound concentration using GraphPad Prism. The EC₅₀ data obtained via this method are shown in Table 1.

### Example C. A2A Tag-Lite® HTRF Assay

Assays were conducted in black low volume 384-well polystyrene plates (Greiner 784076-25) in a final volume of 10 μL. Test compounds were first serially diluted in DMSO and 100 nl added to the plate wells before the addition of other reaction components. The final concentration of DMSO was 1%. Tag-Lite® Adenosine A2A labeled cells (CisBio C1TT1A2A) were diluted 1:5 into Tag-lite buffer (CisBio LABMED) and spun 1200 g for 5 mins. The pellet was resuspended at a volume 10.4× the initial cell suspension volume in Tag-lite buffer, and Adenosine A2A Receptor Red antagonist fluorescent ligand (CisBio L0058RED) added at 12.5 nM final concentration. 10 μl of the cell and ligand mix was added to the assay wells and incubated at room temperature for 45 minutes before reading on a PHERAstar FS plate reader (BMG Labtech) with HTRF 337/620/665 optical module. Percent binding of the fluorescent ligand was calculated; where 100 nM of A2A antagonist control ZM 241385 (Tocris 1036) displaces the ligand 100% and 1% DMSO has 0% displacement. The % binding data versus the log of the inhibitor concentration was fitted to a one-site competitive binding model (GraphPad Prism version 7.02) where the ligand constant=12.5 nM and the ligand Kd=1.85 nM. The K; data obtained via this method are shown in Table 1.

### Example D. A2B Filter Binding Assay

Assays are conducted in deep well polypropylene plates (Greiner 786201) in a final volume of 550 μL. Test compounds are first serially diluted in DMSO and 5.5 ul is then added to the plate wells before the addition of other reaction components. The final concentration of DMSO is 3%. HEK293 cell membranes overexpressing the human adenosine receptor A2B (Perkin Elmer ES-113-M400UA) are diluted to 40 μg/ml in 50 mM HEPES pH 7.0, 5 mM MgCl₂, 1 mM EDTA (Assay buffer). [3H] 8-cyclopentyl-1,3-dipropylxanthine (Perkin Elmer NET974001MC) is diluted in assay buffer+22% DMSO to 24.2 nM, and then further diluted to 1 nM by addition to the diluted membranes. 545 μl of the membrane and ligand mix is added to the assay wells and incubated on a shaker at room temperature for 1 hour. The membrane mix is then filtered over a UniFilter GF/C filter plate (Perkin Elmer 6005174) pre-soaked in 50 mM HEPES pH 6.5, 5 mM MgCl₂, 1 mM EDTA 0.5% BSA and then washed with 5 ml ice cold 50 mM HEPES pH 6.5, 5 mM MgCl₂, 1 mM EDTA 0.2% BSA. 50 μl MicroScint™ cocktail (Perkin Elmer 6013621) is added and plates are read on a Topcount NXT FS (Perkin Elmer). Percent binding of the [3H] ligand is calculated, where 1000 nM of LUF 5834 (Tocris 4603) control displaces the ligand 100% and 3% DMSO has 0% displacement. The % binding data versus the log of the inhibitor concentration is fitted to a one-site competitive binding model (GraphPad Prism version 7.02) where the ligand constant=2 nM and the ligand Kd=13 nM.

### Example E. A1 and A3 SPA Binding Assays

Both assays are conducted in white 384-well polystyrene plates (Greiner 781075) in a final volume of 50 μL. Inhibitors are first serially diluted in DMSO and 100 nL is added to the plate wells before the addition of other reaction components. The final concentration of DMSO is 2%.

Wheatgerm agglutinin-coated yttrium silicate SPA beads (Perkin Elmer RPNQ0023) and CHO-K₁ cell membranes overexpressing each human adenosine receptor are incubated in 50 mM HEPES pH 7.0, 5 mM MgCl₂, 1 mM EDTA (Assay buffer) on a rotary stirrer for 2 hours at 4° C. The beads are pelleted by centrifugation at 6000 g for one minute, and then the supernatant with unbound membrane is discarded. The beads are re-suspended to the original volume in assay buffer. Each radioligand is diluted in assay buffer+22% DMSO at 12.2× the final concentration, and then added to the SPA bead suspension. 50 μl of the SPA bead reaction mix is added to the assay wells and the plates shaken at 600 rpm for 1 hour at room temperature. The beads are then allowed to settle for 1 hour before reading on a Topcount NXT FS (Perkin Elmer). Percent binding of the radiolabeled ligand is calculated, where a control at >100×Ki displaces the ligand 100% and 2% DMSO has 0% displacement. The % binding data versus the log of the inhibitor concentration is fitted to a one-site competitive binding model (GraphPad Prism version 7.02). Assay conditions are provided in the table below.

| Assay Component | A1 | A3 |
|---|---|---|
| SPA beads in Hepes | 3 mg/ml | 1.25 mg/ml |
| buffer | | |
| Membrane | 60 μg/ml | 20 μg/ml |
| | Perkin Elmer ES-010 | Perkin Elemer ES-012 |
| Radioligand | 1 nM [3H] DP-CPX | 0.1 nM [125I] MECA |
| | (Perkin Elmer NET974) | (Perkin Elmer NEX312) |
| | K_{D} = 1 nM | K_{D} = 0.8 nM |
| Control | 1 μM DPCPX | 0.1 μM IB-MECA |
| | (Tocris 0439) | (Tocris 1066) |

Table 1. The A_{2A}_Ki data (Example C) and A_{2B}_cAMP_EC₅₀ data (Example B) are provided below.

| | A_{2A}_Ki | A_{2B}_cAMP_EC₅₀ |
|---|---|---|
| Ex. No. | (nM) | (nM) |
| 1 | † | ††† |
| 2 | ††† | N/A |
| 3 | †† | N/A |
| 4 | † | † |
| 5 | †† | †† |
| 6 | †† | N/A |
| 7 | †† | †† |
| 8 | ††† | N/A |
| 9 | †† | N/A |
| 10 | † | ††† |
| 11 | †† | N/A |
| 12 | †† | N/A |
| 13 | †† | N/A |
| 14 | † | †† |
| 15 | † | ††† |
| 16 | † | ††† |
| 17 | † | †† |
| 18 | †† | †† |
| 19 | † | ††† |
| 20 | † | ††† |
| 21 | † | †††† |
| 22 | †† | †††† |
| 23 | † | N/A |
| 24 | † | † |
| 25 | † | ††† |
| 26 | † | † |
| 27 | † | †† |
| 28 | † | † |
| 29 | † | † |
| 30 | † | † |
| 31 | † | †† |
| 32 | † | ††† |
| 33 | † | ††† |
| 34 | † | † |
| 35 | † | † |
| 36 | † | †† |
| 37 | † | † |
| 38 | † | †† |
| 39 | † | †† |
| 40 | †† | †††† |
| 41 | † | † |
| 42 | † | † |
| 43 | † | † |
| 44 | † | †† |
| 45 | † | † |
| 46 | † | † |
| 47 | † | † |
| 48 | † | †† |
| 49 | † | † |
| 50 | † | † |
| 51 | † | †† |
| 52 | † | † |
| 53 | † | †† |
| 54 | † | † |
| 55 | † | † |
| 56 | † | †† |
| 57 | † | ††† |
| 58 | † | † |
| 59 | † | †† |
| 60 | † | †† |
| 61 | † | † |
| 62 | † | † |
| 63 | † | † |
| 64 | † | † |
| 65 | † | † |
| 66 | † | † |
| 67 | † | ††† |
| 68 | † | ††† |
| 69 | † | †† |
| 70 | † | †† |
| 71 | † | †† |
| 72 | † | †† |
| 73 | † | †† |
| 74 | † | † |
| 75 | † | † |
| 76 | † | † |
| 77 | † | †† |
| 78 | † | † |
| 79 | † | † |
| 80 | † | †† |
| 81 | † | † |
| 82 | † | † |
| 83 | † | †† |
| 84 | † | † |
| 85 | † | † |
| 86 | † | † |
| 87 | † | † |
| 88 | † | † |
| 89 | † | † |
| 90 | † | † |
| 91 | † | † |
| 92 | † | † |
| 93 | † | † |
| 94 | † | † |
| 95 | † | † |
| 96 | † | † |
| 97 | † | † |

† indicates A_{2A}_Ki or A_{2B}_cAMP_EC₅₀ ≤ 10 nM,
†† indicates A_{2A}_Ki or A_{2B}_cAMP_EC₅₀ > 10 nM but ≤ 100 nM,
††† indicates A_{2A}_Ki or A_{2B}_cAMP_EC₅₀ > 100 nM but ≤ 1 μM,
†††† indicates A_{2A}_Ki or A_{2B}_cAMP_EC₅₀ is greater than 1 μM.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula (I): 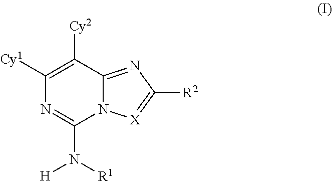
or a pharmaceutically acceptable salt thereof, wherein:
X is CR³;
R¹ is selected from H, C₁₋₆ alkyl, C₂₋₆ alkenyl, C₂₋₆ alkynyl, and C₁₋₆ haloalkyl;
R² is selected from C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{b2})NR^{c2}R^{d2}, NR^{c2}C(═NR^{e2})NR^{c2}R^{d2}, NR^{c2}C(NR^{e2})R^{b2}, NR^{c2}S(O)NR^{c2}R^{d2}, NR^{c2}S(O)R^{b2}, NR^{c2}S(O)₂R^{b2}, NR^{c2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, S(O)₂NR^{c2}R^{d2}, OS(O)(═NR^{e2})R^{b2}, OS(O)₂R^{b2}, SF₅, P(O)R^{f2}R^{g2}, OP(O)(OR^{h2})(ORⁱ²), P(O)(OR^{h2})(ORⁱ²), and BR^{j2}R^{k2}, wherein the C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R² are each optionally substituted with 1, 2, 3, 4, 5, 6, 7, or 8 independently selected R^{C} substituents;
R³ is selected from H, D, halo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a3}, SR^{a3}, NHOR^{a3}, C(O)R^{b3}, C(O)NR^{b3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{b3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, SF₅, P(O)R^{f1}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R³ are each optionally substituted with 1, 2, 3, 4, 5, 6, 7, or 8 independently selected R^{D} substituents;
Cy¹ is C₆₋₁₄ aryl, C₃₋₁₄ cycloalkyl, or 5-14 membered heteroaryl, wherein the C₆₋₁₄ aryl, C₃₋₁₄ cycloalkyl, or 5-14 membered heteroaryl is optionally substituted with 1, 2, 3, 4, 5, 6, 7, or 8 independently selected R^{E} or R^{M} substituents;
Cy² is C₆₋₁₄ aryl, C₃₋₁₄ cycloalkyl, 5-14 membered heteroaryl, or 4-14 membered heterocycloalkyl, wherein the C₆₋₁₄ aryl, C₃₋₁₄ cycloalkyl, 5-14 membered heteroaryl, or 4-14 membered heterocycloalkyl is optionally substituted with 1, 2, 3, 4, 5, 6, 7, or 8 independently selected R^{F} substituents;
each R^{a2}, R^{c2}, R^{d2}, R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a2}, R^{c2}, R^{d2}, R^{a1}, R^{c3}, and R^{d3}, are each optionally substituted with 1, 2, 3, 4, 5, 6, 7, or 8 independently selected R^{G} substituents;
or any R^{c2} and R^{d2} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
or any R^{c3} and R^{d3} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{G} substituents;
each R^{b2} and R^{b3} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b2} and R^{b3} are each optionally substituted with 1, 2, 3, 4, 5, 6, 7, or 8 independently selected R^{G} substituents;
each R^{e2} and R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f2}, R^{g2}, R^{f3}, and R^{g3} is independently selected from H, C₁₋₆ alkyl, C₁₋₆ haloalkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h2}, Rⁱ², R^{h3}, and Rⁱ³ is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j2}, R^{k2}, R^{j3}, and R^{k3} is independently selected from OH, C₁₋₆ alkoxy, and C₁₋₆ haloalkoxy;
or any R^{j2} and R^{k2} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{C}, R^{D}, R^{E}, R^{M}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR^{a4}, SR^{a4}, NHOR^{a4}, C(O)R^{b4}, C(O)NR^{c4}R^{d4}, C(O)NR^{c4}(OR^{a4}), C(O)OR^{a4}, OC(O)R^{b4}, OC(O)NR^{c4}R^{d4}, NR^{c4}R^{d4}, NR^{c4}NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}C(═NR^{e4})R^{b4}, NR^{c4}S(O)R^{b4}, NR^{c4}S(O)NR^{c4}R^{d4}, NR^{c4}S(O)₂R^{b4}, NR^{c4}S(O)(═NR^{e4})R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, OS(O)(═NR^{e4})R^{b4}, OS(O)₂R^{b4}, SF₅, P(O)R^{f4}R^{g4}, OP(O)(OR^{h4})(ORⁱ⁴), P(O)(OR^{h4})(ORⁱ⁴), and BR^{j4}R^{k4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{C}, R^{D}, R^{E}, R^{M}, R^{F}, and R^{G} are each optionally substituted with 1, 2, 3, 4, 5, 6, 7, or 8 independently selected R^{H} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a4}, R^{c4}, and R^{d4}, are each optionally substituted with 1, 2, 3, 4, 5, 6, 7, or 8 independently selected R^{H} substituents;
or, any R^{c4} and R^{d4}, attached to the same N atom, together with the N atom to which they are attached, optionally form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{H} substituents;
each R^{b4} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b4} are each optionally substituted with 1, 2, 3, 4, 5, 6, 7, or 8 independently selected R^{H} substituents;
each R^{e4} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f4} and R^{g4} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h4} and Rⁱ⁴ is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j4} and R^{k4} is independently selected from OH, C₁₋₆ alkoxy, and C₁₋₆ haloalkoxy;
or, any R^{j4} and R^{k4} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, CN, NO₂, OR⁵, SR⁵, NHOR^{a5}, C(O)R^{b5}, C(O)N^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NR^{c5}C(O)NR^{c5}R^{d5}, C(═NR^{b5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)R^{b5}, NR^{c5}S(O)NR^{c5}R^{d5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{H} are each optionally substituted with 1, 2, 3, or 4 independently selected R^{I} substituents;
each R^{a5}, R^{e5} and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a5}, R^{c5}, and R^{d5} are each optionally substituted with 1, 2, 3, or 4 independently selected R^{I} substituents;
or any R^{c5} and R^{d5} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{I} substituents;
each R^{b5} is independently selected from H, C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b5} are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h5} and Rⁱ⁵ is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a6}, SR^{a6}, NHOR^{a6}, C(O)R^{b6}, C(O)NR^{c6}R^{d6}, C(O)NR^{c6}(OR^{a6}), C(O)OR^{a6}, OC(O)R^{b6}, OC(O)NR^{c6}R^{d6}, NR^{c6}R^{d6}, NR^{c6} NR^{c6}R^{d6}, NR^{c6}C(O)R^{b6}, NR^{c6}C(O)OR^{a6}, NR^{c6}C(O)NR^{c5}R^{d6}, C(═NR^{e6})R^{b6}, C(═NR^{b6})NR^{c6}R^{d6}, NR^{c6}C(═NR^{e6})NR^{c6}R^{d6}, NR^{c6}C(═NR^{e6})R^{b6}, NR^{c6}S(O)R^{b6}, NR^{c6}S(O)NR^{c6}R^{d6}, NR^{c6}S(O)₂R^{b6}, NR^{c6}S(O)(═NR^{b6})R^{b6}, NR^{c6}S(O)₂NR^{c6}R^{d6}, S(O)R^{b6} S(O)NR^{c6}R^{d6}, S(O)₂R^{b6}, S(O)₂NR^{c6}R^{d6} OS(O)(═NR^{e6})R^{b6}, OS(O)₂R^{b6}, SF₅, P(O)R^{f6}R^{g6}, OP(O)(OR^{h6})(ORⁱ⁶), P(O)(OR^{h6})(ORⁱ⁶), and BR^{j6}R^{k6}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R¹ are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a6}, R^{c6}, and R^{d6} are each optionally substituted with 1, 2, 3, or 4 independently selected R^{J} substituents;
or any R^{c6} and R^{d6} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{J} substituents;
each R^{b6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b6} are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{f6} and R^{g6} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h6} and Rⁱ⁶ is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j6} and R^{k6} is independently selected from OH, C₁₋₆ alkoxy, and C₁₋₆ haloalkoxy;
or, any R^{j6} and R^{k6} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, CN, NO₂, OR^{a1}, SR^{a7}, NHOR^{a1}, C(O)R^{b7}, C(O)NR^{c7}R^{d7}, C(O)NR^{c7}(OR^{a7}), C(O)OR^{a7}, OC(O)R^{b7}, OC(O)NR^{c7}R^{d7}, NR^{c7}R^{d7}, NR^{c7}NR^{c7}R^{d7}, NR^{c7}C(O)R^{b7} NR^{c7}C(O)OR^{a7}, NR^{c7}C(O)NR^{c7}R^{d7}, C(═NR^{b7})R^{b7}, C(═NR^{e7})NR^{c7}R^{d7}, NR^{c7}C(═NR^{e7})NR^{c7}R^{d7}, NR^{c7}C(═NR^{e7})R^{b7}, NR^{c7}S(O)R^{b7}, NR^{c7}S(O)NR^{c7}R^{d7}, NR^{c7}S(O)₂R^{b7}, NR^{c7}S(O)(═NR^{e7})R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, OS(O)(═NR^{e7})R^{b7}, OS(O)₂R^{b7}, SF₅, P(O)R^{f7}R^{g7}, OP(O)(OR^{h7})(ORⁱ⁷), P(O)(OR^{h7})(ORⁱ⁷), and BR^{j7}R^{k7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{J} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{a7}, R^{c7}, and R^{d7} are each optionally substituted with 1, 2, 3, or 4 independently selected R^{K} substituents;
or any R^{c7} and R^{d7} attached to the same N atom, together with the N atom to which they are attached, form a 5- or 6-membered heteroaryl or a 4-14 membered heterocycloalkyl group, wherein the 5- or 6-membered heteroaryl or 4-14 membered heterocycloalkyl group is optionally substituted with 1, 2, 3, or 4 independently selected R^{K} substituents;
each R^{b7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl- of R^{b7} are each optionally substituted with 1, 2, 3, or 4 independently selected R^{K} substituents;
each R^{e7} is independently selected from H, OH, CN, C₁₋₆ alkyl, C₂₋₆ alkenyl, C₁₋₆ alkoxy, C₁₋₆ haloalkyl, C₁₋₆ haloalkoxy, C₂₋₆ alkynyl, C₆₋₁₄ aryl, C₃₋₁₄ cycloalkyl, 5-14 membered heteroaryl, and 4-14 membered heterocycloalkyl;
each R^{f7} and R^{g7} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{h7} and Rⁱ⁷ is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-;
each R^{j7} and R^{k7} is independently selected from OH, C₁₋₆ alkoxy, and C₁₋₆ haloalkoxy;
or, any R^{j7} and R^{k7} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-; and
wherein any heteroaryl group of any of the above-recited substituents optionally comprises an N-oxide on any ring-forming nitrogen.

2.-8. (canceled)

9. The compound of claim 1, or a pharmaceutically acceptable salt thereof, wherein Cy¹ is phenyl or 5-10 membered heteroaryl, wherein the phenyl or 5-10 membered heteroaryl is optionally substituted with 1, 2, 3, or 4 independently selected R^{E} substituents.

10. The compound of claim 1, or a pharmaceutically acceptable salt thereof, wherein Cy¹ is selected from phenyl, pyridinyl, furanyl, benzofuranyl, and pyrazolyl, each of which is optionally substituted with 1, 2, or 3 substituents selected from C₁₋₃ alkyl, halo, CN, and C₁₋₃ alkoxy.

11.-17. (canceled)

18. The compound of claim 1, or a pharmaceutically acceptable salt thereof, wherein Cy² is selected from C₃₋₆ cycloalkyl, phenyl, 5-10 membered heteroaryl, and 5-10 membered heterocycloalkyl;
wherein the 5-10 membered heteroaryl and 5-10 membered heterocycloalkyl each comprise one, two, or three nitrogen atoms as ring-forming heteroatoms, wherein one of the one or two nitrogen atoms is optionally an N-oxide, and wherein a ring-forming carbon atom is optionally substituted by oxo; and
wherein the C₃₋₆ cycloalkyl, phenyl, 5-10 membered heteroaryl, and 5-10 membered heterocycloalkyl are each optionally substituted with 1, 2, or 3 substituents selected from C₁₋₃ alkyl, C₁₋₃ alkyl-OH, halo, CN, C₁₋₃ alkoxy, and C(O)NH₂.

19.-21. (canceled)

22. The compound of claim 1, or a pharmaceutically acceptable salt thereof, wherein R¹ is selected from H and C₁₋₆ alkyl.

23.-25. (canceled)

26. The compound of claim 1, or a pharmaceutically acceptable salt thereof, wherein R² is selected from C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, (4-14 membered heterocycloalkyl)-C₁₋₆ alkyl-, OR^{a2}, NR^{c2}R^{d2}, C(O)R^{b2}, C(O)NR^{c2}R^{d2}, and C(O)OR^{a2}, wherein the C₆₋₁₄ aryl, C₃₋₁₄ cycloalkyl, 5-14 membered heteroaryl, 4-14 membered heterocycloalkyl, C₆₋₁₄ aryl-C₁₋₆ alkyl-, C₃₋₁₄ cycloalkyl-C₁₋₆ alkyl-, (5-14 membered heteroaryl)-C₁₋₆ alkyl-, and (4-14 membered heterocycloalkyl)-C₁₋₆ alkyl-, are each optionally substituted with 1, 2, or 3 independently selected R^{C} substituents.

27. (canceled)

28. The compound of claim 1, or a pharmaceutically acceptable salt thereof, wherein R² is selected from phenyl, 5-6 membered heteroaryl, C(O)R^{b2}, C(O)NR^{c2}R^{d2}, and C(O)OR^{a2}, wherein the phenyl and 5-6 membered heteroaryl are each optionally substituted with 1 or 2 independently selected R^{C} substituents.

29. The compound of claim 1, or a pharmaceutically acceptable salt thereof, wherein R² is selected from C(O)OEt, CONH₂, and C(O)NHEt.

30. The compound of claim 1, or a pharmaceutically acceptable salt thereof, wherein R² selected from phenyl and 5-6 membered heteroaryl, each of which is optionally substituted with C(O)OMe.

31. The compound of claim 1, or a pharmaceutically acceptable salt thereof, wherein R² is

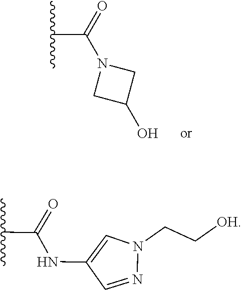 [SMILES: *C(=O)N1CC(O)C1 ; *C(=O)Nc1cnn(CCO)c1]

32.-33. (canceled)

34. The compound of claim 1, or a pharmaceutically acceptable salt thereof, wherein each R^{C} is independently selected from halo, C₁₋₆ alkyl, C₆₋₁₄ aryl, 5-14 membered heteroaryl, (4-14 membered heterocycloalkyl)-C₁₋₆ alkyl-, OR^{a4}, C(O)OR^{a4}, and NR^{c4}R^{d4}, wherein the C₁₋₆ alkyl, 5-14 membered heteroaryl, and (4-14 membered heterocycloalkyl)-C₁₋₆ alkyl- are optionally substituted with 1, 2, or 3 independently selected R^{H} substituents.

35. The compound of claim 1, or a pharmaceutically acceptable salt thereof, wherein each R^{H} is independently selected from halo, oxo, C₁₋₆ alkyl, C₁₋₆ haloalkyl, OR^{a5}, C(O)OR^{a5}, and NR^{c5}S(O)₂R^{b5}.

36. The compound of claim 1, or a pharmaceutically acceptable salt thereof, wherein R³ is selected from H, halo, C₁₋₆ alkyl, C₆₋₁₄ aryl, 5-14 membered heteroaryl, CN, and OR^{a3}, wherein the C₁₋₆ alkyl, C₆₋₁₄ aryl, and 5-14 membered heteroaryl are each optionally substituted with 1, 2, or 3 independently selected R^{D} substituents.

37. The compound of claim 1, or a pharmaceutically acceptable salt thereof, wherein R³ is selected from H, C₁₋₃ alkyl, halo, CN, morpholinomethyl, 4-ethoxyphenyl, 2-hydroxyethoxy, and pyridinyl.

38. (canceled)

39. The compound of claim 1, or a pharmaceutically acceptable salt thereof; wherein:
R¹ is selected from H and C₁₋₆ alkyl;
R² is selected from C₆₋₁₄ aryl, 5-14 membered heteroaryl, C(O)R^{b2}, C(O)NR^{c2}R^{d2}, and C(O)OR^{a2}, wherein the C₆₋₁₄ aryl and 5-14 membered heteroaryl of R² are each optionally substituted with 1, 2, 3, or 4 independently selected R^{C} substituents;
R³ is selected from H, D, halo, C₁₋₆ alkyl, C₁₋₆ haloalkyl, C₆₋₁₄ aryl, 5-14 membered heteroaryl, CN, and OR^{a3}, wherein the C₁₋₆ alkyl, C₆₋₁₄ aryl, and 5-14 membered heteroaryl of R³ are each optionally substituted with 1, 2, 3, or 4 independently selected R^{D} substituents;
Cy¹ is phenyl optionally substituted with 1, 2, 3, or 4 independently selected R^{M} substituents, or C₁₀₋₁₄ aryl or 5-14 membered heteroaryl, wherein the C₁₀₋₁₄ aryl and 5-14 membered heteroaryl of Cy¹ is optionally substituted with 1, 2, 3, or 4 independently selected R^{E} substituents;
Cy² is C₆₋₁₄ aryl, C₃₋₁₄ cycloalkyl, 5-14 membered heteroaryl or 4-14 membered heterocycloalkyl, wherein the C₆₋₁₄ aryl, C₃₋₁₄ cycloalkyl, 5-14 membered heteroaryl and 4-14 membered heterocycloalkyl of Cy² are each optionally substituted with 1, 2, 3, or 4 independently selected R^{F} substituents;
each R^{a2}, R^{b2}, R^{d2}, and R^{a1} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a2}, R^{c2}, R^{d2}, and R^{a3} are each optionally substituted with 1, 2, 3, or 4 independently selected R^{G} substituents;
each R^{b2} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{G} substituents;
each R^{C}, R^{D}, R^{E}, R^{F}, and R^{G} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{c4}R^{d4}, C(O)OR^{a4} OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{e4})NR^{c4}R^{d4}, NR^{c4}C(═NR^{e4})NR^{c4}R^{d4}, NR^{c4}S(O)R^{b4} NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{C}, R^{D}, R^{E}, R^{F}, and R^{G} are each optionally substituted with 1, 2, 3, or 4 independently selected R^{H} substituents;
each R^{M} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{b4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{e4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{M} are each optionally substituted with 1, 2, 3, or 4 independently selected R^{H} substituents;
each R^{a4}, R^{c4}, and R^{d4}, is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4}, are each optionally substituted with 1, 2, 3, or 4 independently selected R^{H} substituents;
each R^{b4} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{H} substituents;
each R^{e4} is independently selected from H, OH, CN, C₁₋₆ alkyl, C₁₋₆ alkoxy, C₁₋₆ haloalkyl, and C₁₋₆ haloalkoxy;
each R^{H} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a5}, SR^{a5}, NHOR^{a5}, C(O)R^{b5}, C(O)NR^{c5}R^{d5}, C(O)OR^{a5}, OC(O)R^{b5} OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{b5})R^{b5}, C(═NR^{e5})NR^{c5}R^{d5}, NR^{c5}C(═NR^{e5})NR^{c5}R^{d5}, NR^{c5}S(O)R^{b5}, NR^{c5}S(O)₂R^{b5}, and NR^{c5}S(O)₂NR^{c5}R^{d5}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, of R^{H} are each optionally substituted with 1, 2, 3, or 4 independently selected R^{I} substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4}, are each optionally substituted with 1, 2, 3, or 4 independently selected R^{I} substituents;
each R^{b5} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{I} substituents;
each R^{e5} is independently selected from H, OH, CN, C₁₋₆ alkyl, C₁₋₆ alkoxy, C₁₋₆ haloalkyl, and C₁₋₆ haloalkoxy;
each R^{I} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a6}, SR^{a6}, NHOR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)OR^{a6}, NR^{c6}C(O)NR^{c6}R^{d6}, C(═NR^{b6})R^{b6}, C(═NR^{b6})NR^{c6}R^{d6}, NR^{c6}C(═NR^{e6})NR^{c6}R^{d6}, NR^{c6}S(O)R^{b6}, NR^{c6}S(O)₂R^{b6}, and NR^{c6}S(O)₂NR^{c6}R^{d6} wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, of R^{H} are each optionally substituted with 1, 2, 3, or 4 independently selected R^{J} substituents;
each R^{a6}, R^{c6}, and R^{d6} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a6}, R^{c6}, and R^{d6} are each optionally substituted with 1, 2, 3, or 4 independently selected R^{J} substituents;
each R^{b6} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{J} substituents;
each R^{e6} is independently selected from H, OH, CN, C₁₋₆ alkyl, C₁₋₆ alkoxy, C₁₋₆ haloalkyl, and C₁₋₆ haloalkoxy;
each R^{J} is independently selected from D, halo, oxo, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a7}, SR^{a7}, NHOR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, C(═NR^{b7})R^{b7}, C(═NR^{e7})NR^{c7}R^{d7}, NR^{c7}C(═NR^{e7})NR^{c7}R^{d7}, NR^{c7}S(O)R^{b7}, NR^{c7}S(O)₂R^{b7}, and NR^{c7}S(O)₂NR^{c7}R^{d7}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, of R^{H} are each optionally substituted with 1, 2, 3, or 4 independently selected R^{K} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a7}, R^{c7}, and R^{d7} are each optionally substituted with 1, 2, 3, or 4 independently selected R^{K} substituents;
each R^{b7} is independently selected from C₁₋₆ alkyl, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{K} substituents;
each R^{e7} is independently selected from H, OH, CN, C₁₋₆ alkyl, C₁₋₆ alkoxy, C₁₋₆ haloalkyl, and C₁₋₆ haloalkoxy;
each R^{K} is independently selected from H, D, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl; and
wherein any heteroaryl group of any of the above-recited substituents optionally comprises an N-oxide on any ring-forming nitrogen.

40. The compound of claim 1, or a pharmaceutically acceptable salt thereof; wherein:
R¹ is selected from H and C₁₋₆ alkyl;
R² is selected from C₆₋₁₄ aryl, 5-14 membered heteroaryl, C(O)R^{b2}, C(O)NR^{c2}R^{d2}, and C(O)OR^{a2}, wherein the C₆₋₁₄ aryl and 5-14 membered heteroaryl of R² are each optionally substituted with 1, 2, 3, or 4 independently selected R^{C} substituents;
R³ is selected from H, D, halo, C₁₋₆ alkyl, C₆₋₁₄ aryl, 5-14 membered heteroaryl, CN, and OR³, wherein the C₁₋₆ alkyl, C₆₋₁₄ aryl, and 5-14 membered heteroaryl of R³ are each optionally substituted with 1, 2, 3, or 4 independently selected R^{D} substituents;
Cy¹ is phenyl optionally substituted with 1, 2, 3, or 4 independently selected R^{M} substituents, or C₁₀₋₁₄ aryl or 5-14 membered heteroaryl, wherein the C₁₀₋₁₄ aryl and 5-14 membered heteroaryl of Cy¹ is optionally substituted with 1, 2, 3, or 4 independently selected R^{E} substituents;
Cy² is C₆₋₁₄ aryl, C₃₋₁₄ cycloalkyl, 5-14 membered heteroaryl or 4-14 membered heterocycloalkyl, wherein the C₆₋₁₄ aryl, C₃₋₁₄ cycloalkyl, 5-14 membered heteroaryl and 4-14 membered heterocycloalkyl of Cy² are each optionally substituted with 1, 2, 3, or 4 independently selected R^{F} substituents;
each R^{a2}, R^{b2}, R^{d2}, and R^{a1} is independently selected from H, C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a2}, R^{c2}, R^{d2}, and R^{a3} are each optionally substituted with 1, 2, 3, or 4 independently selected R^{G} substituents;
each R^{b2} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{G} substituents;
each R^{C}, R^{D}, R^{E}, R^{F}, and R^{G} is independently selected from D, halo, oxo, C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{C}, R^{D}, R^{E}, R^{F}, and R^{G} are each optionally substituted with 1, 2, 3, or 4 independently selected R^{H} substituents;
each R^{M} is independently selected from D, halo, oxo, C₁₋₆ alkyl, C₂₋₆ alkenyl, C₂₋₆ alkynyl, C₆₋₁₄ aryl, C₃₋₁₄ cycloalkyl, 5-14 membered heteroaryl, 4-14 membered heterocycloalkyl, CN, NO₂, OR^{a4}, SR^{a4}, NHOR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)OR^{d4}, NR^{c4}C(O)NR^{c4}R^{d4}, C(═NR^{b4})R^{b4}, C(═NR^{b4})NR^{c4}R^{d4}, NR^{c4}C(═NR^{e4})NR^{c4}R^{d4}, NR^{c4}S(O)R^{b4}, NR^{c4}S(O)₂R^{b4}, and NR^{c4}S(O)₂NR^{c4}R^{d4}, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{M} are each optionally substituted with 1, 2, 3, or 4 independently selected R^{H} substituents;
each R^{a4}, R^{c4}, and R^{d4}, is independently selected from H, C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl of R^{a4}, R^{c4}, and R^{d4}, are each optionally substituted with 1, 2, 3, or 4 independently selected R^{H} substituents;
each R^{b4} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl, wherein the C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl are each optionally substituted with 1, 2, 3, or 4 independently selected R^{H} substituents;
each R^{e4} is independently selected from H, OH, CN, C₁₋₆ alkyl, C₁₋₆ alkoxy, C₁₋₆ haloalkyl, and C₁₋₆ haloalkoxy;
each R^{H} is independently selected from D, halo, oxo, C₁₋₆ alkyl, CN, NO₂, OR^{a5}, SR^{a5}, NHOR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, C(═NR^{e5})R^{b5}, C(═NR^{b5})NR^{c5}R^{d5}, NR^{c5}C(═NR^{b5})NR^{c5}R^{d5}, NR^{c5}S(O)R^{b5}, NR^{c5}S(O)₂R^{b5}, and NR^{c5}S(O)₂NR^{c5}R^{d5}; and
each R^{a5}, R^{c5}, and R^{d5} is independently selected from H, and C₁₋₆ alkyl;
each R^{b5} is independently selected from C₁₋₆ alkyl, C₂₋₆ alkenyl, C₂₋₆ alkynyl, C₆₋₁₄ aryl, C₃₋₁₄ cycloalkyl, 5-14 membered heteroaryl, and 4-14 membered heterocycloalkyl; and
each R^{e5} is independently selected from H and C₁₋₆ alkyl.

41. (canceled)

42. The compound of claim 1, selected from:
8-(2-Methylpyridin-4-yl)-2,7-diphenylimidazo[1,2-c]pyrimidin-5-amine;
Ethyl 5-amino-8-(2-methylpyridin-4-yl)-7-phenylimidazo[1,2-c]pyrimidine-2-carboxylate;
Methyl 5-(5-amino-7-phenyl-8-(pyridin-4-yl)imidazo[1,2-c]pyrimidin-2-yl)isoxazole-3-carboxylate;
5-Amino-8-(2-methylpyridin-4-yl)-7-phenylimidazo[1,2-c]pyrimidine-2-carboxamide;
5-Amino-8-(1-carbamoyl-1,2,3,6-tetrahydropyridin-4-yl)-N-ethyl-7-phenylimidazo[1,2-c]pyrimidine-2-carboxamide;
5-Amino-8-(1-carbamoylpiperidin-4-yl)-N-ethyl-7-phenylimidazo[1,2-c]pyrimidine-2-carboxamide;
5-Amino-7-(3-cyanophenyl)-N-ethyl-3-(2-hydroxyethoxy)-8-(2-methoxypyridin-4-yl)imidazo[1,2-c]pyrimidine-2-carboxamide;
5-Amino-7-(3-cyanophenyl)-N-ethyl-8-(2-methoxypyridin-4-yl)-3-methylimidazo[1,2-c]pyrimidine-2-carboxamide;
5-Amino-7-(3-cyanophenyl)-N-ethyl-8-(2-methoxypyridin-4-yl)-3-(pyridin-2-yl)imidazo[1,2-c]pyrimidine-2-carboxamide;
5-Amino-3-bromo-8-(2,6-dimethylpyridin-4-yl)-N-ethyl-7-phenylimidazo[1,2-c]pyrimidine-2-carboxamide;
5-Amino-3-cyano-8-(2,6-dimethylpyridin-4-yl)-N-ethyl-7-phenylimidazo[1,2-c]pyrimidine-2-carboxamide;
8-(2,6-Dimethylpyridin-4-yl)-N-ethyl-5-(ethylamino)-7-phenylimidazo[1,2-c]pyrimidine-2-carboxamide;
4-(5-Amino-2-(ethylcarbamoyl)-7-phenylimidazo[1,2-c]pyrimidin-8-yl)-2,6-dimethylpyridine 1-oxide;
3-(5-Amino-8-(1-ethyl-6-oxo-1,6-dihydropyridin-3-yl)-2-(3-hydroxyazetidine-1-carbonyl)imidazo[1,2-c]pyrimidin-7-yl)benzonitrile;
5-Amino-7-(3-cyanophenyl)-8-(1-ethyl-6-oxo-1,6-dihydropyridin-3-yl)-N-(1-(2-hydroxyethyl)-1H-pyrazol-4-yl)imidazo[1,2-c]pyrimidine-2-carboxamide;
5-Amino-7-(3-cyanophenyl)-N-ethyl-8-(pyridin-4-yl)imidazo[1,2-c]pyrimidine-2-carboxamide;
5-Amino-7-(3-cyanophenyl)-N-ethyl-8-(3-methylpyridin-4-yl)imidazo[1,2-c]pyrimidine-2-carboxamide;
5-Amino-7-(3-cyanophenyl)-N-ethyl-8-(3-fluoropyridin-4-yl)imidazo[1,2-c]pyrimidine-2-carboxamide;
5-Amino-7-(3-cyanophenyl)-N-ethyl-8-(3-chloropyridin-4-yl)imidazo[1,2-c]pyrimidine-2-carboxamide;
5-Amino-7-(3-cyanophenyl)-N-ethyl-8-(3-methoxypyridin-4-yl)imidazo[1,2-c]pyrimidine-2-carboxamide;
5-Amino-7-(3-cyanophenyl)-N-ethyl-8-(3-cyanopyridin-4-yl)imidazo[1,2-c]pyrimidine-2-carboxamide;
5-Amino-8-(4-carbamoylphenyl)-7-(3-cyanophenyl)-N-ethylimidazo[1,2-c]pyrimidine-2-carboxamide;
5-Amino-7-(3-cyanophenyl)-N-ethyl-8-(pyrazolo[1,5-a]pyridin-3-yl)imidazo[1,2-c]pyrimidine-2-carboxamide;
5-Amino-7-(3-cyanophenyl)-N-ethyl-8-(5-methyl-1H-pyrazol-4-yl)imidazo[1,2-c]pyrimidine-2-carboxamide;
5-Amino-7-(3-cyanophenyl)-N-ethyl-8-(1-ethyl-1H-pyrazol-5-yl)imidazo[1,2-c]pyrimidine-2-carboxamide;
5-Amino-7-(3-cyanophenyl)-N-ethyl-8-(1-isopropyl-1H-pyrazol-5-yl)imidazo[1,2-c]pyrimidine-2-carboxamide;
5-Amino-7-(3-cyanophenyl)-N-ethyl-8-(1-propyl-1H-pyrazol-5-yl)imidazo[1,2-c]pyrimidine-2-carboxamide;
5-Amino-7-(3-cyanophenyl)-N-ethyl-8-(pyrimidin-4-yl)imidazo[1,2-c]pyrimidine-2-carboxamide;
5-Amino-7-(3-cyanophenyl)-8-(2,3-dihydro-[1,4]dioxino[2,3-b]pyridin-8-yl)-N-ethylimidazo[1,2-c]pyrimidine-2-carboxamide; and
5-Amino-7-(3-cyanophenyl)-8-cyclopropyl-N-ethylimidazo[1,2-c]pyrimidine-2-carboxamide;
or a pharmaceutically acceptable salt thereof.

43.-45. (canceled)

46. A pharmaceutical composition comprising a compound of claim 1, or a pharmaceutically acceptable salt thereof, and a pharmaceutically acceptable excipient or carrier.

47.-49. (canceled)

50. A method of treating a disease or disorder in a patient, wherein the disease or disorder is bladder cancer, lung cancer, breast cancer, ovarian cancer, colorectal cancer, pancreatic cancer, prostate cancer, or head and neck cancer.

51.-61. (canceled)

62. A compound, which is selected from:
3-(5-Amino-8-(2,6-dimethylpyridin-4-yl)imidazo[1,2-c]pyrimidin-7-yl)benzonitrile;
7-(2,3-Dihydro-1H-pyrrolo[2,3-b]pyridin-5-yl)-8-(2,6-dimethylpyridin-4-yl)imidazo[1,2-c]pyrimidin-5-amine;
8-(2,6-Dimethylpyridin-4-yl)-3-(morpholinomethyl)-7-phenylimidazo[1,2-c]pyrimidin-5-amine;
7-(3-Methoxyphenyl)-8-(pyridin-4-yl)imidazo[1,2-c]pyrimidin-5-amine;
7,8-Di(pyridin-4-yl)imidazo[1,2-c]pyrimidin-5-amine;
7-(2-Methylfuran-3-yl)-8-(pyridin-4-yl)imidazo[1,2-c]pyrimidin-5-amine;
7-(2-Fluorophenyl)-8-(pyridin-4-yl)imidazo[1,2-c]pyrimidin-5-amine;
7-(Benzofuran-2-yl)-8-(pyridin-4-yl)imidazo[1,2-c]pyrimidin-5-amine;
7-(1-Methyl-1H-pyrazol-4-yl)-8-(2-methylpyridin-4-yl)imidazo[1,2-c]pyrimidin-5-amine; and
3-(4-Ethoxyphenyl)-8-(2-methylpyridin-4-yl)-7-phenylimidazo[1,2-c]pyrimidin-5-amine;
or a pharmaceutically acceptable salt thereof.
